SEQUENCE LISTING (101 sequences)

SEQ ID NO: 1 - protein, 10 aa, Homo sapiens
GYTFTNYVIH

SEQ ID NO: 2 - protein, 10 aa, Homo sapiens
GYSFTNYYIH

SEQ ID NO: 3 - protein, 10 aa, Homo sapiens
GYTFTNYWIH

SEQ ID NO: 4 - protein, 17 aa, Homo sapiens
YIYPYNDGILYNEKFKG

SEQ ID NO: 5 - protein, 17 aa, Homo sapiens
YIDPLNGDTTYNQKFKG

SEQ ID NO: 6 - protein, 17 aa, Homo sapiens
YTDPRTDYTEYNQKFKD

SEQ ID NO: 7 - protein, 8 aa, Homo sapiens
GGYYVPDY

SEQ ID NO: 8 - protein, 8 aa, Homo sapiens
GGYYVYDY

SEQ ID NO: 9 - protein, 8 aa, Homo sapiens
GGKRAMDY

SEQ ID NO: 10 - protein, 8 aa, Homo sapiens
GGRVGLGY

SEQ ID NO: 11 - protein, 16 aa, Homo sapiens
RSRQSIVHTNGNTYLG

SEQ ID NO: 12 - protein, 11 aa, Homo sapiens
RASQDISNYLN

SEQ ID NO: 13 - protein, 11 aa, Homo sapiens
RASQSISNYLN

SEQ ID NO: 14 - protein, 16 aa, Homo sapiens
RSSQNIVQSNGNTYLE

SEQ ID NO: 15 - protein, 7 aa, Homo sapiens
KVSNRFS

SEQ ID NO: 16 - protein, 7 aa, Homo sapiens
YTSRLYS

SEQ ID NO: 17 - protein, 7 aa, Homo sapiens
KVFHRFS

SEQ ID NO: 18 - protein, 9 aa, Homo sapiens
FQGSHVPYT

SEQ ID NO: 19 - protein, 9 aa, Homo sapiens
QQGNTLPWT

SEQ ID NO: 20 - protein, 9 aa, Homo sapiens
FQGSHVPWT

SEQ ID NO: 21 - protein, 117 aa, Homo sapiens
EVQLQQSGPELVKPGASVKMSCKASGYTFTNYVIHWVKRRPGQGLEWIGYIYPYNDGILYNEKFKGKATVTSDKSSSTAYMDLSSLTSEDSAVYYCTRGGYYVPDYWGQGTTLTVSS

SEQ ID NO: 22 - protein, 117 aa, Homo sapiens
EVQLQQSGPELVKPGASVKMSCKASGYTFTNYVIHWVKRRPGQGLEWIGYIYPYNDGILYNEKFKGKATVTSDKSSSTAYMDLSSLTSEDSAVYYCTRGGYYVPDYWGQGTLVTVSS

SEQ ID NO: 23 - protein, 117 aa, Homo sapiens
QVQLVQSGAEVKKPGASVQVSCKASGYTFTNYVIHWLRQAPGQGLEWMGYIYPYNDGILYNEKFKGRVTMTSDTSISTAYMELSSLRSDDTAVYYCARGGYYVPDYWGQATLVTVSS

SEQ ID NO: 24 - protein, 117 aa, Homo sapiens
QVQLVQSGAEVKKPGASVQVSCKASGYTFTNYVIHWLRQAPGQGLEWMGYIYPYNDGILYNEKFKGRVTMTSDTSISTAYMELSSLRSDDTAVYYCARGGYYVYDYWGQATLVTVSS

SEQ ID NO: 25 - protein, 117 aa, Homo sapiens
EVQLVQSGAEVKKPGATVKISCKVSGYTFTNYVIHWVQQAPGKGLEWMGYIYPYNDGILYNEKFKGRVTITADTSTDTAYMELSSLRSEDTAVYYCATGGYYVPDYWGQGTTVTVSS

SEQ ID NO: 26 - protein, 117 aa, Homo sapiens
EVQLVQSGAEVKKPGESLKISCKGSGYTFTNYVIHWVRQMPGKGLEWMGYIYPYNDGILYNEKFKGQVTISADKSISTAYLQWSSLKASDTAMYYCARGGYYVPDYWGQGTTVTVSS

SEQ ID NO: 27 - protein, 117 aa, Homo sapiens
QVQLVQSGAEVKKPGASVKVSCKASGYTFTNYVIHWVRQAPGQGLEWMGYIYPYNDGILYNEKFKGRVTMTTDTSTSTAYMELRSLRSDDTAVYYCARGGYYVPDYWGQGTTVTVSS

SEQ ID NO: 28 - protein, 117 aa, Homo sapiens
EVQLQQSGPELMKPGASVKISCKASGYSFTNYYIHWVNQSHGKSLEWIGYIDPLNGDTTYNQKFKGKATLTVDKSSSTAYMRLSSLTSADSAVYYCARGGKRAMDYWGQGTSVTVSS

SEQ ID NO: 29 - protein, 117 aa, Homo sapiens
QVQLVQSGAEVKKPGASVKVSCKASGYSFTNYYIHWVRQAPGQGLEWMGYIDPLNGDTTYNQKFKGRVTMTRDTSTSTVYMELSSLRSEDTAVYYCARGGKRAMDYWGQGTLVTVSS

SEQ ID NO: 30 - protein, 117 aa, Homo sapiens
QVQLVQSGAEVKKPGSSVKVSCKASGYSFTNYYIHWVRQAPGQGLEWMGYIDPLNGDTTYNQKFKGRVTITADESTSTAYMELSSLRSEDTAVYYCARGGKRAMDYWGQGTLVTVSS

SEQ ID NO: 31 - protein, 117 aa, Homo sapiens
EVQLVQSGAEVKKPGESLKISCKGSGYSFTNYYIHWVRQMPGKGLEWMGYIDPLNGDTTYNQKFKGQVTISADKSISTAYLQWSSLKASDTAMYYCARGGKRAMDYWGQGTLVTVSS

SEQ ID NO: 32 - protein, 117 aa, Homo sapiens
QVQLVQSGAEVKKPGSSVKVSCKASGYSFTNYYIHWVRQAPGQGLEWMGYIDPLNGDTTYAQKFQGRVTITADESTSTAYMELSSLRSEDTAVYYCARGGKRAMDYWGQGTLVTVSS

SEQ ID NO: 33 - protein, 117 aa, Homo sapiens
EVQLVQSGAEVKKPGESLKISCKGSGYSFTNYYIHWVRQMPGKGLEWMGYIDPLNGDTTYSPSFQGQVTISADKSISTAYLQWSSLKASDTAMYYCARGGKRAMDYWGRGTLVTVSS

SEQ ID NO: 34 - protein, 117 aa, Homo sapiens
QVQLVQSGAEVKKPGASVQVSCKASGYSFTNYYIHWLRQAPGQGLEWMGYIDPLNGDTTYNQKFKGRVTMTSDTSISTAYMELSSLRSDDTAVYYCARGGKRAMDYWGQATLVTVSS

SEQ ID NO: 35 - protein, 117 aa, Homo sapiens
QVQLQQFGAELAKPGASVQMSCKASGYTFTNYWIHWVKQRPGQGLEWIGYTDPRTDYTEYNQKFKDKATLAADRSSSTAYMRLSSLTSEDSAVYYCAGGGRVGLGYWGHGSSVTVSS

SEQ ID NO: 36 - protein, 117 aa, Homo sapiens
QVQLVQSGAEVKKPGASVKVSCKASGYTFTNYWIHWVRQAPGQGLEWMGYTDPRTDYTEYNQKFKDRVTMTRDTSTSTVYMELSSLRSEDTAVYYCARGGRVGLGYWGQGTLVTVSS

SEQ ID NO: 37 - protein, 117 aa, Homo sapiens
QVQLVQSGAEVKKPGSSVKVSCKASGYTFTNYWIHWVRQAPGQGLEWMGYTDPRTDYTEYNQKFKDRVTITADESTSTAYMELSSLRSEDTAVYYCARGGRVGLGYWGQGTLVTVSS

SEQ ID NO: 38 - protein, 117 aa, Homo sapiens
EVQLVQSGAEVKKPGESLKISCKGSGYTFTNYWIHWVRQMPGKGLEWMGYTDPRTDYTEYNQKFKDQVTISADKSISTAYLQWSSLKASDTAMYYCARGGRVGLGYWGQGTLVTVSS

SEQ ID NO: 39 - protein, 117 aa, Homo sapiens
QVQLVQSGAEVKKPGSSVKVSCKASGYTFTNYWIHWVRQAPGQGLEWMGYTDPRTDYTEYAQKFQGRVTITADESTSTAYMELSSLRSEDTAVYYCARGGRVGLGYWGQGTLVTVSS

SEQ ID NO: 40 - protein, 117 aa, Homo sapiens
EVQLVQSGAEVKKPGESLKISCKGSGYTFTNYWIHWVRQMPGKGLEWMGYTDPRTDYTEYSPSFQGQVTISADKSISTAYLQWSSLKASDTAMYYCARGGRVGLGYWGQGTLVTVSS

SEQ ID NO: 41 - protein, 112 aa, Homo sapiens
DVLMTQTPLSLPVNLGDQASISCRSRQSIVHTNGNTYLGWFLQKPGQSPKLLIYKVSNRFSGVPDRFSGSGSGTDFTLTISRVEAEDLGVYYCFQGSHVPYTFGGGTKLEIK

SEQ ID NO: 42 - protein, 112 aa, Homo sapiens
DVLMTQTPLSLPVNLGDQASISCRSRQSIVHTNGNTYLGWFLQKPGQSPKLLIYKVSNRFSGVPDRFSGSGSGTDFTLTISRVEAEDLGVYYCFQGSHVPYTFGQGTKVEIK

SEQ ID NO: 43 - protein, 112 aa, Homo sapiens
DIVMTQSPLSLPVTPGEPASISCRSRQSIVHTNGNTYLGWYLQKPGQSPRLLIYKVSNRFSGVPDRFSGSGSGTDFTLKISRVEADDVGIYYCFQGSHVPYTFGQGTKLEIK

SEQ ID NO: 44 - protein, 112 aa, Homo sapiens
DVVMTQSPLSLPVTLGQPASISCRSRQSIVHTNGNTYLGWFQQRPGQSPRRLIYKVSNRFSGVPDRFSGSGSGTDFTLKISRVEAEDVGVYYCFQGSHVPYTFGQGTKLEIK

SEQ ID NO: 45 - protein, 112 aa, Homo sapiens
DIVMTQSPDSLAVSLGERATINCRSRQSIVHTNGNTYLGWYQQKPGQPPKLLIYKVSNRFSGVPDRFSGSGSGTDFTLTISSLQAEDVAVYYCFQGSHVPYTFGQGTKLEIK

SEQ ID NO: 46 - protein, 107 aa, Homo sapiens
DIQMTQTTSSLSASLGDRVTISCRASQDISNYLNWYQQKPDGTVKLLIYYTSRLYSGVPSRFSGSGSGTDYSLTISNLEQEDIATYFCQQGNTLPWTFGGGTKLEIK

SEQ ID NO: 47 - protein, 107 aa, Homo sapiens
DIQMTQSPSSLSASVGDRVTITCRASQDISNYLNWYQQKPGKAPKLLIYYTSRLYSGVPSRFSGSGSGTDFTFTISSLQPEDIATYYCQQGNTLPWTFGQGTKVEIK

SEQ ID NO: 48 - protein, 107 aa, Homo sapiens
DIQMTQSPSSLSASVGDRVTITCRASQSISNYLNWYQQKPGKAPKLLIYYTSRLYSGVPSRFSGSGSGTDFTLTISSLQPEDFATYYCQQGNTLPWTFGQGTKVEIK

SEQ ID NO: 49 - protein, 107 aa, Homo sapiens
DIVMTQSPLSLPVTPGEPASISCRASQDISNYLNWYLQKPGQSPRLLIYYTSRLYSGVPDRFSGSGSGTDFTLKISRVEADDVGIYYCQQGNTLPWTFGQGTKLEIK

SEQ ID NO: 50 - protein, 112 aa, Homo sapiens
DVFMTQTPLSLPVSLGDQASISCRSSQNIVQSNGNTYLEWYLQKPGQSPKLLIYKVFHRFSGVPDRFSGSGSGTDFTLKISRVEAEDLGVYYCFQGSHVPWTFGGGTKVEIK

SEQ ID NO: 51 - protein, 112 aa, Homo sapiens
DVVMTQSPLSLPVTLGQPASISCRSSQNIVQSNGNTYLEWFQQRPGQSPRRLIYKVFHRFSGVPDRFSGSGSGTDFTLKISRVEAEDVGVYYCFQGSHVPYTFGQGTKLEIK

SEQ ID NO: 52 - protein, 112 aa, Homo sapiens
DIVMTQSPDSLAVSLGERATINCRSSQNIVQSNGNTYLEWYQQKPGQPPKLLIYKVFHRFSGVPDRFSGSGSGTDFTLTISSLQAEDVAVYYCFQGSHVPYTFGQGTKLEIK

SEQ ID NO: 53 - protein, 330 aa, Homo sapiens [sequence omitted]

SEQ ID NO: 54 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 56 - protein, 326 aa, Homo sapiens; identical to SEQ ID NO: 55

SEQ ID NO: 57 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 58 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 107 aa, Homo sapiens [sequence omitted]

SEQ ID NO: 62 - protein, 329 aa, Rattus norvegicus
ARTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRIEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 63 - protein, 107 aa, Rattus norvegicus
RADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 64 - protein, 323 aa, Oryctolagus cuniculus
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 65 - protein, 104 aa, Oryctolagus cuniculus
RDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 66 - protein, 293 aa, Oryctolagus cuniculus
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 67 - protein, 219 aa, Homo sapiens
DVLMTQTPLSLPVNLGDQASISCRSRQSIVHTNGNTYLGWFLQKPGQSPKLLIYKVSNRFSGVPDRFSGSGSGTDFTLTISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 219 aa, Homo sapiens
DVLMTQTPLSLPVNLGDQASISCRSRQSIVHTNGNTYLGWFLQKPGQSPKLLIYKVSNRFSGVPDRFSGSGSGTDFTLTISRVEAEDLGVYYCFQGSHVPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 219 aa, Homo sapiens
DIVMTQSPLSLPVTPGEPASISCRSRQSIVHTNGNTYLGWYLQKPGQSPRLLIYKVSNRFSGVPDRFSGSGSGTDFTLKISRVEADDVGIYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 214 aa, Homo sapiens
DIVMTQSPLSLPVTPGEPASISCRASQDISNYLNWYLQKPGQSPRLLIYYTSRLYSGVPDRFSGSGSGTDFTLKISRVEADDVGIYYCQQGNTLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 219 aa, Homo sapiens
DIVMTQSPDSLAVSLGERATINCRSSQNIVQSNGNTYLEWYQQKPGQPPKLLIYKVFHRFSGVPDRFSGSGSGTDFTLTISSLQAEDVAVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 214 aa, Homo sapiens
DIQMTQSPSSLSASVGDRVTITCRASQSISNYLNWYQQKPGKAPKLLIYYTSRLYSGVPSRFSGSGSGTDFTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 219 aa, Homo sapiens
DVVMTQSPLSLPVTLGQPASISCRSRQSIVHTNGNTYLGWFQQRPGQSPRRLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 219 aa, Homo sapiens
DVVMTQSPLSLPVTLGQPASISCRSSQNIVQSNGNTYLEWFQQRPGQSPRRLIYKVFHRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 214 aa, Homo sapiens
DIQMTQSPSSLSASVGDRVTITCRASQDISNYLNWYQQKPGKAPKLLIYYTSRLYSGVPSRFSGSGSGTDFTFTISSLQPEDIATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 214 aa, Mus musculus
DIQMTQTTSSLSASLGDRVTISCRASQDISNYLNWYQQKPDGTVKLLIYYTSRLYSGVPSRFSGSGSGTDYSLTISNLEQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 219 aa, Mus musculus
DVFMTQTPLSLPVSLGDQASISCRSSQNIVQSNGNTYLEWYLQKPGQSPKLLIYKVFHRFSGVPDRFSGSGSGTDFTLKISRVEAEDLGVYYCFQGSHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 444 aa, Mus musculus
EVQLQQSGPELVKPGASVKMSCKASGYTFTNYVIHWVKRRPGQGLEWIGYIYPYNDGILYNEKFKGKATVTSDKSSSTAYMDLSSLTSEDSAVYYCTRGGYYVPDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 444 aa, Homo sapiens
QVQLVQSGAEVKKPGASVQVSCKASGYTFTNYVIHWLRQAPGQGLEWMGYIYPYNDGILYNEKFKGRVTMTSDTSISTAYMELSSLRSDDTAVYYCARGGYYVPDYWGQATLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 444 aa, Homo sapiens
QVQLVQSGAEVKKPGASVQVSCKASGYSFTNYYIHWLRQAPGQGLEWMGYIDPLNGDTTYNQKFKGRVTMTSDTSISTAYMELSSLRSDDTAVYYCARGGKRAMDYWGQATLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 443 aa, Homo sapiens
QVQLVQSGAEVKKPGASVKVSCKASGYTFTNYWIHWVRQAPGQGLEWMGYTDPRTDYTEYNQKFKDRVTMTRDTSTSTVYMELSSLRSEDTAVYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 82 - protein, 443 aa, Homo sapiens
EVQLVQSGAEVKKPGESLKISCKGSGYTFTNYWIHWVRQMPGKGLEWMGYTDPRTDYTEYNQKFKDQVTISADKSISTAYLQWSSLKASDTAMYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 83 - protein, 443 aa, Homo sapiens
QVQLVQSGAEVKKPGSSVKVSCKASGYTFTNYWIHWVRQAPGQGLEWMGYTDPRTDYTEYAQKFQGRVTITADESTSTAYMELSSLRSEDTAVYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 84 - protein, 444 aa, Homo sapiens
QVQLVQSGAEVKKPGASVQVSCKASGYTFTNYVIHWLRQAPGQGLEWMGYIYPYNDGILYNEKFKGRVTMTSDTSISTAYMELSSLRSDDTAVYYCARGGYYVYDYWGQATLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 443 aa, Homo sapiens
QVQLVQSGAEVKKPGSSVKVSCKASGYTFTNYWIHWVRQAPGQGLEWMGYTDPRTDYTEYNQKFKDRVTITADESTSTAYMELSSLRSEDTAVYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 86 - protein, 444 aa, Homo sapiens
EVQLVQSGAEVKKPGESLKISCKGSGYSFTNYYIHWVRQMPGKGLEWMGYIDPLNGDTTYSPSFQGQVTISADKSISTAYLQWSSLKASDTAMYYCARGGKRAMDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 444 aa, Homo sapiens
EVQLVQSGAEVKKPGESLKISCKGSGYTFTNYVIHWVRQMPGKGLEWMGYIYPYNDGILYNEKFKGQVTISADKSISTAYLQWSSLKASDTAMYYCARGGYYVPDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - protein, 444 aa, Homo sapiens
QVQLVQSGAEVKKPGASVKVSCKASGYTFTNYVIHWVRQAPGQGLEWMGYIYPYNDGILYNEKFKGRVTMTTDTSTSTAYMELRSLRSDDTAVYYCARGGYYVPDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 443 aa, Homo sapiens
EVQLVQSGAEVKKPGESLKISCKGSGYTFTNYWIHWVRQMPGKGLEWMGYTDPRTDYTEYSPSFQGQVTISADKSISTAYLQWSSLKASDTAMYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 90 - protein, 444 aa, Homo sapiens
QVQLVQSGAEVKKPGASVKVSCKASGYSFTNYYIHWVRQAPGQGLEWMGYIDPLNGDTTYNQKFKGRVTMTRDTSTSTVYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 91 - protein, 444 aa, Homo sapiens
QVQLVQSGAEVKKPGSSVKVSCKASGYSFTNYYIHWVRQAPGQGLEWMGYIDPLNGDTTYNQKFKGRVTITADESTSTAYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - protein, 444 aa, Homo sapiens
EVQLVQSGAEVKKPGESLKISCKGSGYSFTNYYIHWVRQMPGKGLEWMGYIDPLNGDTTYNQKFKGQVTISADKSISTAYLQWSSLKASDTAMYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 444 aa, Homo sapiens
QVQLVQSGAEVKKPGSSVKVSCKASGYSFTNYYIHWVRQAPGQGLEWMGYIDPLNGDTTYAQKFQGRVTITADESTSTAYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 444 aa, Homo sapiens; identical to SEQ ID NO: 90

SEQ ID NO: 95 - protein, 444 aa, Mus musculus
EVQLQQSGPELMKPGASVKISCKASGYSFTNYYIHWVNQSHGKSLEWIGYIDPLNGDTTYNQKFKGKATLTVDKSSSTAYMRLSSLTSADSAVYYCARGGKRAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 96 - protein, 443 aa, Mus musculus
QVQLQQFGAELAKPGASVQMSCKASGYTFTNYWIHWVKQRPGQGLEWIGYTDPRTDYTEYNQKFKDKATLAADRSSSTAYMRLSSLTSEDSAVYYCAGGGRVGLGYWGHGSSVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 97 - protein, 443 aa, Homo sapiens
QVQLVQSGAEVKKPGASVKVSCKASGYSFTNYYIHWVRQAPGQGLEWMGYIDPLNGDTTYNQKFKGRVTMTRDTSTSTVYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 98 - protein, 443 aa, Homo sapiens
QVQLVQSGAEVKKPGSSVKVSCKASGYSFTNYYIHWVRQAPGQGLEWMGYIDPLNGDTTYNQKFKGRVTITADESTSTAYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 99 - protein, 443 aa, Homo sapiens
EVQLVQSGAEVKKPGESLKISCKGSGYSFTNYYIHWVRQMPGKGLEWMGYIDPLNGDTTYNQKFKGQVTISADKSISTAYLQWSSLKASDTAMYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 100 - protein, 443 aa, Homo sapiens
QVQLVQSGAEVKKPGSSVKVSCKASGYSFTNYYIHWVRQAPGQGLEWMGYIDPLNGDTTYAQKFQGRVTITADESTSTAYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 101 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2022/0313819 A1
(43) Pub. Date: Oct. 6, 2022

(54) COMBINATION THERAPY FOR THE TREATMENT OF SOLID AND HEMATOLOGICAL CANCERS

(71) Applicant: Arch Oncology, Inc., Brisbane, CA (US)

(72) Inventors: Michael DONIO, Ballwin, MO (US); Pamela T. MANNING, Chesterfield, MO (US); Robyn PURO, St. Louis, MO (US); Juan C. ALMAGRO, Cambridge, MA (US); Robert W. KARR, Frontenac, MO (US); Benjamin J. CAPOCCIA, St. Louis, MO (US); Myriam BOUCHLAKA, St. Louis, MO (US); W. Casey WILSON, St. Louis, MO (US); Daniel S. PEREIRA, San Diego, CA (US)

(21) Appl. No.: 17/726,683

(22) Filed: Apr. 22, 2022

Related U.S. Application Data
(63) Continuation of application No. PCT/US2020/056339, filed on Oct. 19, 2020.
(60) Provisional application No. 63/043,998, filed on Jun. 25, 2020.
(60) Provisional application No. 62/944,272, filed on Dec. 5, 2019.
(60) Provisional application No. 62/925,037, filed on Oct. 23, 2019.

(51) Int. Cl. A61K 39/395; C07K 16/28; A61P 35/00; A61K 31/704; A61K 31/69; A61K 33/243; A61K 31/337; A61K 31/573; A61K 31/706; A61K 31/496

(52) U.S. Cl. CPC A61K 39/3955; C07K 16/2803; A61P 35/00; A61K 31/704; A61K 31/69; A61K 33/243; A61K 31/337; C07K 16/2896; C07K 16/2887; A61K 31/573; A61K 31/706; A61K 31/496; C07K 2317/24; C07K 2317/92; C07K 2317/76; C07K 2317/73; A61K 2039/505

(57) ABSTRACT

Methods are provided for using anti-CD47 mAbs as therapeutics for the prevention and treatment of solid and hematological cancers, with other anti-cancer agents, which include but are not limited to proteasome inhibitors, immunomodulatory agents, Bruton's tyrosine kinase (BTK) inhibitors, BCMA-targeting agents, CAR-T cells, anthracyclines, platinums, taxols, cyclophosphamides, topoisomerase inhibitors, anti-metabolites, anti-tumor antibiotics, mitotic inhibitors, alkylating agents, and demethylating agents.

### CROSS-REFERENCE TO RELATED APPLICATIONS

This application is a continuation of International Application No. PCT/US2020/056339, filed Oct. 19, 2020, which claims priority to U.S. Provisional Application No. 62/925,037, filed Oct. 23, 2019, U.S. Provisional Application No. 62/944,272, filed Dec. 5, 2019, and U.S. Provisional Application No. 63/043,998, filed Jun. 25, 2020, each of which is incorporated by reference herein in its entirety for all purposes.

### INCORPORATION BY REFERENCE OF SEQUENCE LISTING

The contents of the text file submitted electronically herewith are incorporated herein by reference in their entirety: A computer readable format copy of the Sequence Listing (filename: ARCO_007_03US_SeqList_ST25.txt, date recorded: Mar. 15, 2022, file size ˜179 Kilobytes).

### FIELD OF THE DISCLOSURE

This disclosure is related generally to anti-CD47 monoclonal antibodies (anti-CD47 mAbs) with distinct functional profiles as described herein, methods to generate anti-CD47 mAbs, and methods of using these anti-CD47 mAbs in combination with anti-cancer agents as therapeutics for the prevention and treatment of solid and hematological cancers.

### BACKGROUND OF THE DISCLOSURE

CD47 is a cell surface receptor comprised of an extracellular IgV set domain, a 5 transmembrane domain, and a cytoplasmic tail that is alternatively spliced. Two ligands bind CD47: signal inhibitory receptor protein α (SIRPα) and thrombospondin-1 (TSP1). CD47 expression and/or activity has been implicated in a number of diseases and disorders. Accordingly, there exists a need for therapeutic compositions and methods for treating diseases and conditions associated with CD47 in humans, including the prevention and treatment of solid and hematological cancers, with a combination of anti-cancer agents.

### SUMMARY OF THE DISCLOSURE

Compositions and methods are provided for the prevention and treatment of solid and hematological cancers, in combination with anti-cancer agents.

The present disclosure describes anti-CD47 mAbs with distinct functional profiles. These antibodies possess distinct combinations of properties selected from the following: 1) exhibit cross-reactivity with one or more species homologs of CD47; 2) block the interaction between CD47 and its ligand SIRPα; 3) increase phagocytosis of human tumor cells; 4) induce death of susceptible human tumor cells; 5) do not induce cell death of human tumor cells; 6) do not have reduced or minimal binding to human red blood cells (hRBCs); 7) have reduced binding to hRBCs; 8) have minimal binding to hRBCs; 9) cause reduced agglutination of hRBCs; 10) cause no detectable agglutination of hRBCs; 11) reverse TSP1 inhibition of the nitric oxide (NO) pathway; 12) do not reverse TSP1 inhibition of the NO pathway; 13) cause loss of mitochondrial membrane potential; 14) do not cause loss of mitochondrial membrane potential; 15) cause an increase in cell surface calreticulin expression on human tumor cells; 16) do not cause an increase in cell surface calreticulin expression on human tumor cells; 17) cause an increase in adenosine triphosphate (ATP) release by human tumor cells; 18) do not cause an increase in adenosine triphosphate (ATP) release by human tumor cells; 19) cause an increase in high mobility group box 1 (HMGB1) release by human tumor cells; 20) do not cause an increase in high mobility group box 1 (HMGB1) release by human tumor cells; 21) cause an increase in type I interferon release by human tumor cells; 22) do not cause an increase in type I interferon release by human tumor cells; 23) cause an increase in C-X-C Motif Chemokine Ligand 10 (CXCL10) release by human tumor cells; 24) do not cause an increase in C-X-C Motif Chemokine Ligand 10 (CXCL10) release by human tumor cells; 25) cause an increase in cell surface protein disulfide-isomerase A3 (PDIA3) expression on human tumor cells; 26) do not cause an increase in cell surface protein disulfide-isomerase A3 (PDIA3) expression on human tumor cells; 27) cause an increase in cell surface heat shock protein 70 (HSP70) expression on human tumor cells; 28) do not cause an increase in cell surface heat shock protein 70 (HSP70) expression on human tumor cells; 29) cause an increase in cell surface heat shock protein 90 (HSP90) expression on human tumor cells; 30) do not cause an increase in cell surface heat shock protein 90 (HSP90) expression on human tumor cells; 31) have reduced binding to normal human cells, which includes, but is not limited to, endothelial cells, skeletal muscle cells, epithelial cells, and peripheral blood mononuclear cells (e.g., human aortic endothelial cells, human skeletal muscle cells, human microvascular endothelial cells, human renal tubular epithelial cells, human peripheral blood CD3+ cells, and human peripheral blood mononuclear cells); 32) do not have reduced binding to normal human cells, which includes, but is not limited to, endothelial cells, skeletal muscle cells, epithelial cells, and peripheral blood mononuclear cells (e.g., human aortic endothelial cells, human skeletal muscle cells, human microvascular endothelial cells, human renal tubular epithelial cells, human peripheral blood CD3+ cells, and human peripheral blood mononuclear cells); 33) have a greater affinity for human CD47 at an acidic pH compared to physiological pH; 34) do not have a greater affinity for human CD47 at an acidic pH compared to physiological pH; and 35) cause an increase in annexin A1 release by human tumor cells. The anti-CD47 mAbs of the disclosure are useful in various therapeutic methods for treating diseases and conditions associated with CD47 in humans and animals, including the prevention and treatment of solid and hematological cancers. The antibodies of the disclosure are also useful as diagnostics to determine the level of CD47 expression in tissue samples. Embodiments of the disclosure include isolated antibodies and immunologically active binding fragments thereof; pharmaceutical compositions comprising one or more of the anti-CD47 mAbs, preferably chimeric or humanized forms of said anti-CD47 mAbs; methods of therapeutic use of such anti-CD47 monoclonal antibodies; and cell lines that produce these anti-CD47 mAbs. Embodiments of the disclosure are useful in various therapeutic methods in combination with anti-cancer agents for treating diseases and conditions associated with the prevention and treatment of solid and hematological cancers.

The embodiments of the disclosure include the anti-CD47 mAbs and immunologically active binding fragments thereof, pharmaceutical compositions comprising one or more of the anti-CD47 mAbs, preferably chimeric or humanized forms of said anti-CD47 mAbs; methods of therapeutic use of such anti-CD47 monoclonal antibodies in combination with anti-cancer agents.

The embodiments of the disclosure include a method of preventing or treating cancer in a subject by administering to the subject a combination of an anti-CD47 antibody, or an antigen binding fragment thereof, and a second anti-cancer agent.

The embodiments of the disclosure include administering a combination of an anti-CD47 antibody or an antigen binding fragment thereof, and a second anti-cancer agent which increases death of tumor cells, compared to monotherapy administration of an anti-CD47 antibody or second anti-cancer agent.

The embodiments of the disclosure include administering a combination of an anti-CD47 antibody, or an antigen binding fragment thereof, as described herein, and a second anti-cancer agent which increases expression of immunogenic cell death (ICD) characteristics, compared to monotherapy administration of an anti-CD47 antibody or second anti-cancer agent.

The embodiments of the disclosure include administering a combination of an anti-CD47 antibody, as described herein, and a second anti-cancer agent which increases cell surface calreticulin expression by human tumor cells, compared to monotherapy administration of an anti-CD47 antibody or second anti-cancer agent.

The embodiments of the disclosure include administering a combination of an anti-CD47 antibody, as described herein, and a second anti-cancer agent which increases release of ATP by human tumor cells, compared to monotherapy administration of an anti-CD47 antibody or second anti-cancer agent.

The embodiments of the disclosure include a second anti-cancer agent which is a proteasome inhibitor.

The embodiments of the disclosure wherein the proteasome inhibitor is chosen from bortezomib, carfilzomib, and ixazomib.

The embodiments of the disclosure include a second anti-cancer agent which is selinexor.

The embodiments of the disclosure include a second anti-cancer agent which is an immunomodulatory agent.

The embodiments of the disclosure include a second anti-cancer agent, which is an immodulatory agent, chosen from lenalidomide or pomalidomide.

The embodiments of the disclosure wherein the lenalidomide is further administered in combination with dexamethasone.

The embodiments of the disclosure wherein the pomalidomide is further administered in combination with dexamethasone.

The embodiments of the disclosure include a second anti-cancer agent which is a Bruton's tyrosine kinase (BTK) inhibitor.

The embodiments of the disclosure wherein the Bruton's tyrosine kinase (BTK) inhibitor is chosen from ibrutinib (PCI-32765), acalabrutinib, and zanubrutinib.

The embodiments of the disclosure include a second anti-cancer agent which is a BCMA-targeting agent.

The embodiments of the disclosure wherein BCMA-targeting agent is chosen from JNJ-4528, teclistamab (JNJ-7957) and belantamab mafodotin (GSK2857916).

The embodiments of the disclosure include a second anti-cancer agent which is a CAR-T cell.

The embodiments of the disclosure wherein the CAR-T cell is chosen from an anti-CD19 CAR-T cell or an anti-BCMA CAR-T cell.

The embodiments of the disclosure include a second anti-cancer agent which is an inhibitor of the B-cell lymphoma-2 protein (BCL-2).

The embodiments of the disclosure wherein the B-cell lymphoma-2 protein (BCL-2) inhibitor is venetoclax.

The embodiments of the disclosure include a second anti-cancer agent which is a chemotherapeutic agent.

The embodiments of the disclosure include a chemotherapeutic agent, which is chosen from the chemotherapeutic agents classes of anthracyclines, platinums, taxols, topoisomerase inhibitors, anti-metabolites, anti-tumor antibiotics, mitotic inhibitors, and alkylating agents.

The embodiments of the disclosure include chemotherapeutic agent class anthracyclines, which is chosen from doxorubicin, epirubicin, daunorubicin, and idarubicin.

The embodiments of the disclosure include an anti-CD47 antibody and a second anti-cancer agent which is doxorubicin.

The embodiments of the disclosure include the chemotherapeutic agent class platinums, which is chosen from oxaliplatin, cisplatin, and carboplatin.

The embodiments of the disclosure include the chemotherapeutic agent class taxols, which is chosen from paclitaxel and docetaxel.

The embodiments of the disclosure include the chemotherapeutic agent class topoisomerase inhibitors, which is chosen, but is not limited to the group consisting of irinotecan, topotecan, etoposide, and mitoxantrone.

The embodiments of the disclosure include the chemotherapeutic agent class anti-metabolites, wherein the anti-metabolite is chosen from 5-FU, capecitabine, cytarabine, gemcitabine, and permetrexed.

The embodiments of the disclosure include the chemotherapeutic agent class mitotic inhibitors, wherein the mitotic inhibitor is chosen from vinorelibine, vinblastine, and vincristine.

The embodiments of the disclosure include the chemotherapeutic agent class alkylating agents, wherein the alkylating agent is temzolomide.

The embodiments of the disclosure include the chemotherapeutic agent class demethylating agents, wherein the demethylating agent is 5-azacitidine.

The embodiments of the disclosure include the anti-CD47 mAbs, or antigen-binding fragments thereof, which are defined herein by reference to specific structural characteristics i.e. specified amino acid sequences of either the CDRs or entire heavy chain or light chain variable domains. All antibodies of the disclosure bind to CD47.

The monoclonal antibodies, or antigen binding fragments thereof may comprise at least one, usually at least three, CDR sequences as provided herein, usually in combination with framework sequences from a human variable region or as an isolated CDR peptide. In some embodiments, an antibody comprises at least one light chain comprising the three light chain CDR sequences provided herein situated in a variable region framework, which may be, without limitation, a murine or human variable region framework, and at least one heavy chain comprising the three heavy chain CDR sequences provided herein situated in a variable region framework, which may be, without limitation, a human or murine variable region framework.

Some embodiments of the disclosure are anti-CD47 mAbs, or antigen binding fragments thereof, comprising a heavy chain variable domain comprising a variable heavy chain CDR1, variable heavy chain CDR2, and a variable heavy chain CDR3, wherein said variable heavy chain CDR1 comprises an amino acid sequence selected from the group consisting of: SEQ ID NO:1, SEQ ID NO:2, SEQ ID NO:3; said variable heavy chain CDR2 comprises an amino acid sequence selected from the group consisting of: SEQ ID NO:4, SEQ ID NO:5, SEQ ID NO:6; and said variable heavy chain CDR3 comprises an amino acid sequence selected from the group consisting of: SEQ ID NO:7, SEQ ID NO:8, SEQ ID NO:9, and SEQ ID NO:10.

The heavy chain variable (VH) domain may comprise any one of the listed variable heavy chain CDR1 sequences (HCDR1) in combination with any one of the variable heavy chain CDR2 sequences (HCDR2) and any one of the variable heavy chain CDR3 sequences (HCDR3). However, certain embodiments of HCDR1 and HCDR2 and HCDR3 are particularly preferred, which derive from a single common V_{H} domain, examples of which are described herein.

The antibody or antigen binding fragment thereof may additionally comprise a light chain variable (V_{L}) domain, which is paired with the VH domain to form an antigen binding domain. Preferred light chain variable domains are those comprising a variable light chain CDR1, variable light chain CDR2, and a variable light chain CDR3, wherein said variable light chain CDR1 comprises an amino acid sequence selected from the group consisting of: SEQ ID NO:11, SEQ ID NO:12, SEQ ID NO:13, SEQ ID NO:14; said variable light chain CDR2 optionally comprises an amino acid sequence selected from the group consisting of: SEQ ID NO:15, SEQ ID NO:16, SEQ ID NO:17; and said variable light chain CDR3 optionally comprises an amino acid sequence selected from the group consisting of: SEQ ID NO:18, SEQ ID NO:19, SEQ ID NO:20.

The light chain variable domain may comprise any one of the listed variable light chain CDR1 sequences (LCDR1) in combination with any one of the variable light chain CDR2 sequences (LCDR2) and any one of the variable light chain CDR3 sequences (LCDR3). However, certain embodiments of LCDR1 and LCDR2 and LCDR3 are particularly preferred, which derive from a single common V_{L} domain, examples of which are described herein.

Any given CD47 antibody or antigen binding fragment thereof comprising a V_{H} domain paired with a V_{L} domain will comprise a combination of 6 CDRs: variable heavy chain CDR1 (HCDR1), variable heavy chain CDR2 (HCDR2), variable heavy chain CDR3 (HCDR3), variable light chain CDR1 (LCDR1), variable light chain CDR2 (LCDR2), and variable light chain CDR1 (LCDR1). Although all combinations of 6 CDRs selected from the CDR sequence groups listed above are permissible, and within the scope of the disclosure, certain combinations of 6 CDRs are provided.

Preferred combinations of 6 CDRs include, but are not limited to, the combinations of variable heavy chain CDR1 (HCDR1), variable heavy chain CDR2 (HCDR2), variable heavy chain CDR3 (HCDR3), variable light chain CDR1 (LCDR1), variable light chain CDR2 (LCDR2), and variable light chain CDR3 (LCDR3) selected from the group consisting of:
(i) HCDR1 comprising SEQ ID NO:1, HCDR2 comprising SEQ ID NO:4, HCDR3 comprising SEQ ID NO:7, LCDR1 comprising SEQ ID NO:11, LCDR2 comprising SEQ ID NO:15, LCDR3 comprising SEQ ID NO: 18;
(ii) HCDR1 comprising SEQ ID NO:1, HCDR2 comprising SEQ ID NO:4, HCDR3 comprising SEQ ID NO:8, LCDR1 comprising SEQ ID NO:11, LCDR2 comprising SEQ ID NO:15, LCDR3 comprising SEQ ID NO:18;
(iii) HCDR1 comprising SEQ ID NO:2, HCDR2 comprising SEQ ID NO:5, HCDR3 comprising SEQ ID NO:9, LCDR1 comprising SEQ ID NO:12, LCDR2 comprising SEQ ID NO:16, LCDR3 comprising SEQ ID NO: 19;
(iv) HCDR1 comprising SEQ ID NO:2, HCDR2 comprising SEQ ID NO:5, HCDR3 comprising SEQ ID NO:9, LCDR1 comprising SEQ ID NO:13, LCDR2 comprising SEQ ID NO:16, LCDR3 comprising SEQ ID NO: 19; and
(v) HCDR1 comprising SEQ ID NO:3, HCDR2 comprising SEQ ID NO:6, HCDR3 comprising SEQ ID NO:10, LCDR1 comprising SEQ ID NO:14, LCDR2 comprising SEQ ID NO:17, LCDR3 comprising SEQ ID NO: 18.

In some embodiments, anti-CD47 mAbs include antibodies or antigen binding fragments thereof, comprising a heavy chain variable domain having an amino acid sequence selected from the group consisting of: the amino acid sequences of SEQ ID NO:21, SEQ ID NO:22, SEQ ID NO:23, SEQ ID NO:24, SEQ ID NO:26, SEQ ID NO:27, SEQ ID NO:28, SEQ ID NO:29, SEQ ID NO:30, SEQ ID NO:31, SEQ ID NO: 32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40 and amino acid sequences exhibiting at least 90%, 95%, 97%, 98%, or 99% sequence identity to one of the recited sequences. Alternatively or in addition, preferred anti-CD47 mAbs including antibodies or antigen binding fragments thereof may comprise a light chain variable domain having an amino acid sequence selected from the group consisting of: the amino acid sequences of SEQ ID NO:41, SEQ ID NO:42, SEQ ID NO:43, SEQ ID NO:44, SEQ ID NO:46, SEQ ID NO:48, SEQ ID NO:49, SEQ ID NO:50, SEQ ID NO:51, and SEQ ID NO:52 and amino acid sequences exhibiting at least 90%, 95%, 97%, 98%, or 99% sequence identity to one of the recited sequences.

Although all possible pairing of V_{H} domains and VL domains selected from the V_{H} and VL domain sequence groups listed above are permissible, and within the scope of the disclosure, certain combinations of V_{H} and VL domains are particularly preferred. Accordingly, preferred anti-CD47 mAbs, or antigen binding fragments thereof, are those comprising a combination of a heavy chain variable domain (VH) and a light chain variable domain (VL), wherein the combination is selected from the group consisting of:
(i) a heavy chain variable domain comprising the amino acid sequence of SEQ ID NO:21 and a light chain variable domain comprising the amino acid sequence SEQ ID NO:41;
(ii) a heavy chain variable domain comprising the amino acid sequence of SEQ ID NO:23 and a light chain variable domain comprising the amino acid sequence SEQ ID NO:43;
(iii) a heavy chain variable domain comprising the amino acid sequence of SEQ ID NO:34 and a light chain variable domain comprising the amino acid sequence SEQ ID NO:49;
(iv) a heavy chain variable domain comprising the amino acid sequence of SEQ ID NO:36 and a light chain variable domain comprising the amino acid sequence SEQ ID NO:52;
(v) a heavy chain variable domain comprising the amino acid sequence of SEQ ID NO:38 and a light chain variable domain comprising the amino acid sequence SEQ ID NO:52;
(vi) a heavy chain variable domain comprising the amino acid sequence of SEQ ID NO:39 and a light chain variable domain comprising the amino acid sequence SEQ ID NO:52;
(vii) a heavy chain variable domain comprising the amino acid sequence of SEQ ID NO:24 and a light chain variable domain comprising the amino acid sequence SEQ ID NO: 43;
(viii) a heavy chain variable domain comprising the amino acid sequence of SEQ ID NO:37 and a light chain variable domain comprising the amino acid sequence SEQ ID NO:52;
(ix) a heavy chain variable domain comprising the amino acid sequence of SEQ ID NO:33 and a light chain variable domain comprising the amino acid sequence SEQ ID NO:48;
(x) a heavy chain variable domain comprising the amino acid sequence of SEQ ID NO:26 and a light chain variable domain comprising the amino acid sequence SEQ ID NO:44;
(xi) a heavy chain variable domain comprising the amino acid sequence of SEQ ID NO:27 and a light chain variable domain comprising the amino acid sequence SEQ ID NO:44;
(xii) a heavy chain variable domain comprising the amino acid sequence of SEQ ID NO: 38 and a light chain variable domain comprising the amino acid sequence SEQ ID NO:51;
(xiii) a heavy chain variable domain comprising the amino acid sequence of SEQ ID NO:39 and a light chain variable domain comprising the amino acid sequence SEQ ID NO:51;
(xiv) a heavy chain variable domain comprising the amino acid sequence of SEQ ID NO:40 and a light chain variable domain comprising the amino acid sequence SEQ ID NO: 52;
(xv) a heavy chain variable domain comprising the amino acid sequence of SEQ ID NO:36 and a light chain variable domain comprising the amino acid sequence SEQ ID NO:51;
(xvi) a heavy chain variable domain comprising the amino acid sequence of SEQ ID NO:29 and a light chain variable domain comprising the amino acid sequence SEQ ID NO:47;
(xvii) a heavy chain variable domain comprising the amino acid sequence of SEQ ID NO:30 and a light chain variable domain comprising the amino acid sequence SEQ ID NO:47;
(xviii) a heavy chain variable domain comprising the amino acid sequence of SEQ ID NO:31 and a light chain variable domain comprising the amino acid sequence SEQ ID NO: 47;
(xix) a heavy chain variable domain comprising the amino acid sequence of SEQ ID NO:32 and a light chain variable domain comprising the amino acid sequence SEQ ID NO:47;
(xx) a heavy chain variable domain comprising the amino acid sequence of SEQ ID NO:33 and a light chain variable domain comprising the amino acid sequence SEQ ID NO:47;
(xxi) a heavy chain variable domain comprising the amino acid sequence of SEQ ID NO:29 and a light chain variable domain comprising the amino acid sequence SEQ ID NO: 48;
(xxii) a heavy chain variable domain comprising the amino acid sequence of SEQ ID NO:30 and a light chain variable domain comprising the amino acid sequence SEQ ID NO:48;
(xxiii) a heavy chain variable domain comprising the amino acid sequence of SEQ ID NO:31 and a light chain variable domain comprising the amino acid sequence SEQ ID NO:48;
(xxiv) a heavy chain variable domain comprising the amino acid sequence of SEQ ID NO:32 and a light chain variable domain comprising the amino acid sequence SEQ ID NO: 48;
(xxv) a heavy chain variable domain comprising the amino acid sequence of SEQ ID NO:26 and a light chain variable domain comprising the amino acid sequence SEQ ID NO:43;
(xxvi) a heavy chain variable domain comprising the amino acid sequence of SEQ ID NO:27 and a light chain variable domain comprising the amino acid sequence SEQ ID NO:43;
(xxvii) a heavy chain variable domain comprising the amino acid sequence of SEQ ID NO:28 and a light chain variable domain comprising the amino acid sequence SEQ ID NO:46;
(xxviii) a heavy chain variable domain comprising the amino acid sequence of SEQ ID NO:35 and a light chain variable domain comprising the amino acid sequence SEQ ID NO:50;
(xxix) a heavy chain variable domain comprising the amino acid sequence of SEQ ID NO:29 and a light chain variable domain comprising the amino acid sequence SEQ ID NO:48;
(xxx) a heavy chain variable domain comprising the amino acid sequence of SEQ ID NO:30 and a light chain variable domain comprising the amino acid sequence SEQ ID NO:48;
(xxxi) a heavy chain variable domain comprising the amino acid sequence of SEQ ID NO:31 and a light chain variable domain comprising the amino acid sequence SEQ ID NO: 48;
(xxxii) a heavy chain variable domain comprising the amino acid sequence of SEQ ID NO:32 and a light chain variable domain comprising the amino acid sequence SEQ ID NO:48;
(xxxiii) a heavy chain variable domain comprising the amino acid sequence of SEQ ID NO:37 and a light chain variable domain comprising the amino acid sequence SEQ ID NO:51; and
(xxxiv) a heavy chain variable domain comprising the amino acid sequence of SEQ ID NO:40 and a light chain variable domain comprising the amino acid sequence SEQ ID NO:51.

In some embodiments, anti-CD47 antibodies or antigen binding fragments thereof may also comprise a combination of a heavy chain variable domain and a light chain variable domain wherein the heavy chain variable domain comprises a V_{H} sequence with at least 85% sequence identity, or at least 90% sequence identity, or at least 95% sequence identity, or at least 97%, 98% or 99% sequence identity, to the heavy chain amino acid sequences shown above in (i) to (xxxiv) and/or the light chain variable domain comprises a V_{L} sequence with at least 85% sequence identity, or at least 90% sequence identity, or at least 95% sequence identity, or at least 97%, 98% or 99% sequence identity, to the light chain amino acid sequences shown above in (i) to (xxxiv). The specific V_{H} and V_{L} pairings or combinations in parts (i) through (xxxiv) may be preserved for anti-CD47 antibodies having V_{H} and V_{L} domain sequences with a particular percentage sequence identity to these reference sequences.

For all embodiments wherein the heavy chain and/or light chain variable domains of the antibodies or antigen binding fragments are defined by a particular percentage sequence identity to a reference sequence, the V_{H} and/or V_{L} domains may retain identical CDR sequences to those present in the reference sequence such that the variation is present only within the framework regions.

In another embodiment, the preferred CD47 antibodies, or antigen binding fragments thereof, are those comprising a combination of a heavy chain (HC) and a light chain (LC), wherein the combination is selected from the group consisting of:
(i) a heavy chain comprising the amino acid sequence of SEQ ID NO:78 and a light chain comprising the amino acid sequence SEQ ID NO:67;
(ii) a heavy chain comprising the amino acid sequence of SEQ ID NO:79 and a light chain comprising the amino acid sequence SEQ ID NO:69;
(iii) a heavy chain comprising the amino acid sequence of SEQ ID NO:80 and a light chain comprising the amino acid sequence SEQ ID NO:70;
(iv) a heavy chain comprising the amino acid sequence of SEQ ID NO:81 and a light chain comprising the amino acid sequence SEQ ID NO:71;
(v) a heavy chain comprising the amino acid sequence of SEQ ID NO:82 and a light chain comprising the amino acid sequence SEQ ID NO:71;
(vi) a heavy chain comprising the amino acid sequence of SEQ ID NO:83 and a light chain comprising the amino acid sequence SEQ ID NO:71;
(vii) a heavy chain comprising the amino acid sequence of SEQ ID NO:84 and a light chain comprising the amino acid sequence SEQ ID NO:69;
(viii) a heavy chain comprising the amino acid sequence of SEQ ID NO:85 and a light chain comprising the amino acid sequence SEQ ID NO:71;
(ix) a heavy chain comprising the amino acid sequence of SEQ ID NO:86 and a light chain comprising the amino acid sequence SEQ ID NO:72;
(x) a heavy chain comprising the amino acid sequence of SEQ ID NO:87 and a light chain comprising the amino acid sequence SEQ ID NO:73;
(xi) a heavy chain comprising the amino acid sequence of SEQ ID NO:88 and a light chain comprising the amino acid sequence SEQ ID NO:73;
(xii) a heavy chain comprising the amino acid sequence of SEQ ID NO:82 and a light chain comprising the amino acid sequence SEQ ID NO:74;
(xiii) a heavy chain comprising the amino acid sequence of SEQ ID NO:83 and a light chain comprising the amino acid sequence SEQ ID NO:74;
(xiv) a heavy chain comprising the amino acid sequence of SEQ ID NO:89 and a light chain comprising the amino acid sequence SEQ ID NO:71;
(xv) a heavy chain comprising the amino acid sequence of SEQ ID NO:81 and a light chain comprising the amino acid sequence SEQ ID NO:74;
(xvi) a heavy chain comprising the amino acid sequence of SEQ ID NO:90 and a light chain comprising the amino acid sequence SEQ ID NO:75;
(xvii) a heavy chain comprising the amino acid sequence of SEQ ID NO:91 and a light chain comprising the amino acid sequence SEQ ID NO:75;
(xviii) a heavy chain comprising the amino acid sequence of SEQ ID NO:92 and a light chain comprising the amino acid sequence SEQ ID NO:75;
(xix) a heavy chain comprising the amino acid sequence of SEQ ID NO:93 and a light chain comprising the amino acid sequence SEQ ID NO:75;
(xx) a heavy chain comprising the amino acid sequence of SEQ ID NO:86 and a light chain comprising the amino acid sequence SEQ ID NO:75;
(xxi) a heavy chain comprising the amino acid sequence of SEQ ID NO:94 and a light chain comprising the amino acid sequence SEQ ID NO:72;
(xxii) a heavy chain comprising the amino acid sequence of SEQ ID NO:91 and a light chain comprising the amino acid sequence SEQ ID NO:72;
(xxiii) a heavy chain comprising the amino acid sequence of SEQ ID NO:92 and a light chain comprising the amino acid sequence SEQ ID NO:31;
(xxiv) a heavy chain comprising the amino acid sequence of SEQ ID NO:93 and a light chain comprising the amino acid sequence SEQ ID NO:72;
(xxv) a heavy chain comprising the amino acid sequence of SEQ ID NO:87 and a light chain comprising the amino acid sequence SEQ ID NO:69;
(xxvi) a heavy chain comprising the amino acid sequence of SEQ ID NO:88 and a light chain comprising the amino acid sequence SEQ ID NO:69;
(xxvii) a heavy chain comprising the amino acid sequence of SEQ ID NO:95 and a light chain comprising the amino acid sequence SEQ ID NO:76;
(xxviii) a heavy chain comprising the amino acid sequence of SEQ ID NO:96 and a light chain comprising the amino acid sequence SEQ ID NO:77;
(xxix) a heavy chain comprising the amino acid sequence of SEQ ID NO:97 and a light chain comprising the amino acid sequence SEQ ID NO:72;
(xxx) a heavy chain comprising the amino acid sequence of SEQ ID NO:98 and a light chain comprising the amino acid sequence SEQ ID NO:72;
(xxxi) a heavy chain comprising the amino acid sequence of SEQ ID NO:99 and a light chain comprising the amino acid sequence SEQ ID NO:72;
(xxxii) a heavy chain comprising the amino acid sequence of SEQ ID NO:100 and a light chain comprising the amino acid sequence SEQ ID NO:72;
(xxxiii) a heavy chain comprising the amino acid sequence of SEQ ID NO:85 and a light chain comprising the amino acid sequence SEQ ID NO:74;
(xxxiv) a heavy chain comprising the amino acid sequence of SEQ ID NO:89 and a light chain comprising the amino acid sequence SEQ ID NO:74;
   wherein the VH amino acid sequence is at least 90%, 95%, 97%, 98% or 99% identical thereto and the a VL amino acid sequence is at least 90%, 95%, 97%, 98% or 99% identical thereto.

In some embodiments, the anti-CD47 antibodies described herein, are also characterized by combinations of properties which are not exhibited by prior art anti-CD47 antibodies proposed for human therapeutic use. Accordingly, the anti-CD47 antibodies described herein are characterized by:
a. binds to human CD47;
b. blocks SIRPα binding to human CD47;
c. increases phagocytosis of human tumor cells; and
d. induces death of susceptible human tumor cells.

In another embodiment described herein, the anti-CD47 antibodies are characterized by:
a. binds to human CD47;
b. blocks SIRPα binding to human CD47;
c. increases phagocytosis of human tumor cells;
d. induces death of susceptible human tumor cells; and
e. causes no detectable agglutination of human red blood cells (hRBCs).

In yet another embodiment described herein, the anti-CD47 antibodies are characterized by:
a. binds to human CD47;
b. blocks SIRPα binding to human CD47;
c. increases phagocytosis of human tumor cells;
d. induces death of susceptible human tumor cells; and
e. causes reduced agglutination of human red blood cells (hRBCs).

In another embodiment described herein, the anti-CD47 antibodies are characterized by:
a. specifically binds to human CD47;
b. blocks SIRPα binding to human CD47;
c. increases phagocytosis of human tumor cells;
d. induces death of susceptible human tumor cells; and
e. has reduced hRBC binding.

In another embodiment described herein, the anti-CD47 antibodies are characterized by:
a. binds to human CD47;
b. blocks SIRPα binding to human CD47;
c. increases phagocytosis of human tumor cells;
d. causes no detectable agglutination of human red blood cells (hRBCs); and
e. has minimal binding to hRBCs.

In another embodiment described herein, the anti-CD47 antibodies are characterized by:
a. binds to human CD47;
b. blocks SIRPα binding to human CD47;
c. increases phagocytosis of human tumor cells;
d. causes detectable agglutination of human red blood cells (hRBCs); and
e. has reduced hRBC binding.

Additional embodiments of the anti-CD47 antibodies described herein, are also characterized by combinations of properties which are not exhibited by prior art anti-CD47 antibodies proposed for human therapeutic use. Accordingly, the anti-CD47 antibodies described herein are further characterized by one or more among the following characteristics:
a. causes an increase in cell surface calreticulin expression by human tumor cells;
b. causes an increase in adenosine triphosphate (ATP) release by human tumor cells;
c. causes an increase in high mobility group box 1 (HMGB1) release by human tumor cells;
d. causes an increase in annexin A1 release by human tumor cells;
e. causes an increase in Type I Interferon release by human tumor cells;
f. causes an increase in C-X-C Motif Chemokine Ligand 10 (CXCL10) release by human tumor cells;
g. causes an increase in cell surface protein disulfide-isomerase A3 (PDIA3) expression by human tumor cells;
h. causes an increase in cell surface heat shock protein 70 (HSP70) expression by human tumor cells; and
i. causes an increase in cell surface heat shock protein 90 (HSP90) expression by human tumor cells.

In another embodiment described herein, the monoclonal antibody, or antigen binding fragment thereof binds to human, non-human primate, mouse, rabbit, and rat CD47.

In yet another embodiment described herein, the monoclonal antibody, or antigen binding fragment thereof specifically also binds to non-human primate CD47, wherein non-human primate may include, but is not limited to, cynomolgus monkey, green monkey, rhesus monkey, and squirrel monkey.

In yet another embodiment described herein, the monoclonal antibody, or antigen binding fragment thereof, has reduced binding to normal human cells, which includes, but is not limited to, endothelial cells, skeletal muscle cells, epithelial cells, and peripheral blood mononuclear cells (e.g., human aortic endothelial cells, human skeletal muscle cells, human microvascular endothelial cells, human renal tubular epithelial cells, human peripheral blood CD3+ cells, and human peripheral blood mononuclear cells).

In yet another embodiment described herein, the monoclonal antibody, or antigen binding fragment thereof, a greater have a greater affinity for human CD47 at acidic pH than at physiological pH.

In some embodiments, the monoclonal antibody, or antigen binding fragment thereof, may additionally possess one or more of the following characteristics: 1) exhibit cross-reactivity with one or more species homologs of CD47; 2) block the interaction between CD47 and its ligand SIRPα; 3) increase phagocytosis of human tumor cells; 4) induce death of susceptible human tumor cells; 5) do not induce cell death of human tumor cells; 6) do not have reduced or minimal binding to human red blood cells (hRBCs); 7) have reduced binding to hRBCs; 8) have minimal binding to hRBCs; 9) cause reduced agglutination of hRBCs; 10) cause no detectable agglutination of hRBCs; 11) reverse TSP1 inhibition of the nitric oxide (NO) pathway; 12) do not reverse TSP1 inhibition of the NO pathway; 13) cause loss of mitochondrial membrane potential; 14) do not cause loss of mitochondrial membrane potential; 15) cause an increase in cell surface calreticulin expression on human tumor cells; 16) do not cause an increase in cell surface calreticulin expression on human tumor cells; 17) cause an increase in adenosine triphosphate (ATP) release by human tumor cells; 18) do not cause an increase in adenosine triphosphate (ATP) release by human tumor cells; 19) cause an increase in high mobility group box 1 (HMGB1) release by human tumor cells; 20) do not cause an increase in high mobility group box 1 (HMGB1) release by human tumor cells; 21) cause an increase in type I interferon release by human tumor cells; 22) do not cause an increase in type I interferon release by human tumor cells; 23) cause an increase in C-X-C Motif Chemokine Ligand 10 (CXCL10) release by human tumor cells; 24) do not cause an increase in C-X-C Motif Chemokine Ligand 10 (CXCL10) release by human tumor cells; 25) cause an increase in cell surface protein disulfide-isomerase A3 (PDIA3) expression on human tumor cells; 26) do not cause an increase in cell surface protein disulfide-isomerase A3 (PDIA3) expression on human tumor cells; 27) cause an increase in cell surface heat shock protein 70 (HSP70) expression on human tumor cells; 28) do not cause an increase in cell surface heat shock protein 70 (HSP70) expression on human tumor cells; 29) cause an increase in cell surface heat shock protein 90 (HSP90) expression on human tumor cells; 30) do not cause an increase in cell surface heat shock protein 90 (HSP90) expression on human tumor cells; 31) have reduced binding to normal human cells, which includes, but is not limited to, endothelial cells, skeletal muscle cells, epithelial cells, and peripheral blood mononuclear cells (e.g., human aortic endothelial cells, human skeletal muscle cells, human microvascular endothelial cells, human renal tubular epithelial cells, human peripheral blood CD3+ cells, and human peripheral blood mononuclear cells); 32) do not have reduced binding to normal human cells, which includes, but is not limited to, endothelial cells, skeletal muscle cells, epithelial cells, and peripheral blood mononuclear cells (e.g., human aortic endothelial cells, human skeletal muscle cells, human microvascular endothelial cells, human renal tubular epithelial cells, human peripheral blood CD3+ cells, and human peripheral blood mononuclear cells); 33) have a greater affinity for human CD47 at an acidic pH compared to physiological pH; 34) do not have a greater affinity for human CD47 at an acidic pH compared to physiological pH; and 35) cause an increase in annexin A1 release by human tumor cells.

Various forms of the anti-CD47 mAbs disclosed are contemplated herein. For example, the anti-CD47 mAbs can be full length humanized antibodies with human frameworks and constant regions of the isotypes, IgA, IgD, IgE, IgG, and IgM, more particularly, IgG1, IgG2, IgG3, IgG4, and in some cases with various mutations to alter Fc receptor function or prevent Fab arm exchange or an antibody fragment, e.g., a F(ab′)2 fragment, a F(ab) fragment, a single chain Fv fragment (scFv), etc., as disclosed herein.

In some embodiments, the anti-CD47 mAbs or antigen-binding fragment thereof increases phagocytosis of human tumor cells and are administered in combination with an opsonizing monoclonal antibody that targets an antigen on a tumor cell.

In some embodiments, the anti-CD47 mAbs or antigen-binding fragment thereof increases phagocytosis of human tumor cells and are administered in combination with an opsonizing monoclonal antibody that targets an antigen on a tumor cell, wherein the opsonizing monoclonal antibody is chosen from rituximab (anti-CD20), trastuzumab (anti-HER2), alemtuzumab (anti-CD52), cetuximab (anti-EGFR), panitumumab (anti-EGFR), ofatumumab (anti-CD20), denosumab (anti-RANKL), pertuzumab (anti-HER2), panitumumab (EGFR), pertuzumab (HER2), elotuzumab (SLAMF7), atezolizumab (anti-PD-L1), avelumab (anti-PD-L1), durvalumab (anti-PD-L1), necitumumab (anti-EGFR), daratumumab (anti-CD38), obinutuzumab (anti-CD20), blinatumomab (anti-CD19/CD3), dinutuximab (anti-GD2), teclistamab (anti-BCMA×CD3), belantamab mafodotin (anti-BCMA antibody drug conjugate).

In some embodiments, the opsonizing monoclonal antibody targets CD20, EGFR, and PD-L1.

In some embodiments, the disclosure provides for a therapeutic combination of an anti-CD47 mAb as disclosed herein, that binds to CD47, blocks SIRPα binding to human CD47; increases phagocytosis of human tumor cells, and induces death of susceptible human tumor cells, and a second therapeutic agent that is an anti-cancer agent, wherein the anti-cancer agent results in increased immunogenic cell death (ICD) of tumor cells and/or tumor cell death of tumor cells. Specific therapeutic combinations of interest include the anti-CD47 mAbs as disclosed herein and anthracylines, e.g. doxorubicin, epirubcin, daunorubicin, and idarubicin, of which the therapeutic combination finds particular use in the treatment of breast cancer, ovarian cancer, gastric cancer, and hepatocellular carcinoma. A therapeutic combination of the anti-CD47 mAbs as disclosed herein and platinums, e.g. oxaliplatin, cisplatin, and carboplatin, finds particular use in the treatment of CRC and NSCLC. A therapeutic combination of the anti-CD47 mAbs as disclosed herein and taxols, e.g. paclitaxel and docetaxel, finds particular use in the treatment of breast cancer, NSCLC, gastric cancer, and prostate cancer. A therapeutic combination of the anti-CD47 mAbs as disclosed herein and cyclophosphamides, finds particular use in the treatment of lymphoma, multiple myeloma, leukemia, ovarian cancer, breast cancer, small cell lung cancer, neuroblastoma, and sarcoma. A therapeutic combination of the anti-CD47 mAbs as disclosed herein and topoisomerase inhibitors, e.g. irinotecan, topotecan, etoposide, and mitoxantrone, finds particular use in the treatment of CRC, small cell lung cancer, pancreatic cancer, ovarian cancer, and NSCLC. A therapeutic combination of the anti-CD47 mAbs as disclosed herein and anti-metabolites, e.g. 5-FU, capecitabine, cytarabine, gemcitabine, and permetrexed, finds particular use in the treatment of ovarian cancer, breast cancer, and gastric cancer. A therapeutic combination of the anti-CD47 mAbs as disclosed herein and anti-tumor antibiotics, e.g. daunorubicin, doxorubicin, epirubicin, idarubicin finds particular use in the treatment of cancer. A therapeutic combination of the anti-CD47 mAbs as disclosed herein and a mitotic inhibitor, e.g. vinorelibine, vinblastine, and vincristine finds particular use in the treatment cancer. A therapeutic combination of the anti-CD47 mAbs as disclosed herein and an alkylating agent, e.g. temozolomide, finds particular use in the treatment of GBM, melanoma, and multiple myeloma. A therapeutic combination of the anti-CD47 mAbs as disclosed herein and a proteasome inhibitor, e.g. bortezomib, carfilzomib, or ixazomib, finds particular use in the treatment of multiple myeloma. In some embodiments, a therapy which provides for a combination of an agent that binds to CD47, blocks SIRPα binding to human CD47; increases phagocytosis of human tumor cells, and induces death of susceptible human tumor cells, and radiation may also achieve additive or synergistic effects for multiple solid and hematological cancer indications.

In some embodiments, pharmaceutical or veterinary compositions comprising one or more of the anti-CD47 mAbs or fragments disclosed herein, optionally chimeric or humanized forms, and a pharmaceutically acceptable carrier, diluent, or excipient.

Some of the embodiments of the disclosure provide a pharmaceutical composition comprising one of the anti-CD47 mAbs or fragments disclosed herein, optionally chimeric or humanized forms, and a pharmaceutically acceptable carrier, diluent, or excipient, in combination with an anti-cancer agent.

Prior to the present disclosure, there was a need to identify anti-CD47 mAbs that possess the functional profiles as described herein. The anti-CD47 mAbs of the present disclosure exhibit distinct combinations of properties, particularly combinations of properties that render the anti-CD47 mAbs particularly advantageous or suitable for use in human therapy, in combination with anti-cancer agents, particularly in the prevention and treatment of solid and hematological cancers.

In some embodiments, the disclosure provides a monoclonal antibody, or an antigen binding fragment thereof, which: binds to human CD47; blocks SIRPα binding to human CD47; increases phagocytosis of human tumor cells; and induces death of human tumor cells; wherein said monoclonal antibody, or an antigen binding fragment thereof, exhibits pH-dependent binding to CD47 present on a cell. In other embodiments, the disclosure provides a monoclonal antibody, or an antigen binding fragment thereof, which: binds to human CD47; blocks SIRPα binding to human CD47; increases phagocytosis of human tumor cells; wherein said monoclonal antibody, or an antigen binding fragment thereof, exhibits pH-dependent binding to CD47 present on a cell. In other embodiments, the disclosure provides a monoclonal antibody, or an antigen binding fragment thereof, which: binds to human CD47; blocks SIRPα, binding to human CD47; increases phagocytosis of human tumor cells; and induces death of human tumor cells; wherein said monoclonal antibody, or an antigen binding fragment thereof, exhibits reduced binding to normal cells. In one embodiment, these cells may be an endothelial cell, a skeletal muscle cell, an epithelial cell, a PBMC or a RBC (e.g., human aortic endothelial cells, human skeletal muscle cells, human microvascular endothelial cells, human renal tubular epithelial cells, human peripheral blood CD3+ cells, human peripheral blood mononuclear cells or human RBC). In other embodiments, the disclosure provides a monoclonal antibody, or an antigen binding fragment thereof, which: binds to human CD47; blocks SIRPα, binding to human CD47; increases phagocytosis of human tumor cells; wherein said monoclonal antibody, or an antigen binding fragment thereof, exhibits reduced binding to normal cells. In one embodiment, these cells may be an endothelial cell, a skeletal muscle cell, an epithelial cell, a PBMC or a RBC (e.g., human aortic endothelial cells, human skeletal muscle cells, human microvascular endothelial cells, human renal tubular epithelial cells, human peripheral blood CD3+ cells, human peripheral blood mononuclear cells or human RBC). In another embodiment, the monoclonal antibody, or an antigen binding fragment thereof, exhibits both pH dependent binding and reduced binding to a cell.

Further scope of the applicability of the present disclosure will become apparent from the detailed description provided below. However, it should be understood that the detailed description and specific examples, while indicating some embodiments of the disclosure, are given by way of illustration only since various changes and modifications within the spirit and scope of the disclosure will become apparent to those skilled in the art from this detailed description.

### BRIEF DESCRIPTION OF THE DRAWINGS

The above and other aspects, features, and advantages of the present disclosure will be better understood from the following detailed descriptions taken in conjunction with the accompanying drawing(s), all of which are given by way of illustration only, and are not limitative of the present disclosure.

FIG. 1A. Binding of VLX4 Humanized mAbs to Human OV10 Cells Expressing human CD47. Binding of VLX4 humanized mAbs (VLX4hum_01 IgG1, VLX4hum_02 IgG1, VLX4hum_01 IgG4 PE, and VLX4hum_02 IgG4 PE) to human CD47 was determined using a OV10 cell line expressing human CD47 (OV10 hCD47) cell-based ELISA. OV10 hCD47 cells were plated into 96 well plates and were confluent at the time of assay. Various concentrations of mAbs were added to the cells for 1 hr. Cells were washed and then incubated with HRP-labelled secondary antibody for 1 hr followed by addition of peroxidase substrate.

FIG. 1B. Binding of VLX4 Humanized mAbs to Human OV10 Cells Expressing human CD47. Binding of VLX4 humanized mAbs (VLX4hum_06 IgG4 PE, VLX4hum_07 IgG4 PE, VLX4hum_12 IgG4 PE, and VLX4hum_13 IgG4 PE) to human CD47 was determined using an OV10 CD47 cell-based ELISA. OV10 hCD47 cells were plated into 96 well plates and were confluent at the time of assay. Various concentrations of VLX4 representative mAbs were added to the cells for 1 hr. Cells were washed and then incubated with HRP-labelled secondary antibody for 1 hr followed by addition of peroxidase substrate.

FIG. 2A. Binding of VLX4 Humanized mAbs to Human RBCs (hRBCs). Binding of VLX4 humanized mAbs (VLX4hum_01 IgG1, VLX4hum_02 IgG1, VLX4hum_01 IgG4 PE, and VLX4hum_02 IgG4PE) to human CD47 was determined using freshly isolated hRBCs. hRBCs were incubated for 60 minutes at 37° C. with various concentrations of VLX4 mAbs, washed and incubated for 1 hr with FITC-labeled donkey anti-human antibody. Cells were washed and antibody binding measured using flow cytometry.

FIG. 2B. Binding of VLX4 Humanized mAbs to Human RBCs. Binding of VLX4 humanized mAbs (VLX4hum_07 IgG4 PE, VLX4hum_12 IgG4 PE, and VLX4hum_13 IgG4 PE) to human CD47 was determined using freshly isolated hRBCs. hRBCs were incubated for 60 minutes at 37° C. with various concentrations of VLX4 mAbs, washed and incubated for 1 hr with FITC-labeled donkey anti-human antibody. Cells were washed and antibody binding measured using flow cytometry.

FIG. 3A. Binding of VLX8 Humanized mAbs to Human OV10 hCD47 Cells. Binding of VLX8 IgG4PE chimera (xi) or humanized mAbs (VLX8hum_01 IgG4PE, VLX8hum_04 IgG4 PE, VLX8hum_07 IgG4 PE, and VLX8hum_09 IgG4 PE) to human CD47 was determined using an OV10 hCD47 cell-based ELISA. OV10 hCD47 cells were plated into 96 well plates and were confluent at the time of assay. Various concentrations of VLX8 representative mAbs were added to the cells for 1 hr. Cells were washed and then incubated with HRP-labelled secondary antibody for 1 hr followed by addition of peroxidase substrate.

FIG. 3B. Binding of VLX8 Humanized mAbs to Human OV10 hCD47 Cells. Binding of VLX8 chimera or humanized mAbs (VLX8hum_06 IgG2, VLX8hum_07 IgG2, VLX8hum_08 IgG2, and VLX8hum_09 IgG2) to human CD47 was determined using an OV10 hCD47 cell-based ELISA. OV10 hCD47 cells were plated into 96 well plates and were confluent at the time of assay. Various concentrations of VLX8 representative mAbs were added to the cells for 1 hr. Cells were washed and then incubated with RP-labelled secondary antibody for 1 hr followed by addition of peroxidase substrate.

FIG. 4A. Binding of VLX8 Humanized mAbs to Human RBCs. Binding of VLX8 IgG4PE xi or humanized mAbs (VLX8hum_01 IgG4PE, VLX8hum_03 IgG4PE, VLX8hum_07 IgG4PE, and VLX8hum_10 IgG4PE) to human CD47 was determined using freshly isolated human RBCs. RBCs were incubated for 1 hr at 37° C. with various concentrations of VLX8 mAbs, washed and incubated for 1 hr with FITC-labeled donkey anti-human antibody. Cells were washed and antibody binding measured using flow cytometry.

FIG. 4B. Binding of VLX8 Humanized mAbs to Human RBCs. Binding of VLX8 IgG4PE xi or humanized mAbs (VLX8hum_06 IgG2, VLX8hum_07 IgG2, VLX8hum_08 IgG2 and VLX8hum_09 IgG2) to human CD47 was determined using freshly isolated human RBCs. RBCs were incubated for 1 hr at 37° C. with various concentrations of VLX8 mAbs, washed and incubated for 1 hr with FITC-labeled donkey anti-human antibody. Cells were washed and antibody binding measured using flow cytometry.

FIG. 5A. Binding of VLX9 Humanized mAbs to Human OV10 hCD47 Cells. Binding of VLX9 IgG2 xi or humanized mAbs (VLX9hum_01 IgG2, VLX9hum_02 IgG2, VLX9hum_03 IgG2, VLX9hum_04 IgG2 and VLX9hum_05 IgG2) to human CD47 was determined using an OV10 human CD47 cell-based ELISA. OV10 hCD47 cells were plated into 96 well plates and were confluent at the time of assay. Various concentrations of mAbs were added to the cells for 1 hr. Cells were washed and then incubated with HRP-labelled secondary antibody for 1 hr followed by addition of peroxidase substrate.

FIG. 5B. Binding of VLX9 Humanized mAbs to Human OV10 hCD47 Cells. Binding of VLX9 IgG2 xi or humanized mAbs (VLX9hum_06 IgG2, VLX9hum_07 IgG2, VLX9hum_08 IgG2, VLX9hum_09 IgG2 and VLX9hum_10 IgG2) to human CD47 was determined using a OV10 hCD47 cell-based ELISA. OV10 hCD47 cells were plated into 96 well plates and were confluent at the time of assay. Various concentrations of mAbs were added to the cells for 1 hr. Cells were washed and then incubated with HRP-labelled secondary antibody for 1 hr followed by addition of peroxidase substrate.

FIG. 6A. Specific Binding of VLX Humanized mAbs to CD47. Binding of VLX humanized mAb VLX4hum_07 IgG4PE to wildtype and CD47 knockout Jurkat cells was determined by flow cytometry. Various concentrations of mAbs were added to 1×10⁴ cells for 1 hr. The cells were washed and then incubated with FITC-labelled secondary antibody for 1 hr. Cells were washed and antibody binding measured using flow cytometry.

FIG. 6B. Specific Binding of VLX Humanized mAbs to CD47. Binding of VLX humanized mAb VLX9hum_04 IgG2 to wildtype and CD47 knockout Jurkat cells was determined by flow cytometry. Various concentrations of mAbs were added to 1×10⁴ cells for 1 hr. The cells were washed and then incubated with FITC-labelled secondary antibody for 1 hr. Cells were washed and antibody binding measured using flow cytometry.

FIG. 7. Binding of VLX9 Humanized mAbs to Human RBCs. Binding of VLX9 IgG2 xi or humanized VLX9 mAbs to human CD47 (VLX9hum_01 IgG2, VLX9hum_02 IgG2 and VLX9hum_07 IgG2) was determined using freshly isolated human hRBCs. RBCs were incubated for 60 minutes at 37° C. with various concentrations of VLX9 mAbs, washed and incubated for 1 hr with FITC-labelled donkey anti-human antibody. Cells were washed and antibody binding measured using flow cytometry.

FIG. 8A. Binding of VLX Humanized mAbs to Human Aortic Endothelial Cells (HAEC). Binding of VLX humanized mAbs (VLX4hum_07 IgG4PE, VLX8hum_10 IgG4PE, VLX8hum_11 IgG4 PE, VLX4hum_01 IgG4PE, VLX9hum_06 IgG2, VLX9hum_08 IgG2, VLX9hum_09 IgG2, VLX9hum_03 IgG2 and VLX9hum_04 IgG2) to HAEC was determined by flow cytometry. HAEC were removed from the flask using acutase. Various concentrations of mAbs were added to 1×10⁴ cells for 1 hr. The cells were washed and then incubated with FITC-labelled secondary antibody for 1 hr followed by measurement of FITC label by flow cytometry.

FIG. 8B. Binding of VLX Humanized mAbs to Skeletal Human Muscle Cells (SkMC). Binding of VLX humanized mAbs (VLX4hum_07 IgG4PE, VLX8hum_10 IgG4PE, VLX8hum_11 IgG4 PE, VLX4hum_01 IgG4PE, VLX9hum_06 IgG2, VLX9hum_08 IgG2, VLX9hum_09 IgG2, VLX9hum_03 IgG2 and VLX9hum_04 IgG2) to SkMc was determined by flow cytometry. SkMC were removed from the flask using acutase. Various concentrations of mAbs were added to 1×10⁴ cells for 1 hr. The cells were washed and then incubated with FITC-labelled secondary antibody for 1 hr followed by measurement of FITC label by flow cytometry.

FIG. 8C. Binding of VLX Humanized mAbs to Human Lung Microvascular Endothelial Cells (HMVEC-L). Binding of VLX humanized mAbs (VLX4hum_07 IgG4PE, VLX8hum_10 IgG4PE, VLX8hum_11 IgG4 PE, VLX4hum_01 IgG4PE, VLX9hum_06 IgG2, VLX9hum_08 IgG2, VLX9hum_09 IgG2, VLX9hum_03 IgG2 and VLX9hum_04 IgG2) to HMVEC-L was determined by flow cytometry. HMVEC-L were removed from the flask using acutase. Various concentrations of mAbs were added to 1×10⁴ cells for 1 hr. The cells were washed and then incubated with FITC-labelled secondary antibody for 1 hr followed by measurement of FITC label by flow cytometry.

FIG. 8D. Binding of VLX Humanized mAbs to Human Renal Tubular Epithelial Cells (RTEC). Binding of VLX humanized mAbs (VLX4hum_07 IgG4PE, VLX8hum_10 IgG4PE, VLX8hum_11 IgG4 PE, VLX4hum_01 IgG4PE, VLX9hum_06 IgG2, VLX9hum_08 IgG2, VLX9hum_09 IgG2, VLX9hum_03 IgG2 and VLX9hum_04 IgG2) to RTEC by flow cytometry. RTEC were removed from the flask using acutase. Various concentrations of mAbs were added to 1×10⁴ cells for 1 hr. The cells were washed and then incubated with FITC-labelled secondary antibody for 1 hr followed by measurement of FITC label by flow cytometry.

FIG. 8E. Binding of VLX Humanized mAbs to Human Peripheral Blood CD3⁺ Cells. Binding of VLX humanized mAbs (VLX4hum_07 IgG4PE, VLX8hum_10 IgG4PE, VLX8hum_11 IgG4 PE, VLX4hum_01 IgG4PE, VLX9hum_06 IgG2, VLX9hum_08 IgG2, VLX9hum_09 IgG2, VLX9hum_03 IgG2 and VLX9hum_04 IgG2) to CD3⁺ cells was determined by flow cytometry. CD3⁺ cells were plated into 96 well plates. Various concentrations of mAbs were added to the cells for 1 hr. Cells were washed and then incubated with FITC-labelled secondary antibody for 1 hr followed by measurement of FITC label by flow cytometry.

FIG. 8F. Binding of VLX Humanized mAbs to Human Peripheral Blood Mononuclear Cells PBMC). Binding of VLX humanized mAbs (VLX4hum_07 IgG4PE, VLX8hum_10 IgG4PE, VLX8hum_11 IgG4 PE, VLX4hum_01 IgG4PE, VLX9hum_06 IgG2, VLX9hum_08 IgG2, VLX9hum_09 IgG2, VLX9hum_03 IgG2 and VLX9hum_04 IgG2) to PBMC was determined by flow cytometry. PBMCs were plated into 96 well plates. Various concentrations of mAbs were added to the cells for 1 hr. Cells were washed and then incubated with FITC-labelled secondary antibody for 1 hr followed by measurement of FITC label by flow cytometry.

FIG. 9A. pH Dependent and pH Independent Binding of Humanized mAb to His-CD47. Binding of VLX9hum_09 IgG2 to human CD47 was determined using a solid-phase CD47 ELISA assay. His-CD47 was adsorbed to microtiter wells, washed and various concentrations of humanized mAbs were added to the wells for 1 hr at pH 6 or 8. The wells were washed and then incubated with HRP-labelled secondary antibody for 1 hour followed by addition of peroxidase substrate.

FIG. 9B. pH Dependent and pH Independent Binding of Humanized mAb to His-CD47. Binding of VLX9hum_04 IgG2 to human CD47 was determined using a solid-phase CD47 ELISA assay. His-CD47 was adsorbed to microtiter wells, washed and various concentrations of humanized mAbs were added to the wells for 1 hr at pH 6 or 8. The wells were washed and then incubated with HRP-labelled secondary antibody for 1 hour followed by addition of peroxidase substrate.

FIG. 9C. pH Dependent and pH Independent Binding of Humanized mAb to His-CD47. Binding of VLX4hum_07 IgG4PE to human CD47 was determined using a solid-phase CD47 ELISA assay. His-CD47 was adsorbed to microtiter wells, washed and various concentrations of humanized mAbs were added to the wells for 1 hr at pH 6 or 8. The wells were washed and then incubated with HRP-labelled secondary antibody for 1 hour followed by addition of peroxidase substrate.

FIG. 9D. pH Dependent and pH Independent Binding of Humanized mAb to His-CD47. Binding of VLX8hum_10 IgG4PE to human CD47 was determined using a solid-phase CD47 ELISA assay. His-CD47 was adsorbed to microtiter wells, washed and various concentrations of humanized mAbs were added to the wells for 1 hr at pH 6 or 8. The wells were washed and then incubated with HRP-labelled secondary antibody for 1 hour followed by addition of peroxidase substrate.

FIG. 10. VLX4, VLX8, and VLX9 Humanized mAbs Block SIRPα binding to CD47 on Human Jurkat Cells. 1.5×10⁶ Jurkat cells were incubated with 5 μg/ml of VLX4, VLX8 and VLX9 CD47 humanized mAbs (VLX4hum_01 IgG4 PE, VLX4hum_07 IgG4 PE, VLX8hum_10 IgG4 PE, VLX4hum_11 IgG4 PE, VLX9hum_03 IgG2, VLX9hum_06 IgG2, and VLX9hum_08 IgG2) or a control antibody in RPMI containing 10% media for 30 min at 37° C. An equal volume of fluorescently labeled SIRPα-Fc fusion protein was added and incubated for an additional 30 min at 37° C. Cells were washed and binding was assessed using flow cytometry.

FIG. 11. VLX4 CD47 Chimeric mAbs Increase Phagocytosis of Human Jurkat Cells by Human Macrophages. Human macrophages were plated at a concentration of 1×10⁴ cells per well in a 96 well plate and allowed to adhere for 24 hrs. 5×10⁴ CFSE (1 μM) labeled human Jurkat cells and 1 μg/ml of the VLX4 chimeric mAbs were added to the macrophage cultures and incubated at 37° C. for 2 hrs. Non-phagocytosed Jurkat cells were removed and macrophage cultures were washed extensively. Macrophages were trypsinized and stained for CD14. Flow cytometry was used to determine the percentage of CD14⁺/CFSE⁺ cells in the total CD14⁺ population.

FIG. 12A. VLX4 Humanized mAbs Increase Phagocytosis of Human Jurkat Cells by Human Macrophages. Human macrophages were plated at a concentration of 1×10⁴ cells per well in a 96 well plate and allowed to adhere for 24 hrs. 5×10⁴ CFSE (1 μM) labeled human Jurkat cells and 1 μg/ml of antibody were added to the macrophage cultures and incubated at 37° C. for 2 hrs. Non-phagocytosed Jurkat cells were removed and macrophage cultures were washed extensively. Macrophages were trypsinized and stained for CD14. Flow cytometry was used to determine the percentage of CD14⁺/CFSE⁺ cells in the total CD14⁺ population.

FIG. 12B. VLX4 Humanized mAbs Increase Phagocytosis of Human Jurkat Cells by Human Macrophages. Human macrophages were plated at a concentration of 1×10⁴ cells per well in a 96 well plate and allowed to adhere for 24 hrs. 5×10⁴ CFSE (1 μM) labeled human Jurkat cells and 1 μg/ml of antibody were added to the macrophage cultures and incubated at 37° C. for 2 hrs. Non-phagocytosed Jurkat cells were removed and macrophage cultures were washed extensively. Macrophages were trypsinized and stained for CD14. Flow cytometry was used to determine the percentage of CD14⁺/CFSE⁺ cells in the total CD14⁺ population.

FIG. 13A. VLX8 CD47 Chimeric mAbs Increase Phagocytosis of Human Jurkat Cells by Human Macrophages. Human macrophages were plated at a concentration of 1×10⁴ cells per well in a 96 well plate and allowed to adhere for 24 hrs. 5×10⁴ CFSE (1 μM) labeled human Jurkat cells and 1 μg/ml of the VLX8 chimeric mAbs were added to the macrophage cultures and incubated at 37° C. for 2 hrs. Non-phagocytosed Jurkat cells were removed and macrophage cultures were washed extensively. Macrophages were trypsinized and stained for CD14. Flow cytometry was used to determine the percentage of CD14⁺/CFSE⁺ cells in the total CD14⁺ population.

FIG. 13B. VLX8 Humanized mAbs Increase Phagocytosis of Human Jurkat Cells by Human Macrophages. Human macrophages were plated at a concentration of 1×10⁴ cells per well in a 96 well plate and allowed to adhere for 24 hrs. 5×10⁴ CFSE (1 μM) labeled human Jurkat cells and 1 μg/ml of antibody were added to the macrophage cultures and incubated at 37° C. for 2 hrs. Non-phagocytosed Jurkat cells were removed and macrophage cultures were washed extensively. Macrophages were trypsinized and stained for CD14. Flow cytometry was used to determine the percentage of CD14⁺/CFSE⁺ cells in the total CD14⁺ population.

FIG. 14A. VLX9 CD47 Chimeric mAbs Increase Phagocytosis of Human Jurkat Cells by Human Macrophages. Human macrophages were plated at a concentration of 1×10⁴ cells per well in a 96 well plate and allowed to adhere for 24 hours. 5×10⁴ CFSE (1 μM) labeled human Jurkat cells and 1 μg/ml of the VLX9 chimeric mAbs were added to the macrophage cultures and incubated at 37° C. for two hours. Non-phagocytosed Jurkat cells were removed and macrophage cultures were washed extensively. Macrophages were trypsinized and stained for CD14. Flow cytometry was used to determine the percentage of CD14+/CFSE+ cells in the total CD14+ population.

FIG. 14B. VLX9 Humanized mAbs Increase Phagocytosis of Human Jurkat Cells by Human Macrophages. Human macrophages were plated at a concentration of 1×10⁴ cells per well in a 96 well plate and allowed to adhere for 24 hours. 5×10⁴ CFSE (1 μM) labeled human Jurkat cells and 1 μg/ml of antibody were added to the macrophage cultures and incubated at 37° C. for two hours. Non-phagocytosed Jurkat cells were removed and macrophage cultures were washed extensively. Macrophages were trypsinized and stained for CD14. Flow cytometry was used to determine the percentage of CD14+/CFSE+ cells in the total CD14+ population.

FIG. 15A. Induction of Cell Death in Human Jurkat Cells by Soluble VLX4 Humanized mAbs. Jurkat cells (1×10⁴) were incubated with 1 μg/ml VLX4 humanized mAbs (VLX4hum_01 IgG1, VLX4hum_01 IgG4PE, VLX4hum_02 IgG1, VLX4hum_02 IgG4PE) in RPMI media for 24 hours at 37° C. Cells were then stained with annexin V and the signal was detected by flow cytometry. The data are shown as % of cells that are annexin V positive (annexin V⁺).

FIG. 15B. Induction of Cell Death in Human Jurkat Cells by Soluble VLX4 Humanized mAbs. Jurkat cells (1×10⁴) were incubated with 1 μg/ml VLX4 humanized mAbs (VLX4hum_01 IgG1, VLX4hum_01 IgG4PE, VLX4hum_02 IgG1, VLX4hum_02 IgG4PE) in RPMI media for 24 hours at 37° C. Cells were then stained with annexin V and 7-AAD and analyzed by flow cytometry. The data are shown as % of the cells that are annexin V positive/7-AAD negative (annexin V⁺/7-AAD⁻).

FIG. 15C. Induction of Cell Death in Human Jurkat Cells by Soluble VLX4 Humanized mAbs. Jurkat cells (1×10⁴) were incubated with 1 μg/ml VLX4 humanized mAbs (VLX4hum_01 IgG1, VLX4hum_01 IgG4PE, VLX4hum_02 IgG1, VLX4hum_02 IgG4PE) in RPMI media for 24 hours at 37° C. Cells were then stained with annexin V and 7-AAD and analyzed by flow cytometry. The data are shown as % of cells that are annexin V positive/7-AAD positive (annexin V⁺/7-AAD⁺).

FIG. 15D. Induction of Cell Death in Human Jurkat Cells by Soluble VLX4 Humanized mAbs. Jurkat cells (1×10⁴) were incubated with 1 μg/ml VLX4 humanized mAbs (VLX4hum_06 IgG4PE, VLX4hum_07 IgG4PE, VLX4hum_08 IgG4PE, VLX4hum_11 IgG4PE, VLX4hum_12 IgG4PE, VLX4hum_13 IgG4PE) in RPMI media for 24 hours at 37° C. Cells were then stained with annexin V and 7-AAD and analyzed by flow cytometry. The data are shown as the % of cells that are annexin V positive (annexin V⁺).

FIG. 15E. Induction of Cell Death in Human Jurkat Cells by Soluble VLX4 Humanized mAbs. Jurkat cells (1×10⁴) were incubated with 1 μg/ml VLX4 humanized mAbs (VLX4hum_06 IgG4PE, VLX4hum_07 IgG4PE, VLX4hum_08 IgG4PE, VLX4hum_11 IgG4PE, VLX4hum_12 IgG4PE, VLX4hum_13 IgG4PE) in RPMI media for 24 hours at 37° C. Cells were then stained with annexin V and 7-AAD by flow cytometry. The data are shown as the % of cells that are annexin V positive/7-AAD negative (annexin V⁺/7-AAD⁻).

FIG. 15F. Induction of Cell Death in Human Jurkat Cells by Soluble VLX4 Humanized mAbs. Jurkat cells (1×10⁴) were incubated with 1 μg/ml VLX4 humanized mAbs (VLX4hum_06 IgG4PE, VLX4hum_07 IgG4PE, VLX4hum_08 IgG4PE, VLX4hum_11 IgG4PE, VLX4hum_12 IgG4PE, VLX4hum_13 IgG4PE) in RPMI media for 24 hours at 37° C. Cells were then stained with annexin V and 7-AAD and analyzed by flow cytometry. The data are shown as the % of cells that are annexin V positive/7-AAD positive (annexin⁺/7-AAD⁺).

FIG. 16A. Induction of Cell Death in Human Jurkat Cells by Soluble VLX8 CD47 Chimeric mAbs. Jurkat cells (1×10⁴) were incubated with 1 μg/ml VLX8 chimeric mAbs (VLX8 IgG1 N297Q xi and VLX8 IgG4PE xi) in RPMI media for 24 hrs at 37° C. Cells were then stained with annexin V and analyzed by flow cytometry. The data are presented as % of cells that are annexin V positive (annexin V⁺).

FIG. 16B. Induction of Cell Death in Human Jurkat Cells by Soluble VLX8 Chimeric mAbs. Jurkat cells (1×10⁴) were incubated with 1 μg/ml VLX8 chimeric mAbs (VLX8 IgG1 N297Q xi and VLX8 IgG4PE xi) in RPMI media for 24 hrs at 37° C. Cells were then stained with annexin V and 7-AAD and analyzed by flow cytometry. The data are presented as the % of cells that are annexin V positive/7-AAD negative (annexin V⁺/7-AAD⁻).

FIG. 16C. Induction of Cell Death in Human Jurkat Cells by Soluble VLX8 Chimeric mAbs. Jurkat cells (1×10⁴) were incubated with 1 μg/ml VLX8 chimeric mAbs (VLX8 IgG1 N297Q xi and VLX8 IgG4PE (xi) in RPMI media for 24 hrs at 37° C. Cells were then stained with annexin V and 7-AAD and analyzed by flow cytometry. The data are presented as the % of cells that are annexin V positive/7-AAD positive (annexin V⁺/7-AAD⁺).

FIG. 16D. Induction of Cell Death in Human Jurkat Cells by Soluble VLX8 Humanized mAbs. Jurkat cells (1×10⁴) were incubated with 1 μg/ml VLX8 humanized mAbs (VLX8hum_02 IgG4PE, VLX8hum_04 IgG4PE, VLX8hum_07 IgG4PE and VLX8hum_08 IgG4PE) and chimeric mAb VLX8 IgG4PE in RPMI media for 24 hrs at 37° C. Cells were then stained with annexin V and analyzed by flow cytometry. The data are presented as the % of cells that are annexin V positive (annexin V⁺).

FIG. 16E. Induction of Cell Death in Human Jurkat Cells by Soluble VLX8 Humanized mAbs. Jurkat cells (1×10⁴) were incubated with 1 μg/ml VLX8 humanized mAbs (VLX8hum_02 IgG4PE, VLX8hum_04 IgG4PE, VLX8hum_07 IgG4PE and VLX8hum_08 IgG4PE) and chimeric mAb VLX8 IgG4PE in RPMI media for 24 hrs at 37° C. Cells were then stained with annexin V and 7-AAD and analyzed by flow cytometry. The data are shown as the % of cells that are annexin V positive/7-AAD negative (annexin V⁺/7-AAD⁻).

FIG. 16F. Induction of Cell Death in Human Jurkat Cells by Soluble VLX8 Humanized mAbs. Jurkat cells (1×10⁴) were incubated with 1 μg/ml VLX8 humanized mAbs (VLX8hum_02 IgG4PE, VLX8hum_04 IgG4PE, VLX8hum_07 IgG4PE and VLX8hum_08 IgG4PE) and chimeric mAb VLX8 IgG4PE in RPMI media for 24 hrs at 37° C. Cells were then stained with annexin V and 7-AAD and analyzed by flow cytometry. The data are shown as the % of cells that are annexin V positive/7-AAD positive (annexin V⁺/7-AAD⁺).

FIG. 17A. Induction of Cell Death of Human Jurkat Cells by Soluble VLX9 Chimeric mAbs. 1×10⁴ Jurkat cells were incubated with 1 μg/ml of the VLX9 CD47 chimeric mAbs (VLX9 IgG1 N297Q xi, VLX9 IgG2 xi and VLX9 IgG4PE xi) in RPMI media for 24 hours 37° C. Cells were then stained with annexin V and the signal analyzed by flow cytometry. The data are shown as % of cells that are annexin V positive (annexin V⁺).

FIG. 17B. Induction of Cell Death of Human Jurkat Cells by Soluble VLX9 Chimeric mAbs. 1×10⁴ Jurkat cells were incubated with 1 μg/ml of the VLX9 CD47 chimeric mAbs (VLX9 IgG1 N297Q xi, VLX9 IgG2 xi and VLX9 IgG4PE xi) in RPMI media for 24 hours 37° C. Cells were then stained with annexin V and 7-AAD and analyzed by flow cytometry. The data are shown as % of cells that are annexin V positive/7-AAD negative (annexin V⁺/7-AAD⁻).

FIG. 17C. Induction of Cell Death of Human Jurkat Cells by Soluble VLX9 Chimeric mAbs. 1×10⁴ Jurkat cells were incubated with 1 μg/ml of the VLX9 CD47 chimeric mAbs (VLX9 IgG1 N297Q xi, VLX9 IgG2 xi and VLX9 IgG4PE xi) in RPMI media for 24 hours 37° C. Cells were then stained with annexin V and 7-AAD and analyzed by flow cytometry. The data are shown as % of cells that are annexin V positive/7-AAD positive (annexin V⁺/7-AAD⁺).

FIG. 17D. Induction of Cell Death in Human Jurkat Cells by Soluble VLX9 Humanized mAbs. Jurkat cells (1×10⁴) were incubated with 1 μg/ml VLX9 humanized mAbs (VLX9hum_01 to 10 IgG1) and chimeric mAb VLX9 IgG2 xi in RPMI media for 24 hours at 37° C. Cells were then stained with annexin V and the signal was detected by flow cytometry. VLX9 IgG2 (xi) is a murine/human chimera. The data are shown as % of cells that are annexin V positive (annexin V⁺).

FIG. 17E. Induction of Cell Death in Human Jurkat Cells by Soluble VLX9 Humanized mAbs. Jurkat cells (1×10⁴) were incubated with 1 μg/ml VLX9 humanized mAbs (VLX9hum_01 to 10 IgG1) and chimeric mAb VLX9 IgG2 xi in RPMI media for 24 hours at 37° C. Cells were then stained with annexin V and 7-AAD and analyzed by flow cytometry. VLX9 IgG2 (xi) is a murine/human chimera. The data are shown as % of cells that are annexin V positive/7-AAD negative (annexin V⁺/7-AAD⁻).

FIG. 17F. Induction of Cell Death in Human Jurkat Cells by Soluble VLX9 Humanized mAbs. Jurkat cells (1×10⁴) were incubated with 1 μg/ml VLX9 humanized mAbs (VLX9hum_01 to 10 IgG1) and chimeric mAb VLX9 IgG2 xi in RPMI media for 24 hours at 37° C. Cells were then stained with annexin V and 7-AAD and analyzed by flow cytometry. VLX9 IgG2 (xi) is a murine/human chimera. The data are shown as the % of cells that are annexin V positive/7-AAD positive (annexin V⁺/7-AAD⁺).

FIG. 18. Induction of Mitochondrial Depolarization in Human Raji Cells by Soluble VLX4, VLX8 and VLX9 Humanized mAbs. 1×10⁵ cells/ml Raji cells were incubated with 10 g/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cells were washed and the change in JC-1 dye fluorescence was assessed using flow cytometry. The data are expressed as % of cells with mitochondrial depolarization.

FIG. 19. Soluble VLX4, VLX8 and VLX9 Humanized mAbs Cause an Increase in Cell Surface Calreticulin Expression on Human Raji Cells. 1×10⁵ cells/ml Raji cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cells were washed and calreticulin expression was assessed using flow cytometry. The data are expressed as % of cells that are calreticulin positive.

FIG. 20. Soluble VLX4, VLX8 and VLX9 Humanized mAbs Cause an Increase in Cell Surface Protein Disulfide-Isomerase A3 (PDIA3) Expression by Human Raji Cells. 1×10⁵ cells/ml Raji cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cells were washed and PDIA3 expression was assessed using flow cytometry. The data are expressed as % of cells that are PDIA3 positive.

FIG. 21. Soluble VLX4, VLX8 and VLX9 Humanized mAbs Increase Cell Surface HSP70 Expression by Human Raji Cells. 1×10⁵ cells/ml Raji cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cells were washed and HSP70 expression was assessed using flow cytometry. The data are expressed as % of cells that are HSP70 positive.

FIG. 22. Soluble VLX4, VLX8 and VLX9 Humanized mAbs Increase Cell Surface HSP90 Expression by Human Raji Cells. 1×10⁵ cells/ml Raji cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cells were washed and HSP90 expression was assessed using flow cytometry. The data are expressed as % of cells that are HSP90 positive.

FIG. 23. Soluble VLX4, VLX8 and VLX9 Humanized mAbs Increase Release of Adenosine Triphosphate (ATP) by Human Raji Cells. 1×10⁵ cells/ml Raji cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cell-free supernatant was collected and analyzed using an ATP determination kit. The data are expressed as pM ATP in the supernatant.

FIG. 24. Soluble VLX4, VLX8 and VLX9 Humanized mAbs Cause an Increase in Release of High Mobility Group Box 1 (HMGB1) by Human Raji Cells. _1×10⁵ cells/ml Raji cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_03 IgG2, VLX9hum_06 IgG2 and VLX9hum_08 IgG2), a negative IgG control antibody or 1 μM of mitoxantrone as a positive control in RPMI media at 37° C. for 24 hours. Cell-free supernatant was collected and analyzed using an HMGB1 immunoassay. The data are expressed as ng/ml of HMGB1 in the supernatant.

FIG. 25. Soluble VLX4, VLX8 and VLX9 Humanized mAbs Increase CXCL10 Release by Human Raji Cells. 1×10⁵ cells/ml Raji cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_03 IgG2, VLX9hum_06 IgG2 and VLX9hum_08 IgG2), a negative IgG control antibody or 1 μM of mitoxantrone as a positive control in RPMI media at 37° C. for 24 hours. Cell-free supernatant was collected and analyzed using an CXCL10 immunoassay. The data are expressed as pg/ml of CXCL10 in the supernatant.

FIG. 26. Induction Mitochondrial Depolarization in Human Jurkat Cells by Soluble VLX4, VLX8 and VLX9 Humanized mAbs. 1×10⁵ cells/ml Jurkat cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cells were washed and the change in JC-1 dye fluorescence was assessed using flow cytometry. The data are expressed as % of cells with mitochondrial depolarization.

FIG. 27. Soluble VLX4, VLX8 and VLX9 Humanized mAbs Increase Cell Surface Calreticulin Expression by Human Jurkat Cells. 1×10⁵ cells/ml Jurkat cells were incubated with g/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cells were washed and calreticulin expression was assessed using flow cytometry. The data are expressed as % of cells that are calreticulin positive.

FIG. 28. Soluble VLX4, VLX8 and VLX9 Humanized mAbs Increase Cell Surface PDIA3 Expression by Human Jurkat Cells. 1×10⁵ cells/ml Jurkat cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cells were washed and PDIA3 expression was assessed using flow cytometry. The data are expressed as % of cells that are PDIA3 positive.

FIG. 29. Soluble VLX4, VLX8 and VLX9 Humanized mAbs Increase Cell Surface HSP70 Expression by Human Jurkat Cells. 1×10⁵ cells/ml Jurkat cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cells were washed and HSP70 expression was assessed using flow cytometry. The data are expressed as % of cells that are HSP70 positive.

FIG. 30. Soluble VLX4, VLX8 and VLX9 Humanized mAbs Increase Cell Surface HSP90 Expression by Human Jurkat Cells. 1×10⁵ cells/ml Jurkat cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cells were washed and HSP90 expression was assessed using flow cytometry. The data are expressed as % of cells that are HSP90 positive.

FIG. 31. Soluble VLX4, VLX8 and VLX9 Humanized mAbs Increase ATP Release by Human Jurkat Cells. 1×10⁵ cells/ml Jurkat cells were incubated with 10 μg/ml of VLX4, VLX8 and VLX9 CD47 humanized mAbs (VLX4hum_01 IgG4 PE, VLX4hum_07 IgG4 PE, VLX8hum_11 IgG4 PE, VLX9hum_06 IgG2, VLX9hum_08 IgG2 and VLX9hum_03 IgG2), a negative IgG control antibody or 1 μM of mitoxantrone as a positive control in RPMI media at 37° C. for 24 hours. Cell-free supernatant was collected and analyzed using an ATP determination kit. The data are expressed as pM ATP in the supernatant.

FIG. 32. Soluble VLX4, VLX8 and VLX9 Humanized mAbs Increase HMGB1 Release by Human Jurkat Cells. 1×10⁵ cells/ml Jurkat cells were incubated with 10 μg/ml of VLX4, VLX8 and VLX9 CD47 humanized mAbs (VLX9hum_01 IgG2, VLX4hum_07 IgG4 PE, VLX8hum_11 IgG4 PE, VLX9hum_03 IgG2, VLX9hum_06 IgG2 and VLX9hum_08 IgG2), a negative IgG control antibody or 1 μM of mitoxantrone as a positive control in RPMI media at 37° C. for 24 hours. Cell-free supernatant was collected and analyzed using an HMGB1 immunoassay. The data are expressed as ng/ml of HMGB1 in the supernatant.

FIG. 33. Soluble VLX4hum_07 IgG4PE Humanized mAb Causes Synergistic or Additive Cell Death of Human Jurkat Cells in Combination with the Chemotherapeutic Agent Doxorubicin. 1×10⁵ cells/ml Jurkat cells were incubated with 0.03-10 μg/ml of VLX4hum_07 IgG4 PE alone, 0.3-100 nM of doxorubicin alone or a combination dose-response matrix of 0.03-10 μg/ml of VLX4hum_07 IgG4PE and 0.3-100 nM of doxorubicin in RPMI media at 37° C. for 24 hours. Cells were then stained with annexin V and 7-AAD and the annexin V positive/7-AAD negative (annexin V+/7-AAD−) cells were quantitated by flow cytometry.

FIG. 34. Soluble VLX4hum_07 IgG4PE Humanized mAb Causes Synergistic or Additive Cell Death of Human Jurkat Cells in Combination with the Chemotherapeutic Agent Doxorubicin. 1×10⁵ cells/ml Jurkat cells were incubated with 0.03-10 μg/ml of VLX4hum_07 IgG4 PE alone, 0.3-100 nM doxorubicin alone or a combination dose-response matrix of 0.03-10 μg/ml of VLX4hum_07 IgG4PE and 0.3-100 nM of doxorubicin in RPMI media at 37° C. for 24 hours. Cells were then stained with annexin V and 7-AAD and the annexin V positive/7-AAD positive (annexin V+/7-AAD+) cells were quantitated by flow cytometry.

FIG. 35. Soluble VLX4hum_07 IgG4PE Humanized mAb Causes Synergistic or Additive Increase in Cell Surface Calreticulin Expression by Human Jurkat Cells in Combination with the Chemotherapeutic Agent Doxorubicin. 1×10⁵ cells/ml Jurkat cells were incubated with 0.03-10 g/ml of VLX4hum_07 IgG4 PE alone, 0.3-100 nM doxorubicin alone or a combination dose-response matrix of 0.03-10 μg/ml of VLX4hum_07 IgG4PE and 0.3-100 nM of doxorubicin in RPMI media at 37° C. for 24 hours. Cells were washed and calreticulin expression was assessed using flow cytometry. The data are expressed as % of cells that are calreticulin positive.

FIG. 36. Soluble VLX4hum_07 IgG4PE Humanized mAb Causes Synergistic and/or Additive ATP Release by Human Jurkat Cells in Combination with the Chemotherapeutic Agent Doxorubicin. 1×10⁵ cells/ml Jurkat cells were incubated with 0.03-10 μg/ml of VLX4hum_07 IgG4 PE alone, 0.3-100 nM doxorubicin alone or a combination dose-response matrix of 0.03-10 g/ml of VLX4hum_07 IgG4PE and 0.3-100 nM of doxorubicin in RPMI media at 37° C. for 24 hours. Cell-free supernatant was collected and analyzed using an ATP determination kit. The data are expressed as pM ATP in the supernatant.

FIG. 37A. Agglutination of hRBCs by VLX4 Humanized mAbs. Hemagglutination was assessed following incubation of hRBCs with various concentrations of humanized VLX4 mAbs (25 μg/mL-0.4 ng/mL) (VLXhum_01 IgG1, VLX4hum_01 IgG4PE). Blood was diluted (1:50) and washed 3 times with PBS/EDTA/BSA. hRBCs were added to U-bottomed 96 well plates with equal volumes of the antibodies (75 μl) and incubated for 3 hrs at 37° C. and overnight at 4° C.

FIG. 37B. Agglutination of hRBCs by VLX8 Chimeric and Humanized mAbs. Hemagglutination was assessed following incubation of hRBCs with various concentrations of humanized VLX4 mAbs (25 μg/mL-0.4 ng/mL) (VLX8hum_01 IgG4PE, VLX8hum_02 IgG4PE, VLX8hum_03 IgG4PE, VLX8hum_08 IgG4PE, VLX8hum_09 IgG4PE, VLX8hum_10 IgG4PE, VLX8hum_11 IgG4PE) and the chimeric mAb VLX8 IgG4PE xi. Blood was diluted (1:50) and washed 3 times with PBS/EDTA/BSA. hRBCs were added to U-bottomed 96 well plates with equal volumes of the antibodies (75 μl) and incubated for 3 hrs at 37° C. and overnight at 4° C.

FIG. 38A. Agglutination of Human RBCs by VLX9 Humanized mAbs. Hemagglutination was assessed following incubation of human RBCs with various concentrations of VLX9 IgG2 chimera (xi) and humanized VLX9 mAbs (VLX9hum_01 IgG2 to VLX9hum_06 IgG2). Blood was diluted (1:50) and washed 3 times with PBS/EDTA/BSA. RBCs were added to U-bottomed 96 well plates with equal volumes of the antibodies (75 μl) and incubated for 3 hrs at 37° C. and overnight at 4° C.

FIG. 38B. Agglutination of Human RBCs by VLX9 Humanized mAbs. Hemagglutination was assessed following incubation of human RBCs with various concentrations of VLX9 IgG2 chimera (xi) and humanized VLX9 mAbs (VLX9hum_06 IgG2 to VLX9hum_10 IgG2). Blood was diluted (1:50) and washed 3 times with PBS/EDTA/BSA. RBCs were added to U-bottomed 96 well plates with equal volumes of the antibodies (75 μl) and incubated for 3 hrs at 37° C. and overnight at 4° C.

FIG. 39. VLX4 Humanized mAb Reduces Tumor Growth in Raji Xenograft Model. Female NSG mice were inoculated subcutaneously in the right flank with 0.1 mL of a 30% RPMI/70% Matrigel™ (BD Biosciences; Bedford, Mass.) mixture containing a suspension of 5×10⁶ Raji tumor cells. Five days following inoculation, tumor volumes were measured and mice with palpable tumor volumes of 31-74 mm³ were randomized into 8-10/group. VLX4hum_07 or PBS (control) administration was initiated at this time. Mice were treated with 5 mg/kg of antibody 5×/week for 4 weeks by intraperitoneal injection. Tumor volumes and body weights were recorded twice weekly.

FIG. 40. VLX8 Humanized mAb Reduces Tumor Growth in Raji Xenograft Model. Female NSG mice were inoculated subcutaneously in the right flank with 0.1 mL of a 30% RPMI/70% Matrigel™ (BD Biosciences; Bedford, Mass.) mixture containing a suspension of 5×10⁶ Raji tumor cells. Five days following inoculation, tumor volumes were measured and mice with palpable tumor volumes of 31-74 mm³ were randomized into 8-10/group. VLX8hum_10 or PBS (control) administration was initiated at this time. Mice were treated with 5 mg/kg of antibody 5×/week for 4 weeks by intraperitoneal injection. Tumor volumes and body weights were recorded twice weekly.

FIG. 41. VLX9 Humanized mAb Reduces Tumor Growth in Raji Xenograft Model. Female NSG mice were inoculated subcutaneously in the right flank with 0.1 mL of a 30% RPMI/70% Matrigel™ (BD Biosciences; Bedford, Mass.) mixture containing a suspension of 5×10⁶ Raji tumor cells. Five days following inoculation, tumor volumes were measured and mice with palpable tumor volumes of 31-74 mm³ were randomized into 8-10/group. VLX9hum_08 IgG2 or PBS (control) administration was initiated at this time. Mice were treated with 5 mg/kg of antibody 5×/week for 4 weeks by intraperitoneal injection. Tumor volumes and body weights were recorded twice weekly.

FIG. 42A. Hemoglobin Levels in Blood Following Administration of a Humanized VLX9 mAb to Cynomolgus Monkeys by Intravenous Infusion. VLX9hum_08 IgG2 or vehicle were administered as a one hour intravenous infusion a dose of 5 mg/kg on day 1 and a dose of 15 mg/kg on day 18. Hemoglobin levels were monitored throughout the study and normalized to control values.

FIG. 42B. RBC Levels in Blood Following Administration of Humanized VLX9 mAbs to Cynomolgus Monkeys by Intravenous Infusion. VLX9hum_08 IgG2 or vehicle was administered as a one hour intravenous infusion a dose of 5 mg/kg on day 1 and a dose of 15 mg/kg on day 18. RBC levels were monitored throughout the study and normalized to control values.

FIG. 43. Induction of Cell Death in Human OV90 Cells by Soluble VLX4hum_07 IgG4 PE Humanized mAbs. 1×10⁵ cells/ml OV90 cells were incubated with 0.03-3 μg/ml of VLX4hum_07 IgG4 PE or 0.42 μM doxorubicin in MBCD/199 media at 37° C. for 24 hours. Cells were then stained with annexin V and 7-AAD and the annexin V positive/7-AAD negative (annexin V+/7-AAD−) cells were quantitated by flow cytometry.

FIG. 44. Induction of Cell Death in Human OV90 Cells by Soluble VLX4hum_07 IgG4 PE Humanized mAbs. 1×10⁵ cells/ml OV90 cells were incubated with 0.03-3 μg/ml of VLX4hum_07 IgG4 PE or 0.42 μM doxorubicin in MBCD/199 media at 37° C. for 24 hours. Cells were then stained with annexin V and 7-AAD and the annexin V positive/7-AAD positive (annexin V+/7-AAD+) cells were quantitated by flow cytometry.

FIG. 45. Induction of Cell Death in Human OV90 Cells by Soluble VLX4hum_07 IgG4 PE Humanized mAbs. 1×10⁵ cells/ml OV90 cells were incubated with 0.03-3 μg/ml of VLX4hum_07 IgG4 PE or 0.42 μM doxorubicin in MBCD/199 media at 37° C. for 24 hours. Cells were washed and calreticulin expression was assessed using flow cytometry. The data are expressed as % of cells that are calreticulin positive.

FIG. 46. Induction of Cell Death in Human OV90 Cells by Soluble VLX9hum_06 IgG2 Humanized mAbs. 1×10⁵ cells/ml OV90 cells were incubated with 1-100 μg/ml of VLX9hum_06 IgG2 or 0.42 μM doxorubicin in MBCD/199 media at 37° C. for 24 hours. Cells were then stained with annexin V and 7-AAD and the annexin V positive/7-AAD negative (annexin V+/7-AAD−) cells were quantitated by flow cytometry.

FIG. 47. Induction of Cell Death in Human OV90 Cells by Soluble VLX9hum_06 IgG2 Humanized mAbs. 1×10⁵ cells/ml OV90 cells were incubated with 1-100 μg/ml of VLX9hum_06 IgG2 or 0.42 μM doxorubicin in MBCD/199 media at 37° C. for 24 hours. Cells were then stained with annexin V and 7-AAD and the annexin V positive/7-AAD positive (annexin V+/7-AAD+) cells were quantitated by flow cytometry.

FIG. 48. Induction of Cell Death in Human OV90 Cells by Soluble VLX9hum_06 IgG2 Humanized mAbs. 1×10⁵ cells/ml OV90 cells were incubated with 1-100 μg/ml of VLX9hum_06 IgG2 or 0.42 μM doxorubicin in MBCD/199 media at 37° C. for 24 hours. Cells were washed and calreticulin expression was assessed using flow cytometry. The data are expressed as % of cells that are calreticulin positive.

FIG. 49. Induction of Cell Death in Human OV90 Cells by Soluble VLX8hum_11 IgG4 PE Humanized mAbs. 1×10⁵ cells/ml OV90 cells were incubated with 0.03-3 μg/ml of VLX8hum_11 IgG4 PE or 0.42 μM doxorubicin in MBCD/199 media at 37° C. for 24 hours. Cells were then stained with annexin V and 7-AAD and the annexin V positive/7-AAD negative (annexin V+/7-AAD−) cells were quantitated by flow cytometry.

FIG. 50. Induction of Cell Death in Human OV90 Cells by Soluble VLX8hum_11 IgG4 PE Humanized mAbs. 1×10⁵ cells/ml OV90 cells were incubated with 0.03-3 μg/ml of VLX8hum_11 IgG4 PE or 0.42 μM doxorubicin in MBCD/199 media at 37° C. for 24 hours. Cells were then stained with annexin V and 7-AAD and the annexin V positive/7-AAD positive (annexin V+/7-AAD+) cells were quantitated by flow cytometry.

FIG. 51. Induction of Cell Death in Human OV90 Cells by Soluble VLX8hum_11 IgG4 PE Humanized mAbs. 1×10⁵ cells/ml OV90 cells were incubated with 0.03-3 μg/ml of VLX8hum_11 IgG4 PE or 0.42 μM doxorubicin in MBCD/199 media at 37° C. for 24 hours. Cells were washed and calreticulin expression was assessed using flow cytometry. The data are expressed as % of cells that are calreticulin positive.

FIG. 52. Soluble VLX4hum_07 IgG4PE Humanized mAb Causes Synergistic or Additive Cell Death of Human OV10/315 Cells in Combination with Doxorubicin. 1×10⁵ cells/ml OV10/315 cells were incubated with 0.03-1 μg/ml of VLX4hum_07 IgG4 PE alone, 0.005-0.42 μM of doxorubicin alone, or a combination dose-response matrix of 0.03-1 μg/ml of VLX4hum_07 IgG4PE and 0.005-0.42 μM of doxorubicin in RPMI media at 37° C. for 24 hours. Cells were then stained with annexin V and 7-AAD and the annexin V positive/7-AAD negative (annexin V+/7-AAD−) cells were quantitated by flow cytometry.

FIG. 53. Soluble VLX4hum_07 IgG4PE Humanized mAb Causes Synergistic or Additive Cell Death of Human OV10/315 Cells in Combination with Doxorubicin. 1×10⁵ cells/ml OV10/315 cells were incubated with 0.03-1 μg/ml of VLX4hum_07 IgG4 PE alone, 0.005-0.42 μM of doxorubicin alone or a combination dose-response matrix of 0.03-1 μg/ml of VLX4hum_07 IgG4PE and 0.005-0.42 μM of doxorubicin in RPMI media at 37° C. for 24 hours. Cells were then stained with annexin V and 7-AAD and the annexin V positive/7-AAD positive (annexin V+/7-AAD+) cells were quantitated by flow cytometry.

FIG. 54. Soluble VLX4hum_07 IgG4PE Humanized mAb Causes Synergistic or Additive Cell Death of Human OV10/315 Cells in Combination with Epirubicin. 1×10⁵ cells/ml OV10/315 cells were incubated with 0.03-1 μg/ml of VLX4hum_07 IgG4 PE alone, 0.005-0.42 μM of epirubicin alone or a combination dose-response matrix of 0.03-1 μg/ml of VLX4hum_07 IgG4PE and 0.005-0.42 μM of epirubicin in RPMI media at 37° C. for 24 hours. Cells were then stained with annexin V and 7-AAD and the annexin V positive/7-AAD negative (annexin V+/7-AAD−) cells were quantitated by flow cytometry.

FIG. 55. Soluble VLX4hum_07 IgG4PE Humanized mAb Causes Synergistic or Additive Cell Death of Human OV10/315 Cells in Combination with Epirubicin. 1×10⁵ cells/ml OV10/315 cells were incubated with 0.03-1 μg/ml of VLX4hum_07 IgG4 PE alone, 0.005-0.42 μM of epirubicin alone or a combination dose-response matrix of 0.03-1 μg/ml of VLX4hum_07 IgG4PE and 0.005-0.42 μM of epirubicin in RPMI media at 37° C. for 24 hours. Cells were then stained with annexin V and 7-AAD and the annexin V positive/7-AAD positive (annexin V+/7-AAD+) cells were quantitated by flow cytometry.

FIG. 56. Soluble VLX4hum_07 IgG4PE Humanized mAb Causes Synergistic or Additive Cell Death of Human OV10/315 Cells in Combination with Docetaxel. 1×10⁵ cells/ml OV10/315 cells were incubated with 0.03-1 μg/ml of VLX4hum_07 IgG4 PE alone, 0.002-0.135 μM of docetaxel alone or a combination dose-response matrix of 0.03-1 μg/ml of VLX4hum_07 IgG4PE and 0.002-0.135 μM of docetaxel in RPMI media at 37° C. for 24 hours. Cells were then stained with annexin V and 7-AAD and the annexin V positive/7-AAD negative (annexin V+/7-AAD−) cells were quantitated by flow cytometry.

FIG. 57. Soluble VLX4hum_07 IgG4PE Humanized mAb Causes Synergistic or Additive Cell Death of Human OV10/315 Cells in Combination with Docetaxel. 1×10⁵ cells/ml OV10/315 cells were incubated with 0.03-1 μg/ml of VLX4hum_07 IgG4 PE alone, 0.002-0.135 μM of docetaxel alone or a combination dose-response matrix of 0.03-1 μg/ml of VLX4hum_07 IgG4PE and 0.002-0.135 μM of docetaxel in RPMI media at 37° C. for 24 hours. Cells were then stained with annexin V and 7-AAD and the annexin V positive/7-AAD positive (annexin V+/7-AAD+) cells were quantitated by flow cytometry.

FIG. 58. Soluble VLX4hum_07 IgG4PE Humanized mAb Causes Synergistic or Additive Cell Death of Human OV10/315 Cells in Combination with Gemcitabine. 1×10⁵ cells/ml OV10/315 cells were incubated with 0.03-1 μg/ml of VLX4hum_07 IgG4 PE alone, 0.003-0.3 μM of gemcitabine alone or a combination dose-response matrix of 0.03-1 μg/ml of VLX4hum_07 IgG4PE and 0.003-0.3 μM of gemcitabine in RPMI media at 37° C. for 24 hours. Cells were then stained with annexin V and 7-AAD and the annexin V positive/7-AAD negative (annexin V+/7-AAD−) cells were quantitated by flow cytometry.

FIG. 59. Soluble VLX4hum_07 IgG4PE Humanized mAb Causes Synergistic or Additive Cell Death of Human OV10/315 Cells in Combination with Gemcitabine. 1×10⁵ cells/ml OV10/315 cells were incubated with 0.03-1 μg/ml of VLX4hum_07 IgG4 PE alone, 0.003-0.3 μM of gemcitabine alone or a combination dose-response matrix of 0.03-1 μg/ml of VLX4hum_07 IgG4PE and 0.003-0.3 μM of gemcitabine in RPMI media at 37° C. for 24 hours. Cells were then stained with annexin V and 7-AAD and the annexin V positive/7-AAD positive (annexin V+/7-AAD+) cells were quantitated by flow cytometry.

FIG. 60. Soluble VLX4hum_07 IgG4PE Humanized mAb Causes Synergistic or Additive Cell Death of Human OV10/315 Cells in Combination with Gemcitabine. 1×10⁵ cells/ml OV10/315 cells were incubated with 0.03-1 μg/ml of VLX4hum_07 IgG4 PE alone, 0.003-0.3 μM of gemcitabine alone or a combination dose-response matrix of 0.03-1 μg/ml of VLX4hum_07 IgG4PE and 0.003-0.3 μM of gemcitabine in RPMI media at 37° C. for 24 hours. Cells were washed and calreticulin expression was assessed using flow cytometry. The data are expressed as % of cells that are calreticulin positive and 7AAD−.

FIG. 61. Soluble VLX4hum_07 IgG4PE Humanized mAb Causes Synergistic or Additive Cell Death of Human OV10/315 Cells in Combination with Irinotecan. 1×10⁵ cells/ml OV10/315 cells were incubated with 0.03-1 μg/ml of VLX4hum_07 IgG4 PE alone, 0.63-51 nM of irinotecan alone or a combination dose-response matrix of 0.03-1 μg/ml of VLX4hum_07 IgG4PE and 0.63-51 nM of irinotecan in RPMI media at 37° C. for 24 hours. Cells were then stained with annexin V and 7-AAD and the annexin V positive/7-AAD negative (annexin V+/7-AAD−) cells were quantitated by flow cytometry.

FIG. 62. Soluble VLX4hum_07 IgG4PE Humanized mAb Causes Synergistic or Additive Cell Death of Human OV10/315 Cells in Combination with Irinotecan. 1×10⁵ cells/ml OV10/315 cells were incubated with 0.03-1 μg/ml of VLX4hum_07 IgG4 PE alone, 0.63-51 nM of irinotecan alone or a combination dose-response matrix of 0.03-1 μg/ml of VLX4hum_07 IgG4PE and 0.63-51 nM of irinotecan in RPMI media at 37° C. for 24 hours. Cells were then stained with annexin V and 7-AAD and the annexin V positive/7-AAD positive (annexin V+/7-AAD+) cells were quantitated by flow cytometry.

FIG. 63. Soluble VLX4hum_07 IgG4PE Humanized mAb Causes Synergistic or Additive Cell Death of Human OV10/315 Cells in Combination with Irinotecan. 1×10⁵ cells/ml OV10/315 cells were incubated with 0.03-1 μg/ml of VLX4hum_07 IgG4 PE alone, 0.63-51 nM of irinotecan alone or a combination dose-response matrix of 0.03-1 μg/ml of VLX4hum_07 IgG4PE and 0.63-51 nM of irinotecan in RPMI media at 37° C. for 24 hours. Cells were washed and calreticulin expression was assessed using flow cytometry. The data are expressed as % of cells that are calreticulin positive and 7AAD−.

FIG. 64. Soluble VLX4hum_07 IgG4PE Humanized mAb Causes Synergistic or Additive Cell Death of Human OV10/315 Cells in Combination with Oxaliplatin. 1×10⁵ cells/ml OV10/315 cells were incubated with 0.03-1 μg/ml of VLX4hum_07 IgG4 PE alone, 0.65-52.8 μM of oxaliplatin alone or a combination dose-response matrix of 0.03-1 μg/ml of VLX4hum_07 IgG4PE and 0.65-52.8 μM of oxaliplatin in RPMI media at 37° C. for 24 hours. Cells were then stained with annexin V and 7-AAD and the annexin V positive/7-AAD negative (annexin V+/7-AAD−) cells were quantitated by flow cytometry.

FIG. 65. Soluble VLX4hum_07 IgG4PE Humanized mAb Causes Synergistic or Additive Cell Death of Human OV10/315 Cells in Combination with Oxaliplatin. 1×10⁵ cells/ml OV10/315 cells were incubated with 0.03-1 μg/ml of VLX4hum_07 IgG4 PE alone, 0.65-52.8 μM of oxaliplatin alone or a combination dose-response matrix of 0.03-1 μg/ml of VLX4hum_07 IgG4PE and 0.65-52.8 μM of oxaliplatin in RPMI media at 37° C. for 24 hours. Cells were then stained with annexin V and 7-AAD and the annexin V positive/7-AAD positive (annexin V+/7-AAD+) cells were quantitated by flow cytometry.

FIG. 66. Soluble VLX9hum_06 IgG4PE Humanized mAb Causes Synergistic or Additive Cell Death of Human Jurkat Cells in Combination with the Chemotherapeutic Agent Doxorubicin. 1×10⁵ cells/ml Jurkat cells were incubated with 1-100 μg/ml of VLX9hum_06 IgG2 alone, 0.005-0.42 μM of doxorubicin alone or a combination dose-response matrix of 1-100 μg/ml of VLX9hum_06 IgG2 and 0.005-0.42 μM of doxorubicin in RPMI media at 37° C. for 24 hours. Cells were then stained with annexin V and 7-AAD and the annexin V positive/7-AAD negative (annexin V+/7-AAD−) cells were quantitated by flow cytometry.

FIG. 67. Soluble VLX9hum_06 IgG4PE Humanized mAb Causes Synergistic or Additive Cell Death of Human Jurkat Cells in Combination with the Chemotherapeutic Agent Doxorubicin. 1×10⁵ cells/ml Jurkat cells were incubated with 1-100 μg/ml of VLX9hum_06 IgG2 alone, 0.005-0.42 μM of doxorubicin alone or a combination dose-response matrix of 1-100 μg/ml of VLX9hum_06 IgG2 and 0.005-0.42 μM of doxorubicin in RPMI media at 37° C. for 24 hours. Cells were then stained with annexin V and 7-AAD and the annexin V positive/7-AAD positive (annexin V+/7-AAD+) cells were quantitated by flow cytometry.

FIG. 68. Soluble VLX9hum_06 IgG4PE Humanized mAb Causes Synergistic or Additive Cell Death of Human Jurkat Cells in Combination with the Chemotherapeutic Agent Doxorubicin. 1×10⁵ cells/ml Jurkat cells were incubated with 1-100 μg/ml of VLX9hum_06 IgG2 alone, 0.005-0.42 μM of doxorubicin alone or a combination dose-response matrix of 1-100 μg/ml of VLX9hum_06 IgG2 and 0.005-0.42 μM of doxorubicin in RPMI media at 37° C. for 24 hours. Cells were washed and calreticulin expression was assessed using flow cytometry. The data are expressed as % of cells that are calreticulin positive and 7AAD−.

FIG. 69. Soluble VLX8hum_11 IgG4PE Humanized mAb Causes Synergistic or Additive Cell Death of Human Jurkat Cells in Combination with the Chemotherapeutic Agent Doxorubicin. 1×10⁵ cells/ml Jurkat cells were incubated with 0.03-3 μg/ml of VLX8hum_11 IgG4 PE alone, 0.005-0.42 μM of doxorubicin alone or a combination dose-response matrix of 0.03-3 μg/ml of VLX8hum_11 IgG4 PE and 0.005-0.42 μM of doxorubicin in RPMI media at 37° C. for 24 hours. Cells were then stained with annexin V and 7-AAD and the annexin V positive/7-AAD negative (annexin V+/7-AAD−) cells were quantitated by flow cytometry.

FIG. 70. Soluble VLX8hum_11 IgG4PE Humanized mAb Causes Synergistic or Additive Cell Death of Human Jurkat Cells in Combination with the Chemotherapeutic Agent Doxorubicin. 1×10⁵ cells/ml Jurkat cells were incubated with 0.03-3 μg/ml of VLX8hum_11 IgG4 PE alone, 0.005-0.42 μM of doxorubicin alone or a combination dose-response matrix of 0.03-3 μg/ml of VLX8hum_11 IgG4 PE and 0.005-0.42 μM of doxorubicin in RPMI media at 37° C. for 24 hours. Cells were then stained with annexin V and 7-AAD and the annexin V positive/7-AAD positive (annexin V+/7-AAD+) cells were quantitated by flow cytometry.

FIG. 71. Soluble VLX8hum_11 IgG4PE Humanized mAb Causes Synergistic or Additive Cell Death of Human Jurkat Cells in Combination with the Chemotherapeutic Agent Doxorubicin. 1×10⁵ cells/ml Jurkat cells were incubated with 0.03-3 μg/ml of VLX8hum_11 IgG4 PE alone, 0.005-0.42 μM of doxorubicin alone or a combination dose-response matrix of 0.03-3 μg/ml of VLX8hum_11 IgG4 PE and 0.005-0.42 μM of doxorubicin in RPMI media at 37° C. for 24 hours. Cells were washed and calreticulin expression was assessed using flow cytometry. The data are expressed as % of cells that are calreticulin positive and 7AAD−.

FIG. 72. Soluble VLX8hum_11 IgG4PE Humanized mAb Causes Synergistic or Additive Cell Death of Human Jurkat Cells in Combination with the Chemotherapeutic Agent Doxorubicin. 1×10⁵ cells/ml Jurkat cells were incubated with 0.03-3 μg/ml of VLX8hum_11 IgG4 PE alone, 0.005-0.42 μM of doxorubicin alone or a combination dose-response matrix of 0.03-3 μg/ml of VLX8hum_11 IgG4 PE and 0.005-0.42 μM of doxorubicin in RPMI media at 37° C. for 24 hours. Cell-free supernatant was collected and analyzed using an HMGB1 ELISA kit. The data are expressed as ng/ml HMGB1 in the supernatant.

FIG. 73. Humanized Anti-CD47 mAb Reduces Tumor Growth in MDA-MB-231 Xenograft Model. Female NSG mice were inoculated orthotopically into the mammary fat pad with 0.2 mL of a 70% RPMI/30% Matrigel™ (BD Biosciences; Bedford, Mass.) mixture containing a suspension of 2×10⁷ MDA-MB-231t tumor cells. Nineteen days following inoculation, tumor volumes were measured and mice with palpable tumor volumes of 55-179 mm were randomized into 10/group. Administration of a humanized anti-CD47 mAb VLX8hum_10 IgG4PE or PBS (control) was initiated at this time. Mice were treated with 5 mg/kg of antibody 5×/week for 5 weeks by intraperitoneal (IP) injection. Tumor volumes and body weights were recorded twice weekly.

FIG. 74. Humanized VLX9hum_06 IgG2 mAb Reduces Tumor Growth and Promotes Complete Regression of Tumors in Combination with Bortezomib in RPMI-8226 Xenograft Model. Female NSG mice were inoculated subcutaneously in the right flank with 0.2 mL of a 70% RPMI/30% Matrigel™ (BD Biosciences; Bedford, Mass.) mixture containing a suspension of 2×10⁷ RPMI-8226 tumor cells. Fifteen days following inoculation, tumor volumes were measured and mice with palpable tumor volumes of 50-100 mm³ were randomized into 10/group. Administration of a VLX9hum_06 IgG2, a control antibody, and bortezomib was initiated at this time. Mice were treated with 10 or 25 mg/kg of antibody once weekly for 6 weeks by intravenously (IV) injection. Bortezomib was administered IV at 1 mg/kg for 3 cycles. Primary assessment of efficacy was monitored by measurement of tumor volumes.

FIG. 75. Humanized VLX9hum06_IgG2 mAb as a Single Agent and in Combination with Bortezomib Promotes Increased Survival of Mice in an RPMI-8226 Xenograft Model. Secondary assessment of efficacy was assessed by monitoring survival of tumor bearing mice in control, VLX9hum_06 IgG2 monotherapy and combination VLX9hum_06 IgG2 with Bortezomib treatment groups.

FIG. 76. VLX9hum_06 IgG2 mAb Increase Phagocytosis of Human SNU-1 Cells by Human Macrophages. Human macrophages were plated at a concentration of 1×10⁴ cells per well in a 96 well plate. 5×10⁴ CFSE (1 μM) labeled human SNU-1 cells was incubated with increasing concentrations of VLX9hum_06 IgG2 and added to the macrophage cultures at 37° C. for two hours. Non-phagocytosed SNU-1 cells were removed and macrophage cultures were washed extensively. Macrophages were trypsinized and stained for CD14. Flow cytometry was used to determine the percentage of CD14+/CFSE+ cells in the total CD14+ population.

FIG. 77A. Soluble VLX9hum_06 IgG2 Humanized mAb Causes Cell Death of Human SNU-1 Gastric Carcinoma cells. 1×10⁵ cells/ml SNU-1 cells were incubated with increasing concentrations of VLX9hum_06 IgG2 in RPMI media at 37° C. for 24 hours. Cells were then stained with annexin V and total annexin V labeling was quantitated by flow cytometry.

FIG. 77B-FIG. 77D. Soluble VLX9hum_06 IgG2 Humanized mAb Causes Additive Cell Death of Human SNU-1, Hs746T, or KATOIII Gastric Carcinoma cells in Combination with the Chemotherapeutic Agent Cisplatin. 1×10⁵ cells/ml SNU-1 (FIG. 77B), Hs746T (FIG. 77C), or KATOIII (FIG. 77D) gastric carcinoma cells were incubated with 100 μg/ml of VLX9hum_06 IgG2 alone, 1.3-3.3 μM of cisplatin alone or a combination of VLX9hum_06 IgG2 and 1.3-33.3 μM of cisplatin in RPMI media at 37° C. for 24 hours. Cells were then stained with annexin V and total annexin V labeling was quantitated by flow cytometry.

FIG. 77E-FIG. 77G. Soluble VLX9hum_06 IgG2 Humanized mAb Causes Additive Cell Death of Human SNU-1, Hs746T, or KATOIII Gastric Carcinoma cells in Combination with the Chemotherapeutic Agent Paclitaxel. 1×10⁵ cells/ml SNU-1 (FIG. 77E), Hs746T (FIG. 77F), or KATOIII (FIG. 77G) gastric carcinoma cells were incubated with 100 μg/ml of VLX9hum_06 IgG2 alone, 0.2-1.1 μM of paclitaxel alone or a combination of VLX9hum_06 IgG2 and 0.2-1.1 μM of paclitaxel in RPMI media at 37° C. for 24 hours. Cells were then stained with annexin V and total annexin V labeling was quantitated by flow cytometry.

FIG. 78. VLX9hum_06 IgG2 mab reduces tumor growth in SNU-1 xenograft model as a single agent and in combination with cisplatin. Female NSG mice were inoculated subcutaneously into the right flank with 0.2 mL of a 70% RPMI/30% Matrigel™ (BD Biosciences; Bedford, Mass.) mixture containing a suspension of 5×10⁶ SNU-1 tumor cells. Eight days following inoculation, tumor volumes were measured and mice with palpable tumor volumes of 50-100 mm3 were randomized into 10/group. IgG2 control, VLX9hum_06 IgG2 alone, cisplatin alone or VLX9hum_06 IgG2 in combination with cisplatin was initiated at this time. Mice were treated with 25 mg/kg of antibody once weekly for 5 weeks by intraperitoneal injection. Cisplatin was administered at 3 mg/kg once weekly for 4 weeks. Tumor volumes and body weights were recorded twice weekly.

FIG. 79A-FIG. 79B. VLX9hum_06 IgG2 inhibits tumor growth in OV90 ovarian carcinoma xenograft models as a single agent and in combination with chemotherapy. Human OV90 ovarian cells were injected subcutaneously into NSG mice (N=10/group). Mice were randomized into 4 treatment groups with an average volume of 71 mm3/group. VLX9hum_06 IgG2 or IgG2 control at 25 mg/kg was administered QD×5 week for 6 weeks. Cisplatin at 5 mg/kg, Paclitaxel at 20 mg/kg or vehicle control (VC) was administered IP. Tumor volume (mm³) was measured twice/week.

FIG. 80. Cytokine and Chemokine Release in the Xengraft OV90 Tumor Micro-Environment (TME). Human OV90 ovarian cells were injected subcutaneously into NSG mice. Anti-CD47 mAb VLX9hum_06 IgG2 or IgG2 control at a concentration of 10 mg/kg were administered IP daily for a total of 5 days. Tumors were excised at 48 hours, 96 hours, or Day 7 post first dose of anti-CD47 mAb VLX9hum_06 IgG2. Tumors (N=3/group) were quantified for murine cytokines (IL-1β and IL-10) and chemokines (MCP-1, IP-10 and MIP-1α).

FIG. 81. Human RPMI-8226 multiple myeloma cells were injected subcutaneously (SC) into NSG mice. IgG2 control (25 mg/kg), VLX9hum_06 IgG2 mAb (10 mg/kg), or VLX9hum_06 IgG2 (25 mg/kg) was administered intravenously (IV) on Day 0. Bortezomib (1 mg/kg) was administered IV on Day 1 and Day 4. Tumors were excised at 96 hrs or on Day 10 post dosing of VLX9hum_06 IgG2. The micrographs show tumors assayed by immunohistochemistry for murine CD11c, a marker of dendritic cells.

FIG. 82. Human RPMI-8226 multiple myeloma cells were injected subcutaneously (SC) into NSG mice. IgG2 control (25 mg/kg), VLX9hum_06 IgG2 mAb (10 mg/kg), or VLX9hum_06 IgG2 (25 mg/kg) was administered intravenously (IV) on Day 0. Bortezomib (1 mg/kg) was administered IV on Day 1 and Day 4. Tumors (N=3/group) were excised at 48 hrs, 96 hrs or on Day 10 post dosing of VLX9hum_06 IgG2 and quantified for murine cytokines and chemokines.

FIG. 83. Pharmacokinetic of VLX9hum_06 IgG2 mAb following intravenous (IV) dosing in RPMI-8226 tumor bearing NSG mice. Dosing of VLX9hum_06 IgG2 mAb is on Day 0 and Day 7.

FIG. 84A-FIG. 84B. Humanized Anti-CD47 mAb VLX9hum_06 IgG2 Reduces Tumor Growth and Promotes Complete Regression of Tumors and Increases Survival in Combination with bortezomib in MM.1S Multiple Myeloma Xenograft Model. Human MM.1S multiple myeloma were implanted subcutaneously into NOD-SCID mice (N=10/group). Mice received 25 mg/kg IgG2 or VLX9hum_06 IgG2 intraperitonealy (IP) on days 0, 7, 14 & 21 with or without Bortezomib (0.75 mg/kg on d0 and d3 and 0.5 mg/kg on d10 and d17) by intravenous (IV) injection. FIG. 84A shows efficacy of single and combination treatment. Tumor volumes were measured twice weekly and plotted versus day(s) post-treatment. FIG. 84B shows secondary assessment of efficacy was assessed by monitoring survival of tumor bearing mice in control, VLX9hum_06 IgG2 monotherapy and combination VLX9hum_06 IgG2 with bortezomib treatment groups.

FIG. 85A-FIG. 85B. Humanized Anti-CD47 mAb VLX9hum_06 IgG2 Promotes Potent Anti-Tumor Efficacy in Combination with daratumumab in MM.1S Multiple Myeloma Xenograft Model. Human MM.1S multiple myeloma cells were implanted subcutaneously into NOD-SCID mice (N=10/group). Mice received 25 mg/kg IgG2 or VLX9hum_06 IgG2 intraperitonealy (IP) on days 0, 7, 14 & 21 with or without Daratumumab (15 mg/kg twice weekly for 6 weeks) by IP injection. FIG. 85A shows efficacy of single and combination treatment. Tumor volumes were measured twice weekly and plotted versus day(s) post-treatment. FIG. 85B shows secondary assessment of efficacy was assessed by monitoring survival of tumor bearing mice in control, VLX9hum_06 IgG2 monotherapy and combination VLX9hum_06 IgG2 with daratumumab treatment groups.

FIG. 86A-FIG. 86B. Humanized Anti-CD47 mAb VLX9hum_06 IgG2 Promotes Potent Anti-Tumor in NCI-H929 Multiple Myeloma Xenograft Model. Human NCI-H929 multiple myeloma cells were implanted subcutaneously into NOD-SCID mice (N=8/group). Mice received 25 mg/kg IgG2 or VLX9hum_06 IgG2 intraperitonealy (IP) on days 0, 7, 14 & 21. FIG. 86A shows efficacy of single and combination treatment. Tumor volumes were measured twice weekly and plotted versus day(s) post-treatment. FIG. 86B shows a spider plot of tumor volumes in individual animals.

FIG. 87A-FIG. 87E. Anti-CD47 mAbs Increase Phagocytosis. VLX9hum_06 IgG2 mAbs increases phagocytosis of KG1, MV411, MOLM13, Ramos, and RAJI tumor cells by human macrophages in a dose dependent fashion compared to an IgG2 control antibody.

FIG. 88. Anti-CD47 mAbs Increase Phagocytosis When Combined With Anti-CD20 mAbs. VLX9hum_06 IgG2 mAbs increased phagocytosis of RAJI cells by human macrophages when combined with anti-CD20 mAbs compared to either agent alone.

FIG. 89A-FIG. 89C. Anti-CD47 mAbs Increase Phagocytosis of Multiple Myeloma Cells. A soluble anti-CD47 mAb increases phagocytosis of MM1.S, L363, and MOLP8 cells by human macrophages in a dose dependent fashion compared to a human IgG2 control antibody.

FIG. 90A-FIG. 90B. Anti-CD47 mAbs Mediated Cell Autonomous Killing of Multiple Myeloma Cells in Combination with Bortezomib. Cell autonomous killing was assessed by treating U266B1 and MOLP8 cells with anti-CD47 mAbs in combination with bortezomib.

FIG. 91A. Humanized Anti-CD47 mAb VLX9hum_06 IgG2 Promotes Potent Anti-Tumor Efficacy in Combination with Lenalidomide in MM.1S Multiple Myeloma Xenograft Model. Human MM.1S multiple myeloma cells were implanted subcutaneously into NOD-SCID mice (N=9/group). Mice received 25 mg/kg IgG2 or VLX9hum_06 IgG2 via IP injection on days 0, 7, 14, 21, and 28 with or without lenalidomide (25 mg/kg on four successive days, then three off, weekly for 5 weeks) via oral gavage (PO). Tumor volumes were measured twice weekly and plotted versus day(s) following the initiation of treatment.

FIG. 91B. Humanized Anti-CD47 mAb VLX9hum_06 IgG2 Promotes Potent Anti-Tumor Efficacy in Combination with Pomalidomide in MM.1S Multiple Myeloma Xenograft Model. Human MM.1S multiple myeloma cells were implanted subcutaneously into NOD-SCID mice (N=9/group). Mice received 25 mg/kg IgG2 or VLX9hum_06 IgG2 via IP injection on days 0, 7, 14, 21, and 28 with or without pomalidomide (10 mg/kg on four successive days, then three off, weekly for 5 weeks) via oral gavage. Tumor volumes were measured twice weekly and plotted versus day(s) following the initiation of treatment.

FIG. 92A. Addition of Dexamethasone Does Not Compromise Potent Anti-Tumor Efficacy Resulting from Combination of Humanized Anti-CD47 mAb VLX9hum_06 IgG2 with Lenalidomide in MM.1S Multiple Myeloma Xenograft Model. Human MM.1S multiple myeloma cells were implanted subcutaneously into NOD-SCID mice (N=9/group). Mice received IgG2 (25 mg/kg) or VLX9hum_06 IgG2 (25 mg/kg) via IP injection on days 0, 7, 14, 21, and 28. Lenalidomide (25 mg/kg, PO) or dexamethasone (0.3 mg/kg, IP) was administered on four successive days, then three off, weekly for 5 weeks. Agent combinations were administered at same dosing frequency as single agent groups. Tumor volumes were measured twice weekly and plotted versus day(s) following the initiation of treatment.

FIG. 92B. Addition of Dexamethasone Does Not Compromise Potent Anti-Tumor Efficacy Resulting from Combination of Humanized Anti-CD47 mAb VLX9hum_06 IgG2 with Pomalidomide in MM.1S Multiple Myeloma Xenograft Model. Human MM.1S multiple myeloma cells were implanted subcutaneously into NOD-SCID mice (N=9/group). Mice received IgG2 (25 mg/kg) or VLX9hum_06 IgG2 (25 mg/kg) via IP injection on days 0, 7, 14, 21, and 28. Pomalidomide (10 mg/kg, PO) or dexamethasone (0.3 mg/kg, IP) was administered on four successive days, then three off, weekly for 5 weeks. Agent combinations were administered at same dosing frequency as single agent groups. Tumor volumes were measured twice weekly and plotted versus day(s) following the initiation of treatment.

FIG. 93A. Humanized Anti-CD47 mAb VLX9hum_06 IgG2 Promotes Accumulation of CD68⁺ and CD11c⁺ Cells at Tumor Periphery in HCI-H929 Multiple Myeloma Xenograft Model. Human NCI-H929 multiple myeloma cells were implanted subcutaneously into NOD-SCID mice (N=3/group). Mice received 25 mg/kg hIgG2 or VLX9hum_06 IgG2, then 96 hours later tumors were harvested, fixed, and immunohistochemical staining for murine CD68 and murine CD11c performed. Arrows denote areas of positive staining cells.

FIG. 93B. Humanized Anti-CD47 mAb VLX9hum_06 IgG2 Promotes Accumulation of CD68⁺ and CD11c⁺ Cells at Tumor Periphery in RPMI-8226 Multiple Myeloma Xenograft Model. Human RPMI-8226 multiple myeloma cells were implanted subcutaneously into NOD-SCID mice (N=3/group). Mice received 25 mg/kg hIgG2 or VLX9hum_06 IgG2, then 96 hours later tumors were harvested, fixed, and immunohistochemical staining for murine CD68 and murine CD11c performed. Arrows denote areas of positive staining cells.

FIG. 94A-FIG. 94B. Humanized Anti-CD47 mAb VLX9hum_06 IgG2 Promotes Potent Anti-Tumor Efficacy at Multiple Dosing Concentrations in NCI-H929 Multiple Myeloma Xenograft Model. Human NCI-H929 multiple myeloma cells were implanted subcutaneously into NOD-SCID mice (N=6/group). Mice received 25 mg/kg hIgG2 or VLX9hum_06 IgG2 at doses of 1, 3, 10, or 25 mg/kg weekly via IP injection. FIG. 94A shows each dose of antibody in a spider plot of tumor volumes in individual animals. Tumor volumes were measured twice weekly and plotted versus day(s) following the initiation of treatment. FIG. 94B shows secondary assessment of efficacy was assessed by monitoring survival of tumor bearing mice in control and various VLX9hum_06 IgG2 treatment groups.

FIG. 95A. Treatment with Humanized Anti-CD47 mAb VLX9hum_06 IgG2 Results in Potent Tumor Growth Inhibition in a Human Multiple Myeloma Xenograft Model of Advanced Disease Burden. Human NCI-H929 multiple myeloma cells were implanted subcutaneously into NOD-SCID mice (N=6/group) then randomized and treatment initiated when tumors reached a volume of 200-1600 mm³. Mice received 25 mg/kg hIgG2 or VLX9hum_06 IgG2 weekly via IP injection. Tumor volumes were measured twice weekly and plotted versus day(s) following the initiation of treatment.

FIG. 95B. Treatment with Humanized Anti-CD47 mAb VLX9hum_06 IgG2 Potently Extends Survival in a Human Multiple Myeloma Xenograft Model of Advanced Disease Burden. Human NCI-H929 multiple myeloma cells were implanted subcutaneously into NOD-SCID mice (N=6/group) then randomized and treatment initiated when tumors reached a volume of 200-1600 mm³. Mice received 25 mg/kg hIgG2 or VLX9hum_06 IgG2 weekly via IP injection. Tumor volumes were measured twice weekly and plotted versus day(s) following the initiation of treatment.

FIG. 96A-FIG. 96C. Anti-CD47 mAbs Increase Phagocytosis When Combined with 5-Azacitidine. Human monocyte derived macrophages were plated at a concentration of 5×10⁴ cells per well in a 96 well plate. 8×10⁴ CFSE (1 μM) labeled human HL-60 (FIG. 96A), MV4-11 (FIG. 96B), or KG-1 (FIG. 96C) acute myeloid leukemia cells were treated with 0.63 or 3 μM 5-azacitidine overnight prior to being incubated with VLX9hum_06 IgG2 and added to the macrophage cultures at 37° C. for two hours. Non-phagocytosed target tumor cells were removed, and macrophage cultures were washed extensively. Macrophages were trypsinized and stained for CD14 prior to analysis by flow cytometry. Percent (%) phagocytosis is calculated as the percent (%) of CFSE+/CD14+ of the total CD14+ macrophages. Figures show single concentrations of each agent alone, or in combination, as optimized per each cell line.

FIG. 97A-FIG. 97C. Anti-CD47 mAbs Increase Phagocytosis When Combined with Venetoclax. Human monocyte derived macrophages were plated at a concentration of 5×10⁴ cells per well in a 96 well plate. 8×10⁴ CFSE (1 μM) labeled human HL-60 (FIG. 97A), MV4-11 (FIG. 97B), or KG-1 (FIG. 97C) acute myeloid leukemia cells were treated with 3 nM, 10 nM, or 0.5 μM venetoclax, respectively, overnight prior to being incubated with VLX9hum_06 IgG2 and added to the macrophage cultures at 37° C. for two hours. Non-phagocytosed target tumor cells were removed, and macrophage cultures were washed extensively. Macrophages were trypsinized and stained for CD14 prior to analysis by flow cytometry. Percent (%) phagocytosis is calculated as the percent (%) of CFSE+/CD14+ of the total CD14+ macrophages. Figures show single concentrations of each agent alone, or in combination, as optimized per each cell line.

FIG. 98A-FIG. 98B. Anti-CD47 mAbs Enhances Cell Killing in Combination with 5-Azacitidine. HL-60 (FIG. 98A) or MV4-11 (FIG. 98B) acute myeloid leukemia cells were incubated with 100 μg/mL VLX9hum_06 IgG2 alone, 5 μM 5-azacitidine alone, or a combination of VLX9hum_06 IgG2 and 5-azacitidine in RPMI media at 37° C. for 24 hours. Cells were washed and then stained with Annexin V PE and SYTOX Blue followed by analysis by flow cytometry.

FIG. 99A-FIG. 99B. Anti-CD47 mAbs Enhances Cell Killing in Combination with Venetoclax. MV4-11 (FIG. 99A) or KG-1 (FIG. 99B) acute myeloid leukemia cells were incubated with 100 μg/mL VLX9hum_06 IgG2 alone, 0.3 or 2.5 μM venetoclax alone, or a combination of VLX9hum_06 IgG2 and venetoclax in RPMI media at 37° C. for 24 hours. Cells were washed and then stained with Annexin V PE and SYTOX Blue followed by analysis by flow cytometry.

FIG. 100A. Anti-CD47 mAbs Enhances DAMP Induction Alone. HL-60 (FIG. 100A) acute myeloid leukemia cells were incubated with 10, 30 or 100 μg/mL VLX9hum_06 IgG2 alone in RPMI media at 37° C. for 24 hours. Cells were washed and then stained for calreticulin and SYTOX Blue followed by analysis by flow cytometry. Cell surface exposure of calreticulin was increased by treatment with VLX9hum_06 IgG2 in a concentration-dependent manner.

FIG. 100B. Anti-CD47 mAbs Enhances DAMP Induction in Combination with 5-Azacitidine. HL-60 (FIG. 100B) acute myeloid leukemia cells were incubated with 100 μg/mL VLX9hum_06 IgG2 alone, 5 μM 5-azacitidine alone, or a combination of VLX9hum_06 IgG2 and 5-azacitidine in RPMI media at 37° C. for 24 hours. Cells were washed and then stained for PDIA3 and SYTOX Blue followed by analysis by flow cytometry. Cell surface exposure of PDIA3 was increased by treatment with VLX9hum_06 IgG2 and further enhanced in combination with 5-azacitidine.

### DETAILED DESCRIPTION OF THE DISCLOSURE

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo or polynucleotide chemistry and hybridization described herein are those well-known and commonly used in the art.

As used herein, the term “CD47”, “integrin-associated protein (IAP)”, “ovarian cancer antigen OA3”, “Rh-related antigen” and “MERG” are synonymous and may be used interchangeably.

The term “anti-CD47 antibody” refer to an antibody of the disclosure which is intended for use as a therapeutic or diagnostic agent, and therefore will typically possess the binding affinity required to be useful as a therapeutic and/or diagnostic agent.

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts” with or directed against is meant that the antibody reacts with one or more antigenic determinants of the desired antigen and does not react with other polypeptides or binds at a much lower affinity (Kd>10⁻⁶). Antibodies include but are not limited to, polyclonal, monoclonal, chimeric, Fab fragments, Fab′ fragments, F(ab′)2 fragments, single chain Fv fragments, and one-armed antibodies.

As used herein, the term “monoclonal antibody” (mAb) as applied to the present antibody compounds refers to an antibody that is derived from a single copy or clone including, for example, any eukaryotic, prokaryotic, or phage clone, and not the method by which it is produced. mAbs of the present disclosure preferably exist in a homogeneous or substantially homogeneous population. Complete mAbs contain 2 heavy chains and 2 light chains.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

As disclosed herein, “antibody compounds” refers to mAbs and antigen-binding fragments thereof. Additional antibody compounds exhibiting similar functional properties according to the present disclosure can be generated by conventional methods. For example, mice can be immunized with human CD47 or fragments thereof, the resulting antibodies can be recovered and purified, and determination of whether they possess binding and functional properties similar to or the same as the antibody compounds disclosed herein can be assessed by the methods disclosed in Examples 3-16, below. Antigen-binding fragments can also be prepared by conventional methods. Methods for producing and purifying antibodies and antigen-binding fragments are well known in the art and can be found, for example, in Harlow and Lane (1988) Antibodies, A Laboratory Manual, Cold Spring Harbor Laboratory Press, Cold Spring Harbor, N.Y., chapters 5-8 and 15.

The monoclonal antibodies encompass antibodies in which a portion of the heavy and/or light chain is identical with, or homologous to, corresponding sequences in murine antibodies, in particular the murine CDRs, while the remainder of the chain(s) is (are) identical with, or homologous to, corresponding sequences in human antibodies. Other embodiments of the disclosure include antigen-binding fragments of these monoclonal antibodies that exhibit binding and biological properties similar or identical to the monoclonal antibodies. The antibodies of the present disclosure can comprise kappa or lambda light chain constant regions, and heavy chain IgA, IgD, IgE, IgG, or IgM constant regions, including those of IgG subclasses IgG1, IgG2, IgG3, and IgG4 and in some cases with various mutations to alter Fc receptor function.

The monoclonal antibodies containing the presently disclosed murine CDRs can be prepared by any of the various methods known to those skilled in the art, including recombinant DNA methods.

Reviews of current methods for antibody engineering and improvement can be found, for example, in P. Chames, Ed., (2012) Antibody Engineering: Methods and Protocols, Second Edition (Methods in Molecular Biology, Book 907), Humana Press, ISBN-10: 1617799734; C. R. Wood, Ed., (2011) Antibody Drug Discovery (Molecular Medicine and Medicinal Chemistry, Book 4), Imperial College Press; R. Kontermann and S. Dubel, Eds., (2010) Antibody Engineering Volumes 1 and 2 (Springer Protocols), Second Edition; and W. Strohl and L. Strohl (2012) Therapeutic antibody engineering: Current and future advances driving the strongest growth area in the pharmaceutical industry, Woodhead Publishing.

Methods for producing and purifying antibodies and antigen-binding fragments are well known in the art and can be found, for example, in Harlow and Lane (1988) Antibodies, A Laboratory Manual, Cold Spring Harbor Laboratory Press, Cold Spring Harbor, N.Y., chapters 5-8 and 15.

A full-length antibody as it exists naturally is a “Y” shaped immunoglobulin (Ig) molecule comprising four polypeptide chains: two identical heavy (H) chains and two identical light (L) chains, interconnected by disulfide bonds. The amino terminal portion of each chain, termed the fragment antigen binding region (FAB), includes a variable region of about 100-110 or more amino acids primarily responsible for antigen recognition via the complementarity determining regions (CDRs) contained therein. The carboxy-terminal portion of each chain defines a constant region (the “Fc” region) primarily responsible for effector function.

The CDRs are interspersed with regions that are more conserved, termed frameworks (“FRs”). Amino acid sequences of many FRs are well known in the art. Each light chain variable region (LCVR) and heavy chain variable region (HCVR) is composed of 3 CDRs and 4 FRs, arranged from amino-terminus to carboxy-terminus in the following order: FR1, CDR1, FR2, CDR2, FR3, CDR3, FR4. The 3 CDRs of the light chain are referred to as “LCDR1, LCDR2, and LCDR3” and the 3 CDRs of the heavy chain are referred to as “HCDR1, HCDR2, and HCDR3.” The CDRs contain most of the residues which form specific interactions with the antigen. The numbering and positioning of CDR amino acid residues within the LCVR and HCVR regions are in accordance with the well-known Kabat numbering convention Kabat et al. (1991) Sequences of Proteins of Immunological Interest, Fifth Edition. NIH Publication No. 91-3242.

As described herein, the “antigen-binding site” can also be defined as the “Hypervariable regions”, “HVRs”, or “HVs”, and refer to the structurally hypervariable regions of antibody variable domains as defined by Chothia and Lesk (Chothia and Lesk, Mol. Biol. 196:901-917, 1987). There are six HVRs, three in VH (H1, H2, H3) and three in VL (L1, L2, L3). We used herein CDRs as defined by Kabat except in H-CDR1, which is extended to include H1.

There are five types of mammalian immunoglobulin (Ig) heavy chains, denoted by the Greek letters α (alpha), δ (delta), ε (epsilon), γ (gamma), and μ (mu), which define the class or isotype of an antibody as IgA, IgD, IgE, IgG, or IgM, respectively. IgG antibodies can be further divided into subclasses, for example, IgG1, IgG2, IgG3, and IgG4.

Each heavy chain type is characterized by a particular constant region with a sequence well known in the art. The constant region is identical in all antibodies of the same isotype, but differs in antibodies of different isotypes. Heavy chains γ, α, and δ have a constant region composed of three tandem immunoglobulin (Ig) domains, and a hinge region for added flexibility. Heavy chains and F have a constant region composed of four Ig domains.

The hinge region is a flexible amino acid stretch that links the Fc and Fab portions of an antibody. This regions contains cysteine residues that can form disulfide bonds, connecting two heavy chains together.

The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, light chains are classified as kappa (κ) or lambda (λ), and are characterized by a particular constant region as known in the art. A light chain has two successive domains: one variable domain at the amino-terminal end, and one constant domain at the carboxy-terminal end. Each antibody contains two light chains that are always identical; only one type of light chain, κ or λ, is present per antibody in mammals.

The Fc region, composed of two heavy chains that contribute three or four constant domains depending on the class of the antibody, plays a role in modulating immune cell activity. By binding to specific proteins, the Fc region ensures that each antibody generates an appropriate immune response for a given antigen. The Fc region also binds to various cell receptors, such as Fc receptors, and other immune molecules, such as complement proteins. By doing this, it mediates different physiological effects, including opsonization, cell lysis, and degranulation of mast cells, basophils and eosinophils.

As used herein, the term “epitope” refers to a specific arrangement of amino acids located on a peptide or protein to which an antibody or antibody fragment binds. Epitopes often consist of a chemically active surface grouping of molecules such as amino acids or sugar side chains, and have specific three dimensional structural characteristics as well as specific charge characteristics. Epitopes can be linear, i.e., involving binding to a single sequence of amino acids, or conformational, i.e., involving binding to two or more sequences of amino acids in various regions of the antigen that may not necessarily be contiguous in the linear sequence.

As used herein, the terms “specifically binds”, “bind specifically”, “specific binding”, and the like as applied to the present antibody compounds refer to the ability of a specific binding agent (such as an antibody) to bind to a target molecular species in preference to binding to other molecular species with which the specific binding agent and target molecular species are admixed. A specific binding agent is said specifically to recognize a target molecular species when it can bind specifically to that target.

As used herein, the term “binding affinity” refers to the strength of binding of one molecule to another at a site on the molecule. If a particular molecule will bind to or specifically associate with another particular molecule, these two molecules are said to exhibit binding affinity for each other. Binding affinity is related to the association constant and dissociation constant for a pair of molecules, but it is not critical to the methods herein that these constants be measured or determined. Rather, affinities as used herein to describe interactions between molecules of the described methods are generally apparent affinities (unless otherwise specified) observed in empirical studies, which can be used to compare the relative strength with which one molecule (e.g., an antibody or other specific binding partner) will bind two other molecules (e.g., two versions or variants of a peptide). The concepts of binding affinity, association constant, and dissociation constant are well known.

As used herein, the term “sequence identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs.

Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc., San Diego, Calif., United States of America), or by visual inspection. See generally, Altschul, S. F. et al., J. Mol. Biol. 215: 403-410 (1990) and Altschul et al. Nucl. Acids Res. 25: 3389-3402 (1997).

One example of an algorithm that is suitable for determining percent sequence identity and sequence similarity is the BLAST algorithm, which is described in (Altschul, S., et al., NCBI NLM NIH Bethesda, Md. 20894; and Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold.

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always; 0) and N (penalty score for mismatching residues; always; 0). For amino acid sequences, a scoring matrix is used to calculate the cumulative score. Extension of the word hits in each direction are halted when: the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

As used herein, the terms “humanized”, “humanization”, and the like, refer to grafting of the murine monoclonal antibody CDRs disclosed herein to human FRs and constant regions. Also encompassed by these terms are possible further modifications to the murine CDRs, and human FRs, by the methods disclosed in, for example, Kashmiri et al. (2005) Methods 36(1):25-34 and Hou et al. (2008) J. Biochem. 144(1):115-120, respectively, to improve various antibody properties, as discussed below.

As used herein, the term “humanized antibodies” refers to mAbs and antigen binding fragments thereof, including the antibody compounds disclosed herein, that have binding and functional properties according to the disclosure similar to those disclosed herein, and that have FRs and constant regions that are substantially human or fully human surrounding CDRs derived from a non-human antibody.

As used herein, the term “FR” or “framework sequence” refers to any one of FRs 1 to 4. Humanized antibodies and antigen binding fragments encompassed by the present disclosure include molecules wherein any one or more of FRs 1 to 4 is substantially or fully human, i.e., wherein any of the possible combinations of individual substantially or fully human FRs 1 to 4, is present. For example, this includes molecules in which FR1 and FR2, FR1 and FR3, FR1, FR2, and FR3, etc., are substantially or fully human. Substantially human frameworks are those that have at least 80% sequence identity to a known human germline framework sequence. Preferably, the substantially human frameworks have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity, to a framework sequence disclosed herein, or to a known human germline framework sequence.

Fully human frameworks are those that are identical to a known human germline framework sequence. Human FR germline sequences can be obtained from the international ImMunoGeneTics (IMGT) database and from The Immunoglobulin FactsBook by Marie-Paule Lefranc and Gerard Lefranc, Academic Press, 2001, the contents of which are herein incorporated by reference in their entirety.

The Immunoglobulin Facts Book is a compendium of the human germline immunoglobulin genes that are used to create the human antibody repertoire, and includes entries for 203 genes and 459 alleles, with a total of 837 displayed sequences. The individual entries comprise all the human immunoglobulin constant genes, and germline variable, diversity, and joining genes that have at least one functional or open reading frame allele, and which are localized in the three major loci. For example, germline light chain FRs can be selected from the group consisting of: IGKV3D-20, IGKV2-30, IGKV2-29, IGKV2-28, IGKV1-27, IGKV3-20, IGKV1-17, IGKV1-16, 1-6, IGKV1-5, IGKV1-12, IGKV1D-16, IGKV2D-28, IGKV2D-29, IGKV3-11, IGKV1-9, IGKV1-39, IGKV1D-39 and IGKV1D-33 and IGKJ1-5 and germline heavy chain FRs can be selected from the group consisting of: IGHV1-2, IGHV1-18, IGHV1-46, IGHV1-69, IGHV2-5, IGHV2-26, IGHV2-70, IGHV1-3, IGHV1-8, IGHV3-9, IGHV3-11, IGHV3-15, IGHV3-20, IGHV3-66, IGHV3-72, IGHV3-74, IGHV4-31, IGHV3-21, IGHV3-23, IGHV3-30, IGHV3-48, IGHV4-39, IGHV4-59 and IGHV5-51 and IGHJ1-6.

Substantially human FRs are those that have at least 80% sequence identity to a known human germline FR sequence. Preferably, the substantially human frameworks have at least 85%, at least 90%, at least 95%, at least 96%, at least 97%, at least 98%, or at least 99% sequence identity, to a framework sequences disclosed herein, or to a known human germline framework sequence.

CDRs encompassed by the present disclosure include not only those specifically disclosed herein, but also CDR sequences having sequence identities of at least 80%, at least 85%, at least 90%, at least 95%, at least 96%, at least 97%, at least 98%, or at least 99% sequence identity to a CDR sequence disclosed herein. Alternatively, CDRs encompassed by the present disclosure include not only those specifically disclosed herein, but also CDR sequences having 1, 2, 3, 4, or 5 amino acid changes at corresponding positions compared to CDR sequences disclosed herein. Such sequence identical, or amino acid modified, CDRs preferably bind to the antigen recognized by the intact antibody.

Humanization began with chimerization, a method developed during the first half of the 1980s (Morrison, S. L., M. J. Johnson, L. A. Herzenberg & V. T. Oi: Chimeric human antibody molecules: mouse antigen-binding domains with human constant region domains. Proc. Natl. Acad. Sci. USA., 81, 6851-5 (1984)), consisting of combining the variable (V) domains of murine antibodies with human constant (C) domains to generate molecules with ˜70% of human content.

The disclosure includes humanized antibodies which can be generated using several different methods, including those described in Almagro et al. Humanization of antibodies. Frontiers in Biosciences. (2008) January 1; 13:1619-33.

In one approach, the parent antibody compound CDRs are grafted into a human framework that has a high sequence identity with the parent antibody compound framework. The sequence identity of the new framework will generally be at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identical to the sequence of the corresponding framework in the parent antibody compound. In the case of frameworks having fewer than 100 amino acid residues, one, two, three, four, five, six, seven, eight, nine, or ten amino acid residues can be changed. This grafting may result in a reduction in binding affinity compared to that of the parent antibody. If this is the case, the framework can be back-mutated to the parent framework at certain positions based on specific criteria disclosed by Queen et al. (1991) Proc. Natl. Acad. Sci. USA 88:2869. Additional references describing methods useful to generate humanized variants based on homology and back mutations include as described in Olimpieri et al. Bioinformatics. 2015 Feb. 1; 31(3):434-435 and U.S. Pat. Nos. 4,816,397, 5,225,539, and 5,693,761; and the method of Winter and co-workers (Jones et al. (1986) Nature 321:522-525; Riechmann et al. (1988) Nature 332:323-327; and Verhoeyen et al. (1988) Science 239:1534-1536.

The identification of residues to consider for back-mutation can be carried out as described below. When an amino acid falls under the following category, the framework amino acid of the human germ-line sequence that is being used (the “acceptor FR”) is replaced by a framework amino acid from a framework of the parent antibody compound (the “donor FR”):

(a) the amino acid in the human FR of the acceptor framework is unusual for human frameworks at that position, whereas the corresponding amino acid in the donor immunoglobulin is typical for human frameworks at that position;

(b) the position of the amino acid is immediately adjacent to one of the CDRs; or

(c) any side chain atom of a framework amino acid is within about 5-6 angstroms (center-to-center) of any atom of a CDR amino acid in a three dimensional immunoglobulin model.

When each of the amino acids in the human FR of the acceptor framework and a corresponding amino acid in the donor framework is generally unusual for human frameworks at that position, such amino acid can be replaced by an amino acid typical for human frameworks at that position. This back-mutation criterion enables one to recover the activity of the parent antibody compound.

Another approach to generating humanized antibodies which exhibit similar functional properties to the antibody compounds in the disclosure involves randomly mutating amino acids within the grafted CDRs without changing the framework, and screening the resultant molecules for binding affinity and other functional properties that are as good as, or better than, those of the parent antibody compounds. Single mutations can also be introduced at each amino acid position within each CDR, followed by assessing the effects of such mutations on binding affinity and other functional properties. Single mutations producing improved properties can be combined to assess their effects in combination with one another.

Further, a combination of both of the foregoing approaches is possible. After CDR grafting, one can back-mutate specific FRs in addition to introducing amino acid changes in the CDRs. This methodology is described in Wu et al. (1999) J. Mol. Biol. 294: 151-162.

Applying the teachings of the present disclosure, a person skilled in the art can use common techniques, e.g., site-directed mutagenesis, to substitute amino acids within the presently disclosed CDR and FR sequences and thereby generate further variable region amino acid sequences derived from the present sequences. Up to all naturally occurring amino acids can be introduced at a specific substitution site. The methods disclosed herein can then be used to screen these additional variable region amino acid sequences to identify sequences having the indicated in vivo functions. In this way, further sequences suitable for preparing humanized antibodies and antigen-binding portions thereof in accordance with the present disclosure can be identified. Preferably, amino acid substitution within the frameworks is restricted to one, two, three, four, or five positions within any one or more of the four light chain and/or heavy chain FRs disclosed herein. Preferably, amino acid substitution within the CDRs is restricted to one, two, three, four, or five positions within any one or more of the three light chain and/or heavy chain CDRs. Combinations of the various changes within these FRs and CDRs described above are also possible.

That the functional properties of the antibody compounds generated by introducing the amino acid modifications discussed above conform to those exhibited by the specific molecules disclosed herein can be confirmed by the methods in Examples disclosed herein.

As described above, to circumvent the problem of eliciting human anti-murine antibody (HAMA) response in patients, murine antibodies have been genetically manipulated to progressively replace their murine content with the amino acid residues present in their human counterparts by grafting their complementarity determining regions (CDRs) onto the variable light (V_{L}) and variable heavy (V_{H}) frameworks of human immunoglobulin molecules, while retaining those murine framework residues deemed essential for the integrity of the antigen-combining site. However, the xenogeneic CDRs of the humanized antibodies may evoke anti-idiotypic (anti-Id) response in patients.

To minimize the anti-Id response, a procedure to humanize xenogeneic antibodies by grafting onto the human frameworks only the CDR residues most crucial in the antibody-ligand interaction, called “SDR grafting”, has been developed, wherein only the crucial specificity determining residues (SDRs) of CDRS are grafted onto the human frameworks. This procedure, described in Kashmiri et al. (2005) Methods 36(1):25-34, involves identification of SDRs through the help of a database of the three-dimensional structures of the antigen-antibody complexes of known structures, or by mutational analysis of the antibody-combining site. An alternative approach to humanization involving retention of more CDR residues is based on grafting of the ‘abbreviated’ CDRs, the stretches of CDR residues that include all the SDRs. Kashmiri et al. also discloses a procedure to assess the reactivity of humanized antibodies to sera from patients who had been administered the murine antibody.

Another strategy for constructing human antibody variants with improved immunogenic properties is disclosed in Hou et al. (2008) J. Biochem. 144(1):115-120. These authors developed a humanized antibody from 4C8, a murine anti-human CD34 monoclonal antibody, by CDR grafting using a molecular model of 4C8 built by computer-assisted homology modelling. Using this molecular model, the authors identified FR residues of potential importance in antigen binding. A humanized version of 4C8 was generated by transferring these key murine FR residues onto a human antibody framework that was selected based on homology to the murine antibody FR, together with the murine CDR residues. The resulting humanized antibody was shown to possess antigen-binding affinity and specificity similar to that of the original murine antibody, suggesting that it might be an alternative to murine anti-CD34 antibodies routinely used clinically.

Embodiments of the present disclosure encompass antibodies created to avoid recognition by the human immune system containing CDRs disclosed herein in any combinatorial form such that contemplated mAbs can contain the set of CDRs from a single murine mAb disclosed herein, or light and heavy chains containing sets of CDRs comprising individual CDRs derived from two or three of the disclosed murine mAbs. Such mAbs can be created by standard techniques of molecular biology and screened for desired activities using assays described herein. In this way, the disclosure provides a “mix and match” approach to create novel mAbs comprising a mixture of CDRs from the disclosed murine mAbs to achieve new, or improved, therapeutic activities.

Monoclonal antibodies or antigen-binding fragments thereof encompassed by the present disclosure that “compete” with the molecules disclosed herein are those that bind human CD47 at site(s) that are identical to, or overlapping with, the site(s) at which the present molecules bind. Competing monoclonal antibodies or antigen-binding fragments thereof can be identified, for example, via an antibody competition assay. For example, a sample of purified or partially purified human CD47 extracellular domain can be bound to a solid support. Then, an antibody compound, or antigen binding fragment thereof, of the present disclosure and a monoclonal antibody or antigen-binding fragment thereof suspected of being able to compete with such disclosure antibody compound are added. One of the two molecules is labeled. If the labeled compound and the unlabeled compound bind to separate and discrete sites on CD47, the labeled compound will bind to the same level whether or not the suspected competing compound is present. However, if the sites of interaction are identical or overlapping, the unlabeled compound will compete, and the amount of labeled compound bound to the antigen will be lowered. If the unlabeled compound is present in excess, very little, if any, labeled compound will bind. For purposes of the present disclosure, competing monoclonal antibodies or antigen-binding fragments thereof are those that decrease the binding of the present antibody compounds to CD47 by about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, or about 99%. Details of procedures for carrying out such competition assays are well known in the art and can be found, for example, in Harlow and Lane (1988) Antibodies, A Laboratory Manual, Cold Spring Harbor Laboratory Press, Cold Spring Harbor, N.Y. Such assays can be made quantitative by using purified antibodies. A standard curve is established by titrating one antibody against itself, i.e., the same antibody is used for both the label and the competitor. The capacity of an unlabeled competing monoclonal antibody or antigen-binding fragment thereof to inhibit the binding of the labeled molecule to the plate is titrated. The results are plotted, and the concentrations necessary to achieve the desired degree of binding inhibition are compared.

Whether mAbs or antigen-binding fragments thereof that compete with antibody compounds of the present disclosure in such competition assays possess the same or similar functional properties of the present antibody compounds can be determined via these methods in conjunction with the methods described in Examples below. In various embodiments, competing antibodies for use in the therapeutic methods encompassed herein possess biological activities as described herein in the range of from about 50% to about 100% or about 125%, or more, compared to that of the antibody compounds disclosed herein. In some embodiments, competing antibodies possess about 50%, about 60%, about 70%, about 80%, about 85%, about 90%, about 91%, about 92%, about 93%, about 94%, about 95%, about 96%, about 97%, about 98%, about 99%, or identical biological activity compared to that of the antibody compounds disclosed herein as determined by the methods disclosed in the Examples presented below.

The mAbs or antigen-binding fragments thereof, or competing antibodies useful in the compositions and methods can be any of the isotypes described herein. Furthermore, any of these isotypes can comprise further amino acid modifications as follows.

The monoclonal antibody or antigen-binding fragment thereof, or competing antibody described herein can be of the human IgG1 isotype.

The human IgG1 constant region of the monoclonal antibody, antigen-binding fragment thereof, or competing antibody described herein can be modified to alter antibody half-life. Antibody half-life is regulated in large part by Fc-dependent interactions with the neonatal Fc receptor (Roopenian and Alikesh, 2007). The human IgG1 constant region of the monoclonal antibody, antigen-binding fragment thereof, or competing antibody can be modified to increase half-life include, but are not limited to amino acid modifications N434A, T307A/E380A/N434A (Petkova et al., 2006, Yeung et al., 2009); M252Y/S254T/T256E (Dall'Acqua et al., 2006); T250Q/M428L (Hinton et al., 2006); and M428L/N434S (Zalevsky et al., 2010).

As opposed to increasing half-life, there are some circumstances where decreased half-life would be desired, such as to reduce the possibility of adverse events associated with high Antibody-Dependent Cellular Cytotoxicity (ADCC) and Complement-Dependent Cytotoxicity (CDC) antibodies (Presta 2008). The human IgG1 constant region of the monoclonal antibody, antigen-binding fragment thereof, or competing antibody described herein can be modified to decrease half-life and/or decrease endogenous IgG include, but are not limited to amino acid modifications I253A (Petkova et al., 2006); P257I/N434H, D376V/N434H (Datta-Mannan et al., 2007); and M252Y/S254T/T256E/H433K/N434F (Vaccaro et al., 2005).

The human IgG1 constant region of the monoclonal antibody, antigen-binding fragment thereof, or competing antibody described herein can be modified to increase or decrease antibody effector functions. These antibody effector functions include, but are not limited to, Antibody-Dependent Cellular Cytotoxicity (ADCC), Complement-Dependent Cytotoxicity (CDC), Antibody-Dependent Cellular Phagocytosis (ADCP), C1q binding, and altered binding to Fc receptors.

The human IgG1 constant region of the monoclonal antibody, antigen-binding fragment thereof, or competing antibody described herein can be modified to increase antibody effector function include, but are not limited to amino acid modifications S298A/E333A/K334 (Shields et al., 2001); S239D/I332E and S239D/A330L/I332E (Lazar et al., 2006); F234L/R292P/Y300L, F234L/R292P/Y300L/P393L, and F243L/R292P/Y300L/V305I/P396L (Stevenhagen et al., 2007); G236A, G236A/S239D/I332E, and G236A/S239D/A330L/I332E (Richards et al., 2008); K326A/E333A, K326A/E333S and K326W/E333S (Idusogie et al., 2001); S267E and S267E/L328F (Smith et al., 2012); H268F/S324T, S267E/H268F, S267E/S234T, and S267E/H268F/S324T (Moore et al., 2010); S298G/T299A (Sazinsky et al., 2008); E382V/M428I (Jung et al., 2010).

The human IgG1 constant region of the monoclonal antibody, antigen-binding fragment thereof, or competing antibody described herein can be modified to decrease antibody effector function include, but are not limited to amino acid modifications N297A and N297Q (Bolt et al., 1993, Walker et al., 1989); L234A/L235A (Xu et al., 2000); K214T/E233P/L234V/L235A/G236-deleted/A327G/P331A/D356E/L358M (Ghevaert et al., 2008); C226S/C229S/E233P/L234V/L235A (McEarchern et al., 2007); S267E/L328F (Chu et al., 2008).

The human IgG1 constant region of the monoclonal antibody, antigen-binding fragment thereof, or competing antibody described herein can be modified to decrease antibody effector function include, but are not limited to amino acid modifications V234A/G237A (Cole et al., 1999); E233D, G237D, P238D, H268Q, H268D, P271G, V309L, A330S, A330R, P331S, H268Q/A330S/V309L/P331S, H268D/A330S/V309L/P331S, H268Q/A330R/V309L/P331S, H268D/A330R/V309L/P331S, E233D/A330R, E233D/A330S, E233D/P271G/A330R, E233D/P271G/A330S, G237D/H268D/P271G, G237D/H268Q/P271G, G237D/P271G/A330R, G237D/P271G/A330S, E233D/H268D/P271G/A330R, E233D/H268Q/P271G/A330R, E233D/H268D/P271G/A330S, E233D/H268Q/P271G/A330S, G237D/H268D/P271G/A330R, G237D/H268Q/P271G/A330R, G237D/H268D/P271G/A330S, G237D/H268Q/P271G/A330S, E233D/G237D/H268D/P271G/A330R, E233D/G237D/H268Q/P271G/A330R, E233D/G237D/H268D/P271G/A330S, E233D/G237D/H268Q/P271G/A330S, P238D/E233D/A330R, P238D/E233D/A330S, P238D/E233D/P271G/A330R, P238D/E233D/P271G/A330S, P238D/G237D/H268D/P271G, P238D/G237D/H268Q/P271G, P238D/G237D/P271G/A330R, P238D/G237D/P271G/A330S, P238D/E233D/H268D/P271G/A330R, P238D/E233D/H268Q/P271G/A330R, P238D/E233D/H268D/P271G/A330S, P238D/E233D/H268Q/P271G/A330S, P238D/G237D/H268D/P271G/A330R, P238D/G237D/H268Q/P271G/A330R, P238D/G237D/H268D/P271G/A330S, P238D/G237D/H268Q/P271G/A330S, P238D/E233D/G237D/H268D/P271G/A330R, P238D/E233D/G237D/H268Q/P271G/A330R, P238D/E233D/G237D/H268D/P271G/A330S, P238D/E233D/G237D/H268Q/P271G/A330S (An et al., 2009, Mimoto, 2013).

The monoclonal antibody or antigen-binding fragment thereof, or competing antibody described herein can be of the human IgG2 isotype.

The human IgG2 constant region of the monoclonal antibody, antigen-binding fragment thereof, or competing antibody described herein can be modified to increase or decrease antibody effector functions. These antibody effector functions include, but are not limited to, Antibody-Dependent Cellular Cytotoxicity (ADCC), Complement-Dependent Cytotoxicity (CDC), Antibody-Dependent Cellular Phagocytosis (ADCP), and C1q binding, and altered binding to Fc receptors.

The human IgG2 constant region of the monoclonal antibody, antigen-binding fragment thereof, or competing antibody described herein can be modified to increase antibody effector function include, but are not limited to the amino acid modification K326A/E333S (Idusogie et al., 2001).

The human IgG2 constant region of the monoclonal antibody, antigen-binding fragment thereof, or competing antibody described herein can be modified to decrease antibody effector function include, but are not limited to amino acid modifications V234A/G237A (Cole et al., 1999); V234A, G237A, P238S, H268A, E233D, G237D, P238D, H268Q, H268D, P271G, V309L, A330S, A330R, P331S, P238S/H268A, V234A/G237A/P238S/H268A/V309L/A330S/P331S, H268Q/A330S/V309L/P331S, H268D/A330S/V309L/P331S, H268Q/A330R/V309L/P331S, H268D/A330R/V309L/P331S, E233D/A330R, E233D/A330S, E233D/P271G/A330R, E233D/P271G/A330S, G237D/H268D/P271G, G237D/H268Q/P271G, G237D/P271G/A330R, G237D/P271G/A330S, E233D/H268D/P271G/A330R, E233D/H268Q/P271G/A330R, E233D/H268D/P271G/A330S, E233D/H268Q/P271G/A330S, G237D/H268D/P271G/A330R, G237D/H268Q/P271G/A330R, G237D/H268D/P271G/A330S, G237D/H268Q/P271G/A330S, E233D/G237D/H268D/P271G/A330R, E233D/G237D/H268Q/P271G/A330R, E233D/G237D/H268D/P271G/A330S, E233D/G237D/H268Q/P271G/A330S, P238D/E233D/A330R, P238D/E233D/A330S, P238D/E233D/P271G/A330R, P238D/E233D/P271G/A330S, P238D/G237D/H268D/P271G, P238D/G237D/H268Q/P271G, P238D/G237D/P271G/A330R, P238D/G237D/P271G/A330S, P238D/E233D/H268D/P271G/A330R, P238D/E233D/H268Q/P271G/A330R, P238D/E233D/H268D/P271G/A330S, P238D/E233D/H268Q/P271G/A330S, P238D/G237D/H268D/P271G/A330R, P238D/G237D/H268Q/P271G/A330R, P238D/G237D/H268D/P271G/A330S, P238D/G237D/H268Q/P271G/A330S, P238D/E233D/G237D/H268D/P271G/A330R, P238D/E233D/G237D/H268Q/P271G/A330R, P238D/E233D/G237D/H268D/P271G/A330S, P238D/E233D/G237D/H268Q/P271G/A330S (An et al., 2009, Mimoto, 2013).

The Fc region of a human IgG2 of the monoclonal antibody, antigen-binding fragment thereof, or competing antibody described herein can be modified to alter isoform and/or agonistic activity, include, but are not limited to amino acid modifications C127S (CH1 domain), C232S, C233S, C232S/C233S, C236S, and C239S (White et al., 2015, Lightle et al., 2010).

The monoclonal antibody or antigen-binding fragment thereof, or competing antibody described herein can be of the human IgG3 isotype.

The human IgG3 constant region of the monoclonal antibody, or antigen binding fragment thereof, wherein said human IgG3 constant region of the monoclonal antibody, or antigen-binding fragment thereof can be modified at one or more amino acid(s) to increase antibody half-life, Antibody-Dependent Cellular Cytotoxicity (ADCC), Complement-Dependent Cytotoxicity (CDC), or apoptosis activity.

The human IgG3 constant region of the monoclonal antibody, or antigen-binding fragment thereof, wherein said human IgG3 constant region of the monoclonal antibody, or antigen-binding fragment thereof can be modified at amino acid R435H to increase antibody half-life.

The monoclonal antibody or antigen-binding fragment thereof, or competing antibody described herein can be of the human IgG4 isotype.

The human IgG4 constant region of the monoclonal antibody, antigen-binding fragment thereof, or competing antibody described herein can be modified to decrease antibody effector functions. These antibody effector functions include, but are not limited to, Antibody-Dependent Cellular Cytotoxicity (ADCC) and Antibody-Dependent Cellular Phagocytosis (ADCP).

The human IgG4 constant region of the monoclonal antibody, antigen-binding fragment thereof, or competing antibody described herein can be modified to prevent Fab arm exchange and/or decrease antibody effector function include, but are not limited to amino acid modifications F234A/L235A (Alegre et al., 1994); S228P, L235E and S228P/L235E (Reddy et al., 2000).

The present disclosure describes synergistic combinations may provide for an improved effectiveness, which effect may be measured by total tumor cell number; length of time to relapse; other clinical efficacy measurement; and other indices of patient health. Alternatively, synergistic combinations for a therapeutic effect that is comparable to the effectiveness of a monotherapy, while reducing adverse side effects, e.g. damage to non-targeted tissues, immune status, and other clinical indices. Synergistic combinations of the present invention combine an agent that is targeted to inhibit or block CD47 function; and an agent that is a chemotherapeutic agent or anti-cancer agent. The combination may be provided with one or more combination of agents, more specifically, an anti-CD47 antibody and a chemotherapeutic agent, e.g. from the chemotherapeutic classes of anthracyclines, platinums, taxols, topoisomerase inhibitors, anti-metabolites, anti-tumor antibiotics, mitotic inhibitors, and alkylating agents.

The term, “combination therapy”, as used herein, refers to those situations in which a subject is simultaneously exposed to two or more therapeutic regimens (e.g., two or more therapeutic agents). In some embodiments, two or more agents may be administered simultaneously; in some embodiments, such agents may be administered sequentially; in some embodiments such agents are administered in overlapping dosing regimens.

The terms, “synergistic” or “synergistic effect”, as used herein, refers to the interaction of two or more therapeutic regimens (e.g., two or more therapeutic agents) to produce a combined effect greater than the sum of their separate effects.

The terms, “additive” or “additive effect”, as used herein, refers to the interaction of two or more therapeutic regimens (e.g., two or more therapeutic agents) used in combination produce a total effect the same as the sum of the individual effects.

The term “tumor”, as used herein, refers to all neoplastic cell growth and proliferation, whether malignant or benign, and all pre-cancerous and cancerous cells and tissues.

The terms “cancer”, “cancerous”, and “tumor” are not mutually exclusive as used herein.

The terms “cancer” and “cancerous” refer to or describe the physiological condition in mammals that is typically characterized by aberrant cell growth/proliferation. Examples of cancers include, but are not limited to, carcinoma, lymphoma (i.e., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

The term “susceptible cancer” as used herein refers to a cancer, cells of which express CD47, and are responsive to treatment with an antibody or antigen binding fragment thereof, or competing antibody or antigen binding fragment thereof, of the present disclosure.

As used herein, term “treating” or “treat” or “treatment” means slowing, interrupting, arresting, controlling, stopping, reducing, or reversing the progression or severity of a sign, symptom, disorder, condition, or disease, but does not necessarily involve a total elimination of all disease-related signs, symptoms, conditions, or disorders. The term “treating” and the like refer to a therapeutic intervention that ameliorates a sign or symptom of a disease or pathological condition after it has begun to develop.

As used herein, term “effective amount” refers to the amount or dose of an antibody compound of the present disclosure which, upon single or multiple dose administration to a patient or organ, provides the desired treatment or prevention.

The precise effective amount for any particular subject will depend upon their size and health, the nature and extent of their condition, and the therapeutics or combination of therapeutics selected for administration. The effective amount for a given patient is determined by routine experimentation and is within the judgment of a clinician. Therapeutically effective amounts of the present antibody compounds can also comprise an amount in the range of from about 0.1 mg/kg to about 150 mg/kg, from about 0.1 mg/kg to about 100 mg/kg, from about 0.1 mg/kg to about 50 mg/kg, or from about 0.05 mg/kg to about 10 mg/kg per single dose administered to a harvested organ or to a patient. Known antibody-based pharmaceuticals provide guidance in this respect. For example, Herceptin™ is administered by intravenous infusion of a 21 mg/ml solution, with an initial loading dose of 4 mg/kg body weight and a weekly maintenance dose of 2 mg/kg body weight; Rituxan™ is administered weekly at 375 mg/m2; for example.

A therapeutically effective amount for any individual patient can be determined by the health care provider by monitoring the effect of the antibody compounds on tumor regression, circulating tumor cells, tumor stem cells or anti-tumor responses. Analysis of the data obtained by these methods permits modification of the treatment regimen during therapy so that optimal amounts of antibody compounds of the present disclosure, whether employed alone or in combination with one another, or in combination with another therapeutic agent, or both, are administered, and so that the duration of treatment can be determined as well. In this way, the dosing/treatment regimen can be modified over the course of therapy so that the lowest amounts of antibody compounds used alone or in combination that exhibit satisfactory efficacy are administered, and so that administration of such compounds is continued only so long as is necessary to successfully treat the patient. Known antibody-based pharmaceuticals provide guidance relating to frequency of administration e.g., whether a pharmaceutical should be delivered daily, weekly, monthly, etc. Frequency and dosage may also depend on the severity of symptoms.

In some embodiments antibody compounds of the present disclosure can be used as medicaments in human and veterinary medicine, administered by a variety of routes including, but not limited to, oral, intravenous, intramuscular, intra-arterial, intramedullary, intraperitoneal, intrathecal, intraventricular, transdermal, transcutaneous, topical, subcutaneous, intratumoral, intranasal, enteral, sublingual, intravaginal, intravesiciular or rectal routes. The compositions can also be administered directly into a lesion such as a tumor. Dosage treatment may be a single dose schedule or a multiple dose schedule. Hypo sprays may also be used to administer the pharmaceutical compositions. Typically, the therapeutic compositions can be prepared as injectables, either as liquid solutions or suspensions. Solid forms suitable for solution in, or suspension in, liquid vehicles prior to injection can also be prepared. Veterinary applications include the treatment of companion/pet animals, such as cats and dogs; working animals, such as guide or service dogs, and horses; sport animals, such as horses and dogs; zoo animals, such as primates, cats such as lions and tigers, bears, etc.; and other valuable animals kept in captivity.

Such pharmaceutical compositions can be prepared by methods well known in the art. See, e.g., Remington: The Science and Practice of Pharmacy, 21st Edition (2005), Lippincott Williams & Wilkins, Philadelphia, Pa., and comprise one or more antibody compounds disclosed herein, and a pharmaceutically or veterinarily acceptable, for example, physiologically acceptable, carrier, diluent, or excipient.

The present disclosure describes anti-CD47 mAbs with distinct functional profiles. These antibodies possess distinct combinations of properties selected from the following: These antibodies possess distinct combinations of properties selected from the following: 1) exhibit cross-reactivity with one or more species homologs of CD47; 2) block the interaction between CD47 and its ligand SIRPα; 3) increase phagocytosis of human tumor cells; 4) induce death of susceptible human tumor cells; 5) do not induce cell death of human tumor cells; 6) do not have reduced or minimal binding to human red blood cells (hRBCs); 7) have reduced binding to hRBCs; 8) have minimal binding to hRBCs; 9) cause reduced agglutination of hRBCs; 10) cause no detectable agglutination of hRBCs; 11) reverse TSP1 inhibition of the nitric oxide (NO) pathway; 12) do not reverse TSP1 inhibition of the NO pathway; 13) cause loss of mitochondrial membrane potential; 14) do not cause loss of mitochondrial membrane potential; 15) cause an increase in cell surface calreticulin expression on human tumor cells; 16) do not cause an increase in cell surface calreticulin expression on human tumor cells; 17) cause an increase in adenosine triphosphate (ATP) release by human tumor cells; 18) do not cause an increase in adenosine triphosphate (ATP) release by human tumor cells; 19) cause an increase in high mobility group box 1 (HMGB1) release by human tumor cells; 20) do not cause an increase in high mobility group box 1 (HMGB1) release by human tumor cells; 21) cause an increase in type I interferon release by human tumor cells; 22) do not cause an increase in type I interferon release by human tumor cells; 23) cause an increase in C-X-C Motif Chemokine Ligand 10 (CXCL10) release by human tumor cells; 24) do not cause an increase in C-X-C Motif Chemokine Ligand 10 (CXCL10) release by human tumor cells; 25) cause an increase in cell surface protein disulfide-isomerase A3 (PDIA3) expression on human tumor cells; 26) do not cause an increase in cell surface protein disulfide-isomerase A3 (PDIA3) expression on human tumor cells; 27) cause an increase in cell surface heat shock protein 70 (HSP70) expression on human tumor cells; 28) do not cause an increase in cell surface heat shock protein 70 (HSP70) expression on human tumor cells; 29) cause an increase in cell surface heat shock protein 90 (HSP90) expression on human tumor cells; 30) do not cause an increase in cell surface heat shock protein 90 (HSP90) expression on human tumor cells; 31) have reduced binding to normal human cells, which includes, but is not limited to, endothelial cells, skeletal muscle cells, epithelial cells, and peripheral blood mononuclear cells (e.g., human aortic endothelial cells, human skeletal muscle cells, human microvascular endothelial cells, human renal tubular epithelial cells, human peripheral blood CD3+ cells, and human peripheral blood mononuclear cells); 32) do not have reduced binding to normal human cells, which includes, but is not limited to, endothelial cells, skeletal muscle cells, epithelial cells, and peripheral blood mononuclear cells (e.g., human aortic endothelial cells, human skeletal muscle cells, human microvascular endothelial cells, human renal tubular epithelial cells, human peripheral blood CD3+ cells, and human peripheral blood mononuclear cells); 33) have a greater affinity for human CD47 at an acidic pH compared to physiological pH; 34) do not have a greater affinity for human CD47 at an acidic pH compared to physiological pH; and 35) cause an increase in annexin A1 release by human tumor cells.

The anti-CD47 antibodies and antigen binding fragments thereof of the present disclosure possess combinations of properties that are distinct from the anti-CD47 antibodies of the prior art. These properties and characteristics will now be described in further detail. As used herein, the term “binds to human CD47” refers to binding with an apparent Kd greater than 50 nM, for example, in a solid phase ELISA assay or cell based assay.

As used herein, the terms “apparent binding affinity and apparent Kd” are determined by non-linear fit (Prism GraphPad software) of the binding data at the various antibody concentrations.

### Binding to CD47 of Different Species

The anti-CD47 antibodies, and antigen binding fragments thereof, of the present disclosure bind human CD47. In certain embodiments, the anti-CD47 antibodies exhibit cross-reactivity with one or more species homologs of CD47, for example CD47 homologs of non-human primate origin. In certain embodiments, the anti-CD47 antibodies and antigen binding fragments thereof of the present disclosure bind to human CD47 and to CD47 of non-human primate, mouse, rat, and/or rabbit origin. The cross-reactivity with other species homologs can be particularly advantageous in the development and testing of therapeutic antibodies. For example, pre-clinical toxicology testing of therapeutic antibodies is frequently carried out in non-human primate species including, but not limited to, cynomolgus monkey, green monkey, rhesus monkey and squirrel monkey. Cross-reactivity with these species homologs can therefore be particularly advantageous for the development of antibodies as clinical candidates.

As used herein, the term “cross-reacts with one or more species homologs of CD47” refers to binding with an apparent Kd greater than 50 nM.

### Blocking the Interaction Between CD47 and SIRPα and Promoting Phagocytosis

CD47, also known as integrin associated protein (IAP), is a 50 kDa cell surface receptor that is comprised of an extracellular N-terminal IgV domain, a five membrane-spanning transmembrane domain, and a short C-terminal intracellular tail that is alternatively spliced.

Two ligands bind to CD47: Signal Regulatory Protein alpha (SIRPα) and Thrombospondin-1 (TSP1). TSP1 is present in plasma and synthesized by many cells, including platelets. SIRPα is expressed on hematopoietic cells, which include macrophages and dendritic cells.

When SIRPα on a phagocyte engages CD47 on a target cell, this interaction prevents phagocytosis of the target cell. The interaction of CD47 and SIRPα effectively sends a “don't eat me” signal to the phagocyte (Oldenborg et al. Science 288: 2051-2054, 2000). Blocking the interaction of SIRPα and CD47 with an anti-CD47 mAb in a therapeutic context can provide an effective anti-cancer treatment by promoting the uptake and clearance of cancer cells by the host's immune system. Thus, an important functional characteristic of some anti-CD47 mAbs is the ability to block the interaction of CD47 and SIRPα, resulting in phagocytosis of CD47 expressing tumor cells by macrophages. Several anti-CD47 mAbs have been shown to block the interaction of CD47 and SIRPα, including B6H12 (Seiffert et al. Blood 94:3633-3643, 1999; Latour et al. J. Immunol. 167: 2547-2554, 2001; Subramanian et al. Blood 107: 2548-2556, 2006; Liu et al. J Biol. Chem. 277: 10028-10036, 2002; Rebres et al. J. Cellular Physiol. 205: 182-193, 2005), BRIC126 (Vernon-Wilson et al. Eur J Immunol. 30: 2130-2137, 2000; Subramanian et al. Blood 107: 2548-2556, 2006), CC2C6 (Seiffert et al. Blood 94:3633-3643, 1999), and 1F7 (Rebres et al. J. Cellular Physiol. 205: 182-193, 2005). B6H12 and BRIC126 have also been shown to cause phagocytosis of human tumor cells by human and mouse macrophages (Willingham et al. Proc Natl Acad Sci USA 109(17):6662-6667, 2012; Chao et al. Cell 142:699-713, 2012; EP 2 242 512 B1). Other existing anti-CD47 mAbs, such as 2D3, does not block the interaction of CD47 and SIRPα (Seiffert et al. Blood 94:3633-3643, 1999; Latour et al. J. Immunol. 167: 2547-2554, 2001; Rebres et al. J. Cellular Physiol. 205: 182-193, 2005), and does not cause phagocytosis of tumor cells (Willingham et al. Proc Natl Acad Sci USA 109(17):6662-6667, 2012; Chao et al. Cell 142:699-713, 2012; EP 2 242 512 B1).

As used herein, the term “blocks SIRPα binding to human CD47” refers to a greater than 50% reduction of SIRPα-Fc binding to CD47 on cells by an anti-CD47 mAb compared to either untreated cells or cells treated with a negative antibody.

The anti-CD47 mAbs of the disclosure described herein, block the interaction of CD47 and SIRPα and increase phagocytosis of human tumor cells.

“Phagocytosis” of cancer cells refers to the engulfment and digestion of such cells by macrophages, and the eventual digestion or degradation of these cancer cells and the release of digested or degraded cellular components extracellularly, or intracellularly to undergo further processing. Anti-CD47 monoclonal antibodies that block SIRPα, binding to CD47 increase macrophage phagocytosis of cancer cells. SIRPα binding to CD47 on cancer cells would otherwise allow these cells to escape macrophage phagocytosis. The cancer cell may be viable or living cancer cells.

As used herein, the term “increases phagocytosis of human tumor cells’ refers to a greater than 2-fold increase in phagocytosis of human tumor cells by human macrophages in the presence of an anti-CD47 mAb compared to either untreated cells or cells treated with a negative control antibody.

### Inducing Death of Tumor Cells

Some soluble anti-CD47 mAbs initiate a cell death program on binding to CD47 on tumor cells, resulting in collapse of mitochondrial membrane potential, loss of ATP generating capacity, increased cell surface expression of phosphatidylserine (detected by increased staining for annexin V) and cell death without the participation of caspases or fragmentation of DNA. Such soluble anti-CD47 mAbs have the potential to treat a variety of solid and hematological cancers. Several soluble anti-CD47 mAbs which have been shown to induce tumor cell death, including MABL-1, MABL-2 and fragments thereof (U.S. Pat. No. 8,101,719; Uno et al. Oncol Rep. 17: 1189-94, 2007; Kikuchi et al. Biochem Biophys Res. Commun. 315: 912-8, 2004), Ad22 (Pettersen et al. J. Immunol. 162: 7031-7040, 1999; Lamy et al. J Biol. Chem. 278: 23915-23921, 2003), and 1F7 (Manna et al. J. Immunol. 170: 3544-3553, 2003; Manna et al. Cancer Research, 64: 1026-1036, 2004). In the previous analyses of MABL-1, MABL-2 and fragments thereof, Ad22 and 1F7, related approaches were used to define apoptosis and cell death induced by these anti-CD47 mAbs. Annexin V and propidium iodide (PI) staining were assessed by flow cytometry to demonstrate that the MABL scFV-15 dimer induced apoptosis of CD47-positive cells, both in the early stage (annexin V⁺, PI⁻) and the late stage (annexin V⁺, PI⁺) (Kikuchi et al. Biochem Biophys Res. Commun. 315: 912-8, 2004). A similar approach was used to show that Ad22 induced an increase in both apoptotic (annexin V⁺, PI⁻) and dead (annexin V⁺, PI⁺) cells (Pettersen et al. J. Immuno. 162: 7031-7040, 1999). Induction of apoptosis by 1F7 was assessed by analyzing annexin V⁺ cells by flow cytometry (Manna et al. J. Immunol. 170: 3544-3553, 2003; Manna et al. Cancer Research, 64: 1026-1036, 2004). Some of the anti-CD47 mAbs of the disclosure described herein induce cell death of human tumor cells.

Phosphatidylserine exposure on the external leaflet of the plasma membrane is widely observed during apoptosis and is the basis for the annexin V binding assay to detect aopototic cell death. It is important to note that, in some systems, phosphatidlylserine exposure and annexin V positivity are reversisble; that is some annexin V⁺ cells are viable and may resume growth and reestablish phospholipid symmetry (Hammill et al. Exp. Cell Res. 251: 16-21, 1999). 7-aminoactinomycin D (7-AAD) is a fluorescent intercalator that undergoes a spectral shift upon association with DNA. Live cells have intact membranes that exclude 7-AAD, whereas dead or apoptotic cells do not exclude 7-AAD.

The terms “inducing cell death” or “kills” and the like, are used interchangeably herein.

As used herein, the term “induces death of human tumor cells” refers to increased binding of annexin V (in the presence of calcium) and increased 7-aminoactinomycin D (7-AAD) or propidium iodide uptake in response to treatment with an anti-CD47 mAb. These features may be quantitated by flow cytometry in three cell populations: annexin V positive (annexin V⁺), annexin V positive/7-AAD negative (annexin V⁺/7-AAD⁻) and annexin V positive/7-AAD positive (annexin V⁺/7-AAD⁺). Induction of cell death is defined by a greater than 2-fold increase in each of the above cell populations in human tumor cells caused by soluble anti-CD47 mAb compared to the background obtained with the negative control antibody, (humanized, isotype-matched antibody) or untreated cells.

Another indicator of cell death is loss of mitochondrial function and membrane potential by the tumor cells as assayed by one of several available measures (potentiometric fluorescent dyes such as DiO-C6 or JC1 or formazan-based assays such as MTT or WST-1).

As used herein, the term “causes loss of mitochondrial membrane potential” refers to a statistically significant (p<0.05 or greater) decrease in mitochondrial membrane potential by a soluble anti-CD47 mAb compared to the background obtained with a negative control, humanized isotype-matched antibody or no treatment.

Induction of cell death refers to the ability of certain of the soluble anti-CD47 antibodies, murine antibodies, chimeric antibodies, humanized antibodies, or antigen-binding fragments thereof (and competing antibodies and antigen-binding fragments thereof) disclosed herein to kill cancer cells via a cell autonomous mechanism without participation of complement or other cells including, but not limited to, T cells, neutrophils, natural killer cells, macrophages, or dendritic cells.

Among the present humanized or chimeric mAbs, those that induce cell death of human tumor cells cause increased annexin V binding similar to the findings reported for anti-CD47 mAbs Ad22 (Pettersen et al. J. Immunol. 166: 4931-4942, 2001; Lamy et al. J. Biol. Chem. 278: 23915-23921, 2003); 1F7 (Manna and Frazier J. Immunol. 170:3544-3553, 2003; Manna and Frazier Cancer Res. 64:1026-1036, 2004); and MABL-1 and 2 (U.S. Pat. No. 7,531,643 B2; U.S. Pat. No. 7,696,325 B2; U.S. Pat. No. 8,101,719 B2).

Cell viability assays are described in NCI/NIH guidance manual that describes numerous types of cell based assays that can be used to assess induction of cell death caused by CD47 antibodies: “Cell Viability Assays”, Terry L Riss, PhD, Richard A Moravec, BS, Andrew L Niles, MS, Helene A Benink, PhD, Tracy J Worzella, MS, and Lisa Minor, PhD. Contributor Information, published May 1, 2013.

Binding to hRBCs

CD47 is expressed on human erythrocytes (hRBCs) (Brown. J Cell Biol. 111: 2785-2794, 1990; Avent. Biochem J., (1988) 251: 499-505; Knapp. Blood, (1989) Vol. 74, No. 4, 1448-1450; Oliveira et al. Biochimica et Biophysica Acta 1818: 481-490, 2012; Petrova P. et al. Cancer Res 2015; 75(15 Suppl): Abstract nr 4271). It has been shown that anti-CD47 mAbs bind to RBCs, including B6H12 (Brown et al. J. Cell Biol., 1990, Oliveira et al. Biochimica et Biophysica Acta 1818: 481-490, 2012, Petrova P. et al. Cancer Res 2015; 75(15 Suppl): Abstract nr 4271), BRIC125 (Avent. Biochem J., (1988) 251: 499-505), BRIC126 (Avent. Biochem J., (1988) 251: 499-505; Petrova P. et al. Cancer Res 2015; 75(15 Suppl): Abstract nr 4271), 5F9 (Uger R. et al. Cancer Res 2014; 74(19 Suppl): Abstract nr 5011, Liu et al. PLoS One. 2015 Sep. 21; 10(9): e0137345; Sikic B. et al. J Clin Oncol 2016; 34 (suppl; abstract 3019)), anti-CD47 antibodies disclosed in US Patent Publication 2014/0161799, WO Publication 2014/093678, US Patent Publication 2014/0363442, and CC2C6 (Petrova P. et al. Cancer Res 2015; 75(15 Suppl): Abstract nr 4271, Uger R. et al. Cancer Res 2014; 74(19 Suppl): Abstract nr 5011). It has also been shown that a SIRPα-Fc fusion protein, which binds to human CD47, has reduced binding to human RBCs compared to other human cells (Uger R. et al. Cancer Res 2014; 74(19 Suppl: Abstract nr 5011; Petrova et al. Clin Cancer Res 23: 1068-1079, 2017). Binding to RBCs can be reduced by generation of bi-specific antibodies with only one CD47 binding arm (Masternak et al. Cancer Res 2015; 75(15 Suppl): Abstract nr 2482). Because some anti-CD47 mAbs have been shown to result in reduction of RBCs when administered to cynomolgus monkeys (Mounho-Zamora B. et al. The Toxicologist, Supplement to Toxicological Sciences, 2015; 144 (1): Abstract 596: 127, Liu et al. PLoS One. 2015 Sep. 21; 10(9): e0137345; Pietsch et al. Cancer Res 2015; 75(15 Suppl): Abstract nr 2470), it is highly desirable to identify anti-CD47 mAbs that do not bind to CD47-expressing RBCs.

As used herein, the terms “red blood cell(s)” and “erythrocyte(s)” are synonymous and used interchangeably herein.

As used herein, the terms “reduced binding to hRBCs”, refers to an apparent Kd of an anti-CD47 mAb binding to a hRBC which is 10-fold or greater than the apparent Kd on a human tumor cell, wherein the tumor cell is an OV10 hCD47 cell (human OV10 ovarian cancer cell line expressing human CD47).

As used herein, the term “no binding” or “NB”, refers to no measurable binding to hRBCs at an anti-CD47 mAb concentration up to and including 50 μg/ml.

Prior to the disclosure described herein, no anti-CD47 mAbs have been reported that do not bind to human RBCs expressing CD47.

Some of the anti-CD47 mAbs, disclosed herein, have reduced or no detectable binding to human RBCs.

### Binding to Human Endothelia Cells and Other Normal Human Cells

In addition to expression/overexpression on most hematological malignancies and solid tumors (Willingham et al, Proc. Natl. Acad. Sci. 2012), CD47 is also expressed, by many but not all, normal cell types, including, but not limited to RBCs (see previous section), lymphocytes and mononuclear cells, endothelial cells, and brain, liver, muscle cells and/or tissues (Brown et al, J. Cell Biol. 1990; Reinhold et al, J. Cell Sci. 1995; Matozaki et al, Cell 2009; Stefanidakis et al, Blood 2008; Xiao et al, Cancer Letters 2015). Because of this expression, it is expected that some anti-CD47 mAbs would bind to these normal cell types/tissues in addition to the cancer cells which are the therapeutic target. It is therefore desirable to identify anti-CD47 mAbs that either do not bind or have reduced binding to some of these normal cells to both reduce potential non-desired effects on these normal cells and also allow more available antibody for binding to the tumor cells.

As used herein, the terms “reduced binding to normal human cells including, but not limited to, endothelial cells, epithelial cells, skeletal muscle cells, peripheral blood mononuclear cells or CD3⁺ T cells” refers to the apparent Kd of an anti-CD47 mAb binding to these cells which is 10-fold or greater than the apparent Kd of the anti-CD47 mAb binding to a human tumor cell, wherein the tumor cell is OV10 hCD47.

As used herein, the term “no binding” or “NB” refers to no measurable binding to normal human cells including, but not limited to, endothelial cells, epithelial cells, skeletal muscle cells, peripheral blood mononuclear cells or CD3⁺ T cells at an anti-CD47 mAb concentration up to and including 30 μg/ml.

### Agglutination of RBCs

Red blood cell (RBC) agglutination or hemagglutination is a homotypic interaction that occurs when RBCs aggregate or clump together following incubation with various agents, including antibodies to RBC antigens and cell surface proteins such as CD47. Many anti-CD47 antibodies have been reported to cause hemagglutination of isolated human RBCs in vitro, in a concentration dependent manner, including B6H12, BRIC126, MABL-1, MABL-2, CC2C6, and 5F9 (Uger R. et al. Cancer Res. 2014; 74(19 Suppl): Abstract nr 5011, U.S. Pat. No. 9,045,541, Uno et al. Oncol Rep. 17: 1189-94, 2007; Kikuchi et al. Biochem Biophys Res. Commun. 315: 912-8, 2004; Sikic B. et al. J Clin Oncol 2016; 34 (suppl; abstract 3019)). This functional effect requires binding to RBCs by an intact, bivalent antibody and can be reduced or eliminated by generating antibody fragments, either a F(ab′) or svFv (Uno et al. Oncol Rep. 17: 1189-94, 2007; Kikuchi et al. Biochem Biophys Res. Commun. 315: 912-8, 2004) or bi-specific antibodies with only one CD47 binding arm (Masternak et al. Cancer Res. 2015; 75(15 Suppl): Abstract nr 2482). Other functional properties of these fragments, including cell killing, were shown to be either reduced or retained in these fragments (Uno et al. Oncol Rep. 17: 1189-94, 2007; Kikuchi et al. Biochem. Biophys. Res. Commun. 315: 912-8, 2004). The mouse antibody 2D3 is an example of an anti-CD47 antibody that binds to CD47 on red blood cells but does not cause hemagglutination (U.S. Pat. No. 9,045,541, Petrova et al. Cancer Res. 2015; 75(15 Suppl): Abstract nr 4271).

Hemagglutination has been shown to be reduced/eliminated by reducing the binding selectively to human RBCs, but not other cells, using a SIRPα-Fc fusion protein (Uger R. et al. Blood 2013; 122(21): 3935). In addition, mouse anti-CD47 mAb 2A1 and humanized versions of 2A1 have been reported to block CD47/SIRPα but do not exhibit hemagglutination activity (U.S. Pat. No. 9,045,541). A small number of a panel of mouse anti-human CD47 antibodies (3 of 23) were reported to not cause hemagglutination of human RBCs (Pietsch E et al. Cancer Res. 2015; 75(15 Suppl): Abstract nr 2470). Therefore, prior to the disclosure described herein, there was a need to identify CD47 mAbs that block SIRPα/CD47 binding, have no detectable or reduced binding to RBCs and/or cause no hemagglutination. The term “agglutination” refers to cellular clumping, while the term “hemagglutination” refers to clumping of a specific subset of cells, i.e., RBCs. Thus, hemagglutination is a type of agglutination.

As used herein, the term “reduced hemagglutination” refers to measurable agglutination activity of hRBCs at anti-CD47 mAb concentrations greater that 1.85 μg/ml, and no measurable activity at concentrations less than or equal to 1.85 μg/ml in a washed RBC assay.

As used herein, the term “no detectable hemagglutination”, refers to no measurable agglutination activity of hRBCs at anti-CD47 mAb concentrations greater or equal to 0.3 μg/ml to a concentration less than or equal to 50 μg/ml in a washed RBC assay.

Some of the anti-CD47 antibodies described herein, cause reduced or no detectable hemagglutination of human RBCs.

### Immunogenic Cell Death

The concept of immunogenic cell death (ICD) has emerged in recent years. This form of cell death, unlike non-immunogenic cell death, stimulates an immune response against antigens from cancer cells. ICD is induced by specific chemotherapy drugs, including anthracyclines (doxorubicin, daurorubicin and mitoxantrone) and oxaliplatin, but not by cisplatin and other chemotherapy drugs. ICD is also induced by bortezomib, cardiac glycosides, photodynamic therapy and radiation Galluzi et al. Nat. Rev. Immunol. 17: 97-111, 2016). The distinctive characteristics of ICD of tumor cells are the release from or exposure on tumor cell surfaces of specific ligands: 1) the pre-apoptotic cell surface exposure of calreticulin, 2) the secretion of adenosine triphosphate (ATP), 3) release of high mobility group box 1 (HMGB1), 4) annexin A1 release, 5) type I interferon release and 6) C-X-C motif chemokine ligand 10 (CXCL10) release. These ligands are endogenous damage-associated molecular patterns (DAMPs), which include the cell death-associated molecules (CDAMs) (Kroemer et al. Annu. Rev. Immunol. 31: 51-72, 2013). Importantly, each of these ligands induced during ICD binds to specific receptors, referred to as pattern recognition receptors (PRRs), that contribute to an anti-tumor immune response. ATP binds the purinergic receptors PY2, G-protein coupled, 2 (P2RY2) and PX2, ligand-gated ion channel, 7 (P2RX7) on dendritic cells causing dendritic cell recruitment and activation, respectively. Annexin A1 binds to formyl peptide receptor 1 (FPR1) on dendritic cells causing dendritic cell homing. Calreticulin expressed on the surface of tumor cells binds to LRP1 (CD91) on dendritic cells promoting antigen uptake by dendritic cells. HMGB1 binds to toll-like receptor 4 (TLR4) on dendritic cells to cause dendritic cell maturation. As a component of ICD, tumor cells release type I interferon leading to signaling via the type I interferon receptor and the release of the CXCL10 which favors the recruitment of effector CXCR3+ T cells Together, the actions of these ligands on their receptors facilitate recruitment of DCs into the tumor, the engulfment of tumor antigens by DCs and optimal antigen presentation to T cells. Kroemer et al. have proposed that a precise combination of the CDAMs mentioned above elicited by ICD can overcome the mechanisms that normally prevent the activation of anti-tumor immune responses (Kroemer et al. Annu Rev Immunol 31: 51-72, 2013; Galluzi et al. Nat. Rev. Immunol. 17: 97-111, 2016). When mouse tumor cells treated in vitro with ICD-inducing modalities are administered in vivo to syngeneic mice, they provide effective vaccination that leads to an anti-tumor adaptive immune response, including memory. This vaccination effect cannot be tested in xenograft tumor models because the mice used in these studies lack a complete immune system. The available data indicate that ICD effects induced by chemotherapy or radiation will promote an adaptive anti-tumor immune response in cancer patients. The components of ICD are described in more detail below.

In 2005, it was reported that tumor cells which were dying in response to anthracycline chemotherapy in vitro caused an effective anti-tumor immune response when administered in vivo in the absence of adjuvant (Casares et al. J. Exp. Med. 202: 16911701, 2005). This immune response protected mice from subsequent re-challenge with viable cells of the same tumor and caused regression of established tumors. Anthracyclines (doxorubicin, daunorubicin and idarubicin) and mitomycin C induced tumor cell apoptosis with caspase activation, but only apoptosis induced by anthracyclines resulted in immunogenic cell death. Caspase inhibition did not inhibit cell death induced by doxorubicin but did suppress the immunogenicity of tumor cells dying in response to doxorubicin. The central roles of dendritic cells and CD8+ T cells in the immune response elicited by doxorubicin-treated apoptotic tumor cells was established by the demonstration that depletion of these cells abolished the immune response in vivo.

Calreticulin is one of the most abundant proteins in the endoplasmic reticulum (ER). Calreticulin was shown to rapidly translocate preapoptotically from the ER lumen to the surface of cancer cells in response to multiple ICD inducers, including anthracyclines (Obeid et al. Nat Med. 13: 54-61, 2007; Kroemer et al. Annu. Rev. Immunol. 31: 51-72, 2013). Blockade or knockdown of calreticulin suppressed the phagocytosis of anthracycline-treated tumor cells by dendritic cells and abolished their immunogenicity in mice. The exposure of calreticulin caused by anthracyclines or oxaliplatin is activated by an ER stress response that involves the phosphorylation of the eukaryotic translation initiation factor eIF2α by the PKR-like ER kinase. Calreticulin, which has a prominent function as an “eat-me” signal (Gardai et al. Cell 123: 321-334, 2005) binds to LRP1 (CD91) on dendritic cells and macrophages resulting in phagocytosis of the calreticulin expressing cell, unless the calreticulin-expressing cell expresses a don't eat me signal, such as CD47. Calreticulin also signals through CD91 on antigen presenting cells to cause the release of proinflammatory cytokines and to program Th17 cell responses. In summary, calreticulin expressed as part of immunogenic cell death stimulates antigen presenting cells to engulf dying cells, process their antigens and prime an immune response.

In addition to calreticulin, protein disulfide-isomerase A3 (PDIA3), also called Erp57, was shown to translocate from the ER to the surface of tumor cells following treatment with mitoxantrone, oxaliplatin and irradiation with UVC light (Panaretakis et al. CellDeath Differ. 15: 1499-1509, 2008; Panaretakis et al. EMBL J. 28: 578-590, 2009). A human ovarian cancer cell line, primary ovarian cancer cells and a human prostate cancer cell line expressed cell-surface calreticulin, HSP70 and HSP90 following treatment with the anthracyclines doxorubicin and idarrubicin (Fucikova et al. Cancer Res. 71: 4821-4833, 2011). HSP70 and HSP90 bind to the PRR LRP1 on antigen presenting cells; the PRR to which PDIA3 binds has not been identified (Galluzi et al. Nat. Rev. Immunol. 17: 97-111, 2016).

TLR4 was shown to be required for cross-presentation of dying tumor cells and to control tumor antigen processing and presentation. Among proteins that were known to bind to and stimulate TLR4, HMGB1 was uniquely released by mouse tumor cells in which ICD was induced by irradiation or doxorubicin (Apetoh et al. Nat. Med. 13: 1050-1059, 2007). The highly efficient induction of an in vivo anti-tumor immune by doxorubicin treatment of mouse tumor cells required the presence of HMGB1 and TLR4, as demonstrated by abrogation of the immune response by inhibition of HMGB1 and knock-out TLR4. These preclinical findings are clinically relevant. Patients with breast cancer who carry a TLR4 loss-of-function allele relapse more quickly after radiotherapy and chemotherapy than those carrying the normal TLR4 allele.

Ghiringhelli et al. showed that mouse tumor cells treated with oxaliplatin, doxorubicin and mitoanthrone in vitro released ATP and that the ATP binds to the purinergic receptors PY2, G-protein coupled, 2 (P2RY2) and PX2, ligand-gated ion channel, 7 (P2RX7) on dendritic cells (Ghiringhelli et al. Nat Med 15: 1170-1178, 2009). Binding of ATP to P2RX7 on DCs triggers the NOD-like receptor family, pyrin domain containing-3 protein (NLRP3)-dependent caspase-1 activation complex (inflammasome), allowing for the secretion of interleukin-1β (IL-1β), which is essential for the priming of interferon-gamma-producing CD8+ T cells by dying tumor cells. Therefore, the ATP-elicited production of IL-1β by DCs appears to be one of the critical factors for the immune system to perceive cell death induced by certain chemotherapy drugs as immunogenic. This paper also reports that HMGB1, at TLR4 agonist, also contributes to the stimulation of the NLRP3 inflammasome in DCs and the secretion of IL-1β. These preclinical results have been shown to have clinical relevance; in a breast cancer cohort, the presence of the P2RX7 loss-of-function allele had a significant negative prognostic impact of metastatic disease-free survival. ATP binding to P2RY2 causes the recruitment of myeloid cells into the tumor microenvironment (Vacchelli et al. Oncoimmunology 5: el 118600, 2016)

Michaud et al demonstrated that autophagy is required for the immunogenicity of chemotherapy-induced cell death (Michaud et al. Science 334: 1573-1577, 2011). Release of ATP from dying tumor cells required autophagy and autophagy-competent, but not autophagy-deficient, mouse tumors attracted dendritic cells and T lymphocytes into the tumor microenvironment in response to chemotherapy that induces ICD.

Ma et al addressed the question of how chemotherapy-induced cell death leads to efficient antigen presentation to T cells (Ma et al. Immunity 38: 729-741, 2013). They found that at specific kind of tumor infiltrating lymphocyte, CD11c+CD11b+Ly6Chi cells, are particularly important for the induction of anticancer immune responses by anthracyclines. ATP released by dying cancer cells recruited myeloid cells into tumors and stimulated the local differentiation of CD11c+CD11b+Ly6Chi cells. These cells were shown to be particularly efficient in capturing and presenting tumor cell antigens and, after adoptive transfer into naïve mice, conferring protection to challenge with living tumor cells of the same cell line.

It has been shown that anthracyclines stimulate the rapid production of type I interferons by tumor cells after activation of TLR3 (Sistugu et al. Nat. Med. 20: 1301-1309, 2014). Type I interferons bind to IFN-□ and IFN-□□receptors on cancer cells and trigger autocrine and paracrine signaling pathways that result in release of CXCL10. Tumors lacking Tlr3 or Ifnar failed to respond to chemotherapy unless type I IFN or CXCL10, respectively, was supplied. These preclinical findings have clinical relevance. A type I IFN-related gene expression signature predicted clinical responses to anthracycline-based chemotherapy in independent cohorts of breast cancer patients.

Another receptor on dendritic cells that is involved in chemotherapy-induced anti-cancer immune response was recently identified: formyl peptide receptor-1, which binds annexin A1 (Vacchelli et al. Science 350: 972-978, 2015). Vacchelli et al designed a screen to identify candidate genetic defects that negatively affect responses to chemotherapy. They identified a loss-of-function allele of the gene encoding formyl peptide receptor 1 (FPR1) that was associated with poor metastatis-free survival and overall survival in breast and colorectal cancer patients receiving adjuvant chemotherapy. The therapeutic effects of anthracyclines were abrogated in tumor-bearing Fpr1−/− mice due to impaired antitumor immunity. FPR1-deficient DCs did not approach dying tumor cells and, therefore, could not elicit antitumor T cell immunity. Two anthracyclines, doxorubicin and mitoxantrone, stimulated the secretion of annexin A1, one of four known ligands of FPR1. FPR1 and annexin A1 promoted stable interactions between dying cancer cells and human or mouse leukocytes.

In addition to anthracyclines and oxaliplatin, other drugs have been shown to induce immunogenic cell death. Cardiac glycosides, including clinically used digoxin and digitoxin, were also shown to be efficient inducers of immunogenic cell death of tumor cells (Menger et al. Sci Transl Med 4: 143ra99, 2012). Other chemotherapy agents and cancer drugs that have been reported to induce DAMP expression or release are bleomycin, bortezomib, cyclophosphamide, paclitaxel, vorinistat and cisplatin (Garg et al. Front. Immunol. 588: 1-24, 2015; Menger et al. Sci. Transl. Med. 4: 143ra99, 2012; Martins et al. Oncogene 30: 1147-1158, 2011). Importantly, these results have clinical relevance. Administration of digoxin during chemotherapy had a significant positive impact on the overall survival of patients with breast, colorectal, head and neck, and hepatocellular cancers, but failed to improve overall survival of lung and prostate cancer patients.

The anti-CD20 monoclonal antibody rituximab has improved outcomes in multiple B-cell malignancies. The success of rituximab, referred to as a type I anti-CD20 mAb, led to the development of type II anti-CD20 mAbs, including obinutuzumab and tositumomab. Cheadle et al investigated the induction of immunogenic cell death by anti-CD20 mAbs (Cheadle et al. Brit. J. Haematol. 162: 842-862, 2013). They found that the cell death induced by obinutuzumab and tositumomab is a form of immunogenic cell death characterized by the release of HMGB1, HSP90 and ATP. A type I anti-CD20 mAb did not cause release of HMGB1, HSP90 and ATP. Incubation of supernatants from a human tumor cell line treated with obinutuzumab caused maturation of human dendritic cells, consistent with the previously described effects of HMGB1 and ATP on dendritic cells. In contrast to the results reported by Cheadle et al, Zhao et al reported that both type I and II anti-CD20 mAbs increased HMGB1 release from human diffuse large B cell lymphoma cell lines, but did not cause ATP release or cell surface expression of calreticulin (Zhao et al. Oncotarget 6: 27817-27831, 2015).

The release from or exposure on tumor cell surfaces of the DAMPs calreticulin, ATP, HMGB1, annexin A1, type I interferon release, CXCL10, PDIA3, HSP70 and/or HSP90 in response to anti-CD47 mAbs has not been reported. As disclosed herein, anti-CD47 mAbs cause release from or exposure on tumor cell surfaces of the DAMPs listed above (characteristics of ICD), an unexpected result. These DAMPS are expected to promote a therapeutically beneficial adaptive anti-tumor immune response. Combining anti-CD47 mAbs that cause DAMP release/expression with chemotherapeutic agents that cause immunogenic cell death effects may result in greater therapeutic benefit than with either agent alone.

As disclosed herein, “causes an increase in cell surface calreticulin expression by human tumor cells” refers to a statistically significant increase (p<0.05 or greater) in calreticulin expression by a soluble anti-CD47 mAb compared to the background obtained with a negative control, humanized isotype-matched antibody or no treatment.

As disclosed herein, the term “the release of” is synonymous with secretion and is defined as the extracellular appearance of ATP, HMGB1, annexin A1, type I interferon and CXCL10.

As disclosed herein, “cause an increase in the release of adenosine triphosphate by human tumor cells” refers to a statistically significant increase (p<0.05 or greater) in ATP in the supernatant caused by a soluble anti-CD47 mAb compared to the background obtained with a negative control, humanized isotype-matched antibody or no treatment.

As disclosed herein, “cause an increase in the release of high mobility group box 1 by human tumor cells” refers to a statistically significant increase (p<0.05 or greater) in HMGB1 in the supernatant caused by a soluble anti-CD47 mAb compared to the background obtained with a negative control, humanized isotype-matched antibody or no treatment.

As disclosed herein, “causes an increase in the release of type I interferon by human tumor cells” refers to a statistically significant increase (p<0.05 or greater) in type I interferon in the supernatant or type I interferon mRNA caused by a soluble anti-CD47 mAb compared to the background obtained with a negative control, humanized isotype-matched antibody or no treatment.

As disclosed herein, “causes an increase in the release of C-X-C Motif Chemokine Ligand 10 (CXCL10) by human tumor cells” refers to a statistically significant increase (p<0.05 or greater) in CXCL10 in the supernatant or CXCL10 mRNA caused by a soluble anti-CD47 mAb compared to the background obtained with a negative control, humanized isotype-matched antibody or no treatment.

As disclosed herein, “causes an increase in cell surface PDIA3 expression by human tumor cells” refers to a statistically significant increase (p<0.05 or greater) in PDIA3 expression by a soluble anti-CD47 mAb compared to the background obtained with a negative control, humanized isotype-matched antibody or no treatment.

As disclosed herein, “causes an increase in cell surface HSP70 expression by human tumor cells” refers to a statistically significant increase (p<0.05 or greater) in HSP70 expression by a soluble anti-CD47 mAb compared to the background obtained with a negative control, humanized isotype-matched antibody or no treatment.

As disclosed herein, “causes an increase in cell surface HSP90 expression by human tumor cells” refers to statistically significant increase (p<0.05 or greater) in HSP90 expression by a soluble anti-CD47 mAb compared to the background obtained with a negative control, humanized isotype-matched antibody or no treatment.

### pH Dependence of Anti-CD47 mAb Binding

Most antibody binding, particularly in the blood compartment and to normal cells occurs at physiological pH (pH 7.2-7.4). Therefore, the binding affinity of therapeutic mAbs is normally assessed in vitro at physiological pH. However, the tumor microenvironment (TME) is more acidic in nature, with pH values below 7.0. This appears to be due to a number of differences including hypoxia, anaerobic glycolysis leading to the production of lactic acid and hydrolysis of ATP (Tannock and Rotin, Cancer Res 1989; Song et al, Cancer Drug Discovery and Development 2006; Chen and Pagel, Advan Radiol 2015). The acidic pH may provide an advantage to the tumor by promoting invasiveness, metastatic behavior, chronic autophagy, resistance to chemotherapies and reduced efficacy of immune cells in the tumor microenvironment (Estrella et al, Cancer Res 2013; Wojtkowiak et al, Cancer Res, 2012; Song et al, Cancer Drug Discovery and Development, 2006; Barar, BioImpacts, 2012). However, the identification of anti-CD47 antibodies with the property of increased binding affinity at acidic pH would confer a therapeutic advantage with higher binding to CD47 on tumor cells within the acidic TME compared to normal cells. Antibodies with pH-dependent properties have been generated with the goal of recycling antibodies. However, in contrast to exhibiting the properties of enhance binding at acidic pH, these bind with high affinity to their target antigen at physiological pH, but release their target at acidic pH (Bonvin et al, mAbs 2015; Igawa and Hattori, Biochem Biophys Acta 2014).

As disclosed herein, “has a greater affinity for CD47 at an acidic pH compared to physiological pH” refers to an apparent Kd that is increased 5-fold or more at acidic pH (<7.2) compared to physiological pH (7.2-7.4).

### Combinations of Functional Properties

In some embodiments of the anti-CD47 antibodies described herein, are also characterized by combinations of properties which are not exhibited by prior art anti-CD47 antibodies proposed for human therapeutic use. Accordingly, the anti-CD47 antibodies described herein are characterized by:
a. binds to human CD47;
b. blocks SIRPα, binding to human CD47;
c. increases phagocytosis of human tumor cells; and
d. induces death of susceptible human tumor cells.

In another embodiment described herein, the anti-CD47 antibodies are characterized by:
a. binds to human CD47;
b. blocks SIRPα, binding to human CD47;
c. increases phagocytosis of human tumor cells;
d. induces death of susceptible human tumor cells; and
e. causes no detectable agglutination of human red blood cells (hRBCs).

In yet another embodiment described herein, the anti-CD47 antibodies are characterized by:
a. binds to human CD47;
b. blocks SIRPα, binding to human CD47;
c. increases phagocytosis of human tumor cells;
d. induces death of susceptible human tumor cells; and
e. causes reduced agglutination of human red blood cells (hRBCs).

In another embodiment described herein, the anti-CD47 antibodies are characterized by:
a. specifically binds to human CD47;
b. blocks SIRPα binding to human CD47;
c. increases phagocytosis of human tumor cells;
d. induces death of susceptible human tumor cells; and
e. has reduced hRBC binding.

In another embodiment described herein, the anti-CD47 antibodies are characterized by:
a. binds to human CD47;
b. blocks SIRPα binding to human CD47;
c. increases phagocytosis of human tumor cells;
d. causes no detectable agglutination of human red blood cells (hRBCs); and
e. has minimal binding to hRBCs.

In another embodiment described herein, the anti-CD47 antibodies are characterized by:
a. specifically binds to human CD47;
b. blocks SIRPα binding to human CD47;
c. increases phagocytosis of human tumor cells;
d. causes no detectable agglutination of human red blood cells (hRBCs); and
e. has reduced hRBC binding.

In another embodiment described herein, the monoclonal antibody, or antigen binding fragment thereof binds to human, non-human primate, mouse, rabbit, and rat CD47.

In yet another embodiment described herein, the monoclonal antibody, or antigen binding fragment thereof specifically also binds to non-human primate CD47, wherein non-human primate may include, but is not limited to, cynomolgus monkey, green monkey, rhesus monkey and squirrel monkey.

In a embodiment described herein, the anti-CD47 monoclonal antibody, or antigen binding fragment thereof, may additionally possess one or more of the following characteristics: 1) exhibit cross-reactivity with one or more species homologs of CD47; 2) block the interaction between CD47 and its ligand SIRPα; 3) increase phagocytosis of human tumor cells; 4) induce death of susceptible human tumor cells; 5) do not induce cell death of human tumor cells; 6) do not have reduced or minimal binding to human red blood cells (hRBCs); 7) have reduced binding to hRBCs; 8) have minimal binding to hRBCs; 9) cause reduced agglutination of hRBCs; 10) cause no detectable agglutination of hRBCs; 11) reverse TSP1 inhibition of the nitric oxide (NO) pathway; 12) do not reverse TSP1 inhibition of the NO pathway; 13) cause loss of mitochondrial membrane potential; 14) do not cause loss of mitochondrial membrane potential; 15) cause an increase in cell surface calreticulin expression on human tumor cells; 16) do not cause an increase in cell surface calreticulin expression on human tumor cells; 17) cause an increase in adenosine triphosphate (ATP) release by human tumor cells; 18) do not cause an increase in adenosine triphosphate (ATP) release by human tumor cells; 19) cause an increase in high mobility group box 1 (HMGB1) release by human tumor cells; 20) do not cause an increase in high mobility group box 1 (HMGB1) release by human tumor cells; 21) cause an increase in type I interferon release by human tumor cells; 22) do not cause an increase in type I interferon release by human tumor cells; 23) cause an increase in C-X-C Motif Chemokine Ligand 10 (CXCL10) release by human tumor cells; 24) do not cause an increase in C-X-C Motif Chemokine Ligand 10 (CXCL10) release by human tumor cells; 25) cause an increase in cell surface protein disulfide-isomerase A3 (PDIA3) expression on human tumor cells; 26) do not cause an increase in cell surface protein disulfide-isomerase A3 (PDIA3) expression on human tumor cells; 27) cause an increase in cell surface heat shock protein 70 (HSP70) expression on human tumor cells; 28) do not cause an increase in cell surface heat shock protein 70 (HSP70) expression on human tumor cells; 29) cause an increase in cell surface heat shock protein 90 (HSP90) expression on human tumor cells; 30) do not cause an increase in cell surface heat shock protein 90 (HSP90) expression on human tumor cells; 31) have reduced binding to normal human cells, which includes, but is not limited to, endothelial cells, skeletal muscle cells, epithelial cells, and peripheral blood mononuclear cells (e.g., human aortic endothelial cells, human skeletal muscle cells, human microvascular endothelial cells, human renal tubular epithelial cells, human peripheral blood CD3+ cells, and human peripheral blood mononuclear cells); 32) do not have reduced binding to normal human cells, which includes, but is not limited to, endothelial cells, skeletal muscle cells, epithelial cells, and peripheral blood mononuclear cells (e.g., human aortic endothelial cells, human skeletal muscle cells, human microvascular endothelial cells, human renal tubular epithelial cells, human peripheral blood CD3+ cells, and human peripheral blood mononuclear cells); 33) have a greater affinity for human CD47 at an acidic pH compared to physiological pH; 34) do not have a greater affinity for human CD47 at an acidic pH compared to physiological pH; and 35) cause an increase in annexin A1 release by human tumor cells.

In some embodiments, a monoclonal antibody, or an antigen binding fragment thereof, is provided, which: binds to human CD47; blocks SIRPα binding to human CD47; increases phagocytosis of human tumor cells; and induces death of human tumor cells; wherein said monoclonal antibody, or an antigen binding fragment thereof, exhibits pH-dependent binding to CD47 present on a cell. In other embodiments, the disclosure provides a monoclonal antibody, or an antigen binding fragment thereof, which: binds to human CD47; blocks SIRPα binding to human CD47; increases phagocytosis of human tumor cells; and induces death of human tumor cells; wherein said monoclonal antibody, or an antigen binding fragment thereof, exhibits reduced binding to normal cells. In some embodiments, a cell to which such an antibody may bind may be of any cell type as described herein. In other embodiments, a monoclonal antibody as described herein, or an antigen binding fragment thereof, may exhibit any combination of characteristics provided in the present disclosure. For example, a monoclonal antibody may beneficially exhibit both pH dependent binding and reduced binding to a cell. These cells may be an endothelial cell, a skeletal muscle cell, an epithelial cell, a PBMC or a RBC (e.g., human aortic endothelial cells, human skeletal muscle cells, human microvascular endothelial cells, human renal tubular epithelial cells, human peripheral blood CD3+ cells, human peripheral blood mononuclear cells or human RBC). Such characteristics may be exhibited individually or in any combination as described herein. As used herein, pH dependent binding of an antibody of the disclosure may refer to altered binding of the antibody at a particular pH, for example an antibody that exhibits increased binding affinity at acidic pH.

### CD47 Antibodies

Many human cancers up-regulate cell surface expression of CD47 and those expressing the highest levels of CD47 are appear to be the most aggressive and the most lethal for patients. Increased CD47 expression is thought to protect cancer cells from phagocytic clearance by sending a “don't eat me” signal to macrophages via SIRPα, an inhibitory receptor that prevents phagocytosis of CD47-bearing cells (Oldenborg et al. Science 288: 2051-2054, 2000; Jaiswal et al. (2009) Cell 138(2):271-851; Chao et al. (2010) Science Translational Medicine 2(63):63ra94). Thus, the increase of CD47 expression by many cancers provides them with a cloak of “selfness” that slows their phagocytic clearance by macrophages and dendritic cells.

Antibodies that block CD47 and prevent its binding to SIRPα, have shown efficacy in human tumor in murine (xenograft) tumor models. Such blocking anti-CD47 mAbs exhibiting this property increase the phagocytosis of cancer cells by macrophages, which can reduce tumor burden (Majeti et al. (2009) Cell 138 (2): 286-99; U.S. Pat. No. 9,045,541; Willingham et al. (2012) Proc Natl Acad. Sci. USA 109(17):6662-6667; Xiao et al. (2015) Cancer Letters 360:302-309; Chao et al. (2012) Cell 142:699-713; Kim et al. (2012) Leukemia 26:2538-2545).

Anti-CD47 mAbs have also been shown to promote an adaptive immune response to tumors in vivo (Tseng et al. (2013) PNAS 110 (27):11103-11108; Soto-Pantoja et al. (2014) Cancer Res. 74 (23): 6771-6783; Liu et al. (2015) Nat. Med. 21 (10): 1209-1215).

However, there are mechanisms by which anti-CD47 mAbs can attack transformed cells that have not yet been exploited in the treatment of cancer. Multiple groups have shown that particular anti-human CD47 mAbs induce cell death of human tumor cells. Anti-CD47 mAb Ad22 induces cell death of multiple human tumor cells lines (Pettersen et al. J. Immunol. 166: 4931-4942, 2001; Lamy et al. J. Biol. Chem. 278: 23915-23921, 2003). AD22 was shown to indice rapid mitochondrial dysfunction and rapid cell death with early phosphatidylserine exposure and a drop in mitochondrial membrane potential (Lamy et al. J. Biol. Chem. 278: 23915-23921, 2003). Anti-CD47 mAb MABL-2 and fragments thereof induce cell death of human leukemia cell lines, but not normal cells in vitro and had an anti-tumor effect in in vivo xenograft models. (Uno et al. (2007) Oncol. Rep. 17 (5): 1189-94). Anti-human CD47 mAb 1F7 induces cell death of human T cell leukemias (Manna and Frazier (2003) J. Immunol. 170: 3544-53) and several breast cancers (Manna and Frazier (2004) Cancer Research 64 (3):1026-36). 1F7 kills CD47-bearing tumor cells without the action of complement or cell mediated killing by NK cells, T cells, or macrophages. Instead, anti-CD47 mAb 1F7 acts via a non-apoptotic mechanism that involves a direct CD47-dependent attack on mitochondria, discharging their membrane potential and destroying the ATP-generating capacity of the cell leading to rapid cell death. It is noteworthy that anti-CD47 mAb 1F7 does not kill resting leukocytes, which also express CD47, but only those cells that are “activated” by transformation. Thus, normal circulating cells, many of which express CD47, are spared while cancer cells are selectively killed by the tumor-toxic CD47 mAb (Manna and Frazier (2003) J. Immunol. 170: 3544-53). This mechanism can be thought of as a proactive, selective and direct attack on tumor cells in contrast to the passive mechanism of causing phagocytosis by simply blocking CD47/SIRPα binding. Importantly, mAb 1F7 also blocks binding of SIRPα to CD47 (Rebres et al et al. J. Cellular Physiol. 205: 182-193, 2005) and thus it can act via two mechanisms: (1) direct tumor toxicity, and (2) causing phagocytosis of cancer cells. A single mAb that can accomplish both functions may be superior to one that only blocks CD47/SIRPα binding.

An additional mechanism by which anti-CD47 mAbs can be exploited in the treatment of cancer is through the promotion of an anti-tumor immune response. The discovery that anti-CD47 mAbs cause tumor cells to release DAMPs that cause maturation, activation and homing of DCs and attraction of T cells connects anti-CD47 mAb treatment to the development of the therapeutically desirable anti-tumor immune response. Anti-CD47 mAbs have not been previously shown to cause tumor cell release of ATP, HMGB1, annexin A1, type I interferons and CXCL10 and tumor cell expression of calreticulin, PDIA3, HSP70 and HSP90.

Following periods of tissue ischemia, the initiation of blood flow causes damage referred to as “ischemia-reperfusion injury” or IRI. IRI contributes to poor outcomes in many surgical procedures where IRI occurs due to the necessity to stop blood flow for a period of time, in many forms/causes of trauma in which blood flow is interrupted and later restored by therapeutic intervention and in procedures required for organ transplantation, cardio/pulmonary bypass procedures, reattachment of severed body parts, reconstructive and cosmetic surgeries and other situations involving stopping and restarting blood flow. Ischemia itself causes many physiological changes that, by themselves would eventually lead to cell and tissue necrosis and death. Reperfusion poses its own set of damaging events including generation of reactive oxygen species, thrombosis, inflammation and cytokine mediated damage. The pathways that are limited by the TSP1-CD47 system are precisely those that would be of most benefit in combating the damage of IRI, including the NO pathway. Thus, blocking the TSP1-CD47 pathway, as with the antibodies disclosed herein, will provide more robust functioning of these endogenous protective pathways. Anti-CD47 mAbs have been shown to reduce organ damage in rodent models of renal warm ishchemia (Rogers et al. J Am Soc Nephrol. 23: 1538-1550, 2012), liver ischemia-reperfusion injury (Isenberg et al. Surgery. 144: 752-761, 2008), renal transplantation (Lin et al. Transplantation. 98: 394-401, 2014; Rogers et al. Kidney Interantional. 90: 334-347, 2016)) and liver transplantation, including steatotic livers (Xiao et al. Liver Transpl. 21: 468-477, 2015; Xiao et al. Transplantation. 100: 1480-1489, 2016). In addition, anti-CD47 mAb caused significant reductions of right ventricular systolic pressure and right ventricular hypertrophy in the monocrotaline model of pulmonary arterial hypertension (Bauer et al. Cardiovasc Res. 93: 682-693, 2012). Studies in skin flap models have shown that modulation of CD47, including with anti-CD47 mAbs, inhibits TSP1-mediated CD47 signaling. This results in increased activity of the NO pathway, resulting in reduced IRI (Maxhimer et al. Plast Reconstr Surg. 124: 1880-1889, 2009; Isenberg et al. Arterioscler Throm Vasc Biol. 27: 2582-2588, 2007; Isenberg et al. Curr Drug Targets. 9: 833-841, 2008; Isenberg et al. Ann Surg. 247: 180-190, 2008)

Anti-CD47 mAbs have also been shown to be efficacious in models of other cardiovascular diseases. In the mouse transverse aortic constriction model of pressure overload left ventricular heart failure, anti-CD47 mAb mitigated cardiac myocyte hypertrophy, decreased left ventricular fibrosis, prevented an increase in left ventricular weight, decreased ventricular stiffness, and normalized changes in the pressure volume loop profile (Sharifi-Sanjani et al. J Am Heart Assoc., 2014). An anti-CD47 mAb ameliorated atherosclerosis in multiple mouse models (Kojima et al. Nature., 2016).

### Cancer Indications

Presently disclosed are anti-CD47 mAbs and antigen binding fragments thereof effective as cancer therapeutics which can be administered to patients, preferably parenterally, with susceptible hematologic cancers and solid tumors including, but not limited to, leukemias, including systemic mastocytosis, acute lymphocytic (lymphoblastic) leukemia (ALL), T cell—ALL, acute myeloid leukemia (AML), myelogenous leukemia, chronic lymphocytic leukemia (CLL), chronic myeloid leukemia (CMIL), myeloproliferative disorder/neoplasm, monocytic cell leukemia, and plasma cell leukemia; multiple myeloma (MM); Waldenstrom's Macroglobulinemia; lymphomas, including histiocytic lymphoma and T cell lymphoma, B cell lymphomas, including Hodgkin's lymphoma and non-Hodgkin's lymphoma, such as low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL; solid tumors, including ovarian cancer, breast cancer, endometrial cancer, colon cancer (colorectal cancer), rectal cancer, bladder cancer, urothelial cancer, lung cancer (non-small cell lung cancer, adenocarcinoma of the lung, squamous cell carcinoma of the lung), bronchial cancer, bone cancer, prostate cancer, pancreatic cancer, gastric cancer, hepatocellular carcinoma (liver cancer, hepatoma), gall bladder cancer, bile duct cancer, esophageal cancer, renal cell carcinoma, thyroid cancer, squamous cell carcinoma of the head and neck (head and neck cancer), testicular cancer, cancer of the endocrine gland, cancer of the adrenal gland, cancer of the pituitary gland, cancer of the skin, cancer of soft tissues, cancer of blood vessels, cancer of brain, cancer of nerves, cancer of eyes, cancer of meninges, cancer of oropharynx, cancer of hypopharynx, cancer of cervix, and cancer of uterus, glioblastoma, medulloblastoma, astrocytoma, glioma, meningioma, gastrinoma, neuroblastoma, myelodysplastic syndrome, and sarcomas including, but not limited to, osteosarcoma, Ewing's sarcoma, leiomyosarcoma, synovial sarcoma, alveolar soft part sarcoma, angiosarcoma, liposarcoma, fibrosarcoma, rhabdomyosarcoma, and chondrosarcoma; and melanoma.

### Treatment of Cancer

As is well known to those of ordinary skill in the art, combination therapies are often employed in cancer treatment as single-agent therapies or procedures may not be sufficient to treat or cure the disease or condition. Conventional cancer treatments often involve surgery, radiation treatment, the administration of a combination of cytotoxic drugs to achieve additive or synergistic effects, and combinations of any or all of these approaches. Especially useful chemotherapeutic and biologic therapy combinations employ drugs that work via different mechanisms of action, increasing cancer cell control or killing, increasing the ability of the immune system to control cancer cell growth, reducing the likelihood of drug resistance during therapy, and minimizing possible overlapping toxicities by permitting the use of reduced doses of individual drugs.

Classes of anti-cancer, anti-tumor, and anti-neoplastic agents useful in the combination therapies encompassed by the present methods are disclosed, for example, in Goodman & Gilman's The Pharmacological Basis of Therapeutics, Twelfth Edition (2010) L. L. Brunton, B. A. Chabner, and B. C. Knollmann Eds., Section VIII, “Chemotherapy of Neoplastic Diseases”, Chapters 60-63, pp. 1665-1770, McGraw-Hill, NY, and include, for example, anthracyclines, platinums, taxols, topoisomerase inhibitors, anti-metabolites, anti-tumor antibiotics, mitotic inhibitors, and alkylating agents, natural products, a variety of miscellaneous agents, hormones and antagonists, targeted drugs, monoclonal antibodies and other protein therapeutics.

In addition to the foregoing, the methods of the present disclosure are related to treatment of cancer indications and further comprises treating the patient via surgery, radiation, and/or administering to a patient in need thereof an effective amount of a chemical small molecule or biologic drug including, but not limited to, a peptide, polypeptide, protein, nucleic acid therapeutic, conventionally used or currently being developed, to treat tumorous conditions. This includes antibodies and antigen-binding fragments, other than those disclosed herein, cytokines, antisense oligonucleotides, siRNAs, and miRNAs.

The therapeutic methods disclosed and claimed herein include the use of the antibodies disclosed herein alone, and/or in combinations with one another, and/or with antigen-binding fragments thereof of the present disclosure that bind to CD47, and/or with competing antibodies exhibiting appropriate biological/therapeutic activity, as well, for example, all possible combinations of these antibody compounds to achieve the greatest treatment efficacy.

In addition, the present therapeutic methods also encompass the use of these anti-CD47 mAbs, antigen-binding fragments thereof, competing antibodies, and combinations thereof further in combination with: (1) any one or more anti-tumor therapeutic treatments selected from surgery, radiation, anti-tumor, anti-neoplastic, anti-cancer agents, and combinations of any of these, or (2) any one or more of anti-tumor biological agents, or (3) equivalents of any of (1) or (2) as would be apparent to one of ordinary skill in the art, in appropriate combination(s) to achieve the desired therapeutic treatment effect for the particular indication.

Antibody and small molecule drugs that increase the immune response to cancer by modulating co-stimulatory or inhibitory interactions that influence the T cell response to tumor antigens, including inhibitors of immune checkpoints and modulators of co-stimulatory molecules, are also of particular interest in the context of the combination therapeutic methods encompassed herein and include, but are not limited to, other anti-CD47 antibodies.

Administration of therapeutic agents that bind to the CD47 protein, for example, antibodies or small molecules that bind to CD47 and prevent interaction between CD47 and SIRPα, are administered to a patient, causing the clearance of cancer cells via phagocytosis.

The therapeutic agent that binds to the CD47 protein is combined with a therapeutic agent such as an antibody, a chemical small molecule or biologic drug disclosed herein, directed against one or more additional cellular targets including, but not limited to, CD70 (Cluster of Differentiation 70), CD200 (OX-2 membrane glycoprotein, Cluster of Differentiation 200), CD154 (Cluster of Differentiation 154, CD40L, CD40 ligand, Cluster of Differentiation 40 ligand), CD223 (Lymphocyte-activation gene 3, LAG3, Cluster of Differentiation 223), KIR (Killer-cell immunoglobulin-like receptors), GITR (TNFRSF18, glucocorticoid-induced TNFR-related protein, activation-inducible TNFR family receptor, AITR, Tumor necrosis factor receptor superfamily member 18), CD28 (Cluster of Differentiation 28), CD40 (Cluster of Differentiation 40, Bp50, CDW40, TNFRSF5, Tumor necrosis factor receptor superfamily member 5, p50), CD86 (B7-2, Cluster of Differentiation 86), CD160 (Cluster of Differentiation 160, BY55, NK1, NK28), CD258 (LIGHT, Cluster of Differentiation 258, Tumor necrosis factor ligand superfamily member 14, TNFSF14, HVEML, HVEM ligand, herpesvirus entry mediator ligand, LTg), CD270 (HVEM, Tumor necrosis factor receptor superfamily member 14, herpesvirus entry mediator, Cluster of Differentiation 270, LIGHTR, HVEA), CD275 (ICOSL, ICOS ligand, Inducible T-cell co-stimulator ligand, Cluster of Differentiation 275), CD276 (B7-H3, B7 homolog 3, Cluster of Differentiation 276), OX40L (OX40 Ligand), B7-H4 (B7 homolog 4, VTCN1, V-set domain-containing T-cell activation inhibitor 1), GITRL (Glucocorticoid-induced tumor necrosis factor receptor-ligand, glucocorticoid-induced TNFR-ligand), 4-1BBL (4-1BB ligand), CD3 (Cluster of Differentiation 3, T3D), CD25 (IL2Rα, Cluster of Differentiation 25, Interleukin-2 Receptor α chain, IL-2 Receptor α chain), CD48 (Cluster of Differentiation 48, B-lymphocyte activation marker, BLAST-1, signaling lymphocytic activation molecule 2, SLAMF2), CD66a (Ceacam-1, Carcinoembryonic antigen-related cell adhesion molecule 1, biliary glycoprotein, BGP, BGP1, BGPI, Cluster of Differentiation 66a), CD80 (B7-1, Cluster of Differentiation 80), CD94 (Cluster of Differentiation 94), NKG2A (Natural killer group 2A, killer cell lectin-like receptor subfamily D member 1, KLRD1), CD96 (Cluster of Differentiation 96, TActILE, T cell activation increased late expression), CD112 (PVRL2, nectin, Poliovirus receptor-related 2, herpesvirus entry mediator B, HVEB, nectin-2, Cluster of Differentiation 112), CD115 (CSF1R, Colony stimulating factor 1 receptor, macrophage colony-stimulating factor receptor, M-CSFR, Cluster of Differentiation 115), CD205 (DEC-205, LY75, Lymphocyte antigen 75, Cluster of Differentiation 205), CD226 (DNAM1, Cluster of Differentiation 226, DNAX Accessory Molecule-1, PTA1, platelet and T cell activation antigen 1), CD244 (Cluster of Differentiation 244, Natural killer cell receptor 2B4), CD262 (DR5, TrailR2, TRAIL-R2, Tumor necrosis factor receptor superfamily member 10b, TNFRSF10B, Cluster of Differentiation 262, KILLER, TRICK2, TRICKB, ZTNFR9, TRICK2A, TRICK2B), CD284 (Toll-like Receptor-4, TLR4, Cluster of Differentiation 284), CD288 (Toll-like Receptor-8, TLR8, Cluster of Differentiation 288), Leukemia Inhibitor Factor (LIF), TNFSF15 (Tumor necrosis factor superfamily member 15, Vascular endothelial growth inhibitor, VEGI, TL1A), TDO2 (Tryptophan 2,3-dioxygenase, TPH2, TRPO), IGF-1R (Type I Insulin-like Growth Factor), GD2 (Disialoganglioside 2), TMIGD2 (Transmembrane and immunoglobulin domain-containing protein 2), RGMB (RGM domain family, member B), VISTA (V-domain immunoglobulin-containing suppressor of T-cell activation, B7-H5, B7 homolog 5), BTNL2 (Butyrophilin-like protein 2), Btn (Butyrophilin family), TIGIT (T cell Immunoreceptor with Ig and ITIM domains, Vstm3, WUCAM), Siglecs (Sialic acid binding Ig-like lectins), i.e., SIGLEC-15, Neurophilin, VEGFR (Vascular endothelial growth factor receptor), ILT family (LIRs, immunoglobulin-like transcript family, leukocyte immunoglobulin-like receptors), NKG families (Natural killer group families, C-type lectin transmembrane receptors), MICA (MHC class I polypeptide-related sequence A), TGFβ (Transforming growth factor β), STING pathway (Stimulator of interferon gene pathway), Arginase (Arginine amidinase, canavanase, L-arginase, arginine transamidinase), EGFRvIII (Epidermal growth factor receptor variant III), and HHLA2 (B7-H7, B7y, HERV-H LTR-associating protein 2, B7 homolog 7), inhibitors of PD-1 (Programmed cell death protein 1, PD-1, CD279, Cluster of Differentiation 279), PD-L1 (B7-H1, B7 homolog 1, Programmed death-ligand 1, CD274, cluster of Differentiation 274), PD-L2 (B7-DC, Programmed cell death 1 ligand 2, PDCDILG2, CD273, Cluster of Differentiation 273), CTLA-4 (Cytotoxic T-lymphocyte-associated protein 4, CD152, Cluster of Differentiation 152), BTLA (B- and T-lymphocyte attenuator, CD272, Cluster of Differentiation 272), Indoleamine 2,3-dioxygenase (IDO, IDO1), TIM3 (HAVCR2, Hepatitis A virus cellular receptor 2, T cell immunoglobulin mucin-3, KIM-3, Kidney injury molecule 3, TIMD-3, T cell immunoglobulin mucin-domain 3), A2A adenosine receptor (ADO receptor), CD39 (ectonucleoside triphosphate diphosphohydrolase-1, Cluster of Differentiation 39, ENTPD1), and CD73 (Ecto-5′-nucleotidase, 5′-nucleotidase, 5′-NT, Cluster of Differentiation 73), CD27 (Cluster of Differentiation 27), ICOS (CD278, Cluster of Differentiation 278, Inducible T-cell Co-stimulator), CD137 (4-1BB, Cluster of Differentiation 137, tumor necrosis factor receptor superfamily member 9, TNFRSF9), OX40 (CD134, Cluster of Differentiation 134), and TNFSF25 (Tumor necrosis factor receptor superfamily member 25), including antibodies, small molecules, and agonists, are also specifically contemplated herein. Additional agents include IL-10 (Interleukin-10, human cytokine synthesis inhibitory factor, CSIF), BCMA, CS1, CD79A, CD79B, CD138, and Galectins.

The therapeutic agent that binds to the CD47 protein can be combined with a therapeutic agent such as an antibody, a chemical small molecule or biologic drug disclosed herein, directed against one or more additional cellular targets including, but not limited to, antigens expressed on the surface of a multiple myeloma cell, e.g., a malignant plasma cell, which include BCMA, CS1, CD38, CD79A, CD79B, CD138, and CD19.

The therapeutic agent that binds to the CD47 protein can be combined with a second therapeutic agent, wherein the second therapeutic agent is a Bruton's tyrosine kinase (BTK) inhibitor.

In some embodiments the Bruton's tyrosine kinase (BTK) inhibitor is chosen from ibrutinib (PCI-32765), acalabrutinib, and zanubrutinib.

The therapeutic agent that binds to the CD47 protein can be combined with a BCMA-targeting agent, wherein the BCMA-targeting agent is chosen from JNJ-4528, teclistamab (JNJ-7957) and belantamab mafodotin (GSK2857916).

The therapeutic agent that binds to the CD47 protein can be combined with a CAR-T cell, wherein the CAR-T cell is chosen from an anti-CD19 CAR-T cell or an anti-BCMA CAR-T cell.

Included in these therapeutic methods is the use of the anti-CD47 mAbs and antigen-binding fragments thereof disclosed herein, in combination with:

YERVOY® (ipilimumab; Bristol-Meyers Squibb) is an example of an approved anti-CTLA-4 antibody.

KEYTRUDA® (pembrolizumab; Merck) and OPDIVO® (nivolumab; Bristol-Meyers Squibb Company) are examples of approved anti-PD-1 antibodies.

TECENTRIQ® (atezolizumab; Roche) is an example of an approved anti-PD-L1 antibody.

BAVENCIO® (avelumab, Merck KGaA, Pfizer, and Eli Lilly and Company), is an example of an approved anti-PD-L1 antibody.

IMIINZI® (durvalumab; Medimmune/AstraZeneca) is an example of an approved monoclonal antibody monoclonal antibody that blocks the interaction of programmed cell death ligand 1 (PD-L1) with the PD-1 and CD80 (B7.1) molecules.

REVLIMID® (lenalidomide; Celgene) is an example of an approved medication that acts as an immunomodulator used to treat multiple myeloma (MM) and myelodysplastic syndromes (MDS). For multiple myeloma, it is used after at least one other treatment, i.e., an anti-CD47 mAb and/or bortezomib, and generally together with dexamethasone.

POMALYST® (pomalidomide; Celgene) is an example of an anti-angiogenic and also acts as an immodulator used as a treatment for relapsed and refractory multiple myeloma.

XPOVIO® (selinexor; Karyopharm Therapeutics) is an example of a selective inhibitor of nuclear export used as an anti-cancer drug. It works by binding to exportin 1 and thus blocking the transport of several proteins involved in cancer-cell growth from the cell nucleus to the cytoplasm, which ultimately arrests the cell cycle and leads to apoptosis.

### EXAMPLES

### Example 1

### Amino Acid Sequences

### Light Chain CDRs

| LCDR1 | LCDR2 | LCDR3 |
|---|---|---|
| Vx4-LCDR1 | Vx4-LCDR2 | Vx4-LCDR3 |
| RSRQSIVHTNGNTYLG | KVSNRFS | FQGSHVPYT |
| (SEQ ID NO: 11) | (SEQ ID NO: 15) | (SEQ ID NO: 18) |
| Vx8-LCDR1 | Vx8-LCDR2 | Vx8-LCDR3 |
| RASQDISNYLN | YTSRLYS | QQGNTLPWT |
| (SEQ ID NO: 12) | (SEQ ID NO: 16) | (SEQ ID NO: 19) |
| Vx8-LCDR1 | | |
| RASQSISNYLN | | |
| (SEQ ID NO: 13) | | |
| Vx9-LCDR1 | Vx9-LCDR2 | Vx9-LCDR3 |
| RSSQNIVQSNGNTYLE | KVFHRFS | FQGSHVPWT |
| (SEQ ID NO: 14) | (SEQ ID NO: 17) | (SEQ ID NO: 20) |

### Heavy Chain CDRs

| HCDR1 | HCDR2 | HCDR3 |
|---|---|---|
| Vx4-HCDR1 | Vx4-HCDR2 | Vx4-HCDR3 |
| GYTFTNYVIH | YIYPYNDGILYNEKFKG | GGYYVPDY |
| (SEQ ID NO: 1) | (SEQ ID NO: 4) | (SEQ ID NO: 7) |
| | | Vx4-HCDR3 |
| | | GGYYVYDY |
| | | (SEQ ID NO: 8) |
| Vx8-HCDR1 | Vx8-HCDR2 | Vx8-HCDR3 |
| GYSFTNYYIH | YIDPLNGDTTYNQKFKG | GGKRAIVIDY |
| (SEQ ID NO: 2) | (SEQ ID NO: 5) | (SEQ ID NO: 9) |
| Vx9-HCDR1 | Vx9-HCDR2 | Vx9-HCDR3 |
| GYTFTNYWIH | YTDPRTDYTEYNQKFKD | GGRVGLGY |
| (SEQ ID NO: 3) | (SEQ ID NO: 6) | (SEQ ID NO: 10) |

Marine Light Chain Variable Domains
>Vx4murL01
(SEQ ID NO: 41)
DVLMTQTPLSLPVNLGDQASISCRSRQSIVHTNGNTYLGWFLQKPGQSPKLLIYKVSNRF
SGVPDRFSGSGSGTDFTLTISRVEAEDLGVYYCFQGSHVPYTFGGGTKLEIK.
>Vx4murL02
(SEQ ID NO: 42)
DVLMTQTPLSLPVNLGDQASISCRSRQSIVHTNGNTYLGWFLQKPGQSPKLLIYKVSNRF
SGVPDRFSGSGSGTDFTLTISRVEAEDLGVYYCFQGSHVPYTFGQGTKVEIK.
>Vx8murL03
(SEQ ID NO: 46)
DIQMTQTTSSLSASLGDRVTISCRASQDISNYLNWYQQKPDGTVKLLIYYTSRLYSGVPS
RFSGSGSGTDYSLTISNLEQEDIATYFCQQGNTLPWTFGGGTKLEIK.
>Vx9murL04
(SEQ ID NO: 50)
DVFMTQTPLSLPVSLGDQASISCRSSQNIVQSNGNTYLEWYLQKPGQSPKLLIYKVFHRF
SGVPDRFSGSGSGTDFTLKISRVEAEDLGVYYCFQGSHVPWTFGGGTKVEIK
Murine Heavy Chain Variable Domains
>Vx4murH01
(SEQ ID NO: 21)
EVQLQQSGPELVKPGASVKMSCKASGYTFTNYVIHWVKRRPGQGLEWIGYIYPYNDGIL
YNEKFKGKATVTSDKSSSTAYMDLSSLTSEDSAVYYCTRGGYYVPDYWGQGTTLTVSS.
>Vx4mur-H02
(SEQ ID NO: 22)
EVQLQQSGPELVKPGASVKMSCKASGYTFTNYVIHWVKRRPGQGLEWIGYIYPYNDGIL
YNEKFKGKATVTSDKSSSTAYMDLSSLTSEDSAVYYCTRGGYYVPDYWGQGTLVTVSS.
>Vx8murH03
(SEQ ID NO: 28)
EVQLQQSGPELMKPGASVKISCKASGYSFTNYYIEWVNQSHGKSLEWIGYIDPLNGDTT
YNQKFKGKATLTVDKSSSTAYMRLSSLTSADSAVYYCARGGKRAMDYWGQGTSVTVSS.
>Vx9murH04
(SEQ ID NO: 35)
QVQLQQFGAELAKPGASVQMSCKASGYTFTNYWIHWVKQRPGQGLEWIGYTDPRTDY
TEYNQKFKDKATLAADRSSSTAYMRLSSLTSEDSAVYYCAGGGRVGLGYWGHGSSVT
VSS
Human Light Chain Variable Domains
>Vx4humL01
(SEQ ID NO: 43)
DIVMTQSPLSLPVTPGEPASISCRSRQSIVHTNGNTYLGWYLQKPGQSPRLLIYKVSNRFS
GVPDRFSGSGSGTDFTLKISRVEADDVGIYYCFQGSHVPYTFGQGTKLEIK
>Vx4humL02
(SEQ ID NO: 44)
DVVMTQSPLSLPVTLGQPASISCRSRQSIVHTNGNTYLGWFQQRPGQSPRRLIYKVSNRF
SGVPDRFSGSGSGTDFTLKISRVEAEDVGVYYCFQGSHVPYTFGQGTKLEIK
>Vx4humL03
(SEQ ID NO: 45)
DIVMTQSPDSLAVSLGERATINCRSRQSIVHTNGNTYLGWYQQKPGQPPKLLIYKVSNRF
SGVPDRFSGSGSGTDFTLTISSLQAEDVAVYYCFQGSHVPYTFGQGTKLEIK
>Vx8humL04
(SEQ ID NO: 47)
DIQMTQSPSSLSASVGDRVTITCRASQDISNYLNWYQQKPGKAPKLLIYYTSRLYSGVPS
RFSGSGSGTDFTFTISSLQPEDIATYYCQQGNTLPWTFGQGTKVEIK.
>Vx8humL05
(SEQ ID NO: 48)
DIQMTQSPSSLSASVGDRVTITCRASQSISNYLNWYQQKPGKAPKLLIYYTSRLYSGVPS
RFSGSGSGTDFTLTISSLQPEDFATYYCQQGNTLPWTFGQGTKVEIK.
>Vx8humL06
(SEQ ID NO: 49)
DIVMTQSPLSLPVTPGEPASISCRASQDISNYLNWYLQKPGQSPRLLIYYTSRLYSGVPDR
FSGSGSGTDFTLKISRVEADDVGIYYCQQGNTLPWTFGQGTKLEIK
>Vx9humL07
(SEQ ID NO: 51)
DVVMTQSPLSLPVTLGQPASISCRSSQNIVQSNGNTYLEWFQQRPGQSPRRLIYKVFHRF
SGVPDRFSGSGSGTDFTLKISRVEAEDVGVYYCFQGSHVPYTFGQGTKLEIK.
>Vx9humL08
(SEQ ID NO: 52)
DIVMTQSPDSLAVSLGERATINCRSSQNIVQSNGNTYLEWYQQKPGQPPKLLIYKVFHRF
SGVPDRFSGSGSGTDFTLTISSLQAEDVAVYYCFQGSHVPYTFGQGTKLEIK.
Human Heavy Chain Variable Domains
>Vx4humH01
(SEQ ID NO: 23)
QVQLVQSGAEVKKPGASVQVSCKASGYTFTNYVIHWLRQAPGQGLEWMGYIYPYNDG
ILYNEKFKGRVTMTSDTSISTAYMELSSLRSDDTAVYYCARGGYYVPDYWGQATLVTV
SS.
>Vx4humH02
(SEQ ID NO: 24)
QVQLVQSGAEVKKPGASVQVSCKASGYTFTNYVIHWLRQAPGQGLEWMGYIYPYNDG
ILYNEKFKGRVTMTSDTSISTAYMELSSLRSDDTAVYYCARGGYYVYDYWGQATLVTV
SS.
>Vx4humH03
(SEQ ID NO: 25)
EVQLVQSGAEVKKPGATVKISCKVSGYTFTNYVIHWVQQAPGKGLEWMGYIYPYNDGI
LYNEKFKGRVTITADTSTDTAYMELSSLRSEDTAVYYCATGGYYVPDYWGQGTTVTVS
S
>Vx4humH04
(SEQ ID NO: 26)
EVQLVQSGAEVKKPGESLKISCKGSGYTFTNYVIHWVRQMPGKGLEWMGYIYPYNDGI
LYNEKFKGQVTISADKSISTAYLQWSSLKASDTAMYYCARGGYYVPDYWGQGTTVTVS
S
>Vx4humH05
(SEQ ID NO: 27)
QVQLVQSGAEVKKPGASVKVSCKASGYTFTNYVIHWVRQAPGQGLEWMGYIYPYNDG
ILYNEKFKGRVTMTTDTSTSTAYMELRSLRSDDTAVYYCARGGYYVPDYWGQGTTVT
VSS
>Vx8humH06
(SEQ ID NO: 29)
QVQLVQSGAEVKKPGASVKVSCKASGYSFTNYYTHWVRQAPGQGLEWMGYIDPLNGD
TTYNQKFKGRVTMTRDTSTSTVYMELSSLRSEDTAVYYCARGGKRAMDYWGQGTLVT
VSS.
>Vx8humH07
(SEQ ID NO: 30)
QVQLVQSGAEVKKPGSSVKVSCKASGYSFTNYYIHWVRQAPGQGLEWMGYIDPLNGD
TTYNQKFKGRVTITADESTSTAYMELSSLRSEDTAVYYCARGGKRAMDYWGQGTLVT
VSS.
>Vx8humH08
(SEQ ID NO: 31)
EVQLVQSGAEVKKPGESLKISCKGSGYSFTNYYIHWVRQMPGKGLEWMGYIDPLNGDT
TYNQKFKGQVTISADKSISTAYLQWSSLKASDTAMYYCARGGKRAMDYWGQGTLVTV
SS.
>Vx8humH09
(SEQ ID NO: 32)
QVQLVQSGAEVKKPGSSVKVSCKASGYSFTNYYIHWVRQAPGQGLEWMGYIDPLNGD
TTYAQKFQGRVTITADESTSTAYMELSSLRSEDTAVYYCARGGKRAMDYWGQGTLVT
VSS.
>Vx8humH10
(SEQ ID NO: 33)
EVQLVQSGAEVKKPGESLKISCKGSGYSFTNYYIHWVRQMPGKGLEWMGYIDPLNGDT
TYSPSFQGQVTISADKSISTAYLQWSSLKASDTAMYYCARGGKRAMDYWGRGTLVTVS
S.
>Vx8humH11
(SEQ ID NO: 34)
QVQLVQSGAEVKKPGASVQVSCKASGYSFTNYYIHWLRQAPGQGLEWMGYIDPLNGD
TTYNQKFKGRVTMTSDTSISTAYMELSSLRSDDTAVYYCARGGKRAMDYWGQATLVT
VSS
>Vx9humH12
(SEQ ID NO: 36)
QVQLVQSGAEVKKPGASVKVSCKASGYTFTNYWIHWVRQAPGQGLEWMGYTDPRTD
YTEYNQKFKDRVTMTRDTSTSTVYMELSSLRSEDTAVYYCARGGRVGLGYWGQGTLV
TVSS.
>Vx9humH13
(SEQ ID NO: 37)
QVQLVQSGAEVKKPGSSVKVSCKASGYTFTNYWIHWVRQAPGQGLEWMGYTDPRTD
YTEYNQKFKDRVTITADESTSTAYMELSSLRSEDTAVYYCARGGRVGLGYWGQGTLVT
VSS.
>Vx9humH14
(SEQ ID NO: 38)
EVQLVQSGAEVKKPGESLKISCKGSGYTFTNYWIHWVRQMPGKGLEWMGYTDPRTDY
TEYNQKFKDQVTISADKSISTAYLQWSSLKASDTAMYYCARGGRVGLGYWGQGTLVT
VSS.
>Vx9humH15
(SEQ ID NO: 39)
QVQLVQSGAEVKKPGSSVKVSCKASGYTFTNYWIHWVRQAPGQGLEWMGYTDPRTD
YTEYAQKFQGRVTITADESTSTAYMELSSLRSEDTAVYYCARGGRVGLGYWGQGTLVT
VSS.
>Vx9humH16
(SEQ ID NO: 40)
EVQLVQSGAEVKKPGESLKISCKGSGYTFTNYWIHWVRQMPGKGLEWMGYTDPRTDY
TEYSPSFQGQVTISADKSISTAYLQWSSLKASDTAMYYCARGGRVGLGYWGQGTLVTV
SS.
Human IgG-Fc
>Human Fc IgG1
(SEQ ID NO: 53)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLEPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDK
SRWQQGNVFSCSVMHEALHNHYTQKSLSLSPGK.
>Human Fc IgG1-N297Q
(SEQ ID NO: 54)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLEPPKPKDTLMISRTPEVTCVVVDVSHEDPEVKFNWYVDGVEVHNAKTKPREEQY
QSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDK
SRWQQGNVFSCSVMHEALHNHYTQKSLSLSPGK.
>Human Fc-IgG2
(SEQ ID NO: 56)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSV
FLFPPKPKDTLMISRTPEVTCVVVDVSHEDPEVQFNWYVDGVEVHNAKTKPREEQFNST
FRVVSVLTVVHQDWLNGKEYKCKVSNKGLPAPIEKTISKTKGQPREPQVYTLPPSREEM
TKNQVSLTCLVKGFYPSDIAVEWESNGQPENNYKTTPPMLDSDGSFFLYSKLTVDKSRW
QQGNVFSCSVMHEALHNHYTQKSLSLSPGK.
>Human Fc-IgG3
(SEQ ID NO: 57)
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDTLMI
SRTPEVTCVVVDVSHEDPEVQFKWYVDGVEVHNAKTKPREEQYNSTFRVVSVLTVLHQ
DWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMH
EALHNRFTQKSLSLSPGK
>Human Fc-IgG4
(SEQ ID NO: 58)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLG.
>Human Fc-IgG4 S228P
(SEQ ID NO: 59)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLG.
>Human Fc-IgG4PE
(SEQ ID NO: 60)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLGK
>Human Fc-IgG4PE′
(SEQ ID NO: 101)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLG
>Human kappa LC
(SEQ ID NO: 61)
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC.
>Rat Fc-IgG2c
(SEQ ID NO: 62)
ARTTAPSVYPLVPGCSGTSGSLVTLGCLVKGYFPEPVTVKWNSGALSSGVHTFPAVLQS
GLYTLSSSVTVPSSTWSSQTVTCSVAHPATKSNLIKRIEPRRPKPRPPTDICSCDDNLGRPS
VFIFPPKPKDILMITLTPKVTCVVVDVSEEEPDVQFSWFVDNVRVFTAQTQPHEEQLNGT
FRVVSTLHIQHQDWMSGKEFKCKVNNKDLPSPIEKTISKPRGKARTPQVYTIPPPREQMS
KNKVSLTCMVTSFYPASISVEWERNGELEQDYKNTLPVLDSDESYFLYSKLSVDTDSW
MRGDIYTCSVVHEALHNHHTQKNLSRSPGK.
>Rat kappa LC
(SEQ ID NO: 63)
RADAAPTVSIFPPSMEQLTSGGATVVCFVNNFYPRDISVKWKIDGSEQRDGVLDSVTDQ
DSKDSTYSMSSTLSLTKVEYERHNLYTCEVVHKTSSSPVVKSFNRNEC.
Rabbit IgG-Fc
>Rabbit IgG
(SEQ ID NO: 64)
GQPKAPSVFPLAPCCGDTPSSTVTLGCLVKGYLPEPVTVTWNSGTLTNGVRTFPSVRQSS
GLYSLSSVVSVTSSSQPVTCNVAHPATNTKVDKTVAPSTCSKPTCPPPELLGGPSVFIFPP
KPKDTLMISRTPEVTCVVVDVSQDDPEVQFTWYINNEQVRTARPPLREQQFNSTIRVVST
LPIAHQDWLRGKEFKCKVHNKALPAPIEKTISKARGQPLEPKVYTMGPPREELSSRSVSL
TCMINGFYPSDISVEWEKNGKAEDNYKTTPAVLDSDGSYFLYSKLSVPTSEWQRGDVFT
CSVMHEALHNHYTQKSISRSPGK.
>Rabbit kappa LC
(SEQ ID NO: 65)
RDPVAPTVLIFPPAADQVATGTVTIVCVANKYFPDVTVTWEVDGTTQTTGIENSKTPQN
SADCTYNLSSTLTLTSTQYNSHKEYTCKVTQGTTSVVQSFNRGDC.
>CD47
(SEQ ID NO: 66)
MWPLVAALLLGSACCGSAQLLFNKTKSVEFTFCNDTVVIPCFVTNMEAQNTTEVYVKW
KFKGRDIYTFDGALNKSTVPTDFSSAKIEVSQLLKGDASLKMDKSDAVSHTGNYTCEVT
ELTREGETIIELKYRVVSWFSPNENILIVIFPIFAILLFWGQFGIKTLKYRSGGMDEKTTALL
VAGLVITVIVIVGAILFVPGEYSLKNATGLGLIVTSTGILILLHYYVFSTAIGLTSFVIAILVI
QVIAYILAVVGLSLCIAACIPMHGPLLISGLSILALAQLLGLVYMKFVE.
Chimera and Human Light Chains
>Vx4murL01 Full length
(SEQ ID NO: 67)
DVLMTQTPLSLPVNLGDQASISCRSRQSIVHTNGNTYLGWFLQKPGQSPKLLIYKVSNRF
SGVPDRFSGSGSGTDFTLTISRVEAEDLGVYYCFQGSHVPYTFGGGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
>Vx4murL01 Full length
(SEQ ID NO: 68)
DVLMTQTPLSLPVNLGDQASISCRSRQSIVHTNGNTYLGWFLQKPGQSPKLLIYKVSNRF
SGVPDRFSGSGSGTDFTLTISRVEAEDLGVYYCFQGSHVPYTFGQGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
>Vx4humL01 Full length LC
(SEQ ID NO: 69)
DIVMTQSPLSLPVTPGEPASISCRSRQSIVHTNGNTYLGWYLQKPGQSPRLLIYKVSNRFS
GVPDRFSGSGSGTDFTLKISRVEADDVGIYYCFQGSHVPYTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC.
>Vx8humL03 Full length LC
(SEQ ID NO: 70)
DIVMTQSPLSLPVTPGEPASISCRASQDISNYLNWYLQKPGQSPRLLIYYTSRLYSGVPDR
FSGSGSGTDFTLKISRVEADDVGIYYCQQGNTLPWTFGQGTKLEIKRTVAAPSVFIFPPSD
EQLKSGTASVVCLLNNFYPREAKVQWKVDNALQSGNSQESVTEQDSKDSTYSLSST
LTLSKADYEKHKVYACEVTHQGLSSPVTKSFNRGEC.
>Vx9humL02 Full length LC
(SEQ ID NO: 71)
DIVMTQSPDSLAVSLGERATINCRSSQNIVQSNGNTYLEWYQQKPGQPPKLLIYKVFHRF
SGVPDRFSGSGSGTDFTLTISSLQAEDVAVYYCFQGSHVPYTFGQGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
>Vx8humL02 Full length LC
(SEQ ID NO: 72)
DIQMTQSPSSLSASVGDRVTITCRASQSISNYLNWYQQKPGKAPKLLIYYTSRLYSGVPS
RFSGSGSGTDFTLTISSLQPEDFATYYCQQGNTLPWTFGQGTKVEIKRTVAAPSVFIFPPS
DEQLKSGTASVVCLLNNFYPREAKVQWKVDNALQSGNSQESVTEQDSKDSTYSLSST
LTLSKADYEKHKVYACEVTHQGLSSPVTKSFNRGEC.
>Vx4humL02 Full length LC
(SEQ ID NO: 73)
DVVMTQSPLSLPVTLGQPASISCRSRQSIVHTNGNTYLGWFQQRPGQSPRRLIYKVSNRF
SGVPDRFSGSGSGTDFTLKISRVEAEDVGVYYCFQGSHVPYTFGQGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
>Vx9humL07 Full length LC
(SEQ ID NO: 74)
DVVMTQSPLSLPVTLGQPASISCRSSQNIVQSNGNTYLEWFQQRPGQSPRRLIYKVFHRF
SGVPDRFSGSGSGTDFTLKISRVEAEDVGVYYCFQGSHVPYTFGQGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
>Vx8humL01 Full length LC
(SEQ ID NO: 75)
DIQMTQSPSSLSASVGDRVTITCRASQDISNYLNWYQQKPGKAPKLLIYYTSRLYSGVPS
RFSGSGSGTDFTFTISSLQPEDIATYYCQQGNTLPWTFGQGTKVEIKRTVAAPSVFIFPPSD
EQLKSGTASVVCLLNNFYPREAKVQWKVDNALQSGNSQESVTEQDSKDSTYSLSST
LTLSKADYEKHKVYACEVTHQGLSSPVTKSFNRGEC.
>Vx8murL03 Full length LC
(SEQ ID NO: 76)
DIQMTQTTSSLSASLGDRVTISCRASQDISNYLNWYQQKPDGTVKLLIYYTSRLYSGVPS
RFSGSGSGTDYSLTISNLEQEDIATYFCQQGNTLPWTFGGGTKLEIKRTVAAPSVFIFPPS
DEQLKSGTASVVCLLNNFYPREAKVQWKVDNALQSGNSQESVTEQDSKDSTYSLSST
LTLSKADYEKHKVYACEVTHQGLSSPVTKSFNRGEC.
>Vx9mur_L04 Full length LC
(SEQ ID NO: 77)
DVFMTQTPLSLPVSLGDQASISCRSSQNIVQSNGNTYLEWYLQKPGQSPKLLIYKVFHRF
SGVPDRFSGSGSGTDFTLKISRVEAEDLGVYYCFQGSHVPWTFGGGTKVEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC.
Chimera and Human Heavy Chains
>Vx4murH01 Full length HC
(SEQ ID NO: 78)
EVQLQQSGPELVKPGASVKMSCKASGYTFTNYVIHWVKRRPGQGLEWIGYIYPYNDGIL
YNEKFKGKATVTSDKSSSTAYMDLSSLTSEDSAVYYCTRGGYYVPDYWGQGTTLTVSS
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLGK.
>Vx4humH01 Full length HC
(SEQ ID NO: 79)
QVQLVQSGAEVKKPGASVQVSCKASGYTFTNYVIHWLRQAPGQGLEWMGYIYPYNDG
ILYNEKFKGRVTMTSDTSISTAYMELSSLRSDDTAVYYCARGGYYVPDYWGQATLVTV
SSASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFEGGP
SVFLFPPKPKDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFLYSRLTVDKS
RWQEGNVFSCSVMHEALHNHYTQKSLSLSLGK.
>Vx8humH11 Full length HC
(SEQ ID NO: 80)
QVQLVQSGAEVKKPGASVQVSCKASGYSFTNYYIHWLRQAPGQGLEWMGYIDPLNGD
TTYNQKFKGRVTMTSDTSISTAYMELSSLRSDDTAVYYCARGGKRAMDYWGQATLVT
VSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGG
PSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDK
SRWQEGNVFSCSVMHEALHNHYTQKSLSLSLGK.
>Vx9humH12 Full length HC
(SEQ ID NO: 81)
QVQLVQSGAEVKKPGASVKVSCKASGYTFTNYWIHWVRQAPGQGLEWMGYTDPRTD
YTEYNQKFKDRVTMTRDTSTSTVYMELSSLRSEDTAVYYCARGGRVGLGYWGQGTLV
TVSSASTKGPSVFPLAPCSRSTSESTAALGCLVKDYFPEPVTVSWNSGALTSGVHTFPAV
LQSSGLYSLSSVVTVPSSNFGTQTYTCNVDHKPSNTKVDKTVERKCCVECPPCPAPPVA
GPSVFLFPPKPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSFFLYSKLTVD
KSRWQQGNVFSCSVMHEALHNHYTQKSLSLSPGK.
>Vx9humH14 Full length HC
(SEQ ID NO: 82)
EVQLVQSGAEVKKPGESLKISCKGSGYTFTNYWIHWVRQMPGKGLEWMGYTDPRTDY
TEYNQKFKDQVTISADKSISTAYLQWSSLKASDTAMYYCARGGRVGLGYWGQGTLVT
VSSASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCCVECPPCPAPPVAG
PSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSR
EEMTKNQVSLTCLVKGFYPSDIAVEWESNGQPENNYKTTPPMLDSDGSFFLYSKLTVDK
SRWQQGNVFSCSVMHEALHNHYTQKSLSLSPGK.
>Vx9humH15 Full length HC
(SEQ ID NO: 83)
QVQLVQSGAEVKKPGSSVKVSCKASGYTFTNYWIHWVRQAPGQGLEWMGYTDPRTD
YTEYAQKFQGRVTITADESTSTAYMELSSLRSEDTAVYYCARGGRVGLGYWGQGTLVT
VSSASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCCVECPPCPAPPVAG
PSVFLEPPKPKDTLMISRTPEVTCVVVDVSHEDPEVQFNWYVDGVEVHNAKTKPREEQF
NSTERVVSVLTVVHQDWLNGKEYKCKVSNKGLPAPIEKTISKTKGQPREPQVYTLPPSR
EEMTKNQVSLTCLVKGFYPSDIAVEWESNGQPENNYKTTPPMLDSDGSFFLYSKLTVDK
SRWQQGNVFSCSVMHEALHNHYTQKSLSLSPGK.
>Vx4humH02 Full length HC
(SEQ ID NO: 84)
QVQLVQSGAEVKKPGASVQVSCKASGYTFTNYVIHWLRQAPGQGLEWMGYIYPYNDG
ILYNEKFKGRVTMTSDTSISTAYMELSSLRSDDTAVYYCARGGYYVYDYWGQATLVTV
SSASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFEGGP
SVFLEPPKPKDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFELYSRLTVDKS
RWQEGNVFSCSVMHEALHNHYTQKSLSLSLGK.
>Vx9humH13 Full length HC
(SEQ ID NO: 85)
QVQLVQSGAEVKKPGSSVKVSCKASGYTFTNYWIHWVRQAPGQGLEWMGYTDPRTD
YTEYNQKFKDRVTITADESTSTAYMELSSLRSEDTAVYYCARGGRVGLGYWGQGTLVT
VSSASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCCVECPPCPAPPVAG
PSVFLEPPKPKDTLMISRTPEVTCVVVDVSHEDPEVQFNWYVDGVEVHNAKTKPREEQF
NSTERVVSVLTVVHQDWLNGKEYKCKVSNKGLPAPIEKTISKTKGQPREPQVYTLPPSR
EEMTKNQVSLTCLVKGFYPSDIAVEWESNGQPENNYKTTPPMLDSDGSFFLYSKLTVDK
SRWQQGNVFSCSVMHEALHNHYTQKSLSLSPGK.
>Vx8humH10 Full length HC
(SEQ ID NO: 86)
EVQLVQSGAEVKKPGESLKISCKGSGYSFTNYYTHWVRQMPGKGLEWMGYIDPLNGDT
TYSPSFQGQVTISADKSISTAYLQWSSLKASDTAMYYCARGGKRAMDYWGRGTLVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPS
VFLEPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSR
WQEGNVFSCSVMHEALHNHYTQKSLSLSLGK.
>Vx4humH04 Full length HC
(SEQ ID NO: 87)
EVQLVQSGAEVKKPGESLKISCKGSGYTFTNYVIHWVRQMPGKGLEWMGYIYPYNDGI
LYNEKFKGQVTISADKSISTAYLQWSSLKASDTAMYYCARGGYYVPDYWGQGTTVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPS
VFLEPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSR
WQEGNVFSCSVMHEALHNHYTQKSLSLSLGK.
>Vx4humH05 Full length HC
(SEQ ID NO: 88)
QVQLVQSGAEVKKPGASVKVSCKASGYTFTNYVIHWVRQAPGQGLEWMGYIYPYNDG
ILYNEKFKGRVTMTTDTSTSTAYMELRSLRSDDTAVYYCARGGYYVPDYWGQGTTVT
VSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGG
PSVFLEPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFELYSRLTVDK
SRWQEGNVFSCSVMHEALHNHYTQKSLSLSLGK.
>Vx9humH16 Full length HC
(SEQ ID NO: 89)
EVQLVQSGAEVKKPGESLKISCKGSGYTFTNYWIHWVRQMPGKGLEWMGYTDPRTDY
TEYSPSFQGQVTISADKSISTAYLQWSSLKASDTAMYYCARGGRVGLGYWGQGTLVTV
SSASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVERKCCVECPPCPAPPVAGPS
VFLEPPKPKDTLMISRTPEVTCVVVDVSHEDPEVQFNWYVDGVEVHNAKTKPREEQFNS
TFRVVSVLTVVHQDWLNGKEYKCKVSNKGLPAPIEKTISKTKGQPREPQVYTLPPSREE
MTKNQVSLTCLVKGFYPSDIAVEWESNGQPENNYKTTPPMLDSDGSFFLYSKLTVDKSR
WQQGNVFSCSVMHEALHNHYTQKSLSLSPGK.
>Vx8humH06 Full length HC
(SEQ ID NO: 90)
QVQLVQSGAEVKKPGASVKVSCKASGYSFTNYYIHWVRQAPGQGLEWMGYIDPLNGD
TTYNQKFKGRVTMTRDTSTSTVYMELSSLRSEDTAVYYCARGGKRAMDYWGQGTLVT
VSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGG
PSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDK
SRWQEGNVFSCSVMHEALHNHYTQKSLSLSLGK.
>Vx8humH07 Full length HC
(SEQ ID NO: 91)
QVQLVQSGAEVKKPGSSVKVSCKASGYSFTNYYIHWVRQAPGQGLEWMGYIDPLNGD
TTYNQKFKGRVTITADESTSTAYMELSSLRSEDTAVYYCARGGKRAMDYWGQGTLVT
VSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGG
PSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDK
SRWQEGNVFSCSVMHEALHNHYTQKSLSLSLGK.
>Vx8humH08 Full length HC
(SEQ ID NO: 92)
EVQLVQSGAEVKKPGESLKISCKGSGYSFTNYYIHWVRQMPGKGLEWMGYIDPLNGDT
TYNQKFKGQVTISADKSISTAYLQWSSLKASDTAMYYCARGGKRAMDYWGQGTLVTV
SSASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFEGGP
SVFLFPPKPKDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFLYSRLTVDKS
RWQEGNVFSCSVMHEALHNHYTQKSLSLSLGK.
>Vx8humH09 Full length HC
(SEQ ID NO: 93)
QVQLVQSGAEVKKPGSSVKVSCKASGYSFTNYYIHWVRQAPGQGLEWMGYIDPLNGD
TTYAQKFQGRVTITADESTSTAYMELSSLRSEDTAVYYCARGGKRAMDYWGQGTLVT
VSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGG
PSVFLEPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFELYSRLTVDK
SRWQEGNVFSCSVMHEALHNHYTQKSLSLSLGK.
>Vx8humH06 Full length HC
(SEQ ID NO: 94)
QVQLVQSGAEVKKPGASVKVSCKASGYSFTNYYTHWVRQAPGQGLEWMGYIDPLNGD
TTYNQKFKGRVTMTRDTSTSTVYMELSSLRSEDTAVYYCARGGKRAMDYWGQGTLVT
VSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGG
PSVFLEPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFELYSRLTVDK
SRWQEGNVFSCSVMHEALHNHYTQKSLSLSLGK.
>Vx8mur-H03 Full length HC
(SEQ ID NO: 95)
EVQLQQSGPELMKPGASVKISCKASGYSFTNYYIHWVNQSHGKSLEWIGYIDPLNGDTT
YNQKFKGKATLTVDKSSSTAYMRLSSLTSADSAVYYCARGGKRAMDYWGQGTSVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPS
VFLEPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSR
WQEGNVFSCSVMHEALHNHYTQKSLSLSLGK.
>Vx9mur-H04 Full length HC
(SEQ ID NO: 96)
QVQLQQFGAELAKPGASVQMSCKASGYTFTNYWIHWVKQRPGQGLEWIGYTDPRTDY
TEYNQKFKDKATLAADRSSSTAYMRLSSLTSEDSAVYYCAGGGRVGLGYWGHGSSVT
VSSASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCCVECPPCPAPPVAG
PSVFLEPPKPKDTLMISRTPEVTCVVVDVSHEDPEVQFNWYVDGVEVHNAKTKPREEQF
NSTERVVSVLTVVHQDWLNGKEYKCKVSNKGLPAPIEKTISKTKGQPREPQVYTLPPSR
EEMTKNQVSLTCLVKGFYPSDIAVEWESNGQPENNYKTTPPMLDSDGSFFLYSKLTVDK
SRWQQGNVFSCSVMHEALHNHYTQKSLSLSPGK.
>Vx8humH06 Full length HC
(SEQ ID NO: 97)
QVQLVQSGAEVKKPGASVKVSCKASGYSFTNYYTHWVRQAPGQGLEWMGYIDPLNGD
TTYNQKFKGRVTMTRDTSTSTVYMELSSLRSEDTAVYYCARGGKRAMDYWGQGTLVT
VSSASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCCVECPPCPAPPVAG
PSVFLEPPKPKDTLMISRTPEVTCVVVDVSHEDPEVQFNWYVDGVEVHNAKTKPREEQF
NSTERVVSVLTVVHQDWLNGKEYKCKVSNKGLPAPIEKTISKTKGQPREPQVYTLPPSR
EEMTKNQVSLTCLVKGFYPSDIAVEWESNGQPENNYKTTPPMLDSDGSFFLYSKLTVDK
SRWQQGNVFSCSVMHEALHNHYTQKSLSLSPGK.
>Vx8humH07 Full length HC
(SEQ ID NO: 98)
QVQLVQSGAEVKKPGSSVKVSCKASGYSFTNYYIHWVRQAPGQGLEWMGYIDPLNGD
TTYNQKFKGRVTITADESTSTAYMELSSLRSEDTAVYYCARGGKRAMDYWGQGTLVT
VSSASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCCVECPPCPAPPVAG
PSVFLEPPKPKDTLMISRTPEVTCVVVDVSHEDPEVQFNWYVDGVEVHNAKTKPREEQF
NSTERVVSVLTVVHQDWLNGKEYKCKVSNKGLPAPIEKTISKTKGQPREPQVYTLPPSR
EEMTKNQVSLTCLVKGFYPSDIAVEWESNGQPENNYKTTPPMLDSDGSFFLYSKLTVDK
SRWQQGNVFSCSVMHEALHNHYTQKSLSLSPGK.
>Vx8humH08 Full length HC
(SEQ ID NO: 99)
EVQLVQSGAEVKKPGESLKISCKGSGYSFTNYYTHWVRQMPGKGLEWMGYIDPLNGDT
TYNQKFKGQVTISADKSISTAYLQWSSLKASDTAMYYCARGGKRAMDYWGQGTLVTV
SSASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVERKCCVECPPCPAPPVAGPS
VFLEPPKPKDTLMISRTPEVTCVVVDVSHEDPEVQFNWYVDGVEVHNAKTKPREEQFNS
TFRVVSVLTVVHQDWLNGKEYKCKVSNKGLPAPIEKTISKTKGQPREPQVYTLPPSREE
MTKNQVSLTCLVKGFYPSDIAVEWESNGQPENNYKTTPPMLDSDGSFFLYSKLTVDKSR
WQQGNVFSCSVMHEALHNHYTQKSLSLSPGK.
>Vx8humH09 Full length HC
(SEQ ID NO: 100)
QVQLVQSGAEVKKPGSSVKVSCKASGYSFTNYYIHWVRQAPGQGLEWMGYIDPLNGD
TTYAQKFQGRVTITADESTSTAYMELSSLRSEDTAVYYCARGGKRAMDYWGQGTLVT
VSSASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCCVECPPCPAPPVAG
PSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSR
EEMTKNQVSLTCLVKGFYPSDIAVEWESNGQPENNYKTTPPMLDSDGSFFLYSKLTVDK
SRWQQGNVFSCSVMHEALHNHYTQKSLSLSPGK.

### Example 2

### Production of CD47 Antibodies

Chimeric antibodies disclosed herein comprise a mouse heavy chain variable domain and a light chain variable domain combined with a human kappa or human Fc IgG1, IgG1-N297Q, IgG2, IgG4, IgG4 S228P, and IgG4 PE constant domains, respectively. These were designed to incorporate a secretion signal and cloned into a mammalian expression system, and transfected into CHO cells to generate chimeric (murine-human) antibodies. The chimeric variants were expressed as full length IgG molecules, secreted into the medium, and purified using protein A.

Multiple methods for humanizing antibodies are well-known to those of ordinary skill in the art. One such method, as used herein, has previously been described (Making and Using Antibodies a Practical Handbook, Second Edition, Ed. Matthew R. Kase, Chapter 15: Humanization of Antibodies, Juan Carlos Almagro et al., CRC Press 2013). As such, the humanized antibodies disclosed herein comprise frameworks derived from the human genome. The collection covers the diversity found in the human germ line sequences, yielding functionally expressed antibodies in vivo. The complementarity determining regions (CDRs) in the light and heavy chain variable regions of the murine and chimeric (murine-human) are described herein and were determined by following commonly accepted rules disclosed in “Protein Sequence and Structure Analysis of Antibody Variable Domains,” In: Antibody Engineering Lab Manual, eds. S. Duebel and R. Kontermann, Springer-Verlag, Heidelberg (2001)). The human light chain variable domains were then designed. The humanized variable domains were then combined with a secretion signal and human kappa and human Fc IgG1, IgG1-N297Q, IgG2, IgG3, IgG4 S228P and IgG4 PE constant domains, cloned into a mammalian expression system, and transfected into CHO cells to generate humanized mAbs. The humanized variants were expressed as full length IgG molecules, secreted into the medium and purified using protein A.

A non-glycosylated version (IgG1-N297Q) was created by site directed mutagenesis of heavy chain position 297 to change the asparagine to glutamine (Human Fc IgG1-N297Q, SEQ ID NO:54). An IgG4 variant was created by site-directed mutagenesis at position 228 to change the serine to proline thereby preventing in vivo Fab arm exchange. An IgG4 double mutant was created by site-directed mutagenesis at positions 228 (serine to proline) and 235 (leucine to glutamate) to prevent Fab arm exchange and to further reduce Fc effector function. IgG2, IgG3, IgG4 S228P, and IgG4PE isotypes were constructed by cloning the heavy chain variable domain in frame with the human IgG2, IgG3, IgG4 S228P, and IgG4PE constant domains (Human Fc-IgG2, SEQ ID NO:56 Human Fc-IgG3, SEQ ID NO:57; Human Fc-IgG4 S228P, SEQ ID NO:59; and Human Fc-IgG4PE, SEQ ID NO:60).

### Example 3

### Binding of CD47 Monoclonal Antibodies (mAbs)

The binding of chimeric (murine-human) and humanized antibodies of the present disclosure was determined by ELISA using OV10 cells transfected with human CD47 (OV10 hCD47) or using freshly isolated human red blood cells (hRBCs), which display CD47 on their surface (Kamel et al. (2010) Blood. Transfus. 8(4):260-266).

Binding activities of VLX4, VLX8, and VLX9 chimeric (xi) and humanized mAbs were determined using a cell-based ELISA assay with human OV10 hCD47cells expressing cell surface human CD47. OV10 hCD47 cells were grown in IMDM medium containing 10% heat inactivated fetal bovine serum (BioWest; S01520). One day before assay, 3×10⁴ cells were plated in 96 well cell bind plates (Corning #3300, VWR #66025-626) and were 95-100% confluent at the time of assay. Cells were washed, various concentrations of purified antibodies added in IMDM and incubated at 37° C. for 1 hr in 95% O₂/5% CO₂. Cells were then washed with media and incubated for an additional hour at 37° C. with HRP labelled secondary anti-human antibody (Promega) diluted 1/2500 in media. Cells were washed three times with PBS, and the peroxidase substrate 3,3′, 5,5′-tetramethylbenzidine was added (Sigma; Catalog #T4444). Reactions were terminated by the addition of HCl to 0.7N, and absorbance at 450 nM determined using a Tecan model Infinite M200 plate reader. The apparent binding affinities of these clones to human OV10 hCD47 cells was determined by non-linear fit (Prism GraphPad software).

Binding activities of chimeric and humanized VLX4, VLX8, and VLX9 mAbs to human CD47 on hRBCs were also determined using flow cytometry. Blood was obtained from normal volunteers and RBCs were washed 3 times with phosphate buffered saline, pH 7.2 containing 2.5 mM EDTA (PBS+E). hRBCs were incubated for 60 min at 37° C. with various concentrations of the chimeric or humanized antibodies in a PBS+E. Cells were then washed with cold PBS+E and incubated for an additional hour on ice with FITC labelled donkey anti-human antibody (Jackson Immuno Research Labs, West Grove, Pa.; Catalogue #709-096-149) in PBS+E. Cells were washed with PBS+E, antibody binding was analyzed using a C6 Accuri Flow Cytometer (Becton Dickinson) and apparent binding affinities determined by non-linear fit (Prism GraphPad software) of the median fluorescence intensities at the various antibody concentrations.

All of the VLX4 chimeric (murine-human) mAbs bound to human OV10 hCD47 tumor cells with apparent affinities in the picomolar (pM) range (Table 1).

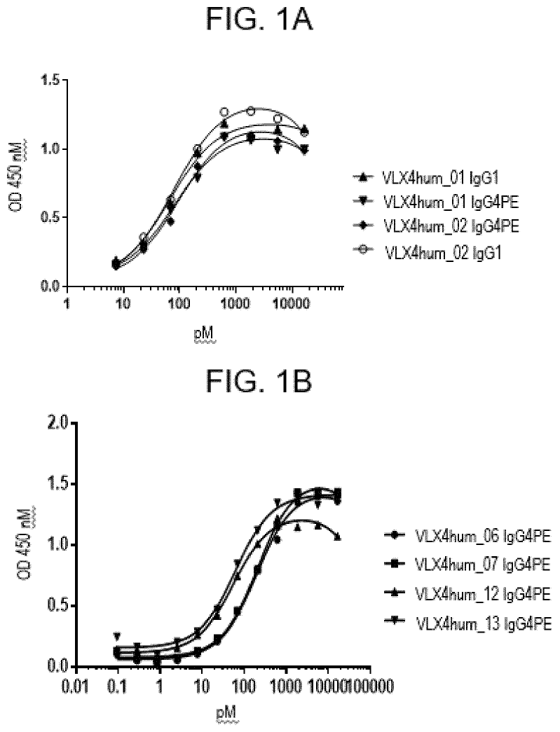

Similarly, the humanized VLX4 mAbs bound to human OV10 hCD47 tumor cells in a concentration-dependent manner (FIG. 1A and FIG. 1B) with apparent binding affinities ranging from the picomolar to low nanomolar range (Table 2).

All of the chimeric VLX4 mAbs bound to human RBCs with apparent Kd values in the picomolar range and these were similar to the K_{d} values obtained for OV10 hCD47 tumor cells by ELISA (Table 1).

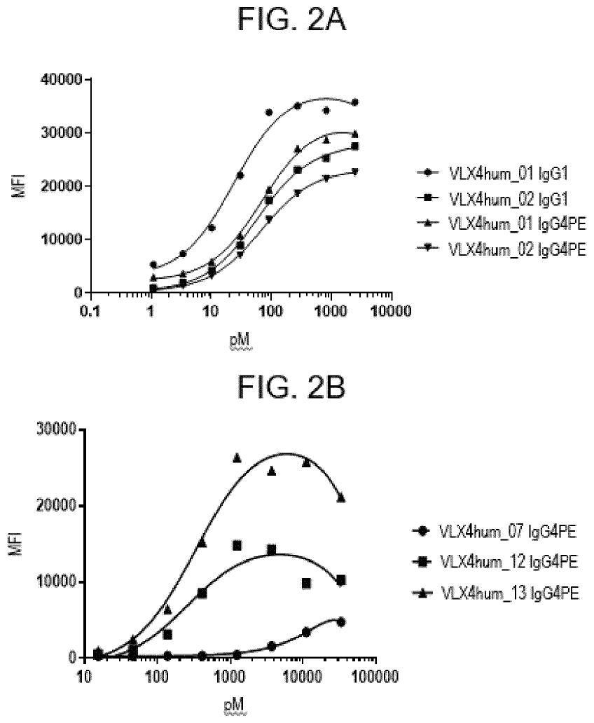

The humanized VLX4 mAbs VLX4hum_01 IgG1 N297Q, VLX4hum_02 IgG1 N297Q, VLX4hum_01 IgG4PE, VLX4hum_02 IgG4PE, VLX4hum_12 IgG4PE, and VLX4hum_13 IgG4PE bound to human RBCs with Kd values similar to those obtained for OV10 hCD47 tumor cells whereas VLX4hum_06 IgG4PE and VLX4hum_07 IgG4 PE exhibited reduced binding to hRBCs (FIG. 2A, FIG. 2B, and Table 2). This differential binding of the humanized mAbs to tumor cells and RBCs was unexpected as the VLX4 IgG4PE chimeric mAb bound with similar apparent Kd values to both tumor and RBC CD47 (Table 1).

As shown in Table 1, all the VLX8 chimeric mAbs bound to human OV10 hCD47 tumor cells in a concentration-dependent manner with apparent affinities in the picomolar (pM) range.

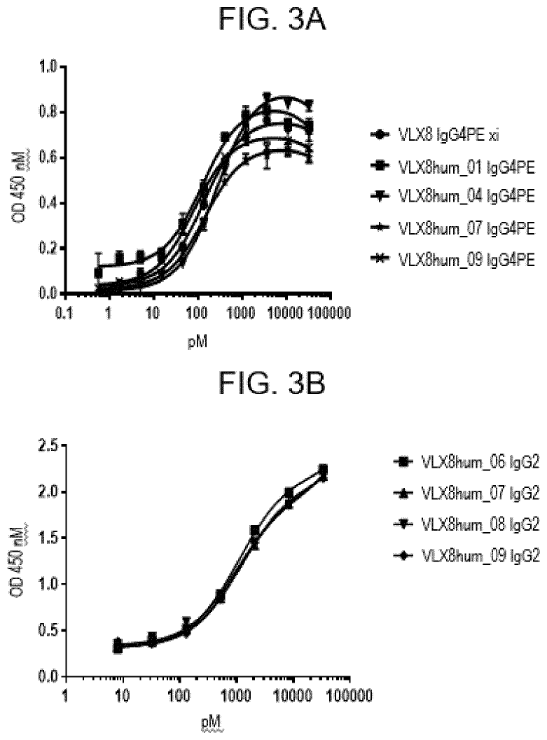

Similarly, the humanized VLX8 mAbs bound to human OV10 hCD47 tumor cells in a concentration-dependent manner (FIG. 3A and FIG. 3B) with apparent affinities in the picomolar range (Table 2).

All the VLX8 chimeric mAbs bound to hRBCs with apparent K_{d} values in the picomolar range and these were similar to the apparent K_{d} values obtained for OV10 hCD47 tumor cells by ELISA (Table 1).

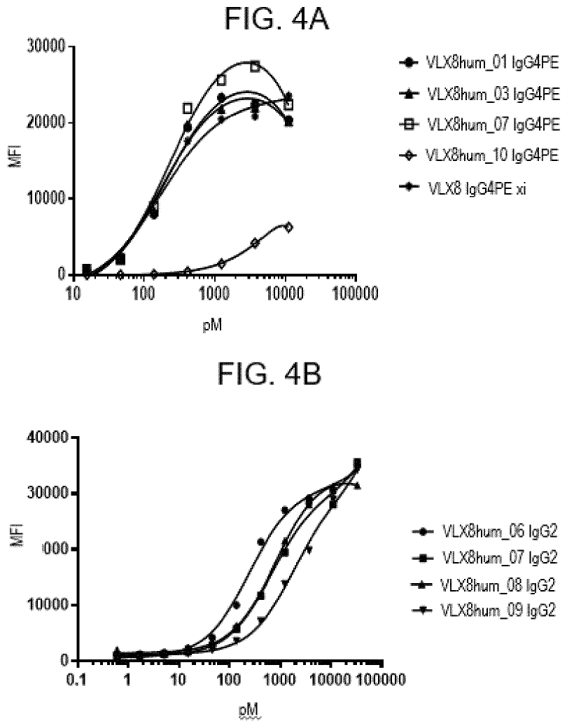

The VLX8 humanized mAbs VLX8hum_01 IgG4PE, VLX8hum_02 IgG4PE, VLX8hum_03 IgG4PE, VLX8hum_04 IgG4PE, VLX8hum_05 IgG4 PE, and VLX8hum_06 IgG4PE, VLX8hum_07 IgG4PE, VLX8hum_08 IgG4 PE, VLX8hum_09 IgG4 PE, VLX8hum_11 IgG4 PE, VLX8hum_06 IgG2, VLX8hum_07 IgG2, VLX8hum_08 and VLX8hum_09 IgG2 IgG2 bound to human RBCs with Kd values similar to the values obtained for OV10 hCD47 tumor cells whereas VLX8hum_10 IgG4PE exhibited reduced to hRBCs (FIG. 4A, FIG. 4B, and Table 2). This differential binding of the humanized mAbs to tumor cells and RBCs was unexpected as the VLX8 IgG4PE chimeric mAb bound with similar apparent Kd values to both tumor and RBC CD47 (Table 1).

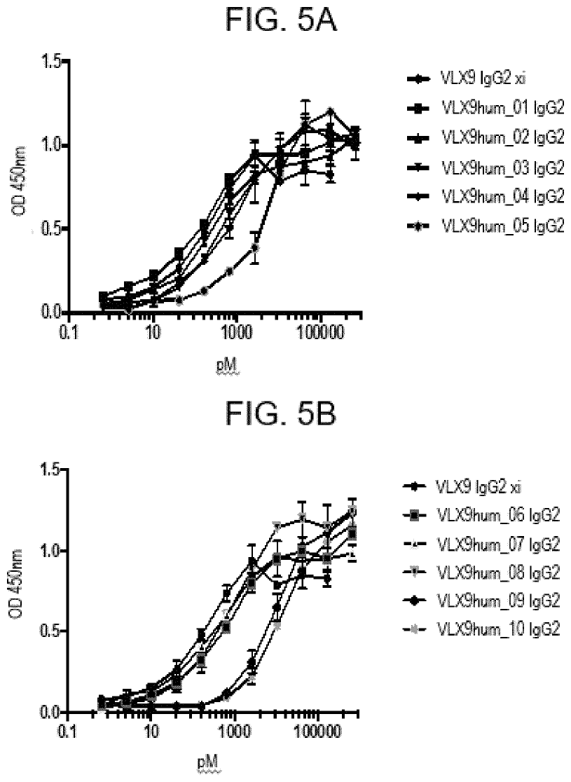

Table 1 shows the apparent binding affinities of VLX9 chimeric mAbs to human OV10 hCD47 cells and to human RBCs. All of the chimeric mAbs bound to OV10 hCD47 tumor cells with apparent binding constants in the picomolar range. Similarly, the humanized VLX9 mAbs bound to human OV10 hCD47 tumor cells in a concentration-dependent manner (FIG. 5A and FIG. 5B) with apparent affinities in the picomolar to nanomolar range (Table 2).

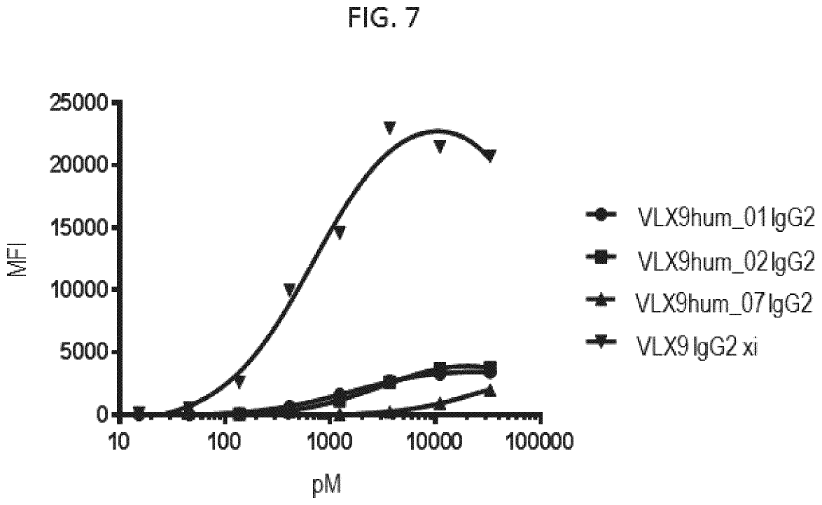

All the VLX9 chimeric mAbs bound to hRBCs with apparent K_{d} values in the picomolar range and these were similar to the apparent K_{d} values obtained for OV10 hCD47 tumor cells by ELISA (Table 1). In contrast to the chimeric mAbs, the VLX9 humanized mAbs VLX9hum_01 IgG2, VLX9hum_02 IgG2 and VLX9hum_07 IgG2 exhibited reduced binding to human RBCs (FIG. 7, Table 2). By contrast, the humanized mAbs VLX9hum_03 IgG2, VLX9hum_04 IgG2, VLX9hum_05 IgG2, VLX9hum_06 IgG2, VLX9hum_08 IgG2, VLX9hum_09 IgG2 and VLX9hum_10 IgG2 exhibited no measureable binding to RBCs up to 5,000 μM (Table 2). This differential binding of the humanized mAbs to tumor cells and RBCs was unexpected as the VLX9 IgG2 chimeric mAbs all bound with similar apparent Kd values to both tumor and RBC CD47 (Table 1).

Specific binding of CD47 humanized mAbs was demonstrated using Jurkat wildtype and Jurkat CD47 knockout (KO) cells. Jurkat wildtype and Jurkat CD47 KO cells were grown in RPMI medium containing 10% heat inactivated fetal bovine serum (BioWest; S01520). The cells were washed and 1×10⁴ cells were resuspended media and incubated with various antibody concentrations for one hour at 370 in 5% CO₂. Cells were then washed twice with 1×PBS and then resuspended 1:1000 in secondary antibody (goat anti-human IgG (H+L) FITC-labelled, Jackson Labs, 109-095-003) for one hour at 37° in 5% CO₂. Cells were then washed twice with 1×PBS and resuspended in 1×PBS. Median fluorescence intensity was determined by flow cytometry and the apparent binding affinities determined by non-linear fit (Prism GraphPad software).

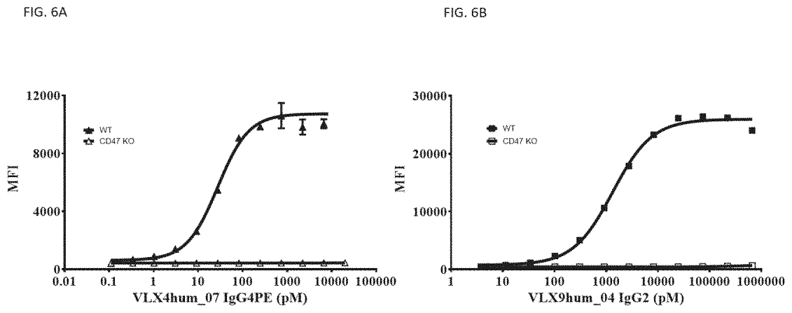

As shown in FIG. 6, VLX4hum_07 IgG4PE (FIG. 6A) and VLX9hum_09 IgG2 (FIG. 6B) bound to Jurkat cells expressing CD47, whereas no binding is observed to Jurkat CD47KO cells.

TABLE 1
Binding of VLX4, VLX8, and VLX9 Chimeric (xi) mAbs to
OV10 hCD47 Cells and Human Red Blood Cells (hRBCs).

| | Kd (pM) | | |
|---|---|---|---|
| | OV10 hCD47 | Kd (pM) | HA |
| | Cell-based ELISA | hRBC | hRBC |
| VLX4 IgG1 (xi) | 315 | 104 | Yes |
| VLX4 IgG1 N297Q (xi) | 258 | 92 | Yes |
| VLX4 IgG2 (xi) | 431 | 184 | Yes |
| VLX4 IgG4 S228P (xi) | 214 | 99 | No |
| VLX4 IgG4 PE(xi) | 225 | 303 | No |
| VLX8 IgG1 N297Q (xi) | 42 | 91 | Yes |
| VLX8 IgG4 PE (xi) | 56 | 77 | Yes |
| VLX9 IgG1 (xi) | 280 | 381 | Yes |
| VLX9 IgG1 N297Q (xi) | 275 | 190 | Yes |
| VLX9 IgG2 (xi) | 880 | 742 | Yes |
| VLX9 IgG4 PE (xi) | 293 | 126 | Yes |

TABLE 2
Binding of VLX4, VLX8, and VLX9 Humanized mAbs to Human
OV10 hCD47 and Human Red Blood Cells (hRBCs).

| | Kd (pM) | | |
|---|---|---|---|
| | OV10 hCD47 | Kd (pM) | HA |
| | Cell-based ELISA | hRBC | hRBC |
| VLX4hum_01 IgG1 | 73 | 23 | Yes |
| VLX4hum_02 IgG1 | 80 | 70 | Yes |
| VLX4hum_01 IgG4 PE | 82 | 80 | No |
| VLX4hum_02 IgG4 PE | 95 | 75 | R*** |
| VLX4hum_06 IgG4 PE | 196 | >33,000** | Yes |
| VLX4hum_07 IgG4 PE | 209 | >33,000** | Yes |
| VLX4hum_12 IgG4 PE | 56 | 263 | Yes |
| VLX4hum_13 IgG4 PE | 62 | 340 | Yes |
| VLX8hum_01 IgG4 PE | 54 | 209 | No |
| VLX8hum_02 IgG4 PE | 50 | 221 | No |
| VLX8hum_03 IgG4 PE | 67 | 183 | No |
| VLX8hum_04 IgG4 PE | 49 | 119 | No |
| VLX8hum_05 IgG4 PE | 68 | 264 | No |
| VLX8hum_06 IgG4 PE | 61 | 274 | Yes |
| VLX8hum_07 IgG4 PE | 24 | 241 | Yes |
| VLX8hum_08 IgG4 PE | 97 | 217 | Yes |
| VLX8hum_09 IgG4 PE | 82 | 336 | Yes |
| VLX8hum_10 IgG4 PE | 183 | >33,000** | Yes |
| VLX8hum_11 IgG4 PE | 90 | 87 | No |
| VLX8hum_06 IgG2 | 403 | 246 | Yes |
| VLX8hum_07 IgG2 | 460 | 671 | Yes |
| VLX8hum_08 IgG2 | 464 | 820 | Yes |
| VLX8hum_09 IgG2 | 680 | 1739 | Yes |
| VLX9hum_01 IgG2 | 162 | 1653** | No |
| VLX9hum_02 IgG2 | 227 | 4103** | No |
| VLX9hum_03 IgG2 | 606 | *MB | No |
| VLX9hum_04 IgG2 | 823 | *MB | No |
| VLX9hum_05 IgG2 | 6372 | *MB | No |
| VLX9hum_06 IgG2 | 547 | *MB | No |
| VLX9hum_07 IgG2 | 341 | >66,000** | ***R |
| VLX9hum_08 IgG2 | 688 | *MB | No |
| VLX9hum_09 IgG2 | 8340 | *MB | No |
| VLX9hum_10 IgG2 | 12232 | *MB | No |

*MB—Minimal biniding; no measurable binding detected at mAb concentration up to 5,000 pM.
**Reduced RBC binding.
***R—Reduced hemagglutination.

Cross-species binding of humanized VLX4, VLX8, and VLX9 mAbs was determined using flow cytometry. Mouse, rat, rabbit or cynomolgus monkey RBCs were incubated for 60 min on at 37° C. with various concentrations of the humanized antibodies in a solution of phosphate buffered saline, pH 7.2, 2.5 mM EDTA (PBS+E). Cells were then washed with cold PBS+E, and incubated for an additional hr on ice with FITC labelled donkey anti-human antibody (Jackson Immuno Research Labs, West Grove, Pa.; Catalogue #709-096-149) in PBS+E. Cells were washed with PBS+E, and antibody binding analyzed using a C6 Accuri Flow Cytometer (Becton Dickinson).

Table 3 shows the apparent binding affinities of the humanized VLX4 and VLX8 mAbs to RBCs from mouse, rat, and cynomolgus monkey determined by non-linear fit (Prism GraphPad software) of the median fluorescence intensities at various antibody concentrations. This data demonstrates that humanized VLX4 and VLX8 mAbs bind to mouse, rat, rabbit (data not shown) and cynomolgus monkey RBCs with apparent Kd values in the picomolar to nanomolar range.

TABLE 3
Binding of VLX4 and VLX8 Humanized mAbs to Mouse,
Rat and Cynomolgus Monkey RBCs.

| | | | Kd (pM) |
|---|---|---|---|
| | Kd (pM) | Kd (pM) | Cynomolgus |
| | Mouse RBC | Rat RBC | Monkey RBC |
| VLX4hum_01 IgG4 PE | 13001 | 30781 | 56 |
| VLX4hum_07 IgG4 PE | 15192 | 14274 | 13522 |
| VLX8hum_11 IgG4 PE | 9123 | 8174 | 55 |

### Example 4

### Binding of Humanized Anti-CD47 mAbs Determined by Surface Plasmon Resonance

Binding of soluble anti-CD47 mAbs to recombinant human His-CD47 was measured in vitro by surface plasmon resonance on a Biacore 2000. An Anti-Human IgG (GE Lifesciences) was amine coupled to a CM5 chip on flow cells 1 and 2. The humanized mAbs VLX4hum_07 IgG4PE, VLX8hum_11 IgG4PE, VLX9hum_08 IgG2 or VLX9hum_03 IgG2 diluted in HBS-EP⁺ running buffer (pH 7.2) were captured onto flow cell 2. Multi-cycle kinetics were determined using 0 to 1000 nM His-tagged human CD47 (Acro Biosystems) diluted in HBS-EP⁺ running buffer (pH 7.2) with contact time of 180 seconds and dissociation time of 300 seconds. A 1:1 binding model was employed for kinetic analysis of binding curves. The on-rate, off-rate and Dissociation constants for VLX4hum_07 IgG4PE, VLX8hum_11 IgG4PE, VLX9hum_08 IgG2 and VLX9hum_03 IgG2 are shown in Table 4.

TABLE 4
Binding of VLX4, VLX8 and VLX9 Humanized mAbs to Human
Recombinant His-CD47 by Surface Plasmon Resonance at pH 7.2.

| | kₐ | k_{d} | K_{D} (nM) |
|---|---|---|---|
| VLX4hum_07 IgG4PE | 1.7e⁵ | 8.7e⁻⁴ | 5.1 |
| VLX8hum_11 IgG4PE | 6.8e⁵ | 7.9e⁻⁴ | 1.2 |
| VLX9_08 IgG2 | 7.6e⁴ | 6.5e⁻⁴ | 8.6 |
| VLX9_03 IgG2 | 6.5e⁴ | 7.3e⁻⁴ | 11.1 |

### Example 5

### Differential Binding of Anti-CD47 mAbs

Some soluble CD47 antibodies described herein have been shown to differentially bind to normal cells. This additional property of selective binding is expected to have advantages compared to mAbs that bind with equal affinity to normal and tumor cells. Anti-CD47 mAbs with such reduced binding have not been described.

Binding by soluble anti-CD47 mAbs is measured in vitro. Binding activities of VLX4, VLX8, and VLX9 humanized mAbs were determined using a flow cytometry based binding assay with human aortic endothelial cells (HAEC), skeletal muscle cells (SkMC), human lung microvascular endothelial cells (HMVEC-L), renal tubular epithelial cells (RTEC), CD3⁺ cells or peripheral blood mononuclear cells (PBMC). HAEC, SkMC, HMVEC-L and RTEC cells were purchased from Lonza and cultured according to the manufacturer's recommendations. Adherent cells were removed from the culture flask with accutase, resuspended in the recommended media and 1×10⁴ cells were incubated with various antibody concentrations for one hour at 37°, 5% CO₂. For non-adherent cells, 1×10⁴ cells were resuspended in the recommended media and incubated with various antibody concentrations for one hour at 37°, 5% CO₂. Cells were then washed twice with 1×PBS and then resuspended 1:1000 in secondary antibody (goat anti-human IgG (H+L)—FITC, Jackson Labs, 109-095-003) for one hour at 37° C., 5% CO₂.

PBMC were isolated by ficoll gradient and were incubated with an FcR blocking reagent (Miltenyi Biotec) for 10 min at 4° C. per manufacturer's recommendation immediately preceeding the addition of various concentrations of antibodies diluted in PBS. CD3 cells were detected using an allophycocyanin (APC)-labelled anti-CD3 antibody (BD BioSciences) which was added at the same time as the FITC-labelled goat anti-human IgG (H+L) antibody. Cells were washed twice with 1×PBS and antibody binding was assessed by flow cytometry analysis.

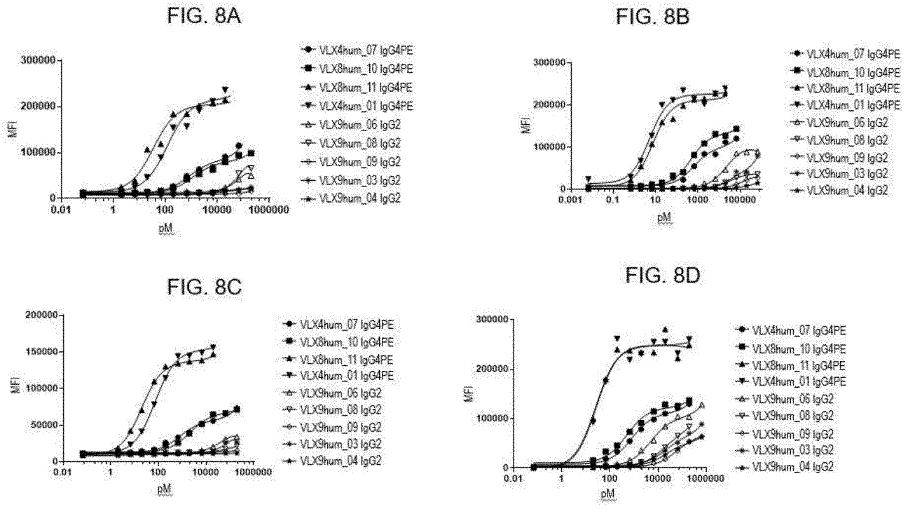

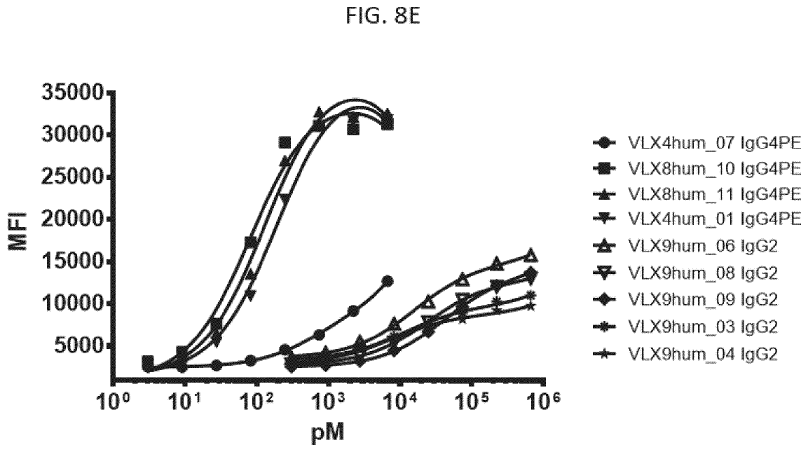

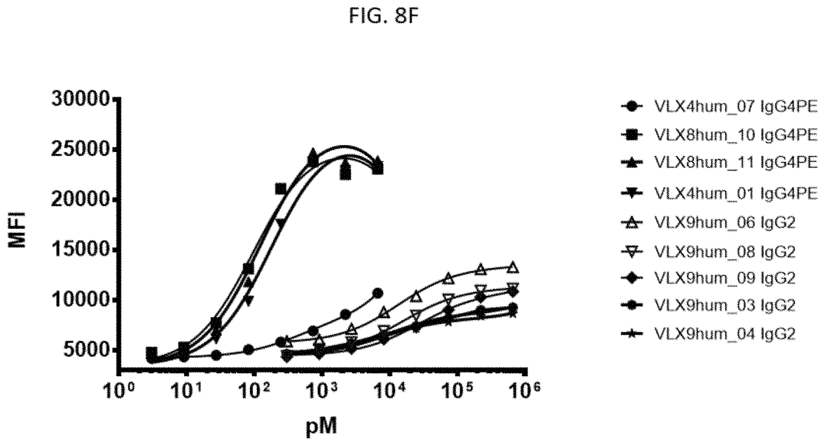

As shown in FIG. 8A, VLX4 and VLX8 humanized mAbs bound to HAEC cells whereas VLX9 humanized mAbs had reduced or minimal binding to HAEC cells as compared to tumor cells (Table 5). VLX9 humanized mAbs also showed reduced binding to SkMC cells (FIG. 8B), reduced or minimal binding to HMVEC-L cells (FIG. 8C), reduced binding to RPTEC cells (FIG. 8D) as compared to binding to tumor cells (Table 5). Reduced binding of VLX9 humanized mAbs was also observed to CD3⁺ cells (FIG. 8E) and PBMC (FIG. 8F) as compared to tumor cells (Table 5). This selective binding imparts an additional desirable antibody property and potential therapeutic benefit in the treatment of cancer.

TABLE 5
VLX4, VLX8 and VLX9 Humanized mAbs Binding to Normal Cells.

| | Kd (pM) | | | | | | | |
|---|---|---|---|---|---|---|---|---|
| | OV10 hCD47 | Kd | Kd | Kd | Kd | Kd | Kd | Kd |
| | Cell-based | (pM) | (pM) | (pM) | (pM) | (pM) | (pM) | (pM) |
| | ELISA | hRBC | HAEC | HMVEC-L | SKMC | RPTEC | CD3⁺ | PBMC |
| VLX4hum_01 IgG4 PE | 82 | 80 | 118 | 72 | 5 | 26 | 220 | 269 |
| VLX4hum_07 IgG4 PE | 209 | >33,000** | 747 | 792 | 630 | 784 | 440 | 499 |
| VLX8hum_10 IgG4 PE | 183 | >33,000** | 1104 | 2113** | 461 | 491 | 91 | 106 |
| VLX8hum_11 IgG4 PE | 90 | 87 | 34 | 20 | 7 | 26 | 144 | 156 |
| VLX9hum_03 IgG2 | 606 | MB* | MB* | >200,000** | >200,000** | >200,000** | 10863** | 10232** |
| VLX9hum_04 IgG2 | 823 | MB* | MB* | MB* | >200,000** | >200,000** | 7426** | 7619** |
| VLX9hum_06 IgG2 | 547 | MB* | >200,000** | 71619** | 23483** | 4847** | 19354** | 17904** |
| VLX9hum_08 IgG2 | 688 | MB* | >200,000** | >200,000** | 34783** | >200,000** | 28287** | 24486** |
| VLX9hum_09 IgG2 | 8340 | MB* | MB* | MB* | MB* | >200,000** | 56146** | 48348** |

*MB—Minimal binding, no measureable binding detected at mAb concentration up to 5,000 pM.
**Reduced binding.

### Example 6

### pH Dependent and Independent Binding of Humanized Anti-CD47 mAbs

Some soluble anti-CD47 mAbs described herein have been shown to bind tumor cells at acidic pH with greater affinity compared to physiologic pH. This additional property is expected to have advantages compared to mAbs that bind at similar affinities to CD47 at both acidic and physiologic pH, in part due to the acidic nature of the tumor microenvironment (Tannock and Rotin, Cancer Res 1989; Song et al. Cancer Drug Discovery and Development 2006; Chen and Pagel, Advan Radiol 2015).

Binding by soluble anti-CD47 mAbs to immobilized recombinant human CD47 and to human CD47 expressed on cells was measured in vitro. For the in vitro binding to recombinant CD47, His-CD47 (AcroBiosystems) was adsorbed to high-binding microtiter plates overnight at 4° C. The wells were washed and varying concentrations of anti-CD47 mAbs were added to the wells in buffers with a of either pH 6 or pH 8 for 1 hour. The wells were washed and then incubated with HRP-labelled secondary antibody for 1 hour at pH 6 or pH 8 followed by addition of peroxidase substrate. The apparent affinities were calculated using non-linear fit model (Graphpad Prism).

For analysis of pH dependent binding by surface plasmon resonance using a Biacore 2000, an Anti-Human IgG (GE Lifesciences) was amine coupled to a CM5 chip on flow cells 1 and 2. An Fc-tagged human CD47 (Acro Biosystems) was diluted in PBS-EP⁺ running buffer (pH 7.5, 6.5 or 6.0) and captured onto flow cell 2. Multi-cycle kinetics were determined using 0 to 100 nM VLX8hum_11 Fab or VLX9hum_08 Fab diluted in PBS-EP⁺ running buffer (pH 7.5, 6.5 or 6.0) with contact time of 180 seconds and dissociation time of 300 seconds. A 1:1 binding model was employed for kinetic analysis of binding curves.

For the in vitro binding to cells expressing CD47, Jurkat cells were grown in RPMI medium containing 10% heat inactivated fetal bovine serum (BioWest; S01520). The cells were washed and 1×10⁴ cells were resuspended in PBS supplementated with 2% FBS at either pH 7.4 or 6.5 and incubated with various antibody concentrations for 1 hour at 37° C. Cells were then washed twice and resuspended with 1:1000 of secondary antibody (goat anti-human IgG (H+L) labelled with Alexa488, JacksonImmunoresearch) for 1 hour at 37° C. at pH 6 or pH 8. Cells were then washed twice and the median fluorescence intensity was determined by flow cytometry. The apparent binding affinities were determined by non-linear fit (Prism GraphPad software).

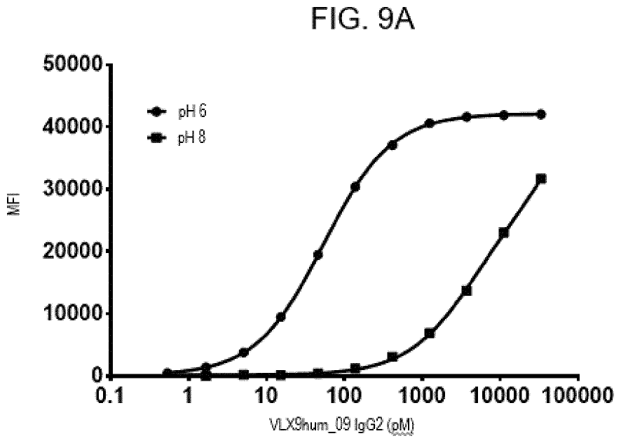

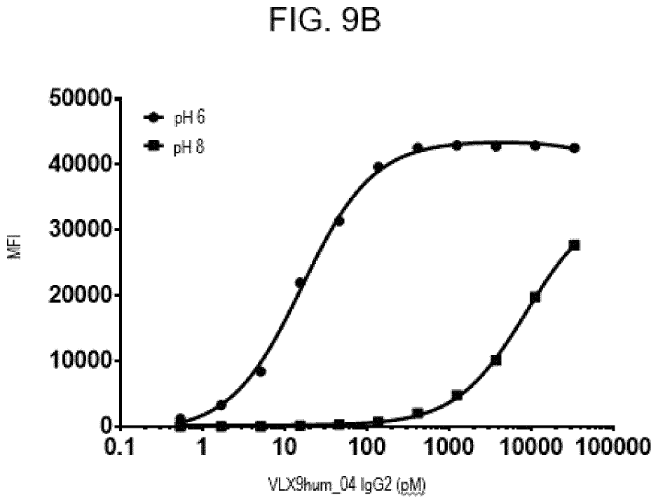

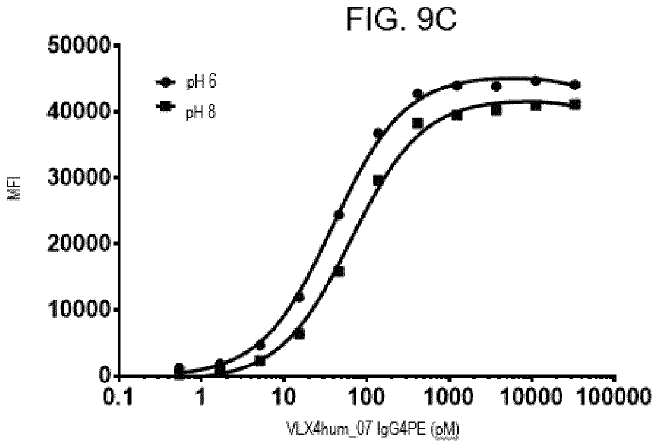

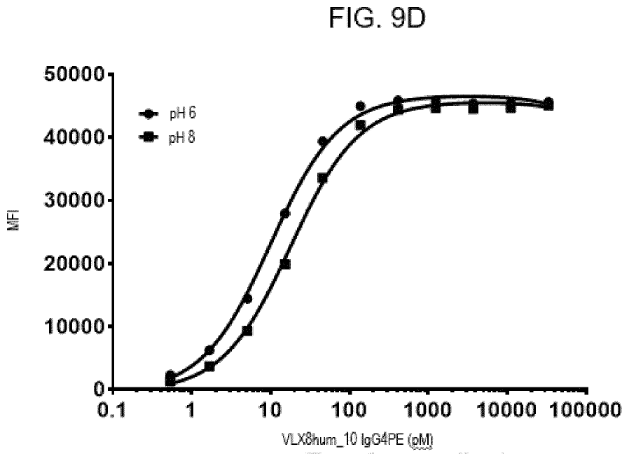

As shown in FIG. 9A and FIG. 9B, the soluble VLX9 humanized mAbs (VLX9hum_09 IgG2 and VLX9hum_04 IgG2) bound to His-CD47 with greater affinity at the more acidic pH 6.0 than at pH 8.0. Neither VLX4hum_07 IgG4PE (FIG. 9C) nor VLX8hum_10 IgG4PE (FIG. 9D) displayed pH dependent binding. In addition, the murine VLX9 antibody and VLX9 chimeric antibodies containing human Fc from isotypes IgG1, IgG2 and IgG4PE did not display pH dependence (Table 6) whereas VLX9hum_04 as either an IgG1, IgG2 or an IgG4PE demonstrated greater binding to His-CD47 at acidic pH (Table 7). The apparent binding affinities for additional humanized mAbs to recombinant human CD47 are shown in Table 8. All humanized VLX9 mAbs exhibited pH dependent binding whereas the VLX4 and VLX8 humanized mAbs did not. To determine the effect of pH on on-rates, off-rates and dissociation constants, Biacore analysis was performed for humanized mAbs VLX8hum_11 Fab fragment and VLX9hum_08 Fab at pH 6, pH 6.5 and pH 7.5. The VLX9hum_08 Fab exhibited pH dependent binding that increased with decreasing pH whereas the VLX8hum_11 Fab did not. The on-rate, off-rate and dissociation constants for VLX8hum_11 Fab and VLX9hum_08 Fab are shown in Table 9. Table 10 illustrates the pH dependent binding exhibited by VLX9hum_04 IgG2 to CD47 expressed on Jurkat cells. No pH dependent binding was exhibited by VLX4hum_07 IgG4PE. This pH dependence of the VLX9 humanized mAbs imparts an additional desirable antibody property and therapeutic benefit in the treatment of cancer.

TABLE 6
Murine VLX9 and mouse-human chimeric
VLX9 Binding to CD47 is not pH Dependent.

| | KD (pM) | KD (pM) |
|---|---|---|
| | pH 6 | pH 8 |
| VLX9 IgG (murine) | 91 | 76 |
| VLX9 IgG1-N297Q (xi) | 99 | 135 |
| VLX9 IgG2 (xi) | 130 | 137 |
| VLX9 IgG4PE (xi) | 133 | 160 |

TABLE 7
VLX9hum_04 Humanized mAbs Bind to CD47 in a pH
Dependent Manner and Binding is not Isotype Specific.

| | KD (pM) | KD (pM) |
|---|---|---|
| | pH 6 | pH 8 |
| VLX9hum_04 Ig1-N297Q | 215 | >33,000 |
| VLX9hum_04 IgG2 | 470 | >33,000 |
| VLX9hum_04 IgG4PE | 256 | >33,000 |

TABLE 8
pH Dependent and Independent
Binding of VLX4, VLX8 and VLX9 Humanized mAbs.

| | KD (pM) pH 6 | KD (pM) pH 8 |
|---|---|---|
| VLX9hum_03 IgG2 | 48 | >33,000 |
| VLX9hum_04 IgG2 | 43 | >33,000 |
| VLX9hum_06 IgG2 | 61 | >33,000 |
| VLX9hum_08 IgG2 | 65 | >33,000 |
| VLX9hum_09 IgG2 | 138 | >33,000 |
| VLX4hum_07 IgG4PE | 63 | 92 |
| VLX4hum_01 IgG4PE | 47 | 75 |
| VLX8hum_10 IgG4PE | 52 | 79 |
| VLX8hum_11 IgG4PE | 64 | 92 |

TABLE 9
pH Independent and Dependent Binding of VLX8hum_11 Fab and
VLX9hum_08 Fab to Recombinant Human CD47.

| | kₐ | k_{d} | K_{D} (nM) |
|---|---|---|---|
| VLX8hum_11 Fab | 1.35e⁶ | 2.29e⁻³ | 1.7 nM |
| (pH 7.5) | | | |
| VLX8hum_11 Fab | 2.14e⁶ | 2.78e⁻³ | 1.3 nM |
| (pH 6.5) | | | |
| VLX8hum_11 Fab | 1.64e⁶ | 2.63e⁻³ | 1.6 nM |
| (pH 6.0) | | | |
| VLX9hum_08 Fab | 1.43e⁵ | 1.13e⁻² | 79 nM |
| (pH 7.5) | | | |
| VLX9hum_08 Fab | 1.74e⁵ | 9.74e⁻⁴ | 5.6 nM |
| (pH 6.5) | | | |
| VLX9hum_08 Fab | 1.95e⁵ | 9.94e⁻⁴ | 5.1 nM |
| (pH 6.0) | | | |

TABLE 10
pH Dependent and Independent Binding of VLX4 and
VLX9 Humanized mAbs to Jurkat Cells.

| | KD (pM) | KD (pM) |
|---|---|---|
| | pH 6.5 | pH 7.4 |
| VLX4hum_07 IgG4PE | 69 | 23 |
| VLX9hum_04 IgG2 | 231 | 1526 |

### Example 7

### CD47 Antibodies Block CD47/SIRPα Binding

To assess the effect of humanized CD47 mAbs on binding of CD47 to SIRPα in vitro the following method is employed using the binding of fluorescently-labelled SIRPα-Fc fusion protein to CD47 expressing Jurkat cells.

SIRPα-Fc fusion protein (R&D Systems, cat #4546-SA) was labelled using an Alexa Fluor® antibody labelling kit (Invitrogen Cat No. A20186) according to the manufacturers specifications. 1.5×10⁶ Jurkat cells were incubated with humanized mAbs (5 μg/ml), a human control antibody in RPMI containing 10% media or media alone for 30 min at 37° C. An equal volume of fluorescently labelled SIRPα-Fc fusion protein was added and incubated for an additional 30 min at 37° C. Cells were washed once with PBS and the amount of labelled SIRPα-Fc bound to the Jurkat cells analyzed by flow cytometry.

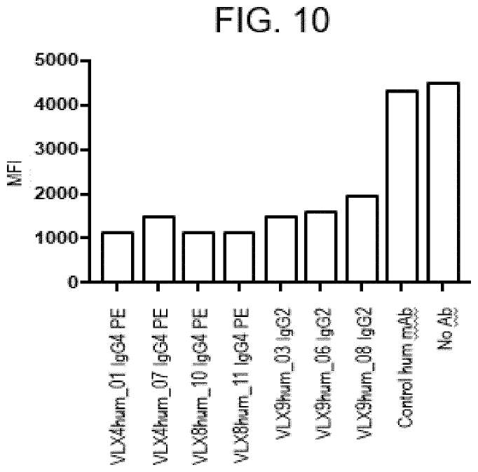

As shown in FIG. 10, the humanized VLX4, VLX8 and VLX9 mAbs (VLX4hum_01 IgG4PE, VLX4hum_07 IgG4PE, VLX8hum_10 IgG4PE, VLX8hum_11 IgG4PE, VLX9hum_03 IgG2, VLX9hum_06 IgG2 and VLX9hum_08 IgG2) blocked the interaction of CD47 expressed on the Jurkat cells with soluble SIPRα, while the human control antibody (which does not bind to CD47) or media alone, did not block the CD47/SIRPα interaction.

### Example 8

### CD47 Antibodies Increase Phagocytosis

To assess the effect of chimeric (murine-human) and humanized VLX4, VLX8, and VLX9 CD47 mAbs on phagocytosis of tumor cells by macrophages in vitro the following method is employed using flow cytometry (Willingham et al. (2012) Proc Natl Acad Sci USA 109(17):6662-7 and Tseng et al. (2013) Proc Natl Acad Sci USA 110(27):11103-8).

Human derived macrophages were derived from leukapheresis of healthy human peripheral blood and incubated in AIM-V media (Life Technologies) for 7-10 days. For the in vitro phagocytosis assay, macrophages were re-plated at a concentration of 1×10⁴ cells per well in 100 ul of AIM-V media in a 96-well plate and allowed to adhere for 24 hrs. Once the effector macrophages adhered to the culture dish, the target human cancer cells (Jurkat) were labelled with 1 μM 5(6)-Carboxyfluorescein diacetate N-succinimidyl ester (CFSE; Sigma Aldrich) and added to the macrophage cultures at a concentration of 5×10⁴ cells in 1 ml of AIM-V media (5:1 target to effector ratio). VLX4, VLX8, and VLX9 CD47 mAbs (1 μg/ml) were added immediately upon mixture of target and effector cells and allowed to incubate at 37° C. for 2-3 hours. After 2-3 hrs, all non-phagocytosed cells were removed and the remaining cells washed three times with phosphate buffered saline (PBS; Sigma Aldrich). Cells were then trypsinized, collected into microcentrifuge tubes, and incubated in 100 ng of allophycocyanin (APC) labelled CD14 antibodies (BD Biosciences) for 30 minutes, washed once, and analyzed by flow cytometry (Accuri C6; BD Biosciences) for the percentage of CD14⁺ cells that were also CFSE⁺ indicating complete phagocytosis.

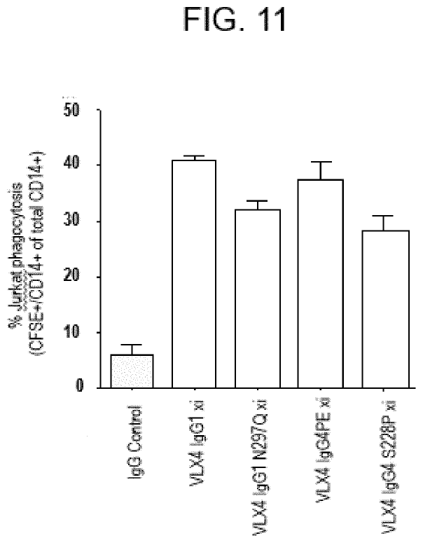

As shown in FIG. 11, the VLX4 chimeric mAbs VLX4 IgG1 xi, VLX4 IgG1 N297Q xi, VLX4 IgG4PE xi, and VLX4 IgG4 S228P xi increased phagocytosis of Jurkat cells by human macrophages by blocking the CD47/SIRPα interaction. This enhanced phagocytosis is independent of Fc function.

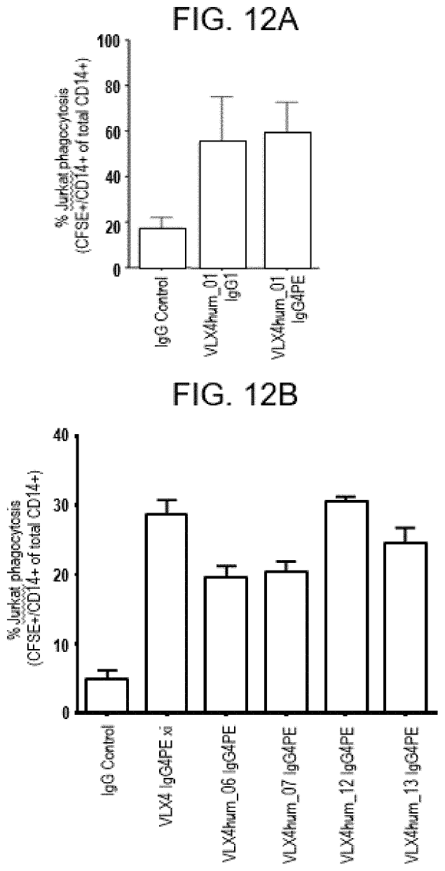

Similarly, as shown in FIG. 12A and FIG. 12B, humanized mAbs VLX4hum_01 IgG1, VLX4hum_01 IgG4PE, VLX4hum_06 IgG4PE, VLX4hum_07 IgG4PE, VLX4hum_12 IgG4PE, and VLX4hum_13 IgG4PE increased phagocytosis of Jurkat cells by human macrophages by blocking the CD47/SIRPα interaction. This enhanced phagocytosis is independent of Fc function.

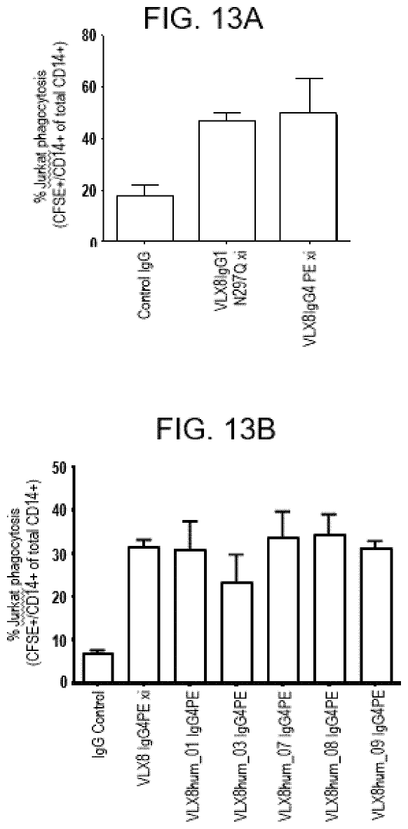

As shown in FIG. 13A, the VLX8 chimeric mAbs VLX8 IgG1 N297Q xi and VLX8 IgG4PE xi increase phagocytosis of Jurkat cells by human macrophages by blocking the CD47/SIRPα interaction. This enhanced phagocytosis is independent of Fc function.

Similarly, as shown in FIG. 13B, humanized mAbs VLX8hum_01 IgG4PE, VLX8hum_03 IgG4PE, VLX8hum_07 IgG4PE, VLX8hum_08 IgG4PE, and VLX8hum_09 IgG4PE and chimeric mAb VLX8 IgG4PE xi increased phagocytosis of Jurkat cells by human macrophage by blocking the CD47/SIRPα interaction.

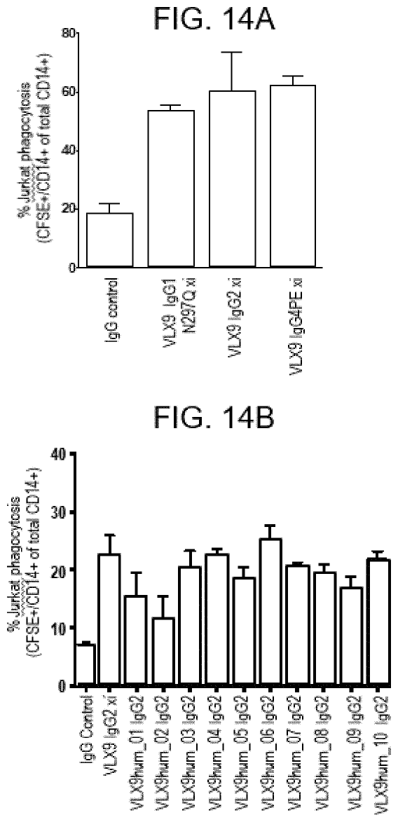

As shown in FIG. 14A, the VLX9 IgG1 N297Q xi, VLX9 IgG2 xi and VLX9 IgG4PE xi chimeric mAbs all increased phagocytosis of Jurkat cells by human macrophages by blocking the CD47/SIRPα interaction. This enhanced phagocytosis is independent of Fc effector function. Similarly as shown in FIG. 14B, all of the humanized VLX9 IgG2 mAbs (VLX9hum_01 to 10 IgG2) increased phagocytosis of Jurkat cells.

### Example 9

### Induction of Cell Death by Soluble CD47 Antibodies

Some soluble CD47 antibodies have been shown to induce selective cell death of tumor cells. This additional property of selective toxicity to cancer cells is expected to have advantages compared to mAbs that only block SIRPα binding to CD47.

Induction of cell death by soluble anti-CD47 mAbs is measured in vitro (Manna et al. (2003) J. Immunol. 107 (7): 3544-53, Kikuchi et al. Biochem Biophys Res. Commun. 315: 912-8, 2004), Pettersen et al. J. Immuno. 162: 7031-7040, 1999), Manna et al. Cancer Research, 64: 1026-1036, 2004). For the in vitro cell death assay, 1×10⁵ transformed human T cells (Jurkat cells) were incubated with soluble humanized VLX4, VLX8, and VLX9 CD47 mAbs (1 μg/ml) for 24 hrs at 37° C. As cell death occurs, mitochondrial membrane potential is decreased, the inner leaflet of the cell membrane is inverted, exposing phosphatidylserines (PS), and propidium iodide (PI) or 7-aminoactinomycin D (7-AAD) is able to incorporate into nuclear DNA. In order to detect these cellular changes, cells were then stained with fluorescently labelled annexin V and PI or 7-aminoactinomycin D (7-AAD) (BD Biosciences) and the signal detected using an Accuri C6 flow cytometer (BD Biosciences). The increase in PS exposure is determined by measuring the percent increase in annexin V signal and the percent of dead cells by measuring the percent increase in PI or 7-AAD signal. Annexin V positive (annexin V⁺) or annexin V positive/7-AAD negative (annexin V⁺/7-AAD⁻) cells are observed in early stages of cell death and annexin V positive/7-AAD positive (annexin V⁺/7-AAD⁺) cells are dead cells. Importantly for therapeutic purposes, these mAbs induce cell death of tumor cells directly and do not require complement or the intervention of other cells (e.g., NK cells, T cells, or macrophages) to kill. Thus, the mechanism is independent of both other cells and of Fc effector function. Therefore, therapeutic antibodies developed from these mAbs can be engineered to reduce Fc effector functions such as ADCC and CDC and thereby limit the potential for side effects common to humanized mAbs with intact Fc effector functions.

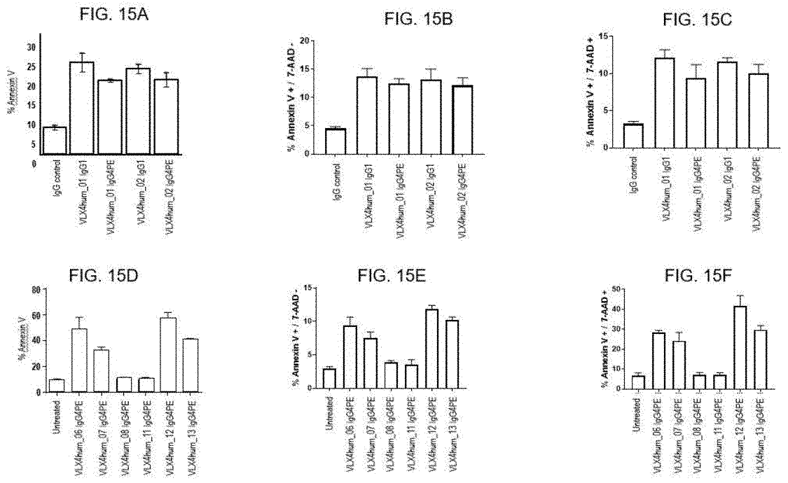

As shown in FIGS. 15A-15F, the soluble VLX4 humanized mAbs induced increased PS exposure and cell death of Jurkat cells as measured by increased % of the cells that are annexin V⁺ (FIG. 15A and FIG. 15D), annexin V⁺/7-AAD⁻ (FIG. 15B and FIG. 15E), or annexin V⁺/7-AAD⁺ (FIG. 15C and FIG. 15F). The humanized mAbs VLX4hum_01 IgG1, VLX4hum_01 IgG4PE, VLX4hum_02 IgG1, VLX4hum_02 IgG4PE, VLX4hum_06 IgG4 PE, VLX4hum_07 IgG4PE, VLX4hum_12 IgG4PE, and VLX4hum_13 IgG4PE caused increased PS exposure and cell death. In contrast, the humanized mAbs VLX4hum_08 IgG4PE and VLX4hum_11 IgG4PE did not cause increased PS exposure and cell death of Jurkat cells. Induction of cell death and the promotion of phagocytosis of susceptible cancer cells imparts an additional desirable antibody property and potential therapeutic benefit in the treatment of cancer.

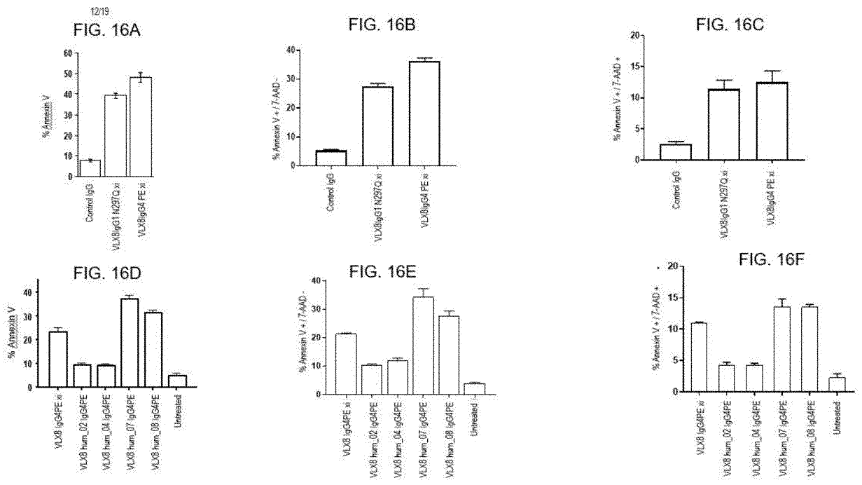

As shown in FIGS. 16A-16F, the soluble VLX8 chimeric and humanized mAbs induced increased PS exposure and cell death of Jurkat cells as measured by the % of the cells that are annexin V⁺ (FIGS. 16A, 16D), annexin V⁺/7-AAD⁻ (FIGS. 16B, 16E), or annexin V⁺/7-AAD⁺ (FIGS. 16C, 16F). The chimeric mAbs, VLX8 IgG1 N297Q xi and VLX8 IgG4PE xi, and the humanized mAbs, VLX8hum_07 IgG4PE and VLX8hum_08 IgG4PE, induced increased PS exposure and cell death of Jurkat cells. In contrast, the humanized mAbs VLX8hum_02 IgG4PE and VLX8hum_04 IgG4PE did not cause increased PS exposure and cell death of Jurkat cells. Induction of cell death and the promotion of phagocytosis of susceptible cancer cells imparts an additional desirable antibody property and potential therapeutic benefit in the treatment of cancer.

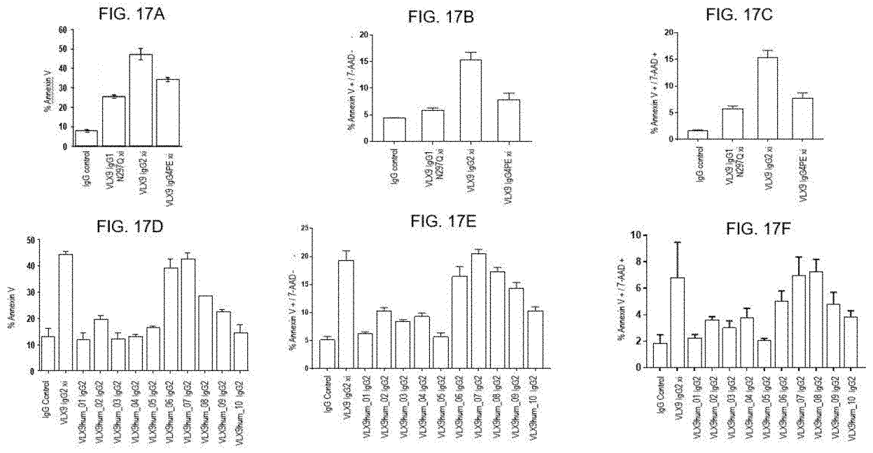

As shown in FIGS. 17A-17F, the soluble VLX9 chimeric and humanized antibodies induced increased PS exposure and cell death of Jurkat cells as measured by % of the cells that are annexin V⁺ (FIG. 17A and FIG. 17D), annexin V⁺/7-AAD⁻ (FIG. 17B and FIG. 17E), or annexin V⁺/7-AAD⁺ (FIG. 17C and FIG. 17F). The chimeric VLX9 IgG2xi mAb and the humanized mAbs VLX9hum_06 IgG2, VLX9hum_07 IgG2, VLX9hum_08 IgG2, and VLX9hum_09 IgG2 induced increased PS exposure and cell death of Jurkat cells. In contrast, the humanized mAbs VLX9hum_01 IgG2, VLX9hum_02 IgG2, VLX9hum_03 IgG2, VLX9hum_04 IgG2, VLX9hum_05 IgG2 and VLX9hum_010 IgG2 did not cause increased PS exposure and cell death of Jurkat cells. Induction of cell death and the promotion of phagocytosis of susceptible cancer cells imparts an additional desirable antibody property and potential therapeutic benefit in the treatment of cancer. Importantly, chimeric and humanized mAbs that cause cell death of tumor cells do not cause cell death of normal cells.

### Example 10

### Damage-Associated Molecular Pattern (DAMP) Expression and Release, Mitochondrial Depolarization and Cell Death Caused by Humanized Anti-CD47 mAb

Humanized Anti-CD47 mAbs Cause Loss of Mitochondrial Membrane Potential

These experiments demonstrate that humanized anti-CD47 mAbs of the present disclosure exhibit the ability to induce the loss of mitochondrial membrane potential in tumor cell as described previously (Manna and Frazier, 2014 Journal of Immunology 170(7):3544-3553).

Loss of mitochondrial membrane potential in the tumor cell was determined using JC-1 dye (Thermo; Catalogue #M34152). Human Raji lymphoma cells (ATCC, Manassas, Va.; Catalog #CCL-86) or other cells types that express sufficient levels of CD47 will be used. Cells were grown in RPMI-1640 medium containing 10% (v/v) heat inactivated fetal bovine serum (BioWest; Catalogue #S01520), 100 units/mL penicillin, 100 μg mL streptomycin (Sigma; Catalogue #P4222) at densities less than 1×10⁶ cells/mL. For this assay, Raji cells were plated in 96 well tissue culture plates at a density of 1×10⁵ cells/ml RPMI-1640 medium containing 10% (v/v) heat inactivated fetal bovine serum (BioWest; Catalog #S01520), 100 units/mL penicillin, 100 μg/mL streptomycin (Sigma; #P4222).

The humanized antibodies (VLX4hum_01 IgG4PE, VLX4hum_07 IgG4PE, VLX8hum_11 IgG4PE, VLX9hum_06 IgG2 VLX9hum_08 IgG2 and VLX9hum_03 IgG2) as disclosed herein, purified from transient transfections in CHO cells as described above, as well as the control chimeric antibody, were added at a final concentration of 10 μg/ml. As a positive control for loss of mitochondrial membrane potential, cells were treated with 1 μM of chemotherapeutic anthracycline mitoxantrone. The cells were incubated at 37° C. for 24 hours, after which the cells were harvested, washed twice with PBS, and incubated for 30 minutes with JC-1 dye as described above, diluted 1:2000 in PBS. After 30 minutes the cells were washed twice with PBS, resuspended in 100 μl of PBS, and analyzed for the percent of cells that shift their fluorescence emission from red to green by flow cytometry (Accuri C6, Becton Dickinson, Franklin Lakes, N.J.). Results are presented as means±SEM and analyzed for statistical significance using ANOVA in GraphPad Prism 6.

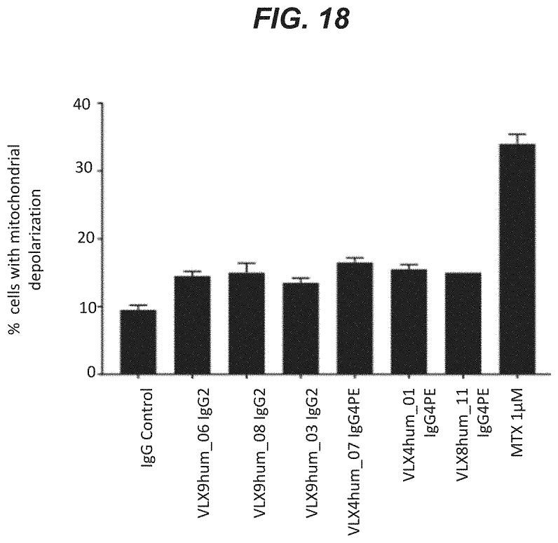

Some of the chimeric or humanized antibodies induce the loss of mitochondrial membrane potential in the tumor cell. As shown in FIG. 18, the percent of cells with mitochondrial membrane depolarization in all anti-CD47 mAb treated cultures was significantly increased (p<0.05) compared to an isotype control. This increase in the amount of mitochondrial membrane depolarization demonstrates that anti-CD47 chimeric or humanized antibodies induce mitochondrial depolarization that leads to cell death in human tumor cells.

Humanized Anti-CD47 mAbs Cause Increase in Cell Surface Calreticulin Expression

These experiments demonstrate that humanized anti-CD47 mAbs of the present disclosure exhibit the ability to expose the endoplasmic reticulum resident chaperone calreticulin on the surface of the tumor cell as, for example, described previously using chemotherapeutic anthracyclines such as doxorubicin and mitoxantrone, as disclosed by Obeid et al. (2007) Nat. Med. 13(1):54-61.

Cell surface exposure of calreticulin was determined using a rabbit monoclonal antibody against calreticulin conjugated to Alexa Fluor 647 (Abcam; Catalogue #ab196159). Human Raji lymphoma cells (ATCC, Manassas, Va.; Catalog #CCL-86) or other cells types that express sufficient levels of CD47 will be used. Cells were grown in RPMI-1640 medium containing 10% (v/v) heat inactivated fetal bovine serum (BioWest; Catalogue #S01520), 100 units/mL penicillin, 100 μg mL streptomycin (Sigma; Catalogue #P4222) at densities less than 1×10⁶ cells/mL. For this assay, cells were plated in 96 well tissue culture plates at a density of 1×10⁵ cells/ml RPMI-1640 medium containing 10% (v/v) heat inactivated fetal bovine serum (BioWest; Catalog #S01520), 100 units/mL penicillin, 100 μg/mL streptomycin (Sigma; #P4222).

The humanized antibodies (VLX4hum_01 IgG4PE, VLX4hum_07 IgG4PE, VLX8hum_11 IgG4PE, VLX9hum_06 IgG2, VLX9hum_08 IgG2 VLX9hum_03 IgG2) as disclosed herein, purified from transient transfections in CHO cells as described above, as well as the control chimeric antibody, were added at a final concentration of 10 μg/ml. As a positive control for calreticulin exposure, cells were treated with 1 μM of chemotherapeutic anthracycline mitoxantrone. The cells were incubated at 37° C. for 24 hours, after which the cells were harvested, washed twice with PBS, and incubated for 30 minutes with anti-calreticulin antibody as described above, diluted 1:200 in PBS. After 30 minutes the cells were washed twice with PBS, resuspended in 100 μl of PBS, and analyzed for the mean fluorescence intensity of the anti-calreticulin antibody signal as well as the percent of cells that stain positive for cell surface calreticulin by flow cytometry (Accuri C6, Becton Dickinson, Franklin Lakes, N.J.). Results are presented as means SEM and analyzed for statistical significance using ANOVA in GraphPad Prism 6.

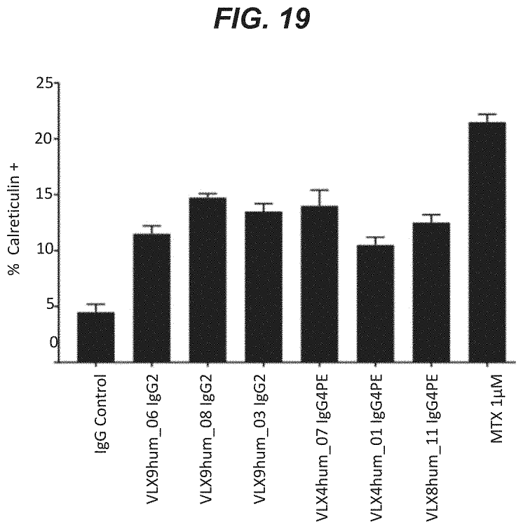

As shown in FIG. 19, the humanized antibodies induced the preapoptotic exposure of calreticulin on the tumor cell surface. The percent of calreticulin positive cells in all anti-CD47 mAb treated cultures was significantly increased (p<0.05) compared to an isotype control. This increase in the exposure of calreticulin on the cell surface demonstrates that some of the humanized antibodies induce DAMPs from tumor cells that can lead to phagocytosis of tumor cells and processing of tumor antigen by innate immune cells.

Humanized Anti-CD47 mAbs Cause Increased Protein Disulfide-Isomerase 3 (PDIA3) Expression

These experiments demonstrate that humanized anti-CD47 mAbs of the present disclosure exhibit the ability to expose the endoplasmic reticulum resident chaperone PDIA3 on the surface of the tumor cell as, for example, described previously using chemotherapeutic anthracyclines such as doxorubicin and mitoxantrone, as disclosed by Panaretakis et al. (2008) Cell Death & Differentiation 15:1499-1509.

Cell surface exposure of PDIA3 was determined using a mouse monoclonal antibody against PDIA3 conjugated to FITC (Abcam; Catalogue #ab183396). Human Raji lymphoma cells (ATCC, Manassas, Va.; Catalog #CCL-86) or other cells types that express sufficient levels of CD47 will be used. Cells were grown in RPMI-1640 medium containing 10% (v/v) heat inactivated fetal bovine serum (BioWest; Catalogue #S01520), 100 units/mL penicillin, 100 μg mL streptomycin (Sigma; Catalogue #P4222) at densities less than 1×10⁶ cells/mL. For this assay, cells were plated in 96 well tissue culture plates at a density of 1×10⁵ cells/ml RPMI-1640 medium containing 10% (v/v) heat inactivated fetal bovine serum (BioWest; Catalog #S01520), 100 units/mL penicillin, 100 μg/mL streptomycin (Sigma; #P4222).

The humanized antibodies (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) as disclosed herein, purified from transient transfections in CHO cells as described above, as well as the control chimeric antibody, were added at a final concentration of 10 μg/ml. As a positive control for PDIA3 exposure, cells were treated with 1 μM of chemotherapeutic anthracycline mitoxantrone. The Raji cells were incubated at 37° C. for 24 hours, after which the cells were harvested, washed twice with PBS, and incubated for 30 minutes with anti-PDIA3 antibody as described above, diluted 1:200 in PBS. After 30 minutes the cells were washed twice with PBS, resuspended in 100 μl of PBS, and analyzed for the mean fluorescence intensity of the anti-PDIA3 antibody signal as well as the percent of cells that stain positive for cell surface calreticulin by flow cytometry (Accuri C6, Becton Dickinson, Franklin Lakes, N.J.). Results are presented as means±SEM and analyzed for statistical significance using ANOVA in GraphPad Prism 6.

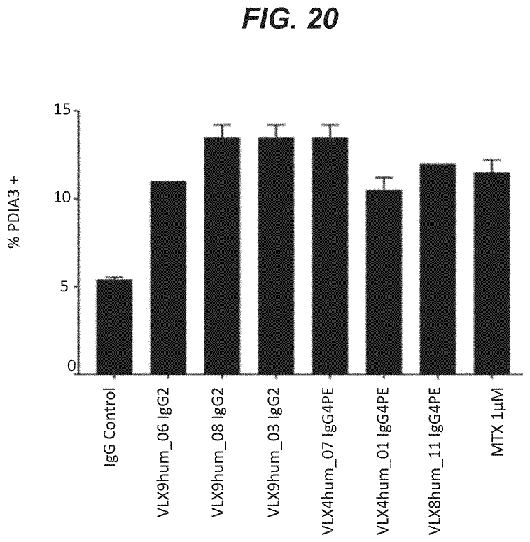

Some of the chimeric or humanized antibodies induce the preapoptotic exposure of PDIA3 on the tumor cell surface. As shown in FIG. 20, the percent of PDIA3 positive cells in all the soluble anti-CD47 mAb treated cultures was significantly increased (p<0.05) compared to the background obtained with a negative control, humanized isotype-matched antibody. This increase in the exposure of PDIA3 on the cell surface demonstrates that some of the chimeric or humanized antibodies induce DAMPs from tumor cells that can lead to phagocytosis of tumor cells and processing of tumor antigen by innate immune cells.

Humanized Anti-CD47 mAbs Cause Increased Cell Surface HSP70 Expression

These experiments demonstrate that humanized anti-CD47 mAbs of the present disclosure exhibit the ability to expose the endoplasmic reticulum resident chaperone HSP70 on the surface of the tumor cell as, for example, described previously using chemotherapeutic anthracyclines such as doxorubicin and mitoxantrone, as disclosed by Fucikova et al. (2011) Cancer Research 71(14):4821-4833.

Cell surface exposure of HSP70 was determined using a mouse monoclonal antibody against HSP70 conjugated to Phycoerythrin (Abcam; Catalogue #ab65174). Human Raji lymphoma cells (ATCC, Manassas, Va.; Catalog #CCL-86) or other cells types that express sufficient levels of CD47 were used. Cells were grown in RPMI-1640 medium containing 10% (v/v) heat inactivated fetal bovine serum (BioWest; Catalogue #S01520), 100 units/mL penicillin, 100 μg mL streptomycin (Sigma; Catalogue #P4222) at densities less than 1×10⁶ cells/mL. For this assay, cells were plated in 96 well tissue culture plates at a density of 1×10⁵ cells/ml RPMI-1640 medium containing 10% (v/v) heat inactivated fetal bovine serum (BioWest; Catalog #S01520), 100 units/mL penicillin, 100 μg/mL streptomycin (Sigma; #P4222).

The humanized antibodies (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) as disclosed herein, purified from transient transfections in CHO cells as described above, as well as the control chimeric antibody, were added at a final concentration of 10 μg/ml. As a positive control for HSP70 exposure, Raji cells were treated with 1 μM of chemotherapeutic anthracycline mitoxantrone. The cells were incubated at 37° C. for 24 hours, after which the cells were harvested, washed twice with PBS, and incubated for 30 minutes with anti-HSP70 antibody as described above, diluted 1:200 in PBS. After 30 minutes the cells were washed twice with PBS, resuspended in 100 μl of PBS, and analyzed for the mean fluorescence intensity of the anti-HSP70 antibody signal as well as the percent of cells that stain positive for cell surface calreticulin by flow cytometry (Accuri C6, Becton Dickinson, Franklin Lakes, N.J.). Results are presented as means SEM and analyzed for statistical significance using ANOVA in GraphPad Prism 6.

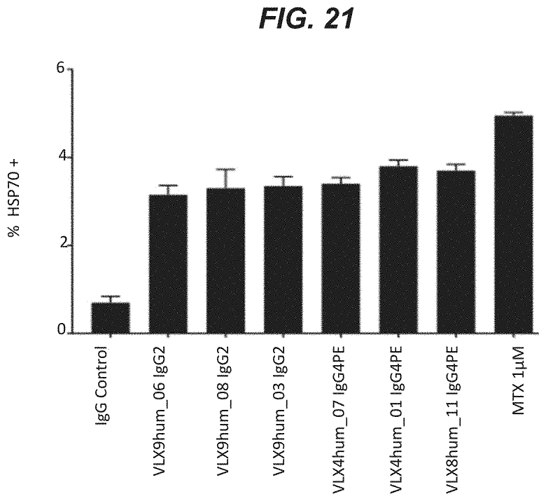

Some of the chimeric or humanized antibodies induce the preapoptotic exposure of HSP70 on the tumor cell surface. As shown in FIG. 21, the percent of HSP70 positive cells in all anti-CD47 mAb treated cultures was significantly increased (p<0.05) compared to those seen in isotype control treated cultures. This increase in the exposure of HSP70 on the cell surface demonstrates that some of the chimeric or humanized antibodies induce DAMPs from tumor cells and can lead to phagocytosis of tumor cells and processing of tumor antigen by innate immune cells.

Humanized Anti-CD47 mAbs Cause Increased Cell Surface HSP90 Expression

These experiments demonstrate that humanized anti-CD47 mAbs of the present disclosure expose the endoplasmic reticulum resident chaperone HSP70 on the surface of the tumor cell as, for example, described previously using chemotherapeutic anthracyclines such as doxorubicin and mitoxantrone, as disclosed by Fucikova et al. (2011) Cancer Research 71(14):4821-4833.

Cell surface exposure of HSP90 was determined using a mouse monoclonal antibody against HSP70 conjugated to Phycoerythrin (Abcam; Catalogue #ab65174). Human Raji lymphoma cells (ATCC, Manassas, Va.; Catalog #CCL-86) or other cells types that express sufficient levels of CD47 were used. Cells are grown in RPMI-1640 medium containing 10% (v/v) heat inactivated fetal bovine serum (BioWest; Catalogue #S01520), 100 units/mL penicillin, 100 μg mL streptomycin (Sigma; Catalogue #P4222) at densities less than 1×10⁶ cells/mL. For this assay, cells were plated in 96 well tissue culture plates at a density of 1×10⁵ cells/ml RPMI-1640 medium containing 10% (v/v) heat inactivated fetal bovine serum (BioWest; Catalog #S01520), 100 units/mL penicillin, 100 μg/mL streptomycin (Sigma; #P4222).

The humanized antibodies (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) as disclosed herein, purified from transient transfections in CHO cells as described above, as well as the control chimeric antibody, were added at a final concentration of 10 μg/ml. As a positive control for HSP90 exposure, cells were treated with 1 μM of chemotherapeutic anthracycline mitoxantrone. The Raji cells were incubated at 37° C. for 24 hours, after which the cells were harvested, washed twice with PBS, and incubated for 30 minutes with anti-HSP70 antibody as described above, diluted 1:200 in PBS. After 30 minutes the cells were washed twice with PBS, resuspended in 100 μl of PBS, and analyzed for the mean fluorescence intensity of the anti-HSP70 antibody signal as well as the percent of cells that stain positive for cell surface calreticulin by flow cytometry (Accuri C6, Becton Dickinson, Franklin Lakes, N.J.). Results are presented as means±SEM and analyzed for statistical significance using ANOVA in GraphPad Prism 6.

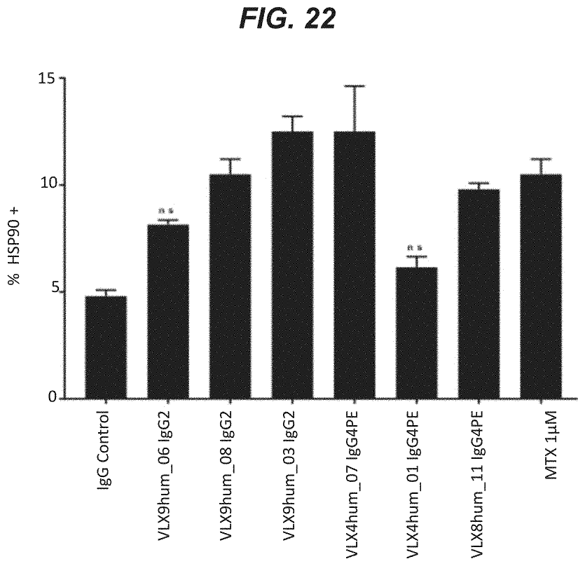

Some of the chimeric or humanized antibodies induce the preapoptotic exposure of HSP90 on the tumor cell surface. As shown in FIG. 22, the percent of HSP90 positive cells in soluble anti-CD47 mAb-treated cultures was significantly increased (p<0.05) compared to the background obtained with a negative control, humanized isotype-matched antibody, except for VLXhum_06 IgG2 and VLX4hum_01 IgG4PE (ns, not significant). This increase in the exposure of HSP90 on the cell surface demonstrates that some of the chimeric or humanized antibodies induce DAMPs from tumor cells and can lead to phagocytosis of tumor cells and processing of tumor antigen by innate immune cells.

Humanized Anti-CD47 mAbs Cause Increased ATP Release

These experiments demonstrate that humanized anti-CD47 mAbs of the present disclosure induce increased release of adenosine triphosphate (ATP) from the tumor cell as described previously using anthracycline chemotherapy drugs (Martins et al., 2014 Cell Death and Differentiation 21:79-91).

Release of ATP from the tumor cell is determined by quantitative bioluminescence assay as described by the manufacturer (Molecular Probes; Catalogue #A22066). Human Raji lymphoma cells (ATCC, Manassas, Va.; Catalog #CCL-86) or other cells types that express sufficient levels of CD47 were used. Cells were grown in RPMI-1640 medium containing 10% (v/v) heat inactivated fetal bovine serum (BioWest; Catalogue #501520), 100 units/mL penicillin, 100 μg mL streptomycin (Sigma; Catalogue #P4222) at densities less than 1×10⁶ cells/mL. For this assay, cells were plated in 96 well tissue culture plates at a density of 1×10⁵ cells/ml RPMI-1640 medium containing 10% (v/v) heat inactivated fetal bovine serum (BioWest; Catalog #S01520), 100 units/mL penicillin, 100 μg/mL streptomycin (Sigma; #P4222).

The humanized antibodies (VLX4hum_01 IgG4PE, VLX4hum_07 IgG4PE, VLX8hum_11 IgG4PE, VLX9hum_06 IgG2, VLX9hum_08 IgG2 and VLX9hum_03) as disclosed herein, purified from transient transfections in CHO cells as described above, as well as the control chimeric antibody, were added at a final concentration of 10 μg/ml. As a positive control for ATP release, cells were treated with 1 μM of chemotherapeutic anthracycline mitoxantrone. The cells were incubated at 37° C. for 24 hours, after which the cell-free supernatant was collected and stored at −80° C. After all samples have been collected, 10 μl of each sample was tested by the ATP determination kit as described above. Final concentrations were determined by comparing experimental values to a standard curve and displayed as the concentration of ATP (μM) released by tumor cells in response to antibody treatment. Results are presented as means SEM and analyzed for statistical significance using ANOVA in GraphPad Prism 6.

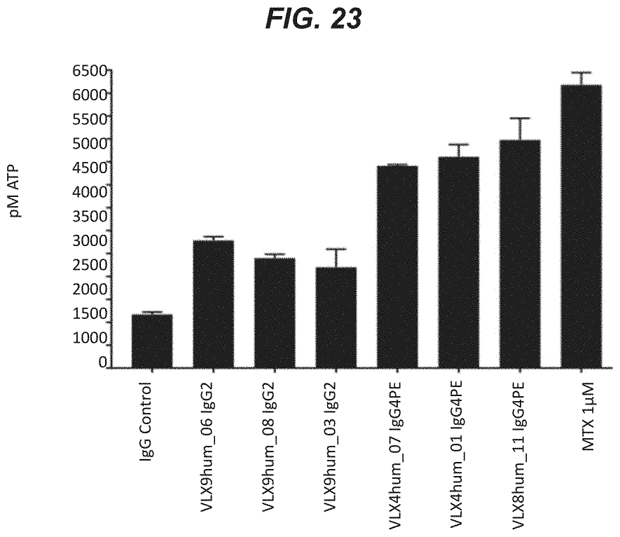

The humanized antibodies increased the release of ATP from the tumor cells. As shown in FIG. 23, the amount of released ATP in all anti-CD47 mAb treated cultures was significantly increased (p<0.05) compared to an isotype control. This increase in the release of ATP demonstrates that some of the chimeric or humanized antibodies induce the release of ATP from tumor cells and can lead to dendritic cell migration through its cognate purinergic receptors.

Humanized Anti-CD47 mAbs Cause HMGB1 Release

These experiments demonstrate that humanized anti-CD47 mAbs of the present disclosure increase the release of the non-histone chromatin protein high-mobility group box 1 (HMGB1) from the tumor cell as described previously using chemotherapy agents, such as oxaliplatin (Tesniere et al., 2010 Oncogene, 29:482-491) and mitoxantrone (Michaud et al., 2011 Science 334:1573-1577).

Release of HMGB1 protein from the tumor cell was determined by enzyme immunoassay as described by the manufacturer (IBL International; Hamburg, Germany, Catalogue #ST51011). Human Raji lymphoma cells (ATCC, Manassas, Va.; Catalog #CCL-86) or other cells types that express sufficient levels of CD47 were used. Cells will be grown in RPMI-1640 medium containing 10% (v/v) heat inactivated fetal bovine serum (BioWest; Catalogue #S01520), 100 units/mL penicillin, 100 μg mL streptomycin (Sigma; Catalogue #P4222) at densities less than 1×10⁶ cells/mL. For this assay, cells were plated in 96 well tissue culture plates at a density of 1×10⁵ cells/ml RPMI-1640 medium containing 10% (v/v) heat inactivated fetal bovine serum (BioWest; Catalog #S01520), 100 units/mL penicillin, 100 μg/mL streptomycin (Sigma; #P4222).

The humanized antibodies (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) as disclosed herein, purified from transient transfections in CHO cells as described above, as well as the control chimeric antibody, will then be added at a final concentration of 10 μg/ml. As a positive control for HMGB1 release, Raji cells were treated with 1 μM of chemotherapeutic anthracycline mitoxantrone. The cells were incubated at 37° C. for 24 hours, after which the cell-free supernatant was collected and stored at −80° C. After all samples have been collected, 10 μl of each sample was tested by HMGB1 ELISA as described above. Final concentrations were determined by comparing experimental values to a standard curve and reported as the concentration of HMGB1 (ng/ml) released by tumor cells in response to antibody treatment. Results are presented as means±SEM and analyzed for statistical significance using ANOVA in GraphPad Prism 6.

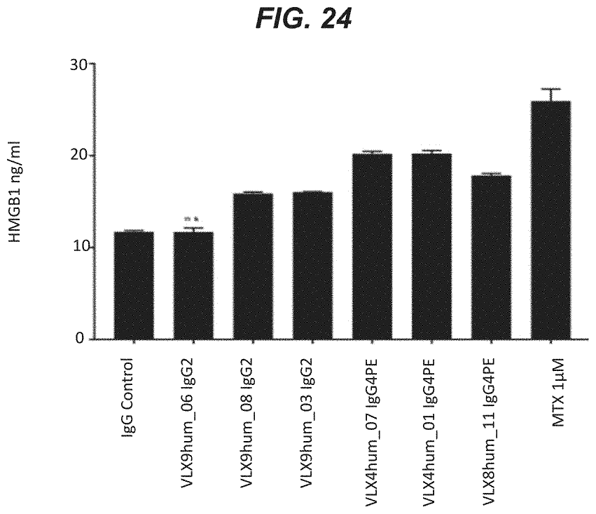

As shown in FIG. 24, the humanized antibodies increased the release of HMGB1 protein from the tumor cells. The amount of released HMGB1 protein in all anti-CD47 mAb treated cultures was significantly increased (p<0.05) compared to an isotype control, except for VLX9hum_06 IgG2 (ns, not significant). This increase in the release of HMGB1 demonstrates that some of the chimeric or humanized antibodies induce release of DAMPs from tumor cells and can lead to dendritic cell activation.

Humanized Anti-CD47 mAbs Cause CXCL10 Release

These experiments demonstrate that humanized anti-CD47 mAbs of the present disclosure increase the production and release of the chemokine CXCL10 from the human tumor cells as described previously using anthracycline chemotherapy drugs (Sistigu et al., 2014 Nat. Med. 20(11):1301-1309).

Release of the CXCL10 from the tumor cell was determined by enzyme immunoassay as described by the manufacturer (R&D Systems; Catalogue #DIP100). Human Raji lymphoma cells (ATCC, Manassas, Va.; Catalog #CCL-86) or other cells types that express sufficient levels of CD47 will be used. Cells were grown in RPMI-1640 medium containing 5% (v/v) heat inactivated fetal bovine serum (BioWest; Catalogue #S01520), 100 units/mL penicillin, 100 μg mL streptomycin (Sigma; Catalogue #P4222) at densities less than 1×10⁶ cells/mL. For this assay, cells were plated in 96 well tissue culture plates at a density of 1×10⁵ cells/ml RPMI-1640 medium containing 5% (v/v) heat inactivated fetal bovine serum (BioWest; Catalog #S01520), 100 units/mL penicillin, 100 □g/mL streptomycin (Sigma; #P4222).

The humanized antibodies (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) as disclosed herein, purified from transient transfections in CHO cells as described above, as well as the control chimeric antibody, were added at a final concentration of 10 μg/ml. As a positive control for CXCL10 release, Raji cells were treated with 1 μM of the chemotherapeutic anthracycline mitoxantrone. The cells were incubated at 37° C. for 24 hours, after which the cell-free supernatant was collected and stored at −80° C. After all samples have been collected, 10 μl of each sample was tested by the CXCL10 ELISA as described above. Final concentrations were determined by comparing experimental values to a standard curve and displayed as the concentration of CXCL10 (pg/ml) released by tumor cells in response to antibody treatment.

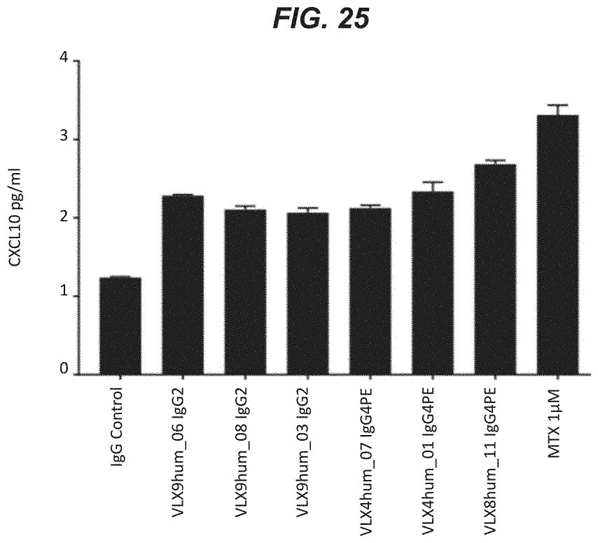

Some of the chimeric or humanized antibodies induce release of CXCL10 by human tumor cells. As shown in FIG. 25, the amount of released CXCL10 in all anti-CD47 mAb treated cultures significantly increased (p<0.05) compared to an isotype control. This increase in the release of CXCL10 demonstrates that some of the chimeric or humanized antibodies induce the release of CXCL10 from tumor cells and suggest a role in the recruitment of immune cells to the tumor.

### Example 11

### Damage-Associated Molecular Pattern (DAMP) Expression and Release, Mitochondrial Depolarization and Cell Death Caused by Humanized Anti-CD47 mAbs

These studies were conducted as described in Example 10, except that the human Jurkat T ALL cell line (ATCC, Manassas, Va.; Catalog #TIB-152) was used.

Humanized Anti-CD47 mAbs Cause Loss of Mitochondrial Membrane Potential

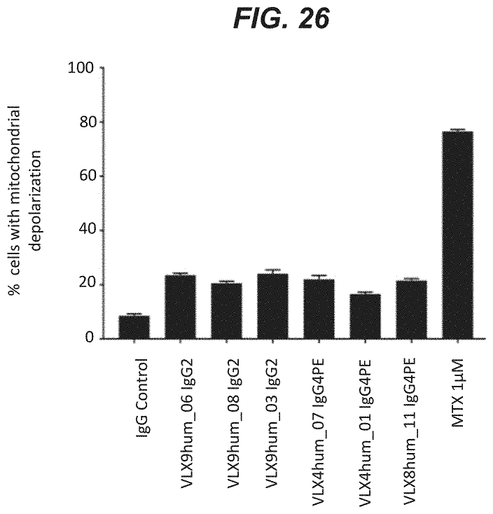

As shown in FIG. 26, the humanized mAbs (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) caused a significant increase in the percent of cells with mitochondrial membrane depolarization (p<0.05) compared to an isotype control. This increase in the amount of mitochondrial membrane depolarization demonstrates that some of the chimeric or humanized antibodies induce cell death in human tumor cells.

Humanized Anti-CD47 mAbs Cause Increase in Cell Surface Calreticulin Expression

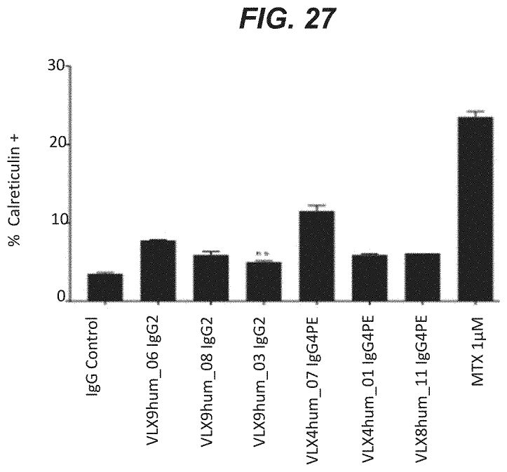

As shown in FIG. 27, the humanized antibodies (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) induced the preapoptotic exposure of calreticulin on the tumor cell surface. The percent of calreticulin positive cells in all anti-CD47 mAb treated cultures were significantly increased (p<0.05) compared to an isotype control, except VLX9hum_03 IgG2 (ns). This increase in the exposure of calreticulin on the cell surface demonstrated that some of the humanized antibodies induce DAMPs from tumor cells and can lead to phagocytosis of tumor cells and processing of tumor antigen by innate immune cells.

Humanized Anti-CD47 mAbs Cause Increase in Cell Surface PDIA3 Expression

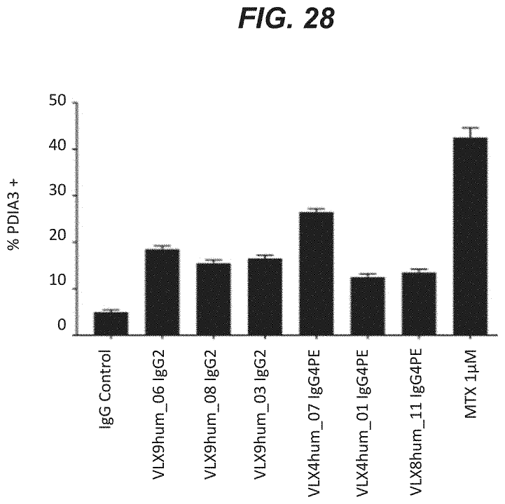

As shown in FIG. 28, the percent of PDIA3 positive cells in soluble anti-CD47 mAb (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) treated cultures were significantly increased (p<0.05) compared to the background obtained with a negative control, humanized isotype-matched antibody. This increase in the exposure of PDIA3 on the cell surface demonstrates that some of the chimeric or humanized antibodies induce DAMPs from tumor cells and can lead to phagocytosis of tumor cells and processing of tumor antigen by innate immune cells.

Humanized Anti-CD47 mAbs Cause Increase in Cell Surface HSP70 Expression

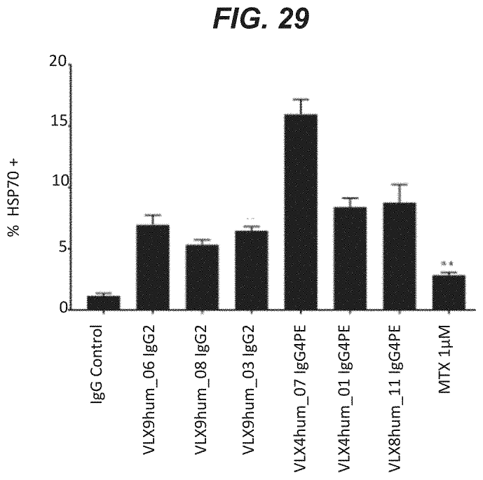

As shown in FIG. 29, the percent of HSP70 positive cells in anti-CD47 mAb (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) treated cultures were significantly increased (p<0.05) compared to those seen in isotype control treated cultures. Although each of the anti-CD47 mAbs caused a statistically significant increase in HSP70 expression, mitoxantrone did not. This increase in the exposure of HSP70 on the cell surface demonstrates that some of the chimeric or humanized antibodies induce DAMPs from tumor cells and can lead to phagocytosis of tumor cells and processing of tumor antigen by innate immune cells.

Humanized Anti-CD47 mAbs Cause Increase in Cell Surface HSP90 Expression

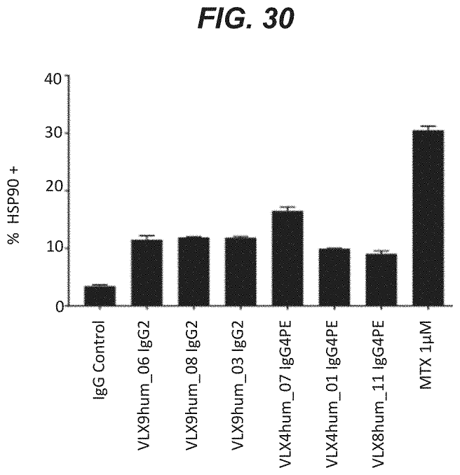

As shown in FIG. 30, the percent of HSP90 positive cells in soluble anti-CD47 mAb (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) treated cultures were significantly increased (p<0.05) compared to the background obtained with a negative control, humanized isotype-matched antibody. This increase in the exposure of HSP90 on the cell surface demonstrates that some of the chimeric or humanized antibodies induce DAMPs from tumor cells and can lead to phagocytosis of tumor cells and processing of tumor antigen by innate immune cells.

Humanized Anti-CD47 mAbs Cause Increase in ATP Release

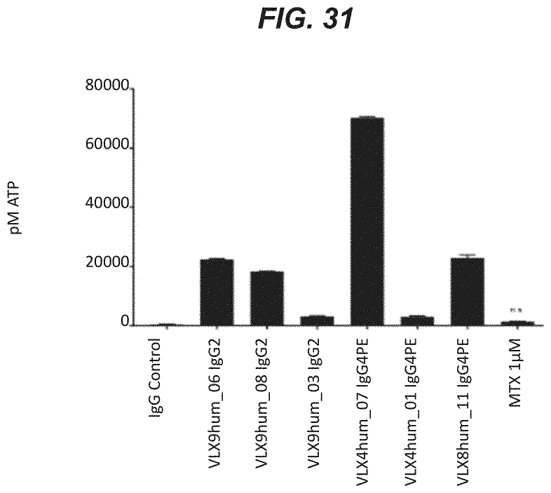

As shown in FIG. 31, the amount of released ATP in humanized anti-CD47 mAb (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) treated cultures was significantly increased (p<0.05) compared to an isotype control. Although each of the anti-CD47 mAbs caused a statistically significant increase in HSP70 expression, mitoxantrone did not (ns). This increase in the release of ATP will demonstrates that some of the chimeric or humanized antibodies induce the release of ATP from tumor cells and can lead to dendritic cell migration through its cognate purinergic receptors.

Humanized Anti-CD47 mAbs Cause Increase in HMGB1 Release

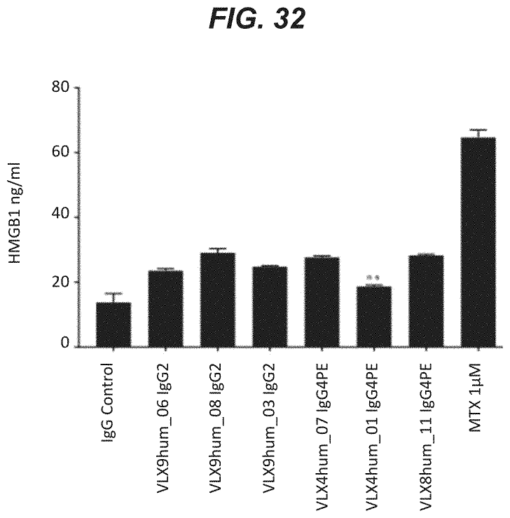

As shown in FIG. 32, the amount of released HMGB1 protein in anti-CD47 mAb (VLX4hum_01 IgG4PE, VLX4hum_07 IgG4PE, VLX8hum_11 IgG4PE, VLX9hum_06 IgG2, VLX9hum_08 IgG2 and VLX9hum_03 IgG2) treated cultures was significantly increased (p<0.05) compared to an isotype control, except for VLX4hum_01 IgG4PE (ns). This increase in the release of HMGB1 demonstrates that some of the chimeric or humanized antibodies induce DAMPs from tumor cells and can lead to dendritic cell activation.

### Example 12

### Combination Treatment with Humanized Anti-CD47 mAb and Chemotherapy Results in Additive or Synergistic Effects

These experiments demonstrate that humanized anti-CD47 mAbs of the present disclosure cause additive or synergistic activities when combined with clinically relevant chemotherapeutic agents to induce immunogenic cell death effects in human tumor cells.

Combination drug additivity/synergism was determined by combining increasing concentrations of humanized anti-CD47 mAb VLX4hum_07 IgG4 PE and doxorubicin (Sigma, PHR1789). Human Jurkat cells (ATCC, Manassas, Va.; Catalog #TIB-152) or other cells types that express sufficient levels of CD47 were used. Cells were grown in RPMI-1640 medium containing 10% (v/v) heat inactivated fetal bovine serum (BioWest; Catalogue #S01520), 100 units/mL penicillin, 100 μg mL streptomycin (Sigma; Catalogue #P4222) at densities less than 1×10⁶ cells/mL. For this assay, cells were plated in 96 well tissue culture plates at a density of 1×10⁵ cells/ml RPMI-1640 medium containing 10% (v/v) heat inactivated fetal bovine serum (BioWest; Catalog #S01520), 100 units/mL penicillin, 100 □g/mL streptomycin (Sigma; #P4222).

Jurkat cells were incubated with 0.03-10 μg/ml of VLX4hum_07 IgG4 PE alone, 0.3-100 nM of doxorubicin alone or a combination dose-response matrix of 0.03-10 μg/ml of VLX4hum_07 IgG4 PE and 0.3-100 nM of doxorubicin in RPMI media at 37° C. for 24 hours, after which the cells were harvested and analyzed for phosphatidylserine using annexin V, 7-AAD, ER stress marker calreticulin on the cell surface. The supernatant was harvested for analysis of ATP release (as described above). Results are presented as means±SEM.

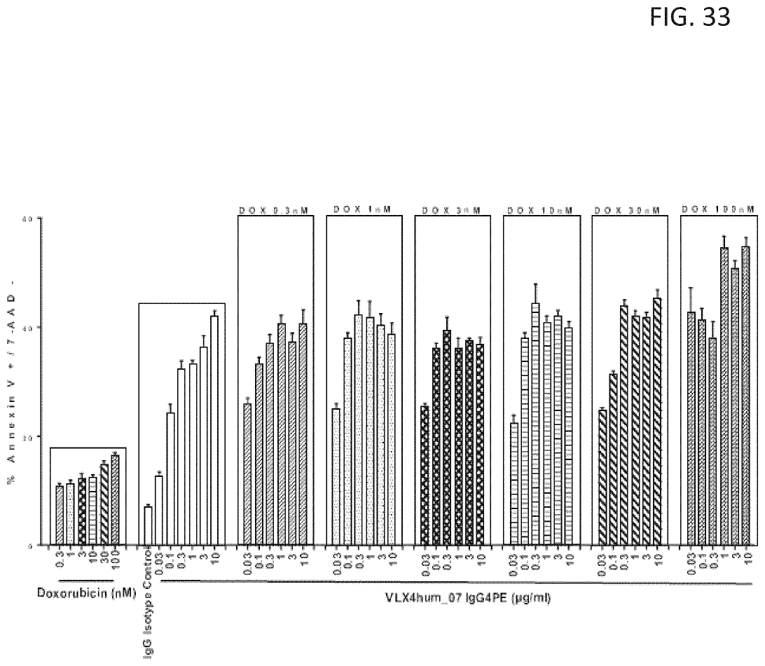

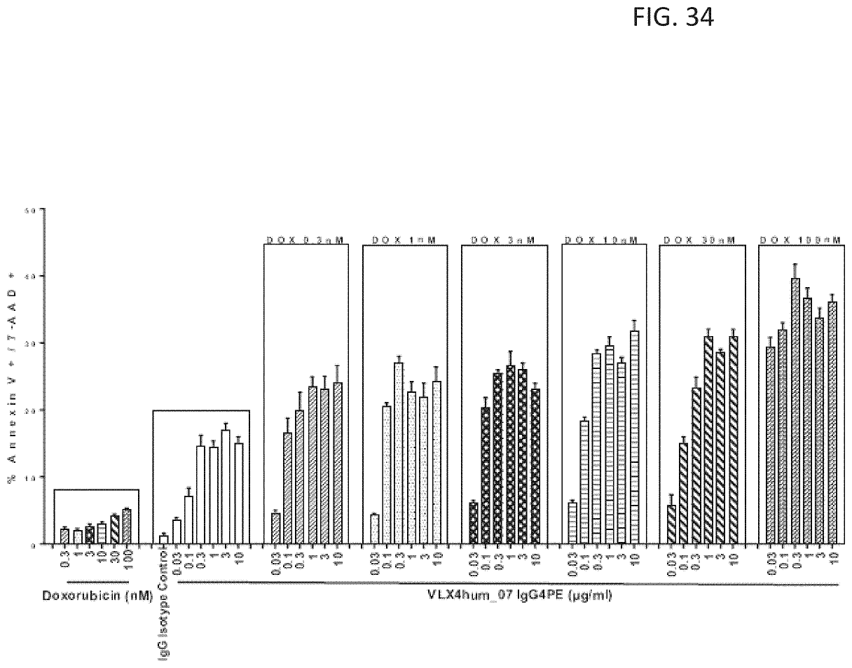

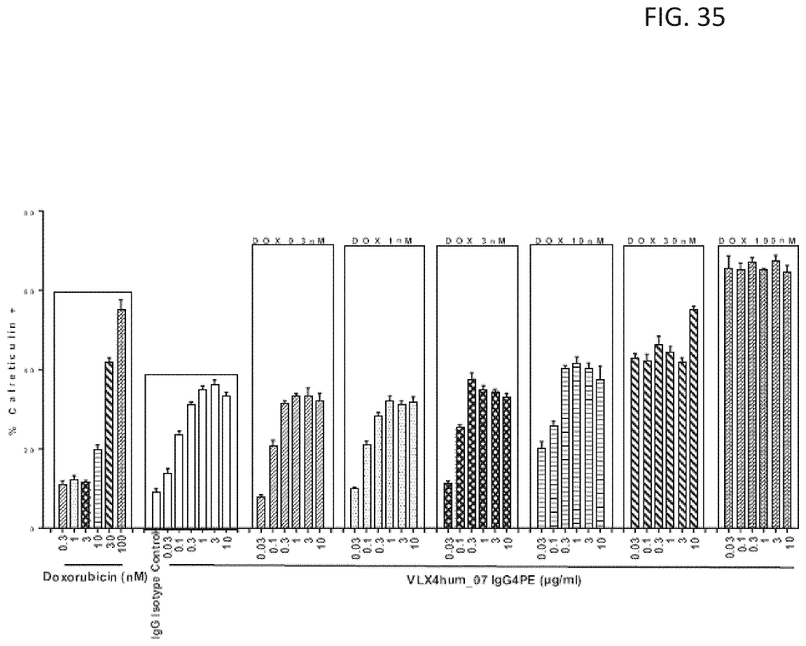

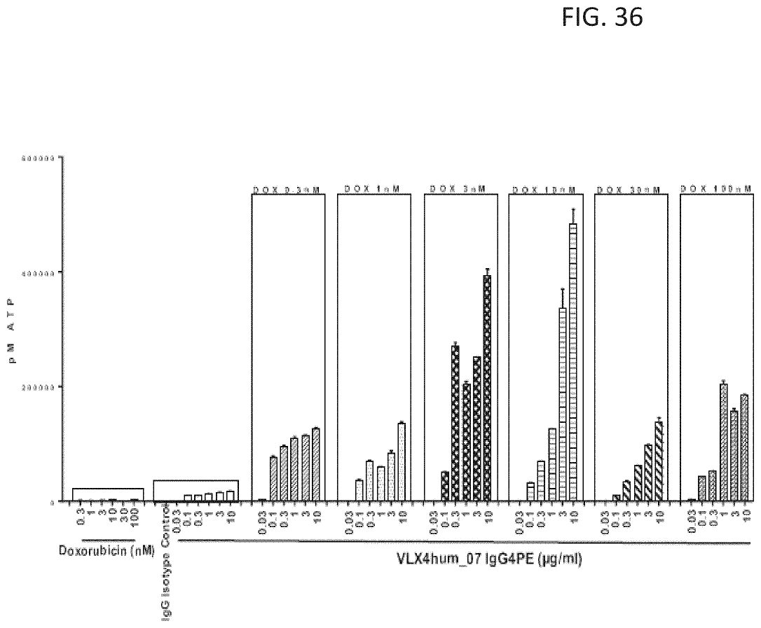

As shown in FIG. 33, some combinations of VLX4hum_07 IgG4PE and doxorubicin cause additive or synergistic effects on the percent of annexin V positive/7-AAD negative (annexin V+/7-AAD−) cells. As shown in FIG. 34, some combinations of VLX4hum_07 IgG4PE and doxorubicin cause additive or synergistic effects on the percent of annexin V positive/7-AAD positive (annexin V+7-AAD+) dead cells. As shown in FIG. 35, some combinations of VLX4hum_07 IgG4PE and doxorubicin cause additive or synergistic effects on the percent of calreticulin positive cells. As shown in FIG. 36, some combinations of VLX4hum_07 IgG4PE and doxorubicin cause additive or synergistic effects on the amount of ATP released.

### Example 13

### Hemagglutination of Human Red Blood Cells (hRBCs)

Many CD47 antibodies, including B6H12, BRIC126, MABL1, MABL2, CC2C6, 5F9, have been shown to cause hemagglutination (HA) of washed RBCs in vitro or in vivo (Petrova P. et al. Cancer Res 2015; 75(15 Suppl): Abstract nr 4271; U.S. Pat. No. 9,045,541; Uno et al. Oncol Rep. 17: 1189-94, 2007; Kikuchi et al. Biochem Biophys Res. Commun. 315: 912-8, 2004; Sikic B. et al. J Clin Oncol 2016; 34 (suppl; abstract 3019)). Hemagglutination of hRBCs was assessed following incubation of hRBCs with various concentrations of chimeric and humanized VLX4, VLX8, and VLX9 mAbs in vitro essentially as described by Kikuchi et al. Biochem Biophys Res. Commun (2004) 315:912-918. Blood was obtained from healthy donors, diluted (1:50) in PBS/1 mM EDTA/BSA and washed 3 times with PBS/EDTA/BSA. hRBCs were added to U-bottomed 96 well plates with equal volumes of the antibodies (75 μl of each) and incubated for 3 hrs at 37° C. and overnight at 4° C. A tight RBC pellet is observed with antibodies that do not cause hemagglutination, and a diffuse, hazy pattern is observed with antibodies that cause hemagglutination.

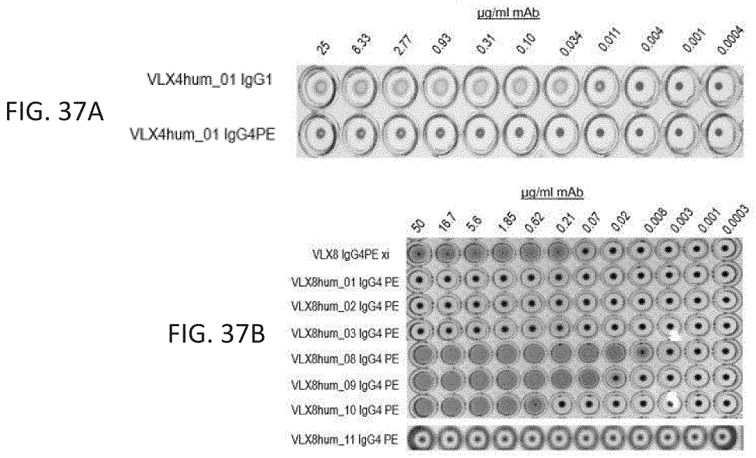

As shown in FIG. 37A and Tables 1 and 2, The VLX4hum_01 IgG1 caused visible hemagglutination of hRBCs, whereas the humanized VLX4hum_01 IgG4PE mAb did not (mAb concentrations 50 □g/ml to 0.3 ng/ml). The lack of detectable hemagglutination by VLX4hum_01 IgG4 PE imparts an additional desirable antibody property and potential therapeutic benefit in the treatment of cancer.

As shown in FIG. 37B and Tables 1 and 2, the chimeric antibody VLX8 IgG4PE (xi) and the humanized antibodies VLX8hum_08 IgG4PE, VLX8hum_09 IgG4PE, and VLX8hum_10 IgG4PE caused visible hemagglutination of hRBCs, whereas the VLX8 humanized Abs VLX8hum_01 IgG4PE, VLX8hum_02 IgG4 PE, VLX8hum_03 IgG4 PE and VLX8hum_11 IgG4PE did not (mAb concentrations 50 □g/ml to 0.3 ng/ml).

The lack of detectable hemagglutination by humanized antibodies VLX4hum_01 IgG4PE, VLX8hum_01 IgG4PE, VLX8hum_02 IgG4 PE, VLX8hum_03 IgG4 PE and VLX8hum_11 IgG4 PE imparts an additional desirable antibody property and a potential therapeutic benefit in the treatment of cancer.

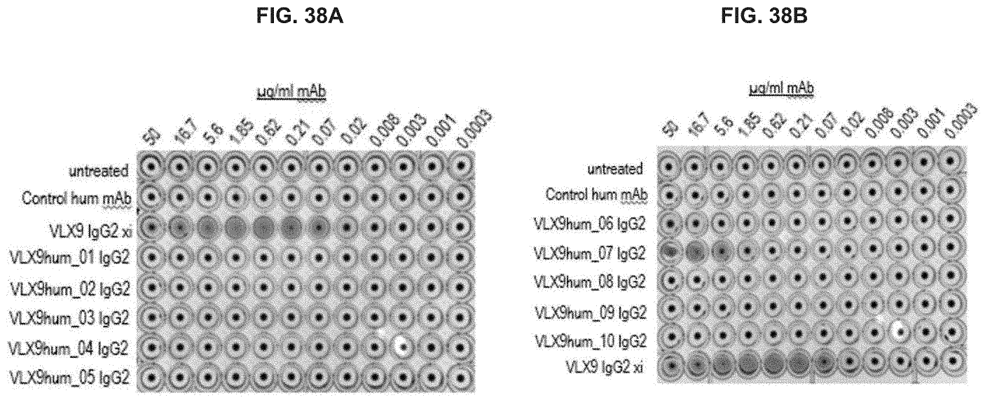

As shown in FIG. 38A and FIG. 38B, the chimeric antibody VLX9 IgG2 xi caused visible hemagglutination of hRBCs, whereas all of the humanized VLX9 mAbs except for VLX9hum_07 IgG2, did not cause detectable hemagglutination (at concentrations from 50 ug/ml to 0.3 pg/ml). However, the amount of detectable hemagglutination caused by VLX9hum_07 was reduced compared to the VLX9 IgG2 chimeric mAb. Again, the reduced or lack of detectable hemagglutination by the VLX9 humanized mAbs imparts an additional desirable antibody property and a potential therapeutic benefit in the treatment of cancer.

### Example 14

### Anti-Tumor Activity In Vivo

The purpose of this experiment was to demonstrate that VLX4, VLX8 and VLX9 humanized antibodies, exemplified by VLX4_07 IgG4PE, VLX8_10 IgG4PE and VLX9hum_08 IgG2, reduce tumor burden in vivo in a mouse xenograft model of lymphoma.

Raji human Burkitt's lymphoma cells (ATCC #CCL-86, Manassas, Va.) were maintained in RPMI-1640 (Lonza; Walkersville, Md.) supplemented with 10% Fetal Bovine Serum (FBS; Omega Scientific; Tarzana, Calif.) within a 5% CO2 atmosphere. Cultures were expanded in tissue culture flasks.

Female NSG (NOD-Cg-Prkdc^{scid}I12rg^{tm1Wjl}/SzJ) were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NSG mice were inoculated subcutaneously in the right flank with 0.1 mL of a 30% RPMI/70% Matrigel™ (BD Biosciences; Bedford, Mass.) mixture containing a suspension of 5×10⁶ Raji tumor cells. Five days following inoculation, digital calipers were used to measure width and length diameters of the tumor. Tumor volumes were calculated utilizing the formula: tumor volume (mm³)=(a×b²/2) where ‘b’ is the smallest diameter and ‘a’ is the largest diameter. Mice with palpable tumor volumes of 31-74 mm³ were randomized into 8-10/group and VLX9hum_08 or PBS (control) administration was initiated at this time. Mice were treated with 5 mg/kg of antibody 5×/week for 4 weeks by intraperitoneal injection. Tumor volumes and body weights were recorded twice weekly.

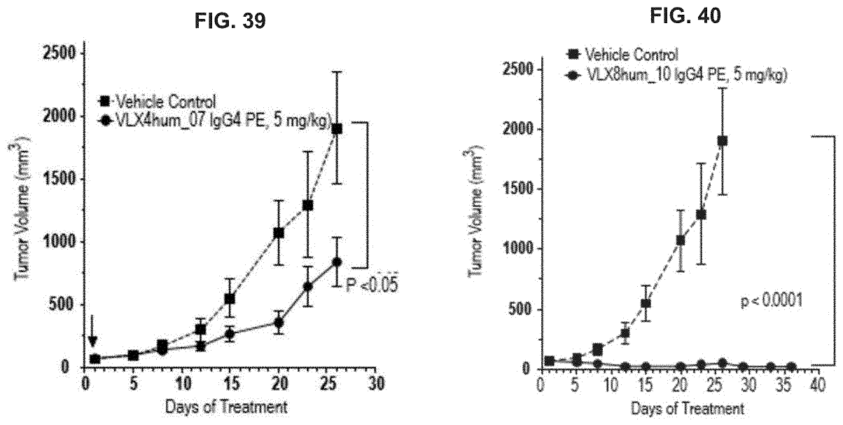

As shown in FIG. 39, treatment with the humanized VLX4hum_07 IgG4PE significantly reduced tumor growth of the Raji tumors (p<0.05, two-way ANOVA), demonstrating anti-tumor efficacy in vivo.

As shown in FIG. 40, treatment with the humanized anti-CD47 mAb, VLX8hum_10 IgG4PE significantly reduced (p<0.0001, two-way ANOVA) tumor growth of the Raji tumors, demonstrating anti-tumor efficacy in vivo.

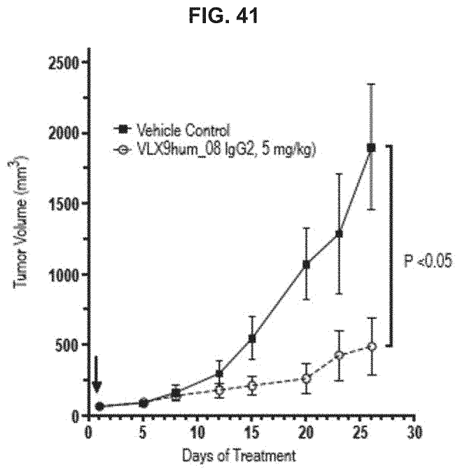

As shown in FIG. 41, treatment with the humanized anti-CD47 mAb, VLX9hum_08 IgG2 significantly reduced (p<0.05, two-way ANOVA) tumor growth of the Raji tumors, demonstrating anti-tumor efficacy in vivo.

### Example 15

### Effect on Circulating Red Blood Cell Parameters

The purpose of this experiment is to demonstrate that VLX9 humanized antibodies that do not bind to human RBC in vitro (Table 2), exemplified by hum1017_08 IgG2, do not cause a reduction in either hemoglobin (Hg) or circulating RBCs following administration to cynomolgus monkeys.

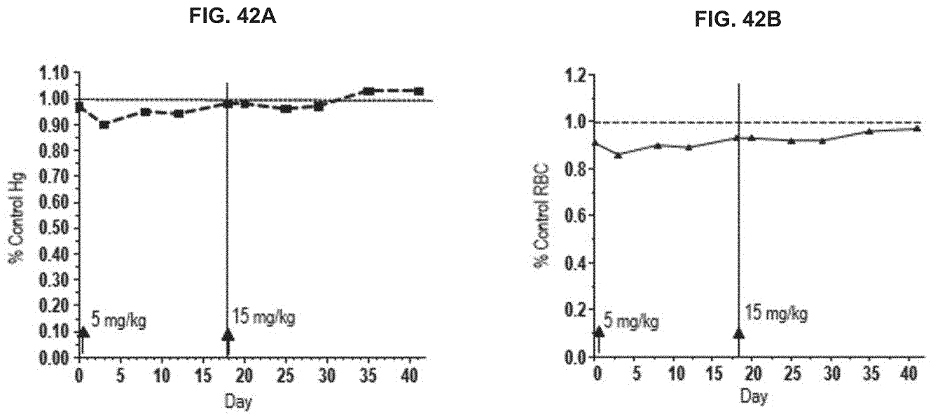

Female Chinese cynomolgus monkeys (Charles River Laboratories, Houston, Tex.) 2.5-3 kg were used in accordance with the Institutional Animal Care and Use guidelines. VLX9hum_08 IgG2 or vehicle (PBS) was administered as a 1 hour intravenous infusion on day 1 at a dose of 5 mg/kg and on day 18 at a dose of 15 mg/kg (3 animals/group). Hematological parameters were measured throughout the study on days −7, −3 (not shown), pre-dose, 3, 8, 12, 18 (pre-dose), 20, 25, 29, 35 and 41 and compared/normalized to the means values of control animals. The pre-treatment RBC and Hg values on day 0 in the VLX9hum_08 IgG2 group were lower than the control group. Following treatment with either dose of VLX9hum_08 IgG2, there were minimal changes (<10%) in Hg (FIG. 42A) or RBC counts (FIG. 42B) compared to the control group demonstrating that VLX9hum_08 IgG2 causes minimal reductions in RBC hematological parameters when administered to cynomolgus monkeys.

### Example 16

### Antibodies to CD47 Regulate Nitric Oxide Signaling

TSP1 binding to CD47 activates the heterotrimeric G protein Gi, which leads to suppression of intracellular cyclic AMP (cAMP) levels. In addition, the TSP1/CD47 pathway opposes the beneficial effects of the nitric oxide (NO) pathway in all vascular cells. The NO pathway consists of any of three nitric oxide synthase enzymes (NOS I, NOS II and NOS III) that generate bioactive gas NO using arginine as a substrate. NO can act within the cell in which it is produced or in neighboring cells, to activate the enzyme soluble guanylyl cyclase that produces the messenger molecule cyclic GMP (cGMP). The proper functioning of the NO/cGMP pathway is essential for protecting the cardiovascular system against stresses including, but not limited to, those resulting from wounding, inflammation, hypertension, metabolic syndrome, ischemia, and ischemia-reperfusion injury (IRI). In the context of these cellular stresses, the inhibition of the NO/cGMP pathway by the TSP1/CD47 system exacerbates the effects of stress. This is a particular problem in the cardiovascular system where both cGMP and cAMP play important protective roles. There are many cases in which ischemia and reperfusion injury cause or contribute to disease, trauma, and poor outcomes of surgical procedures.

The purpose of these experiment will be to demonstrate that humanized anti-CD47 mAbs of the present disclosure exhibit the ability to reverse TSP1-mediated inhibition of NO-stimulated cGMP synthesis as, for example, described previously using mouse monoclonal antibodies to CD47 as disclosed by Isenberg et al. (2006) J. Biol. Chem. 281:26069-80, or alternatively other downstream markers of or effects resulting from NO signaling, for example smooth muscle cell relaxation or platelet aggregation as described previously by Miller et al. (2010) Br J. Pharmacol. 159: 1542-1547.

The method employed that will be to measure cGMP as described by the manufacturer (CatchPoint Cyclic-GMP Fluorescent Assay Kit, Molecular Devices, Sunnyvale, Calif.). Jurkat JE6.1 cells (ATCC, Manassas, Va.; Catalog #TIB-152) or other cells types that retain the NO/cGMP signaling pathway when grown in culture and exhibit a robust and reproducible inhibitory response to TSP1 ligation of CD47 will be used. Cells will be grown in Iscove's modified Dulbeccco's medium containing 5% (v/v) heat inactivated fetal bovine serum (BioWest; Catalogue #S01520), 100 units/mL penicillin, 100 μg mL streptomycin (Sigma; Catalogue #P4222) at densities less than 1×106 cells/mL. For the cGMP assay, cells will be plated in 96 well tissue culture plates at a density of 1×10⁵ cells/ml in Iscoves modified Dulbecco's medium containing 5% (v/v) heat inactivated fetal bovine serum (BioWest; Catalog #S01520), 100 units/mL penicillin, 100 μg/mL streptomycin (Sigma; #P4222) for 24 hours and then transferred to serum free medium overnight.

The humanized antibodies as disclosed herein, purified from transient transfections in CHO cells as described above in Example 3, as well as the control chimeric antibody, will then be added at a final concentration of 20 ng/ml, followed 15 minutes later by 0 or 1 μg/ml human TSP1 (Athens Research and Technology, Athens, Ga., Catalogue #16-20-201319). After an additional 15 minutes, the NO donor, diethylamine (DEA) NONOate (Cayman Chemical, Ann Arbor, Mich., Catalog #82100), will be added to half the wells at a final concentration of 1 μM. Five minutes later, the cells will be lysed with buffer supplied in the cGMP kit, and aliquots of each well assayed for cGMP content.

It is anticipated that some of the chimeric or humanized antibodies will reverse TSP1 inhibition of cGMP. Reversal will be complete (>80%) or intermediate (20%-80%). This reversal of TSP1 inhibition of cGMP will demonstrate that they have the ability to increase NO signaling and suggest utility in protecting the cardiovascular system against stresses including, but not limited to, those resulting from wounding, inflammation, hypertension, metabolic syndrome, ischemia, and ischemia-reperfusion injury (IRI). Additional assay systems, for example smooth muscle cell contraction, will also be expected to show that some of the chimeric or humanized antibody clones reverse the inhibitory actions of TSP on downstream effects resulting from the activation of NO signaling.

### Example 17

### Induction of Cell Death and DAMP Expression by Soluble CD47 Antibodies

Some soluble CD47 antibodies have been shown to induce selective cell death of tumor cells. This additional property of selective toxicity to cancer cells is expected to have advantages compared to mAbs that only block SIRPα binding to CD47.

Induction of cell death by soluble anti-CD47 mAbs is measured in vitro (Manna et al. J. Immunol. 170: 3544-3553, 2003; Manna et al. Cancer Research, 64: 1026-1036, 2004). For the in vitro cell death assay, 1×10⁵ transformed human ovary cells (OV90 cells, ATCC, Manassas, Va.; Catalog #CRL-11732) were incubated with the soluble humanized CD47 mAbs, VLX4hum_07 IgG4 PE (0.03-3 μg/ml), VLX9hum_06 IgG2 CD47 (1-100 μg/ml), and VLX8hum_11 IgG4 PE (0.03-3 μg/ml) for 24 hrs at 37° C. As cell death occurs, mitochondrial membrane potential is decreased, the inner leaflet of the cell membrane is inverted, exposing phosphatidylserines (PS) and calreticulin on the cell surface, and propidium iodide (PI) or 7-aminoactinomycin D (7-AAD) is able to incorporate into nuclear DNA. In order to detect these cellular changes, cells were then stained with fluorescently labeled annexin V and PI or 7-aminoactinomycin D (7-AAD) (BD Biosciences), and a rabbit monoclonal antibody against calreticulin conjugated to Alexa Flour 647 (Abcam; Catalogue #ab196159) and the signal detected using an Attune flow cytometer (Life Technologies). The increase in PS exposure is determined by measuring the percent increase in annexin V signal and the percent of dead cells by measuring the percent increase in PI or 7-AAD signal. Annexin V positive (annexin V⁺) or annexin V positive/7-AAD negative (annexin V⁺/7-AAD⁻) cells are observed in early stages of cell death and annexin V positive/7-AAD positive (annexin V⁺/7-AAD⁺) cells are dead cells. Calreticulin (CRT) exposure is determined by measuring the percent increase in calreticulin positive cells that have not incorporated PI or 7-AAD (calreticulin⁺/7-AAD⁻). Importantly for therapeutic purposes, these mAbs induce cell death of tumor cells directly and do not require complement or the intervention of other cells (e.g., NK cells, T cells, or macrophages) to kill. Thus, the mechanism is independent of both other cells and of Fc effector function. Therefore, therapeutic antibodies developed from these mAbs can be engineered to reduce Fc effector functions such as ADCC and CDC and thereby limit the potential for side effects common to humanized mAbs with intact Fc effector functions.

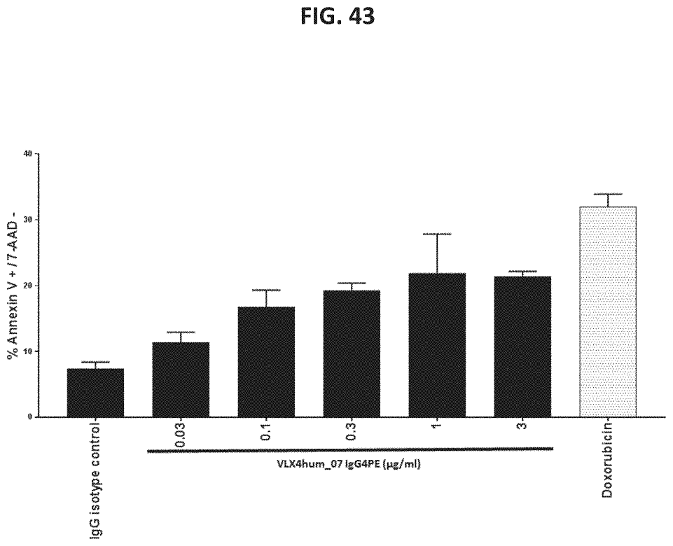

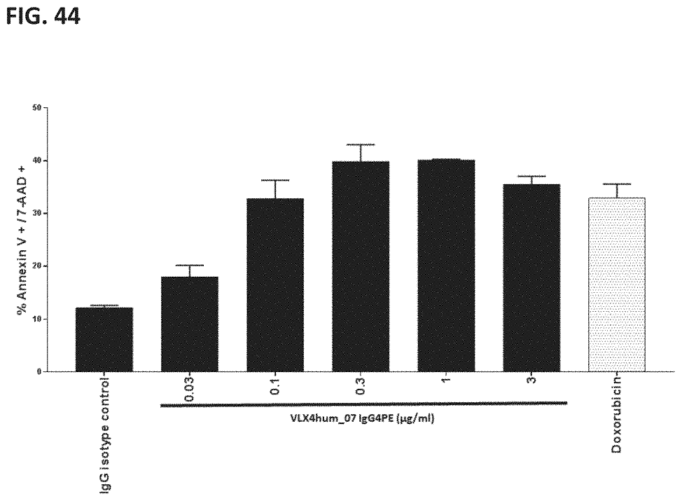

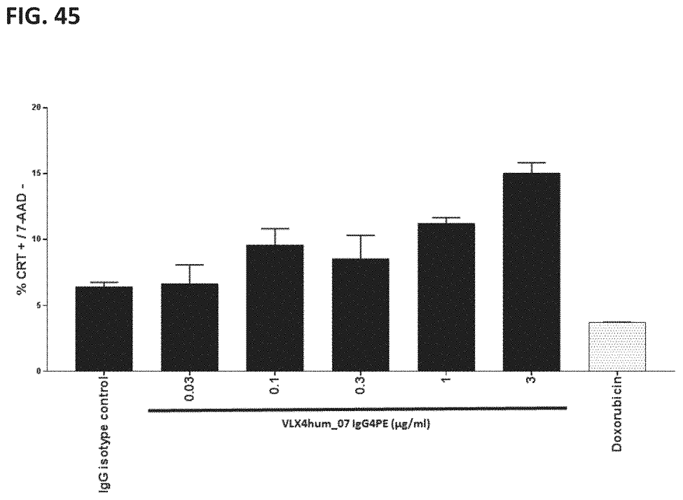

As shown in FIGS. 43-45, the soluble VLX4hum_07 IgG4 PE humanized mAbs induced increased PS exposure and cell death of OV90 cells as measured by increased % of the cells that are annexin V⁺/7-AAD⁻ (FIG. 43) and annexin V⁺/7-AAD⁺ (FIG. 44). The percent of cells that are CRT+/7-AAD− (FIG. 45) in anti-CD47 antibody treated cultures was significantly increased (p<0.05 or greater) compared to isotype control.

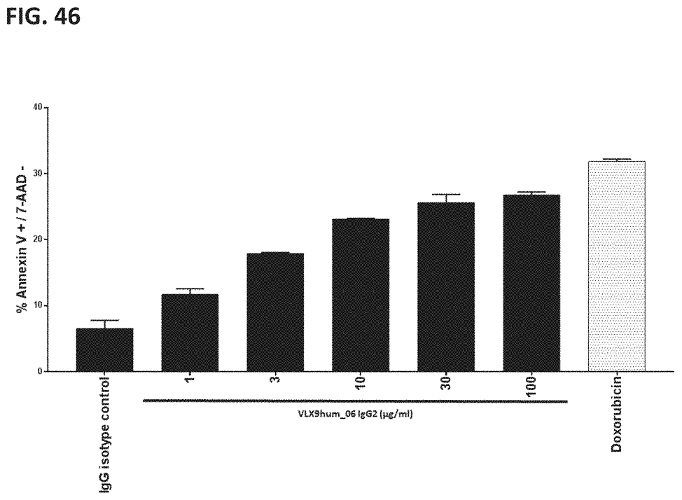

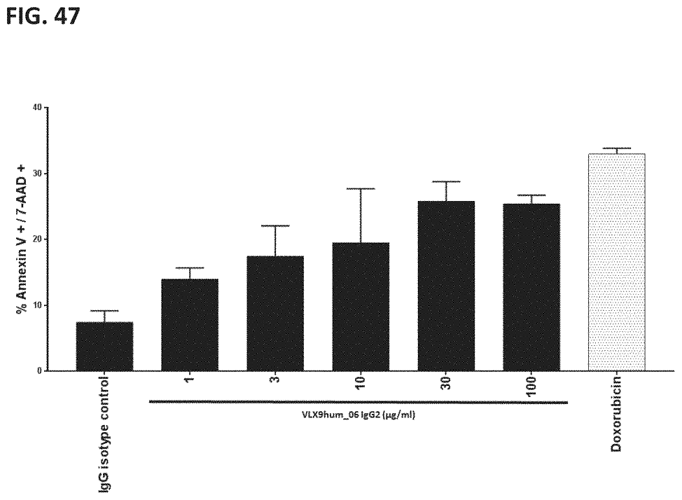

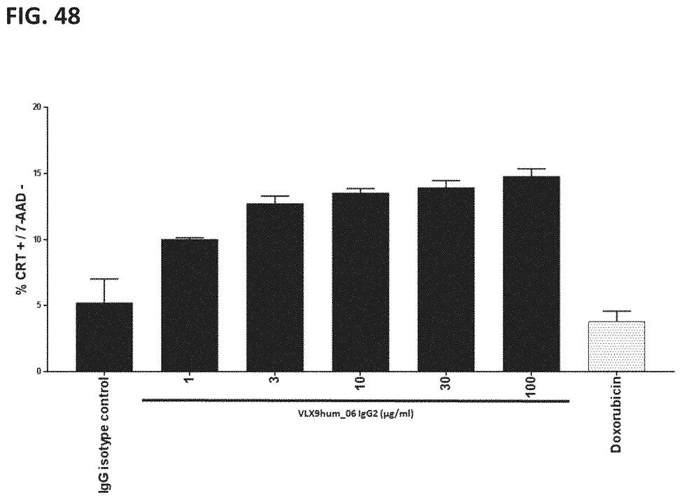

As shown in FIGS. 46-48, the soluble VLX9hum_06 IgG2 humanized mAbs induced increased PS exposure and cell death of OV90 cells as measured by increased % of the cells that are annexin V+/7-AAD− (FIG. 46) and annexin V+/7-AAD+ (FIG. 47). The percent of cells that are CRT+/7-AAD− (FIG. 48) in anti-CD47 antibody treated cultures was significantly increased (p<0.05 or greater) compared to isotype control.

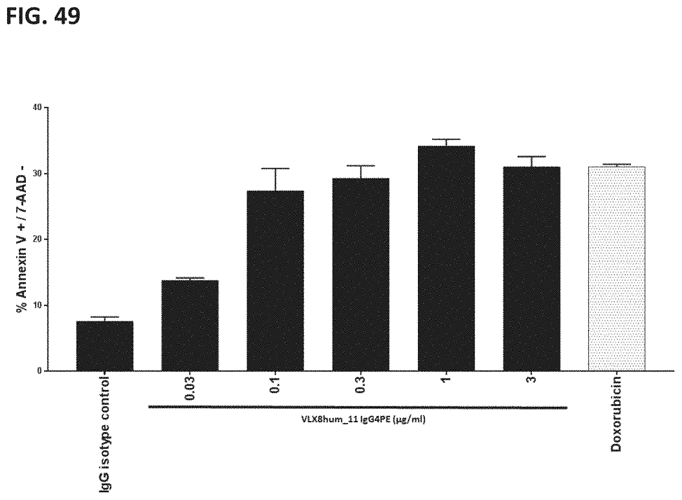

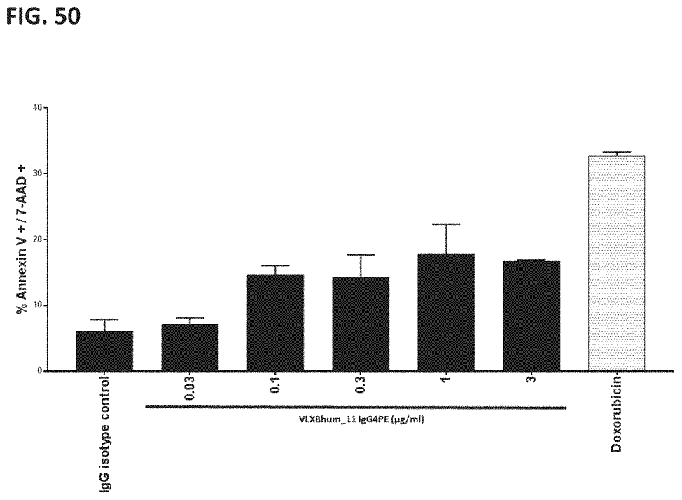

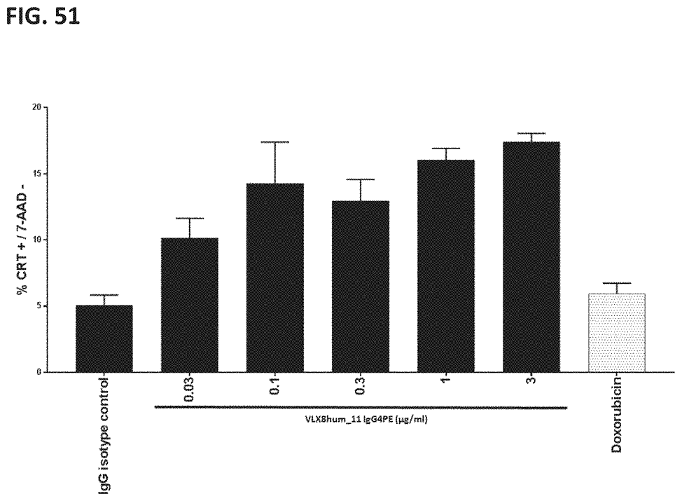

As shown in FIGS. 49-51, the soluble VLX8hum_11 IgG4 PE humanized mAbs induced increased PS exposure and cell death of OV90 cells as measured by increased % of the cells that are annexin V+/7-AAD− (FIG. 49) and annexin V+/7-AAD+ (FIG. 50). The percent of cells that are CRT+/7-AAD− (FIG. 51) in anti-CD47 antibody treated cultures was significantly increased (p<0.05 or greater) compared to isotype control.

Induction of cell death, DAMP expression, and the promotion of phagocytosis of susceptible cancer cells imparts an additional desirable antibody property and therapeutic benefit in the treatment of cancer. This increase in the exposure of calreticulin on the cell surface demonstrate that VLX4hum_07 IgG4 PE, VLX9hum_06 IgG2, and VLX8hum_11 IgG4 PE humanized CD47 mAbs induce DAMPs from tumor cells suggesting further utility in stimulating the phagocytosis of tumor cells and processing of tumor antigen by innate immune cells.

### Example 18

### Combination Treatment of a Humanized Anti-CD47 mAb (VLX4hum_07 IgG4 PE) and Chemotherapy Results in Additive or Synergistic Effects

These experiments demonstrate that humanized anti-CD47 mAbs of the present disclosure cause additive or synergistic activities when combined with clinically relevant chemotherapeutic agents to induce immunogenic cell death effects in human tumor cells.

Combination drug additivity/synergism was determined by combining increasing concentrations of humanized anti-CD47 mAb VLX4hum_07 IgG4 PE with doxorubicin (Sigma, PHR1789), epirubicin (Sigma, E9406), docetaxel (Sigma, 01885), gemcitabine (Sigma, 1288463), irinotecan (Sigma, I1406), oxaliplatin (Sigma, PHR1528). Human OV10/315 cells (Gao and Lindberg, Journal of Biological Chemistry, 1996) were used. Cells were grown in RPMI-1640 medium containing 10% (v/v) heat inactivated fetal bovine serum (BioWest; Catalogue #S01520), 100 units/mL penicillin, 100 μg mL streptomycin (Sigma; Catalogue #P4222) at densities less than 1×10⁶ cells/mL. For this assay, cells were plated in 96 well tissue culture plates at a density of 1×10⁵ cells/ml RPMI-1640 medium containing 10% (v/v) heat inactivated fetal bovine serum (BioWest; Catalog #S01520), 100 units/mL penicillin, 100 μg/mL streptomycin (Sigma; #P4222).

OV10/315 cells were incubated with 0.03-1 μg/ml of VLX4hum_07 IgG4 PE alone, 0.05-0.42 μM of doxorubicin alone or a combination dose-response matrix of 0.03-1 μg/ml of VLX4hum_07 IgG4 PE and 0.05-0.42 μM of doxorubicin in RPMI media at 37° C. for 24 hours, after which the cells were harvested and analyzed for phosphatidylserine using annexin V, and DNA exposure by 7-AAD. Results are presented as means±SEM.

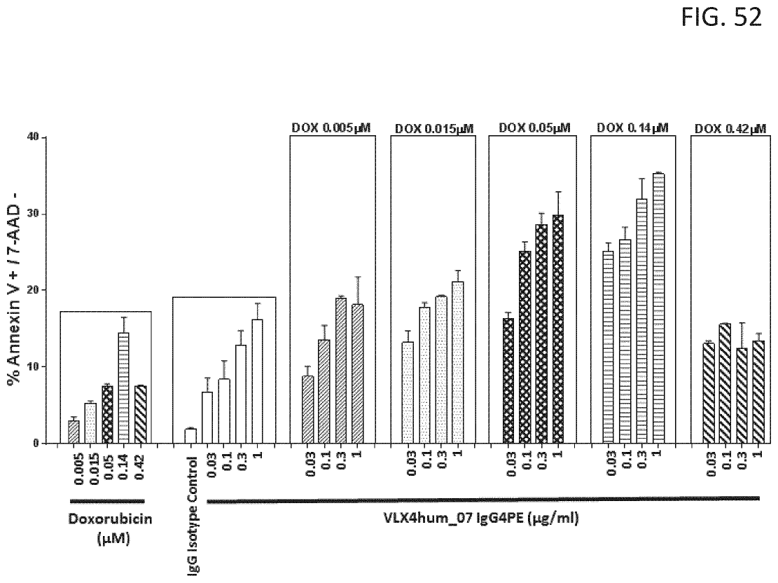

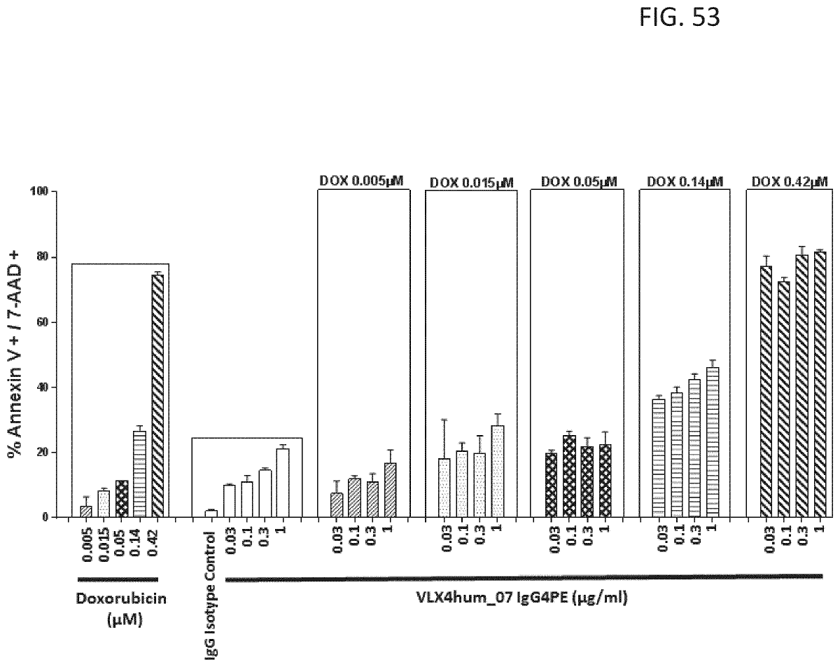

As shown in FIG. 52, some combinations of VLX4hum_07 IgG4PE and doxorubicin cause additive or synergistic effects on the percent of annexin V positive/7-AAD negative (annexin V+/7-AAD−) cells. As shown in FIG. 53, some combinations of VLX4hum_07 IgG4PE and doxorubicin cause additive or synergistic effects on the percent of annexin V positive/7-AAD positive (annexin V+7-AAD+) dead cells.

OV10/315 cells were incubated with 0.03-1 μg/ml of VLX4hum_07 IgG4 PE alone, 0.05-0.42 μM of epirubicin alone or a combination dose-response matrix of 0.03-1 μg/ml of VLX4hum_07 IgG4 PE and 0.05-0.42 μM of epirubicin in RPMI media at 37° C. for 24 hours, after which the cells were harvested and analyzed for phosphatidylserine using annexin V, and DNA exposure by 7-AAD. Results are presented as means±SEM.

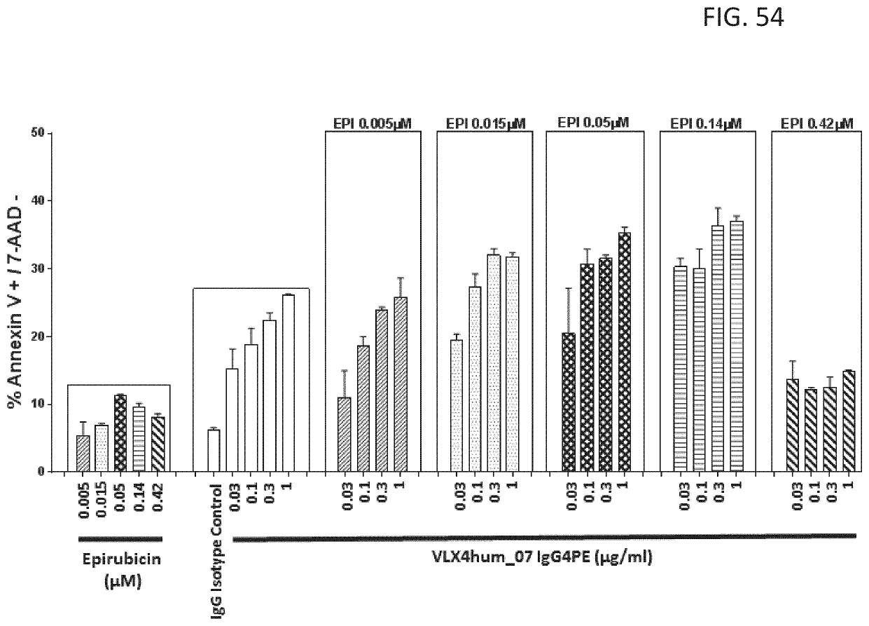

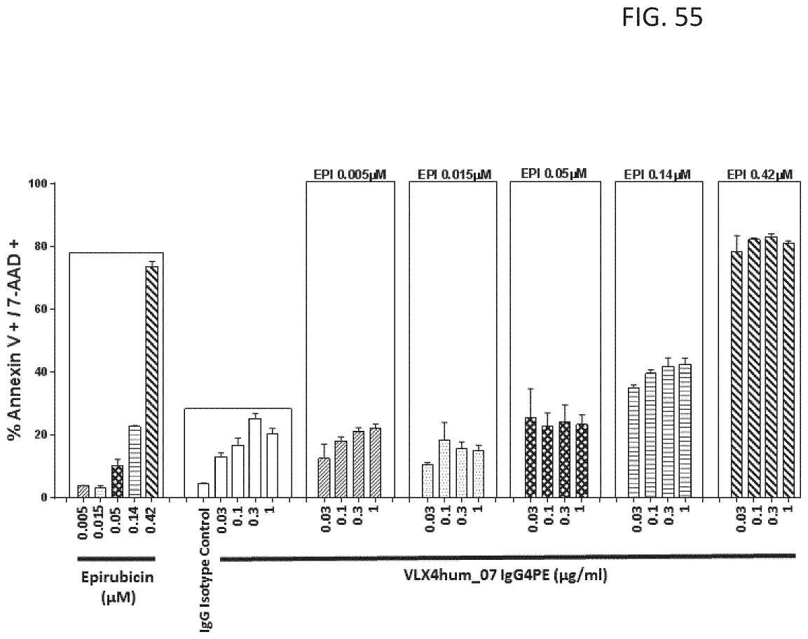

As shown in FIG. 54, some combinations of VLX4hum_07 IgG4PE and epirubicin cause additive or synergistic effects on the percent of annexin V positive/7-AAD negative (annexin V+/7-AAD−) cells. As shown in FIG. 55, some combinations of VLX4hum_07 IgG4PE and epirubicin cause additive or synergistic effects on the percent of annexin V positive/7-AAD positive (annexin V+7-AAD+) dead cells.

OV10/315 cells were incubated with 0.03-1 μg/ml of VLX4hum_07 IgG4 PE alone, 0.002-0.135 μM of docetaxel alone or a combination dose-response matrix of 0.03-1 μg/ml of VLX4hum_07 IgG4 PE and 0.002-0.135 μM of docetaxel in RPMI media at 37° C. for 24 hours, after which the cells were harvested and analyzed for phosphatidylserine using annexin V, and DNA exposure by 7-AAD. Results are presented as means±SEM.

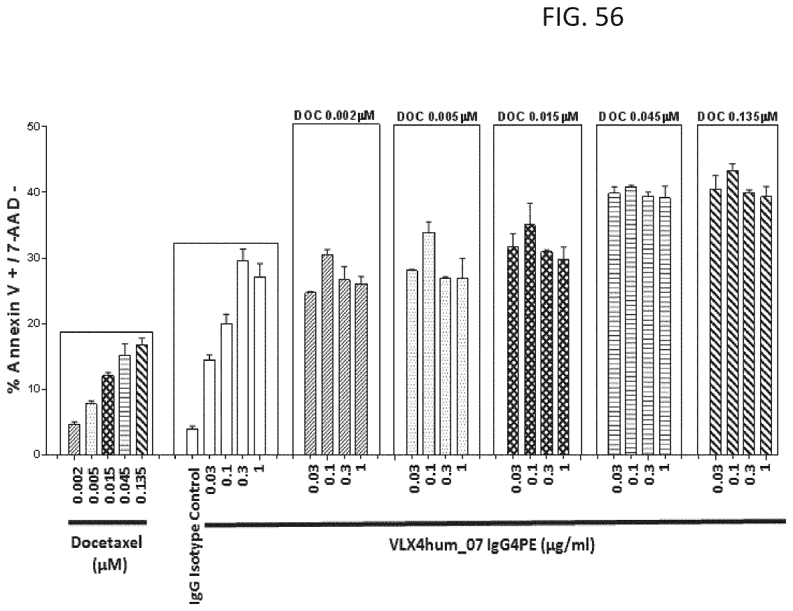

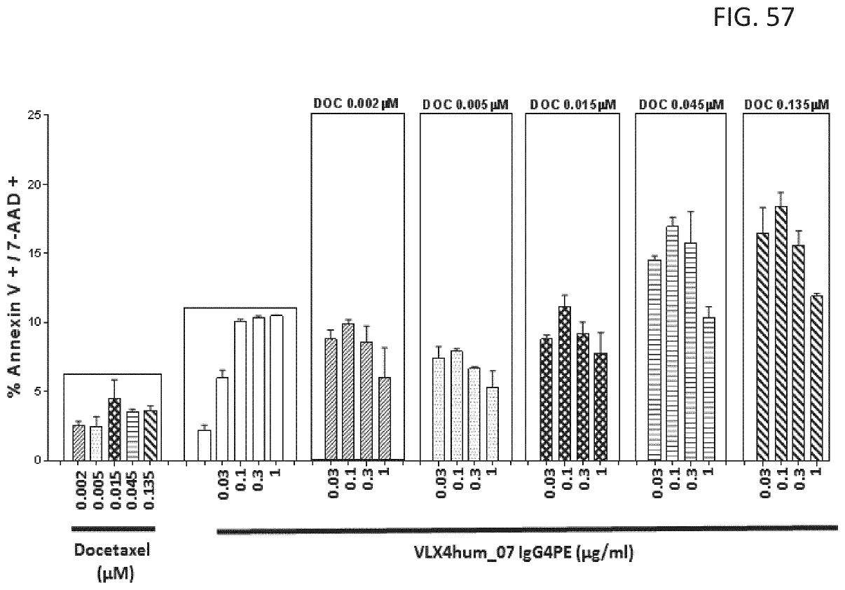

As shown in FIG. 56, some combinations of VLX4hum_07 IgG4PE and docetaxel cause additive or synergistic effects on the percent of annexin V positive/7-AAD negative (annexin V+/7-AAD−) cells. As shown in FIG. 57, some combinations of VLX4hum_07 IgG4PE and docetaxel cause additive or synergistic effects on the percent of annexin V positive/7-AAD positive (annexin V+7-AAD+) dead cells.

OV10/315 cells were incubated with 0.03-1 μg/ml of VLX4hum_07 IgG4 PE alone, 0.003-0.3 μM of gemcitabine alone or a combination dose-response matrix of 0.03-1 μg/ml of VLX4hum_07 IgG4 PE and 0.003-0.3 μM of gemcitabine in RPMI media at 37° C. for 24 hours, after which the cells were harvested and analyzed for phosphatidylserine using annexin V, 7-AAD, ER stress marker calreticulin on the cell surface. Results are presented as means±SEM.

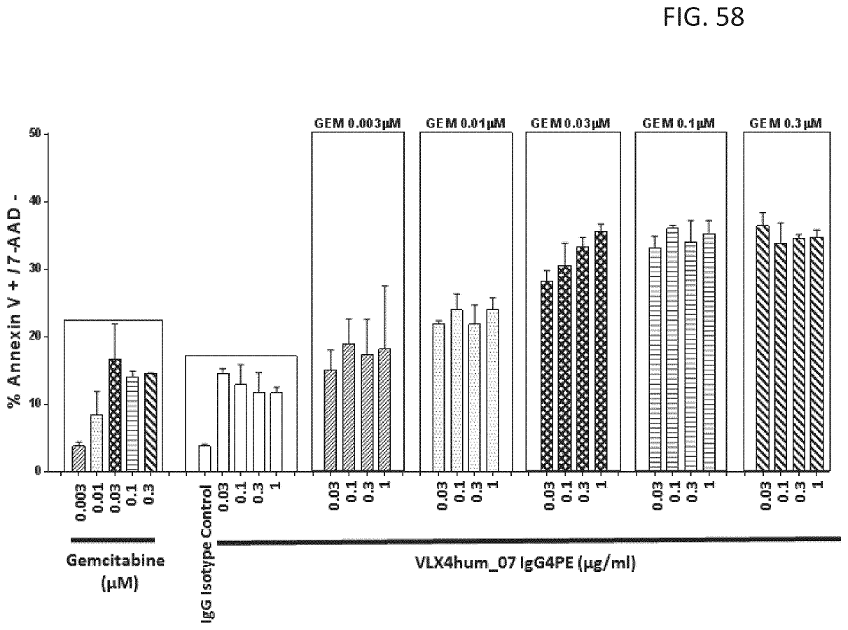

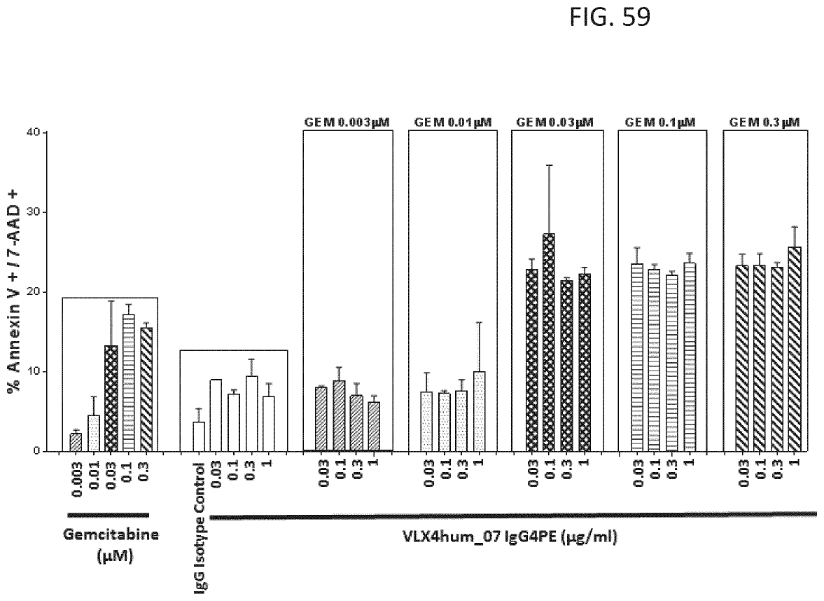

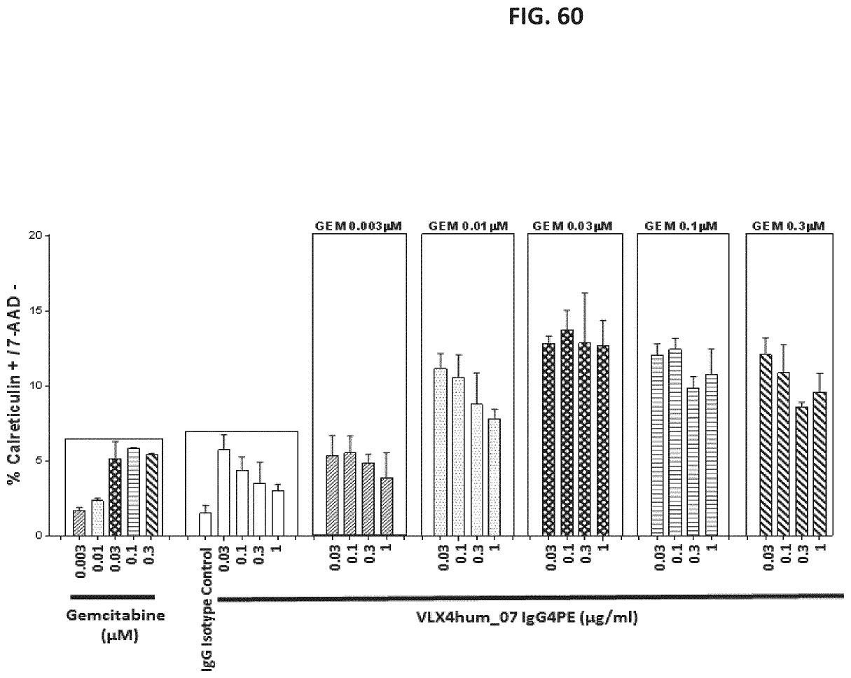

As shown in FIG. 58, some combinations of VLX4hum_07 IgG4PE and gemcitabine cause additive or synergistic effects on the percent of annexin V positive/7-AAD negative (annexin V+/7-AAD−) cells. As shown in FIG. 59, some combinations of VLX4hum_07 IgG4PE and gemcitabine cause additive or synergistic effects on the percent of annexin V positive/7-AAD positive (annexin V+7-AAD+) dead cells. As shown in FIG. 60, some combinations of VLX4hum_07 IgG4PE and gemcitabine cause additive or synergistic effects on the percent of calreticulin positive cells.

OV10/315 cells were incubated with 0.03-1 μg/ml of VLX4hum_07 IgG4 PE alone, 0.63-51 nM of irinotecan alone or a combination dose-response matrix of 0.03-1 μg/ml of VLX4hum_07 IgG4 PE and 0.63-51 nM of irinotecan in RPMI media at 37° C. for 24 hours, after which the cells were harvested and analyzed for phosphatidylserine using annexin V, 7-AAD, ER stress marker calreticulin on the cell surface. Results are presented as means±SEM.

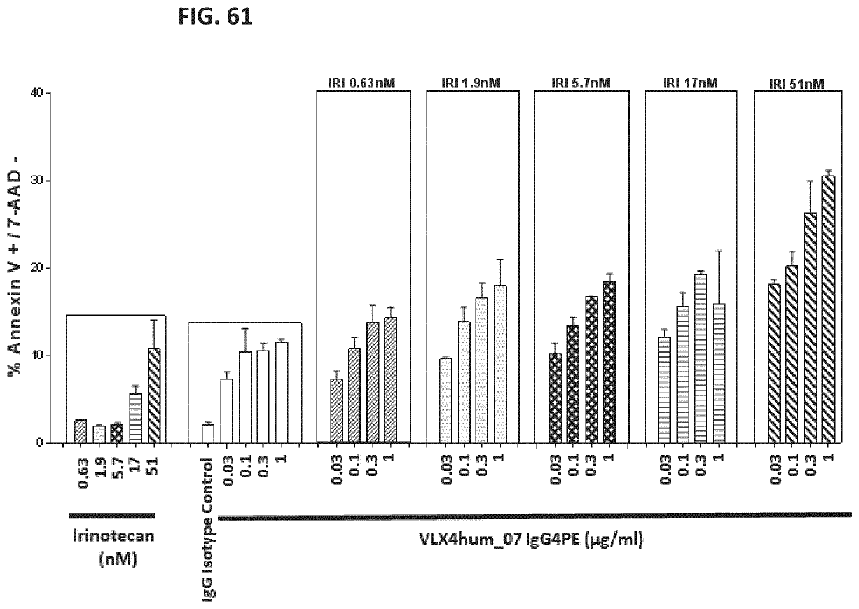

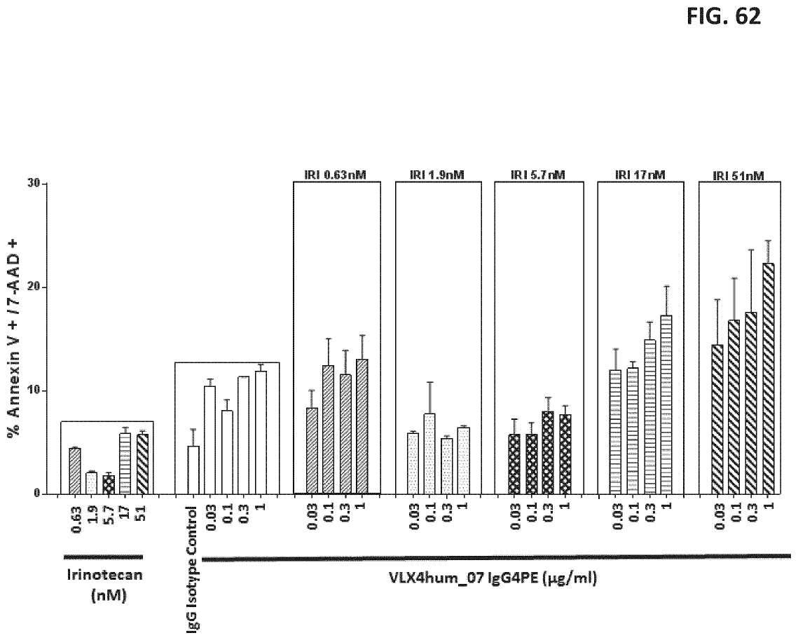

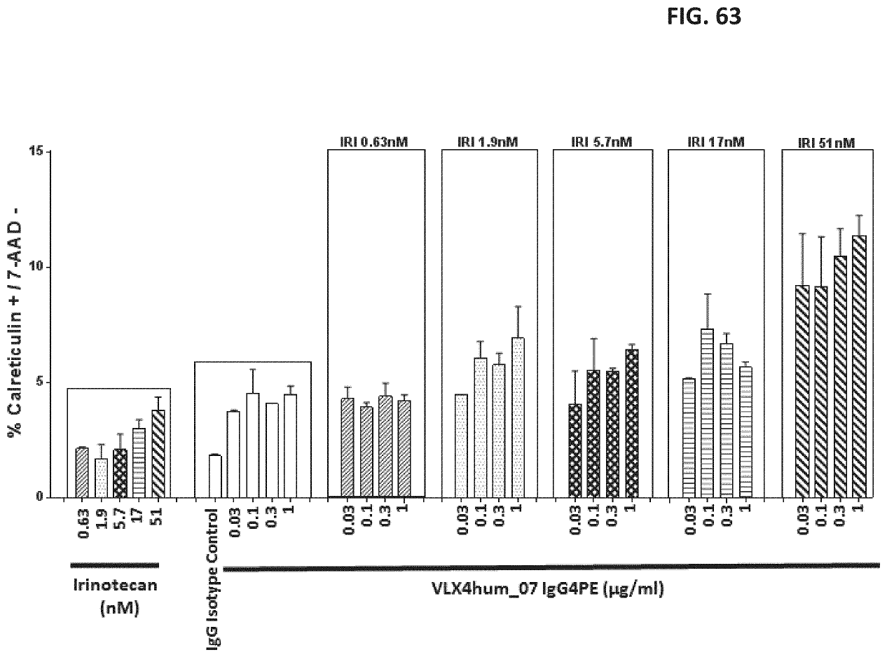

As shown in FIG. 61, some combinations of VLX4hum_07 IgG4PE and irinotecan cause additive or synergistic effects on the percent of annexin V positive/7-AAD negative (annexin V+/7-AAD−) cells. As shown in FIG. 62, some combinations of VLX4hum_07 IgG4PE and irinotecan cause additive or synergistic effects on the percent of annexin V positive/7-AAD positive (annexin V+7-AAD+) dead cells. As shown in FIG. 63, some combinations of VLX4hum_07 IgG4PE and irinotecan cause additive or synergistic effects on the percent of calreticulin positive cells.

OV10/315 cells were incubated with 0.03-1 μg/ml of VLX4hum_07 IgG4 PE alone, 0.65-52.8 μM of oxaliplatin alone or a combination dose-response matrix of 0.03-1 μg/ml of VLX4hum_07 IgG4 PE and 0.65-52.8 μM of oxaliplatin in RPMI media at 37° C. for 24 hours, after which the cells were harvested and analyzed for phosphatidylserine using annexin V, and DNA exposure by 7-AAD. Results are presented as means±SEM.

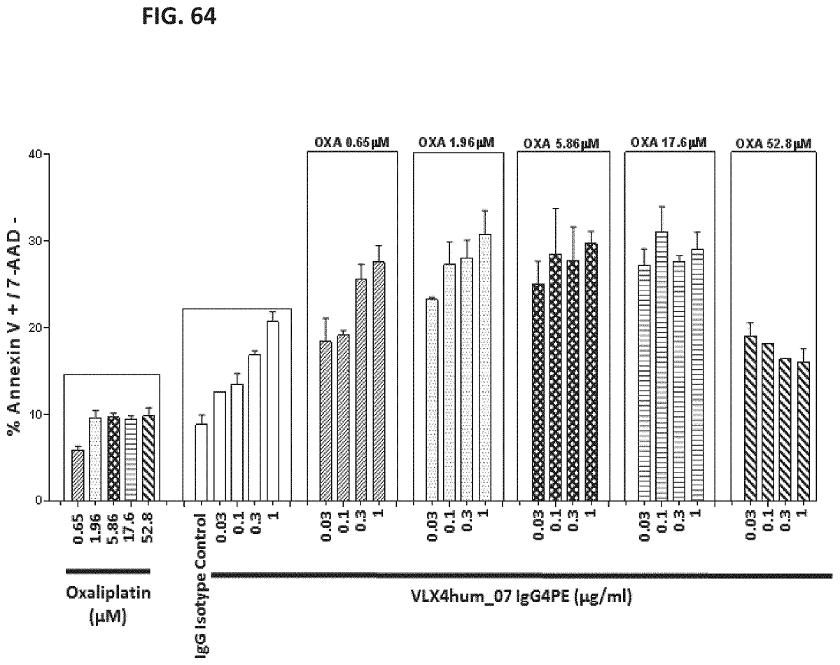

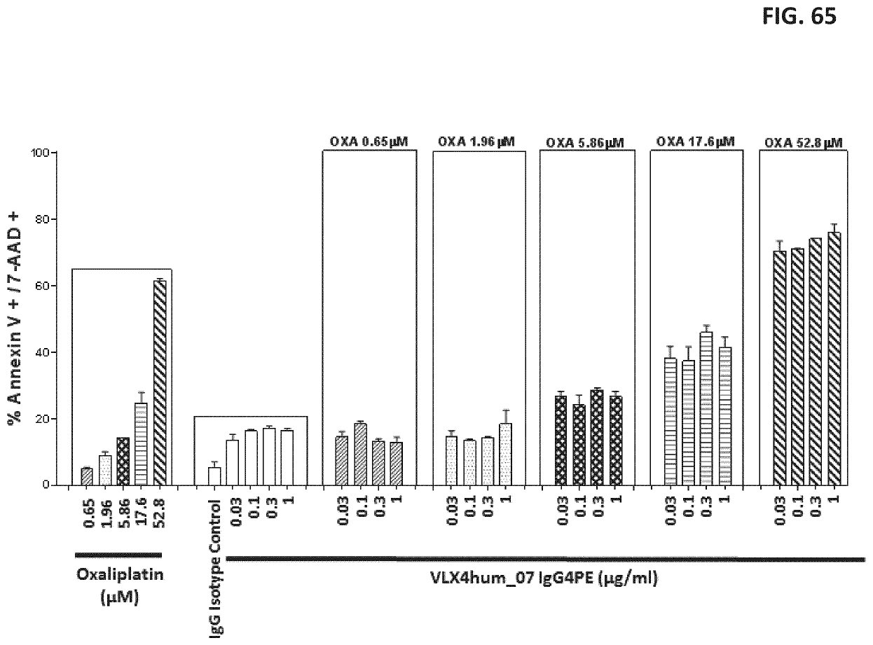

As shown in FIG. 64, some combinations of VLX4hum_07 IgG4PE and oxaliplatin cause additive or synergistic effects on the percent of annexin V positive/7-AAD negative (annexin V+/7-AAD−) cells. As shown in FIG. 65, some combinations of VLX4hum_07 IgG4PE and oxaliplatin cause additive or synergistic effects on the percent of annexin V positive/7-AAD positive (annexin V+7-AAD+) dead cells.

### Example 19

### Combination Treatment of a Humanized Anti-CD47 mAb (VLX9hum_06 IgG2) and Chemotherapy Results in Additive or Synergistic Effects

These experiments demonstrate that humanized anti-CD47 mAbs of the present disclosure cause additive or synergistic activities when combined with clinically relevant chemotherapeutic agents to induce immunogenic cell death effects in human tumor cells.

Combination drug additivity/synergism was determined by combining increasing concentrations of humanized anti-CD47 mAb VLX9hum_06 IgG2 and doxorubicin (Sigma, PHR1789). Human Jurkat T ALL cell line (ATCC, Manassas, Va.; Catalog #TIB-152) were used. Cells were grown in RPMI-1640 medium containing 10% (v/v) heat inactivated fetal bovine serum (BioWest; Catalogue #S01520), 100 units/mL penicillin, 100 μg mL streptomycin (Sigma; Catalogue #P4222) at densities less than 1×106 cells/mL. For this assay, cells were plated in 96 well tissue culture plates at a density of 1×105 cells/ml RPMI-1640 medium containing 10% (v/v) heat inactivated fetal bovine serum (BioWest; Catalog #S01520), 100 units/mL penicillin, 100 μg/mL streptomycin (Sigma; #P4222).

Jurkat cells were incubated with 1-100 μg/ml of VLX9hum_06 IgG2 alone, 0.005-0.42 μM of doxorubicin alone or a combination dose-response matrix of 1-100 μg/ml of VLX9hum_06 IgG2 and 0.005-0.42 μM of doxorubicin in RPMI media at 37° C. for 24 hours, after which the cells were harvested and analyzed for phosphatidylserine using annexin V, 7-AAD, ER stress marker calreticulin on the cell surface. Results are presented as means±SEM.

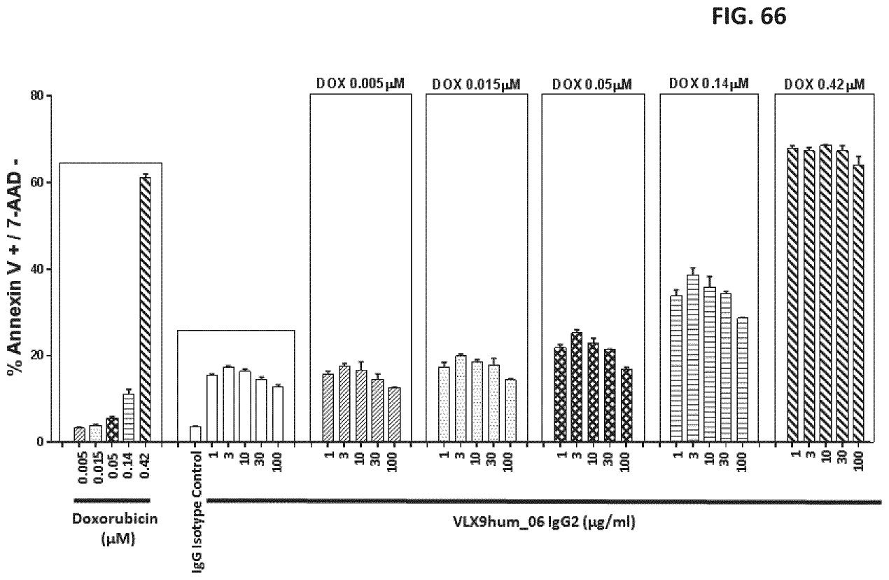

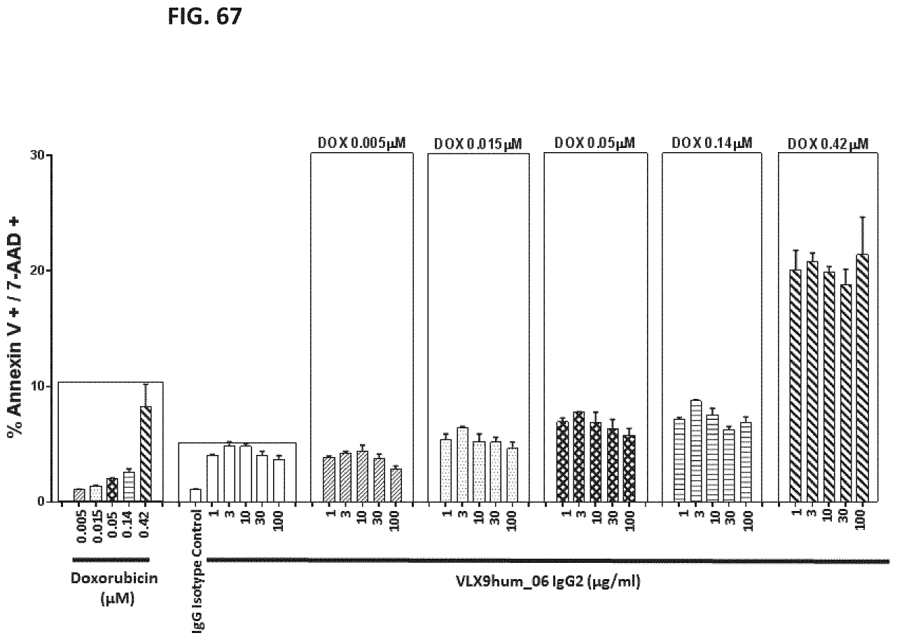

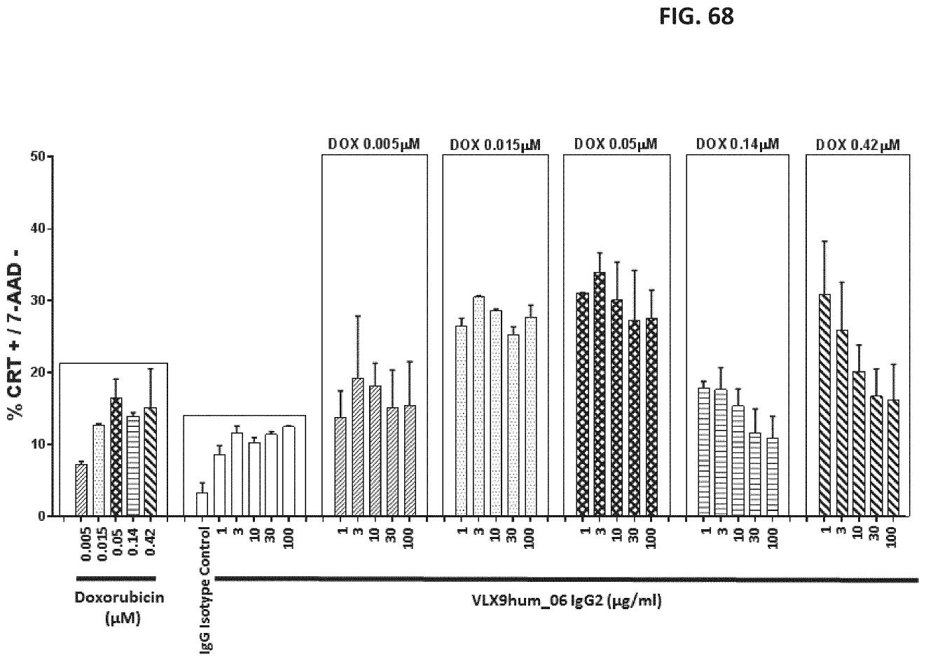

As shown in FIG. 66, some combinations of VLX4hum_07 IgG4PE and doxorubicin cause additive or synergistic effects on the percent of annexin V positive/7-AAD negative (annexin V+/7-AAD−) cells. As shown in FIG. 67, some combinations of VLX4hum_07 IgG4PE and doxorubicin cause additive or synergistic effects on the percent of annexin V positive/7-AAD positive (annexin V+7-AAD+) dead cells. As shown in FIG. 68, some combinations of VLX4hum_07 IgG4PE and doxorubicin cause additive or synergistic effects on the percent of calreticulin positive cells.

### Example 20

### Combination Treatment with Humanized Anti-CD47 mAb (VLX8hum_11 IgG4 PE) and Chemotherapy Results in Additive or Synergistic Effects

These experiments demonstrate that humanized anti-CD47 mAbs of the present disclosure cause additive or synergistic activities when combined with clinically relevant chemotherapeutic agents to induce immunogenic cell death effects in human tumor cells.

Combination drug additivity/synergism was determined by combining increasing concentrations of humanized anti-CD47 mAb VLX8hum_11 IgG4 PE and doxorubicin (Sigma, PHR1789). Human Jurkat T ALL cell line (ATCC, Manassas, Va.; Catalog #TIB-152) were used. Cells were grown in RPMI-1640 medium containing 10% (v/v) heat inactivated fetal bovine serum (BioWest; Catalogue #S01520), 100 units/mL penicillin, 100 μg mL streptomycin (Sigma; Catalogue #P4222) at densities less than 1×106 cells/mL. For this assay, cells were plated in 96 well tissue culture plates at a density of 1×105 cells/ml RPMI-1640 medium containing 10% (v/v) heat inactivated fetal bovine serum (BioWest; Catalog #S01520), 100 units/mL penicillin, 100 μg/mL streptomycin (Sigma; #P4222).

Jurkat cells were incubated with 0.03-3 μg/ml of VLX8hum_11 IgG4 PE alone, 0.005-0.42 μM of doxorubicin alone or a combination dose-response matrix of 0.03-3 μg/ml of VLX8hum_11 IgG4 PE and 0.005-0.42 μM of doxorubicin in RPMI media at 37° C. for 24 hours, after which the cells were harvested and analyzed for phosphatidylserine using annexin V, 7-AAD, ER stress marker calreticulin on the cell surface, and cell supernatant was analyzed for HMGB1 release. Results are presented as means±SEM.

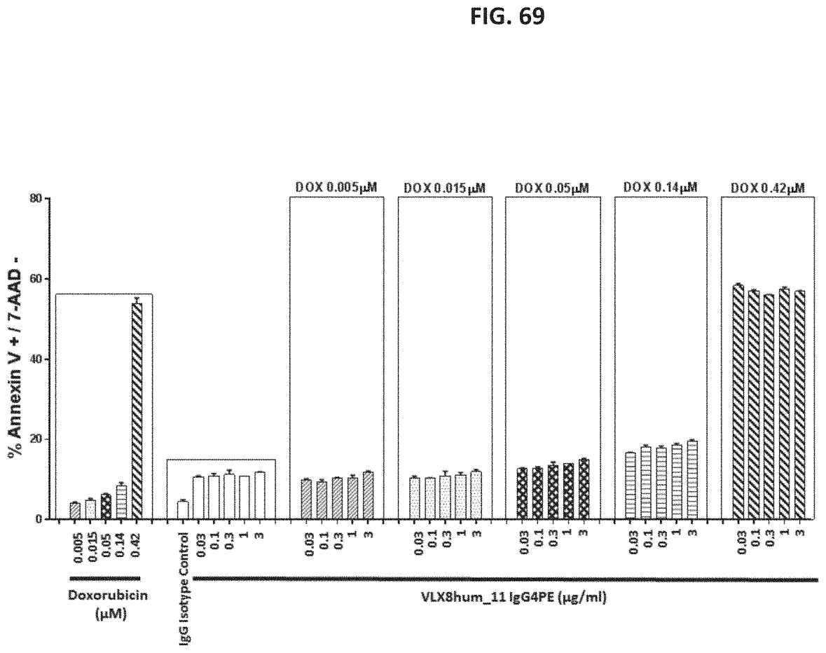

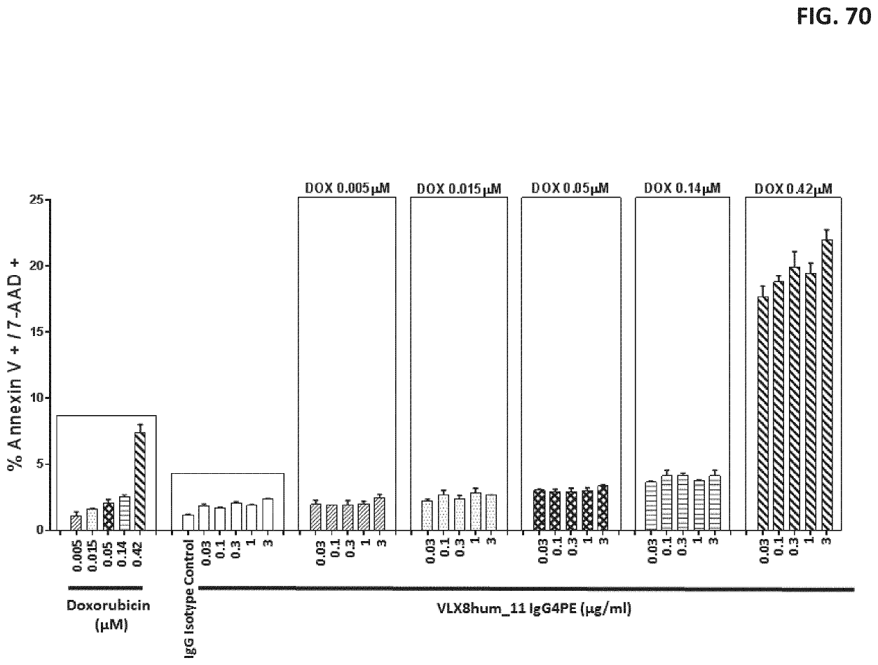

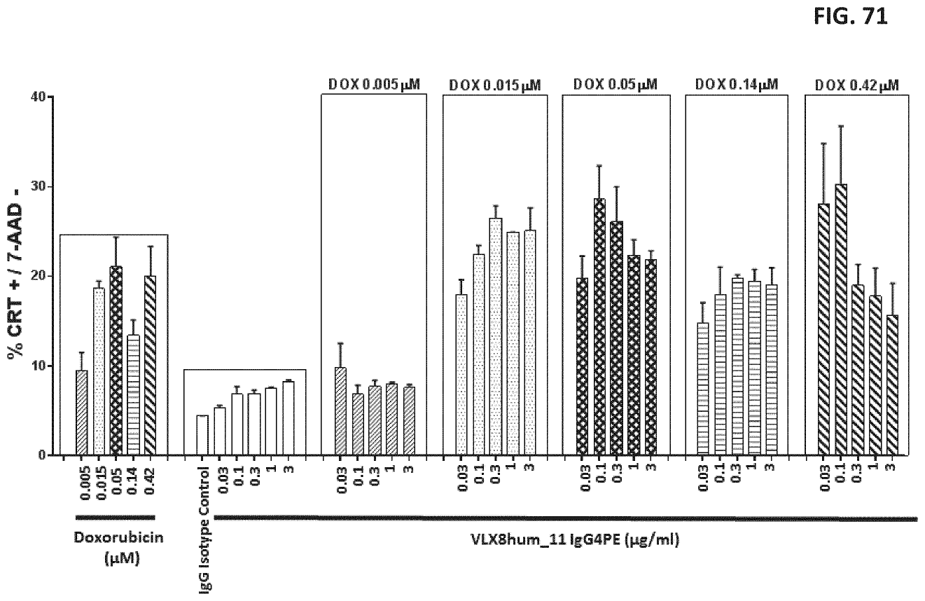

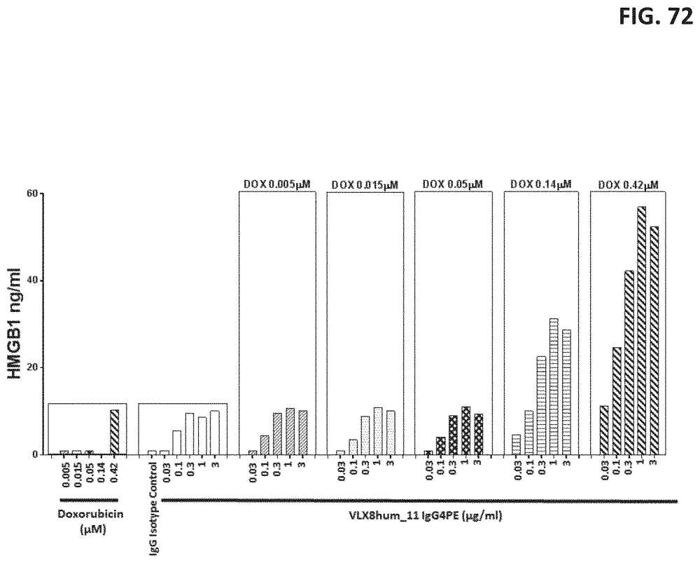

As shown in FIG. 69, some combinations of VLX8hum_11 IgG4 PE and doxorubicin cause additive or synergistic effects on the percent of annexin V positive/7-AAD negative (annexin V+/7-AAD−) cells. As shown in FIG. 70, some combinations of VLX8hum_11 IgG4 PE and doxorubicin cause additive or synergistic effects on the percent of annexin V positive/7-AAD positive (annexin V+7-AAD+) dead cells. As shown in FIG. 71, some combinations of VLX8hum_11 IgG4 PE and doxorubicin cause additive or synergistic effects on the percent of calreticulin positive cells (calreticulin+/7-AAD−). As shown in FIG. 72, some combinations of VLX8hum_11 IgG4 PE and doxorubicin cause additive or synergistic effects on the amount of HMGB1 release.

### Example 21

### pH Dependent and Independent Binding of Humanized Anti-CD47 mAbs

Some soluble anti-CD47 mAbs have been shown to bind tumor cells at acidic pH with greater affinity than at physiologic pH. This additional property is expected to have advantages compared to mAbs that bind at similar affinities to CD47 at both acidic and physiologic pH, due to the acidic nature of the tumor microenvironment (Tannock and Rotin, Cancer Res. 1989; Song et al, Cancer Drug Discovery and Development 2006; Chen and Pagel, Advan. Radiol. 2015). Binding of soluble anti-CD47 mAbs to recombinant Fc-CD47 was measured in vitro by surface plasmon resonance on a Biacore 2000. An Anti-Human IgG (GE Lifesciences) was amine coupled to a CM5 chip on flow cells 1 and 2. The recombinant Fc-CD47 diluted in PBS-EP⁺ was captured onto flow cells 1 and 2. Multi-cycle kinetics were determined using 0 to 1000 nM humanized mAbs VLX4hum_01 Fab, VLX8hum_11 Fab or VLX9hum_08 Fab diluted in HBS-EP⁺ running buffer pH 7.5, 7, 6.5 or 6 with contact time of 180 seconds and dissociation time of 300 seconds. A 1:1 binding model was employed for kinetic analysis of binding curves. The on-rate, off-rate and Dissociation constants for VLX4hum_01 Fab, VLX8hum_11 Fab and VLX9hum_08 Fab are shown in Table 7 and demonstrate that VLX9hum_08 has pH dependent binding to CD47, whereas VLX4hum_01 and VLX8hum_11 do not. This pH dependence imparts an additional desirable antibody property and therapeutic benefit in the treatment of cancer.

TABLE 11
Binding of VLX4 Fab, VLX8 Fab and VLX9 Fab Humanized mAbs
to Recombinant Fc-CD47 by Surface Plasmon Resonance.

| pH | Fab | K_{D} (nM) | kₐ (M⁻¹s⁻¹) | k_{d} (s⁻¹) |
|---|---|---|---|---|
| 7.5 | VLX4hum_01 | 2.0 | 1.1e⁰⁶ | 2.1e⁻⁰³ |
| 7.0 | VLX4hum_01 | 1.3 | 1.7e⁰⁶ | 2.18e⁻⁰³ |
| 6.5 | VLX4hum_01 | 2.7 | 1.1e⁰⁶ | 3.06e⁻⁰³ |
| 6.0 | VLX4hum_01 | 2.0 | 1.3e⁰⁶ | 2.55e⁻⁰³ |
| 7.5 | VLX9hum_08 | 79 | 1.4e⁰⁶ | 1.13e⁻⁰² |
| 7.0 | VLX9hum_08 | 27 | 1.3e⁰⁵ | 3.56e⁻⁰³ |
| 6.5 | VLX9hum_08 | 5.6 | 1.7e⁰⁵ | 9.74e⁻⁰⁴ |
| 6.0 | VLX9hum_08 | 5.1 | 1.9e⁰⁵ | 9.94e⁻⁰⁴ |
| 7.5 | VLX8hum_11 | 1.7 | 1.3e⁰⁶ | 2.29e⁻⁰³ |
| 7.0 | VLX8hum_11 | 1.5 | 1.4e⁰⁶ | 2.17e⁻⁰³ |
| 6.5 | VLX8hum_11 | 1.3 | 2.1e⁰⁶ | 2.78e⁻⁰³ |
| 6.0 | VLX8hum_11 | 1.6 | 1.6e⁰⁶ | 2.63e⁻⁰³ |

### Example 22

### Anti-Tumor Activity In Vivo in Human Xenograft Model

These experiments were performed to show that a humanized anti-CD47 antibody, as exemplified by VLX8hum_10 IgG4PE, reduce tumor burden in vivo in a mouse xenograft model of triple negative breast cancer.

MDA-MB-231 triple negative breast cancer cells (Catalog #HTB-26™ Manassas, Va.)) were maintained in RPMI-1640 (Lonza; Walkersville, Md.) supplemented with 10% Fetal Bovine Serum (FBS; Omega Scientific; Tarzana, Calif.) within a 5% CO₂ atmosphere. Cultures were expanded in tissue culture flasks.

Female NSG (NOD-Cg-Prkdc^{scid}I12rg^{tm1Wjl}/SzJ) were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NSG mice were inoculated orthotopically in the mammary fat pad with 0.2 mL of a 70% RPMI/30% Matrigel™ (BD Biosciences; Bedford, Mass.) mixture containing a suspension of 2×10⁷ MDA-MB-231 tumor cells. Nineteen days following inoculation, fifty mice with palpable tumor volumes of 55-179 mm³ were randomized into five groups of ten mice, by random equilibration. Tumor volumes were calculated utilizing the formula: tumor volume (mm³)=(a×b²/2) where ‘b’ is the smallest diameter and ‘a’ is the largest diameter. VLX9hum_08 or PBS (control) administration was initiated at this time. Mice were treated with 15 mg/kg of antibody 5×/week for 5 weeks by intraperitoneal injection. Tumor volumes and body weights were recorded twice weekly.

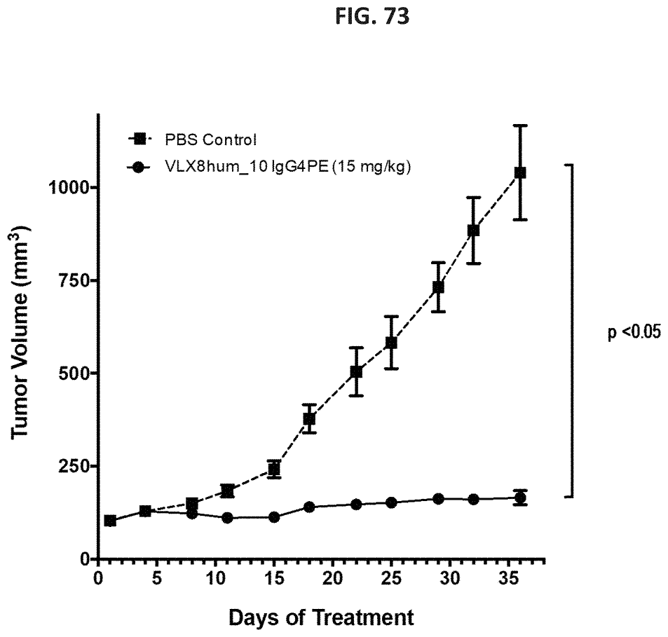

As shown in FIG. 73, treatment with the humanized VLX8hum_10 IgG4 PE significantly reduced tumor growth of the MDA-MB-231 tumors (p<0.05, ANOVA), demonstrating anti-tumor efficacy in vivo.

### Example 23

### Anti-Tumor Activity of VLX9hum_06 IgG2 and Proteasome Inhibitors in a Xenograft Mouse Model (RPMI-8226) of Multiple Myeloma

This disclosure demonstrates the anti-tumor properties of a humanized anti-CD47 antibody (VLX9hum_06 IgG2) as a single agent and in combination with bortezomib which reduce tumor burden in a xenograft multiple myeloma NSG mouse model. RPMI-8226 human multiple myeloma cells (ATCC #CCL-155, Manassas, Va.) were maintained in RPMI-1640 (Lonza; Walkersville, Md.) supplemented with 10% Fetal Bovine Serum (FBS; Omega Scientific; Tarzana, Calif.) and 1% Penicillin/Streptomycin (Corning, Manassas, Va.) within a 5% CO2 atmosphere. Cultures were expanded in tissue culture flasks.

Female NSG (NOD-Cg-Prkdc^{scid}I12rg^{tm1Wjl}/SzJ) were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NSG mice were inoculated subcutaneously in the right flank with 0.1 mL of a 30% RPMI/70% Matrigel™ (BD Biosciences; Bedford, Mass.) mixture containing a suspension of 1×10⁷ RPMI-8226 tumor cells. Fifteen days following inoculation, mice were randomized. The test articles human IgG2 (hIgG2), anti-CD47 mAb, (VLX9hum_06 IgG2), and Bortezomib (LC Labs, Woburn, Mass.) were administered by intravenous (IV) injection. hIgG2 (25 mg/kg) and an anti-CD47 mAb VLX9hum_06 IgG2 (concentrations of 10 mg/kg or 25 mg/kg) were administered on Days 0, 7, 14, 21, 28, and 35, while Bortezomib (1 mg/kg) was administered on Day 1, 4, and 12.

Mean tumor growth inhibition (TGI) was calculated at Day 48 (the final day all mice were on study) utilizing the following formula. Of note, mice exhibiting tumor shrinkage were excluded from the TGI calculations.

$$TGI = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

Individual tumor shrinkage (TS) was calculated at Day 48 using the formula below for tumors that showed regression relative to Day 0. The mean TS of each group was calculated and reported.

$$TS = [ 1 - \frac{( Tumor Volume ( Final ) )}{( Tumor Volume ( Day 0 ) )} ] \times 100 \%$$

Differences in Day 48 tumor volumes were confirmed using a one-way ANOVA, unpaired, parametric with the Tukey's Multiple Comparison test with Welch's correction. A two-tailed Student's t-test with Welch's correction was also used to verify any differences between each group and the vehicle control.

Increase in survival fractions were confirmed by the log rank test with a comparison of each group to the vehicle control group. For statistical analysis purpose, any mouse sacrificed as an long term survivor (LTS) was assigned a death day of Day 99.

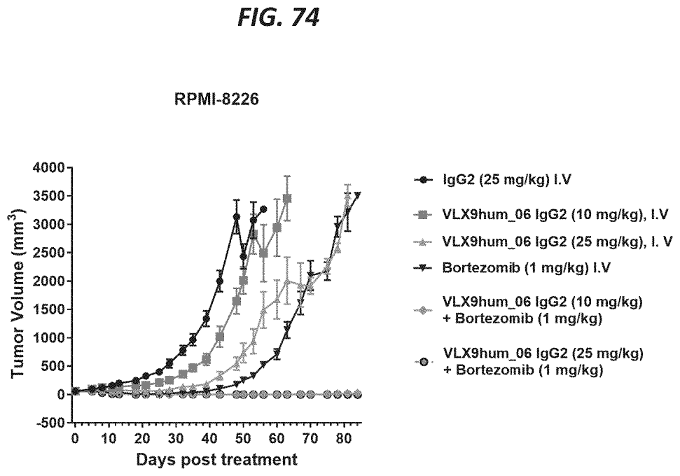

Primary Assessment of Efficacy Based on Tumor Volume. As shown in FIG. 74, primary efficacy assessment based on tumor growth inhibition (TGI) resulted in statistically significant anti-tumor activity in all groups when compared to the hIgG2 vehicle control group with 10 mg/kg VLX9hum_06 IgG2 (48.3% TGI), 25 mg/kg VLX9hum_06 IgG2 (84.2% TGI), or 1 mg/kg bortezomib (96% TGI), demonstrating single agent anti-tumor efficacy in vivo. Combination treatment resulted in statistically significant decreases in tumor volumes when compared to both respective single agent groups (p<0.0001, 2-way ANOVA). Profound anti-tumor efficacy was seen in the all combination groups, which resulted in complete tumor regression of 95% of mice by Day 48 and achieved 100% complete responders by end of the study. No tumor shrinkage was recorded with monotherapy, whereas a mean tumor shrinkage (TS) of 100% and 95.4% was observed at doses of 10 mg/kg anti-CD47 mAb VLX9hum_06 IgG2+Bortezomib or 25 mg/kg anti-CD47 mAb VLX9hum_06 IgG2+Bortezomib, respectively, when compared to the hIgG2 vehicle control, demonstrating synergistic anti-tumor efficacy of combination agents in vivo. By Day 50, 100% of mice in the both combination treatment groups were tumor free resulting 100% TS as shown in FIG. 74.

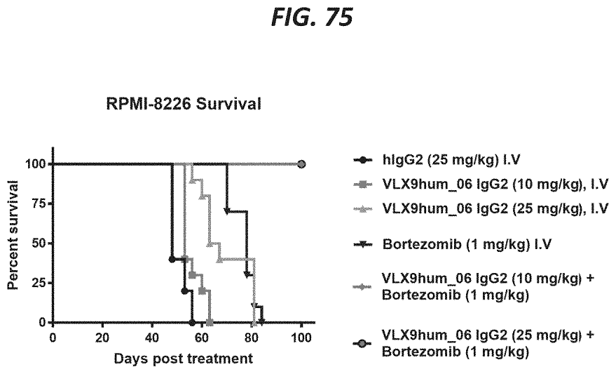

As shown in FIG. 75, secondary assessment of efficacy was assessed by increased survival in treatment groups compared to the hIgG2 vehicle control. All treatment groups resulted in a statistically significant increase in survival when compared to the hIgG2 vehicle control group (p<0.05, Log rank test). Combination treatment with anti-CD47 mAb VLX9hum_06 IgG2+Bortezomib resulted in a statistically significant increase in survival (p<0.05, Log rank survival) when compared to their respective single agent anti-CD47 mAb group and to Bortezomib alone group. Anti-CD47 VLX9hum_06 IgG2+Bortezomib combination groups resulted in the longest survival (99 Days) when all mice were euthanized as long-term survivors as shown in FIG. 75.

As shown in FIG. 75, treatment with the anti-CD47 mAb VLX9hum_06 IgG2 at 10 mg/kg produced a median survival of 53 days (Min: 53, Max: 63) while anti-CD47 mAb VLX9hum_06 IgG2 at 25 mg/kg produced a median survival of 67 Days (Min: 56, Max: 81). A significant increase in survival (p<0.0001) was observed in both anti-CD47 mAb VLX9hum_06 IgG2 monotherapy groups when compared to the hIgG2 vehicle control.

Treatment with Bortezomib 1 mg/kg produced a median survival of 78 Days (Min: 70, Max: 84). A significant increase in survival (p<0.0001) was observed when compared to the hIgG2 vehicle control as shown in FIG. 75.

Treatment with either anti-CD47 mAb VLX9hum_06 IgG2 at doses of 10 mg/kg+Bortezomib 1 mg/kg or anti-CD47 mAb VLX9hum_06 IgG2 25 mg/kg+Bortezomib 1 mg/kg produced a median survival of 99 Days (Min: 99, Max: 99). A significant increase in survival (p<0.0001) was observed when compared to the hIgG2 vehicle control, anti-CD47 mAb VLX9hum_06 IgG2 25 mg/kg, and Bortezomib 1 mg/kg monotherapy groups. By Day 50, 100% of mice in the combination treatment groups resulted in complete response, mice were sacrificed as long-term survivors on Day 99 as shown in FIG. 75.

### Example 24

### VLX9hum_06 IgG2 Increases Phagocytosis of SNU-1 Cells

To assess the effect of anti-CD47 mAbs on phagocytosis of SNU-1 gastric tumor cells by macrophages in vitro the following method is employed using flow cytometry.

Human-derived macrophages were obtained by leukapheresis of human peripheral blood and incubated in tissue culture grade flasks in AIM-V (ThermoFisher, 12055091) with 10% fetal bovine serum (BioWest; Catalog #S01520), and 50 ng/ml macrophage colony-stimulating factor (M-CSF), for seven days after adherence. For in vitro phagocytosis assays, 3×10⁴ macrophages (effector cells) per 100 μL AIM-V media were plated per well in a 96-well tissue culture treated plate. Target cells were labeled with 1 μM 5(6)-carboxyfluorescein N-hydroxysuccinimidyl ester (CFSE) according to the manufacturer protocol (ThermoFisher, C1157). CFSE-labeled target cells at 8×10⁴ cells/100 μL AIM-V media without serum including an 8-fold serial dilution series of an anti-CD47 VLX9hum_06 IgG2 mAb (0.04-30 μg/mL) or 10 μg/mL of the negative control were added to the macrophage cultures and incubated at 37° C. for 3 hours. Macrophages were washed twice with 1×PBS and detached from the tissue culture plate using Accutase® (Sigma, St. Louis, Mo.; SCR005). Cells were stained with Alexa Fluor 647 conjugated anti-human CD14 antibodies (BD biosciences) and analyzed by flow cytometry using an Attune NxT flow cytometer (Life Technologies) for the percentage of CD14-positive macrophages that are positive for CFSE.

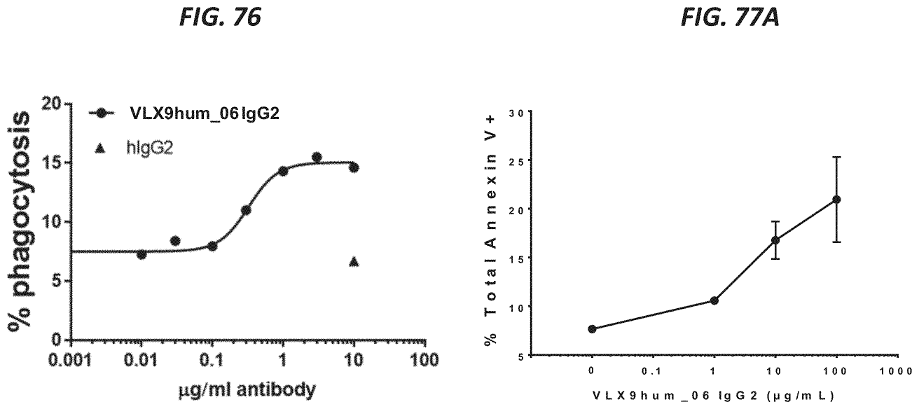

As shown in FIG. 76, the soluble anti-CD47 mAb VLX9hum_06 IgG2 increased phagocytosis of SNU-1 cells by human macrophages in a concentration dependent manner compared to a human IgG2 control antibody.

### Example 25

### VLX9hum_06 IgG2 Mediated Cell Autonomous Killing Alone and in Combination with Cisplatin and Paclitaxel

To assess the effect of anti-CD47 mAbs on cell autonomous death of gastric tumor cells in combination with cisplatin and paclitaxel in vitro the following method is employed using flow cytometry.

Cell autonomous killing after treatment was assessed by cell surface phosphatidylserine exposure. To determine phosphatidylserine exposure after treatment with an anti-CD47 mAb alone or in combination with cisplatin, 5×10⁵ human tumor cells were treated with increasing concentrations of anti-CD47 mAb, cisplatin, or anti-CD47 mAb in combination with cisplatin or anti-CD47 mAb in combination with paclitaxel in complete media both containing 10% (v/v) heat inactivated fetal bovine serum (BioWest; Catalog #S01520), 100 units/mL penicillin, 100 μg/mL streptomycin (Sigma; #P4222). Cells were incubated for 24 hours at 37° C. and 5% CO₂. As cell death occurs, the inner leaflet of the cell membrane is inverted, exposing phosphatidylserine (PS). To detect changes in membrane permeability, cells were stained with fluorescently labeled annexinV (BD biosciences). The percentage of annexinV+ of the total cell population was determined by flow cytometry (Attune NxT flow cytometer, Life Technologies).

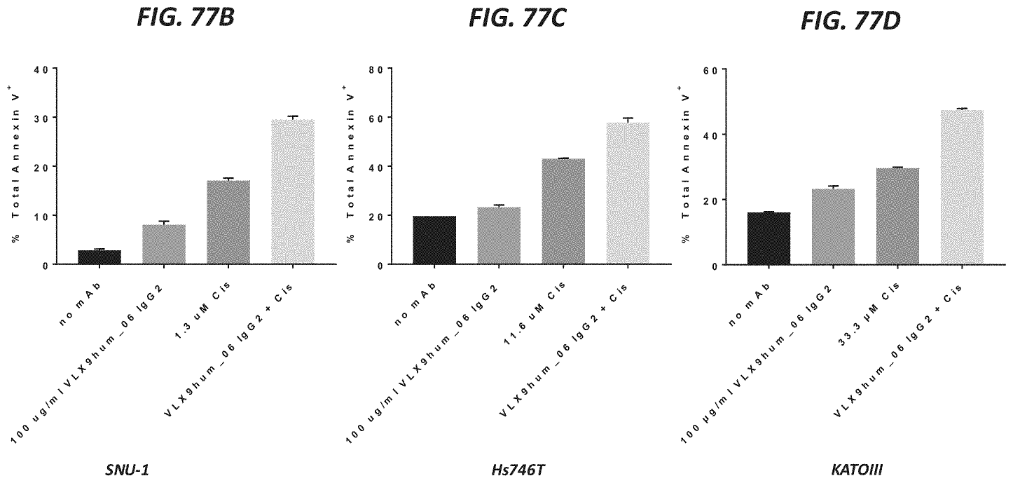

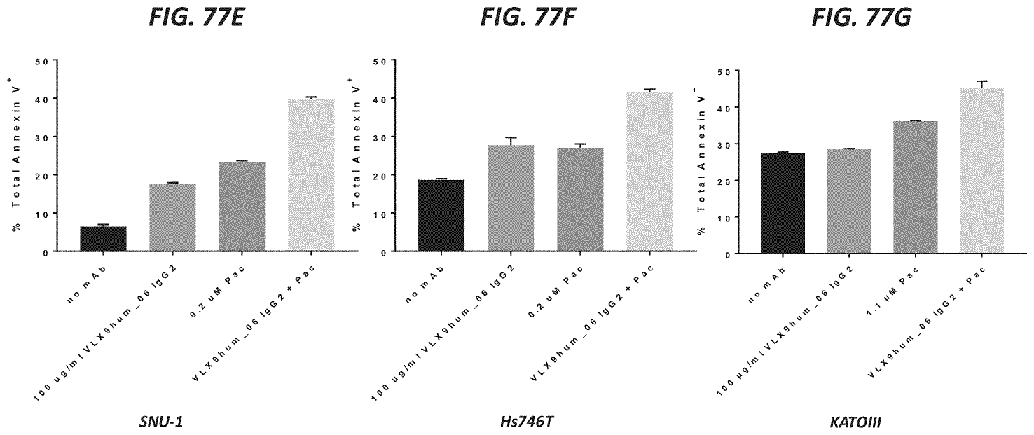

As shown in FIG. 77A, soluble anti-CD47 mAb VLX9hum_06 IgG2 increased cell autonomous death of SNU-1 cells in a concentration dependent manner without the addition of other agents. As shown in FIG. 77B-FIG. 77D, an additive increase in cell autonomous death by anti-CD47 mAb VLX9hum_06 IgG2 in combination with cisplatin was observed in SNU-1 as shown in FIG. 77B, Hs746T as shown in FIG. 77C, or KATOIII as shown in FIG. 77D, gastric carcinoma cells as compared to single agent treatment. Similarly, as shown in FIG. 77E-FIG. 77G, an additive increase cell autonomous death by anti-CD47 mAb VLX9hum_06 IgG2 in combination with paclitaxel was observed in SNU-1 as shown in FIG. 77E, Hs746T as shown in FIG. 77F, or KATOIII as shown in FIG. 77G, gastric carcinoma cells as compared to single agent treatment.

### Example 26

### Anti-Tumor Activity of VLX9hum_06 IgG2 as a Single Agent or in Combination with Cisplatin in SNU-1 Gastric Carcinoma Xenograft in NSG Mice

The disclosure provided herein demonstrates the anti-tumor properties of anti-CD47 mAb VLX9hum_06 IgG2 alone and in combination with cisplatin reduces tumor burden in a xenograft gastric carcinoma model in NSG mice.

Female NSG mice (NOD-Cg-PrkdcscidI12rgtm1Wjl/SzJ, Jackson Laboratories) were inoculated subcutaneously in the right flank with 0.1 mL of a 30% RPMI/70% Matrigel™ (BD Biosciences; Bedford, Mass.) mixture containing a suspension of 5×10⁶ SNU-1 gastric carcinoma cells (ATCC). Eight days following inoculation, digital calipers were used to measure width and length diameters of the tumor. Tumor volumes were calculated utilizing the formula: tumor volume (mm³)=(a×b²/2) where ‘b’ is the smallest diameter and ‘a’ is the largest diameter. Mice with palpable tumor volumes of 50-100 mm³ were randomized into 10 mice/group and administered anti-CD47 mAb VLX9hum_06 IgG2, cisplatin, control IgG2, or a combination of anti-CD47 mAb VLX9hum_06 IgG2 with cisplatin. Mice were treated with 25 mg/kg of antibody once a week for 5 weeks (Q7D×5) by intraperitoneal injection (IP) and/or with 3 mg/kg of cisplatin once weekly for 4 weeks (Q7D×4). Tumor volumes and body weights were recorded twice weekly.

Mean tumor growth inhibition (TGI) was calculated utilizing the following formula. Mice exhibiting tumor shrinkage (TS) were excluded from the TGI calculations.

$$TGI = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

Significant differences in tumor volume were confirmed using a two-way ANOVA, unpaired, parametric with the Tukey's Multiple Comparison test.

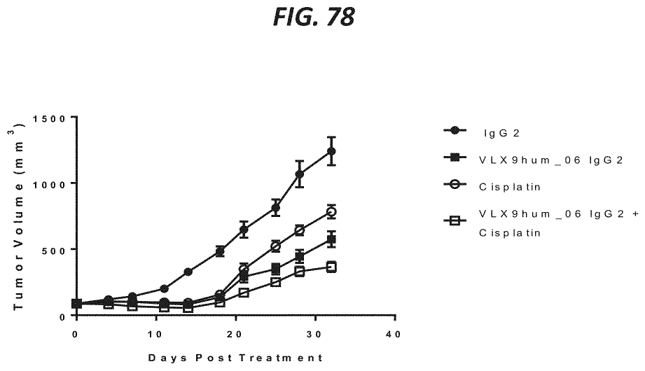

As shown in FIG. 78, treatment with the humanized anti-CD47 mAb VLX9hum_06 IgG2, significantly reduced (p<0.0001, two-way ANOVA) tumor growth of the SNU-1 tumors with tumor growth inhibition of 57.8%, demonstrating anti-tumor efficacy in vivo. Treatment with cisplatin alone resulted in a more modest reduction in tumor burden with 39.7% TGI (p<0.0001, two-way ANOVA). The combination of the anti-CD47 mAb VLX9hum_06 IgG2 with cisplatin demonstrated significant additive tumor growth inhibition (TGI=75.9) compared to control (p<0.0001, two-way ANOVA) and single agent cisplatin (p<0.0001, two-way ANOVA) and VLX9hum_06 IgG2 (p=0.0003, two-way ANOVA) treatment.

### Example 27

### Anti-Tumor Activity of VLX9hum_06 IgG2 as a Single Agent or in Combination with Cisplatin or Paclitaxel in an OV90 Ovarian Xenograft in NSG Mice

The disclosure provided herein demonstrates the anti-tumor properties of the anti-CD47 mAb VLX9hum_06 IgG2 as a single agent and in combination with cisplatin reduces tumor burden in a xenograft gastric carcinoma model in NSG mice.

Female NSG mice (NOD-Cg-PrkdcscidI12rgtm1Wjl/SzJ, Jackson Laboratories) were inoculated subcutaneously in the right flank with 0.1 mL of a 30% RPMI/70% Matrigel™ (BD Biosciences; Bedford, Mass.) mixture containing a suspension of 5×10⁶ OV90 ovarian carcinoma cells (ATCC). Digital calipers were used to measure width and length diameters of the tumor. Tumor volumes were calculated utilizing the formula: tumor volume (mm³)=(a×b²/2) where ‘b’ is the smallest diameter and ‘a’ is the largest diameter. Mice with palpable tumor volumes of 50-100 mm³ were randomized into 10 mice/group. Mice were treated with 10 mg/kg of antibody (VLX9hum_06 IgG2 or control antibody) five days per week for a total of 6 weeks (QD5×6) by intraperitoneal injection (IP). Cisplatin (5 mg/kg) was administered IP for a total of three doses (day 0, day 7 and day 28), either as a single agent or in combination with the anti-CD47 mAb VLX9hum_06 IgG2. Paclitaxel (20 mg/kg/dose) was administered by IP route for a total of four doses (day 0, day 7, day 14 and day 21) either as single agent or in combination with the anti-CD47 mAb VLX9hum_06 IgG2. Tumor volumes and body weights were recorded twice weekly.

Mean tumor growth inhibition (TGI) was calculated utilizing the following formula. Mice exhibiting tumor shrinkage (TS) were excluded from the TGI calculations.

$$TGI = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

Significant differences in tumor volume were confirmed using a two-way ANOVA, unpaired, parametric with the Tukey's Multiple Comparison test.

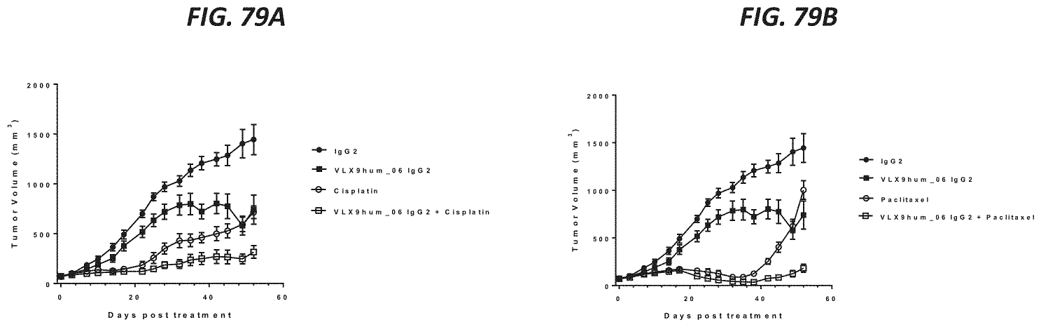

As shown in FIG. 79, VLX9hum_06 IgG2 exhibited statistically significant inhibition of tumor growth in OV90 human ovarian xenograft model at 10 mg/kg daily dosing with 51.3% tumor growth inhibition by Day 52 as shown in FIG. 79A and FIG. 79B).

Cisplatin also resulted in significant tumor inhibition in OV90 model as monotherapy (53.1% TGI). Importantly, combination of the anti-CD47 mAb VLX9hum_06 IgG2 and cisplatin resulted in statistically significant anti-tumor activity when compared to the single agent treatment (p<0.0001, 2-way ANOVA) with combination tumor growth inhibition of 79.4% TGI at Day 52 (FIG. 79A).

Paclitaxel treatment led to significant tumor inhibition with 32.1% TGI as shown in FIG. 79B, p<0.0001). In combination with anti-CD47 mAb VLX9hum_06 IgG2, paclitaxel treatment significantly enhanced anti-tumor activity when compared to the single agent treatment (p<0.0001, 2-way ANOVA) resulting in 89% TGI at day 52 as shown in FIG. 79B.

### Example 28

### Treatment with VLX9hum_06 IgG2 Results in Increased ProInflammatory Cytokine Secretion in the Tumor Micro-Environment in an Ovarian Xenograft Model

To assess cytokine secretion within the tumor microenvironment described in Example 27, NSG mice were treated IP daily with VLX9hum_06 IgG2 mAbs or IgG2 control mAbs at a concentration of 10 mg/kg for a total of 5 days. Tumors were excised at 48h, 96h and 168h after initial treatment in a satellite group of animals. Tumors (N=3/group) were quantified for murine cytokines (IL-1β and IL-10) and chemokines (MCP-1, IP-10 and MIP-1α) with a Meso Scale Discovery custom cytokine plate (MSD, Gaithersburg, Md., USA) according to the manufacturer's instructions. The plates were analyzed on the MSD Sector 2400 Imager (MSD). Statistics were generated using a 2-way ANOVA.

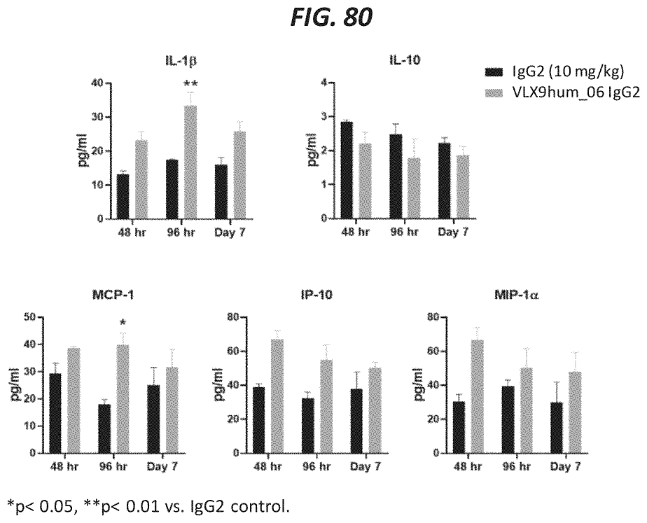

Monotherapy with the VLX9hum_06 IgG2 anti-CD47 mAb led to a significant increase in the release of the pro-inflammatory cytokine IL-1β and chemokine MCP-1 as shown in FIG. 80. There were increases in MIP-1α and IP-10 (or CXCL-10) chemokines, albeit not significant at the collected timepoints. No differences were observed for in IL-10, a known an immune suppressive cytokine, between mice treated with the VLX9hum_06 IgG2 anti-CD47 mAb and IgG2 control.

### Example 29

### VLX9hum_06 IgG2 mAbs Induce Dendritic Cell (DC) Recruitment to the Host Tumor Microenvironment (TME) which Increases the Release of Pro-Inflammatory Cytokines and Chemokines in a Xenograft Mouse Model (RPMI-8226)

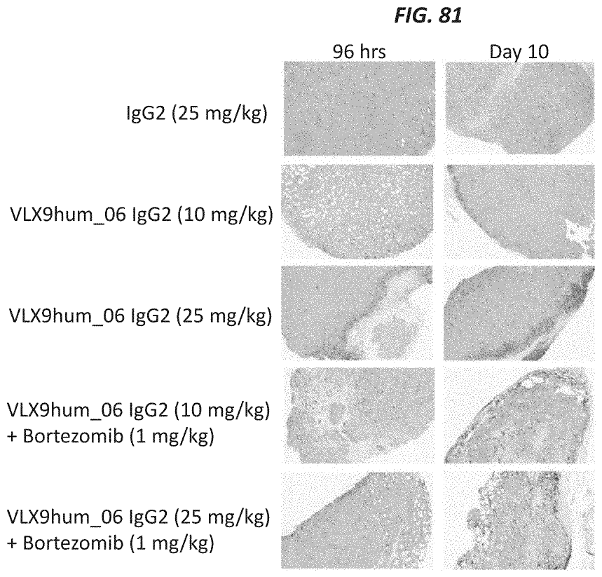

To evaluate the effects of anti-CD47 mAb monotherapy and anti-CD47 mAb combination therapy with bortezomib on the host tumor microenvironment (TME), NSG mice were treated with 1) VLX9hum_06 IgG2 mAbs 2) IgG2 control mAbs 3) VLX9hum_06 IgG2 mAbs+bortezomib 4) IgG2 control mAbs+bortezomib as described in Example 23. RPMI-8226 tumors were collected and evaluated using immunohistochemistry (IHC) at 96 hrs and 10 days and for release of cytokines/chemokines at 48 hrs, 96 hrs and 10 days (N=3 tumors/group) following the initiation of treatment. The IHC data showed a dose-dependent increase in murine CD11c⁺ DC tumor infiltrates (brown staining) following the administration of VLX9hum_06 IgG2 mAbs. VLX9hum_06 IgG2 mAbs and bortezomib combination therapy resulted in elevated tumor DC recruitment comparable to VLX9hum_06 IgG2 mAbs monotherapy at the early time points (96 hrs and Day 10) following dosing. Representative images from one animal from each group, e.g., 96 hrs and Day 10, are shown in FIG. 81.

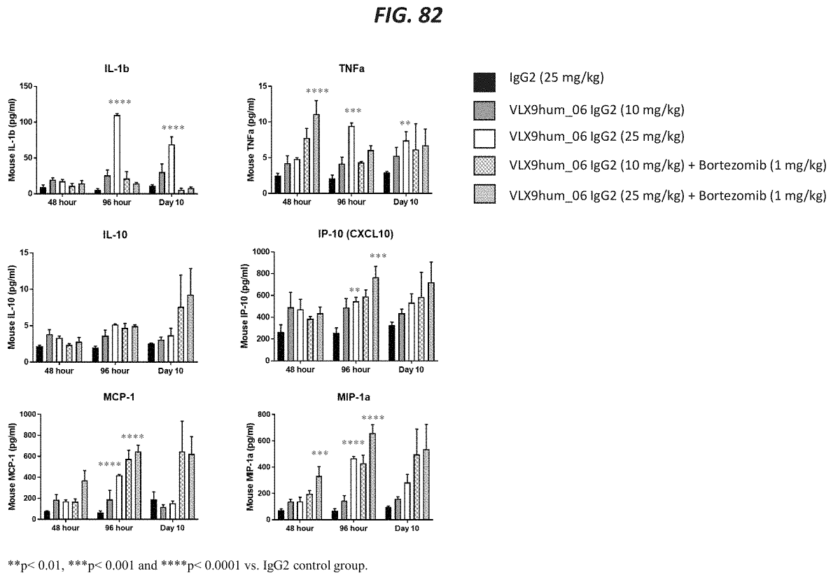

The VLX9hum_06 IgG2 monotherapy led to a significant increase in the release of pro-inflammatory cytokines IL-1β and TNF-α, and the release of chemokines, IP-10 (or CXCL10), MCP-1, and MIP-1α. The pro-inflammatory cytokines and chemokines correlate with increased tumor DC recruitment and cause a local effect on tumor growth inhibition. The VLX9hum_06 IgG2 mAbs+bortezomib combination therapy further enhanced the production of some of these cytokines and chemokines, which included TNF-α, IP-10, MCP-1, and MIP-1α, correlating with increased tumor immune cell infiltrates and mechanisms of anti-tumor activity of VLX9hum_06 IgG2mAb by phagocytosis and proteasome inhibition with bortezomib as shown in FIG. 82. IL-10, a cytokine with immune suppressive function, was slightly increased but not significantly different in the anti-CD47 mAb treated mice compared to the IgG2 control group as shown in FIG. 82. There were no differences detected in murine IL-6, IL-12, p70, and MIP-2 (data not shown) between all the groups.

### Example 30

### Anti-CD47 mAbs Pharmacokinetics in Mice Bearing RPMI-8226 Human Multiple Myeloma Tumor Xenograft

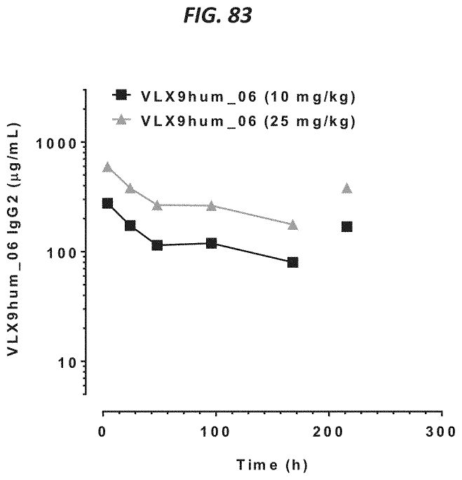

The pharmacokinetics of VLX9hum_06 IgG2 mAb were characterized in human multiple myeloma RPMI-8226 tumor bearing NSG mice when administered by intravenous injection at dose levels of 10 mg/kg and 25 mg/kg once weekly. FIG. 83 shows the PK profile for VLX9hum_06 IgG2 mAb dosing at 10 mg/kg and 25 mg/kg, respectively, by intravenous (IV) injection. The Cmax could not be calculated since serum was not collected at early timepoints (2-15 min post VLX9hum_06 IgG2 mAb dosing). The increase in Co (from 304 μg/mL to 654 μg/mL) was roughly proportional to the increased VLX9hum_06 IgG2 mAb dose administered at 10 mg/kg and 25 mg/kg. The serum half-life was comparable between 10 mg/kg and 25 mg/kg weekly IV dosing, 48.3% TGI was observed with 10 mg/kg VLX9hum_06 IgG2 mAb monotherapy versus 84.2% TGI when dosing at 25 mg/kg IV at day 48. The half-life of VLX9hum_06 IgG2mAb mAb in the RPMI-8226 model is similar to our previously observed half-life in the Raji human B-cell lymphoma xenograft. The PK data along with TGI results suggest that an VLX9hum_06 IgG2mAb mAb exposure of ˜250 ug/mL is required for efficacy in RPMI-8226-multiple myeloma xenografted mice (once/week dosing) as described in Example 23.

### Example 31

### Anti-Tumor Activity of Anti-CD47 Antibodies and Proteasome Inhibitors in a Xenograft Mouse Model (MM.1S) of Multiple Myeloma

The anti-tumor properties of a humanized anti-CD47 antibody (VLX9hum_06 IgG2) and in combination with bortezomib resulting in reduced tumor burden in a xenograft MM.1S multiple myeloma NSG mouse model were evaluated. MM.1S human multiple myeloma cells (ATCC #CRL-2974, Manassas, Va.) were maintained in RPMI-1640 (Lonza; Walkersville, Md.) supplemented with 10% Fetal Bovine Serum (FBS; Omega Scientific; Tarzana, Calif.) and 1% Penicillin/Streptomycin (Corning, Manassas, Va.) within a 5% CO2 atmosphere. Cultures were expanded in tissue culture flasks.

Female NSG (NOD-Cg-Prkdc^{scid}I12rg^{tm1Wjl}/SzJ) were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NSG mice were inoculated subcutaneously in the right flank with 0.1 mL of a 30% RPMI/70% Matrigel™ (BD Biosciences; Bedford, Mass.) mixture containing a suspension of 5×10⁶ MM.1S tumor cells. Nineteen days following inoculation, mice were randomized. The test articles human IgG2 (hIgG2), anti-CD47 mAb (VLX9hum_06 IgG2) were administered by intraperitoneal (IP) injection and Bortezomib (LC Labs, Woburn, Mass.) was administered by intravenous (IV) injection. hIgG2 (25 mg/kg) or an anti-CD47 mAb VLX9hum_06 IgG2 were administered weekly for 4 weeks and bortezomib was administered on day 1 and 3 at a dose of 0.75 mg/kg and on days 10 and 17 at a dose of 0.5 mg/kg.

Mean tumor growth inhibition (TGI) was calculated at Day 20 (the final day all mice were on study) utilizing the following formula. Of note, mice exhibiting tumor shrinkage were excluded from the TGI calculations.

$$TGI = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

Individual tumor shrinkage (TS) was calculated at Day 20 using the formula below for tumors that showed regression relative to Day 0. The mean TS of each group was calculated and reported.

$$TS = [ 1 - \frac{( Tumor Volume ( Final ) )}{( Tumor Volume ( Day 0 ) )} ] \times 100 \%$$

All statistical analyses in the xenograft study were performed with Prism GraphPad® software. Differences in Day 20 tumor volumes were confirmed using a two-way ANOVA, unpaired, parametric with the Tukey's Multiple Comparison test with Welch's correction.

Increase in survival fractions were confirmed by the log rank test with a comparison of each group to the vehicle control group.

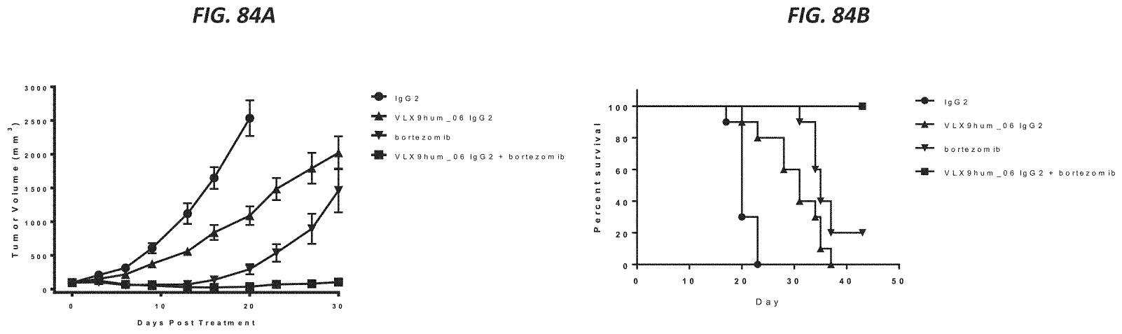

As shown in FIG. 84A, the primary efficacy assessment based on tumor growth inhibition (TGI) resulted in statistically significant anti-tumor activity in all groups compared to the hIgG2 vehicle control group. Both the 25 mg/kg VLX9hum_06 IgG2 (59.3% TGI) or bortezomib (91.8% TGI), groups demonstrated single agent anti-tumor efficacy. Additionally, combination treatment with VLX9hum_06 IgG2 and bortezomib resulted in statistically significant decreases in tumor volumes when compared to single agent treatment with VLX9hum_06 IgG2 (p<0.0001, 2-way ANOVA). Anti-tumor efficacy was seen in the combination group, which resulted in complete tumor regression of 60% of mice by Day 20. No tumor shrinkage was recorded with monotherapy, whereas a mean tumor shrinkage (TS) of 62.2% was observed in the 25 mg/kg anti-CD47 mAb VLX9hum_06 IgG2+ bortezomib group compared to the hIgG2 vehicle control, demonstrating synergistic anti-tumor efficacy of combination agents in vivo. By Day 37, 70% of mice in the combination treatment groups resulted in a complete response and continued to demonstrate complete regression by Day 43 following treatment as shown in FIG. 84A.

As shown in FIG. 84B, the secondary assessment of efficacy was assessed by increased survival in treatment groups compared to the hIgG2 vehicle control. All treatment groups resulted in a statistically significant increase in survival when compared to the hIgG2 vehicle control group (p<0.05, Log rank test). Combination treatment with anti-CD47 mAb VLX9hum_06 IgG2+bortezomib resulted in a statistically significant increase in survival (p<0.05, Log rank survival) when compared to their respective single agent anti-CD47 mAb group and to Bortezomib alone group. Anti-CD47 VLX9hum_06 IgG2+ Bortezomib combination groups resulted in the longest duration of survival as shown in FIG. 84B.

As shown in FIG. 84B, treatment with the anti-CD47 mAb VLX9hum_06 IgG2 mg/kg produced a median survival of 31 days (Min:20, Max: 35). A significant increase in survival (p<0.0001) was observed in anti-CD47 mAb VLX9hum_06 IgG2 treated animals when compared to the hIgG2 vehicle control.

Treatment with bortezomib produced a median survival of 35 Days. A significant increase in survival (p<0.0001) was observed when compared to the hIgG2 vehicle control as shown in FIG. 84B.

Treatment with anti-CD47 mAb VLX9hum_06 IgG2+ Bortezomib extended survival greater than 43 days post initial dose as shown in FIG. 84B.

### Example 32

### Anti-Tumor Activity of Anti-CD47 Antibodies and CD38 Targeting Antibodies in a Xenograft Mouse Model (MM.1S) of Multiple Myeloma

The anti-tumor properties of a humanized anti-CD47 antibody (VLX9hum_06 IgG2) and in combination with anti-CD38 monoclonal antibody daratumumab which reduce tumor burden in a xenograft MM.1S multiple myeloma NSG mouse model were evaluated MM.1S human multiple myeloma cells (ATCC #CRL-2974, Manassas, Va.) were maintained in RPMI-1640 (Lonza; Walkersville, Md.) supplemented with 10% Fetal Bovine Serum (FBS; Omega Scientific; Tarzana, Calif.) and 1% Penicillin/Streptomycin (Corning, Manassas, Va.) within a 5% CO2 atmosphere. Cultures were expanded in tissue culture flasks.

Female NSG (NOD-Cg-Prkdc^{scid}I12rg^{tm1Wjl}/SzJ) were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NSG mice were inoculated subcutaneously in the right flank with 0.1 mL of a 30% RPMI/70% Matrigel™ (BD Biosciences; Bedford, Mass.) mixture containing a suspension of 5×10⁶ MM.1S tumor cells. Nineteen days following inoculation, mice were randomized. The test articles human IgG2 (hIgG2), anti-CD47 mAb (VLX9hum_06 IgG2), and daratumumab (Myoderm, Norristown, Pa.) were administered by intravenous (IP) injection. hIgG2 (25 mg/kg) or an anti-CD47 mAb VLX9hum_06 IgG2 were administered weekly for 4 weeks, while daratumumab was administered twice weekly at a dose of 15 mg/kg for 6 weeks.

Mean tumor growth inhibition (TGI) was calculated at Day 20 utilizing the following formula. Of note, mice exhibiting tumor shrinkage were excluded from the TGI calculations.

$$TGI = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

All statistical analyses in the xenograft study were performed with Prism GraphPad® software. Differences in Day 20 tumor volumes were confirmed using a two-way ANOVA, unpaired, parametric with the Tukey's Multiple Comparison test with Welch's correction.

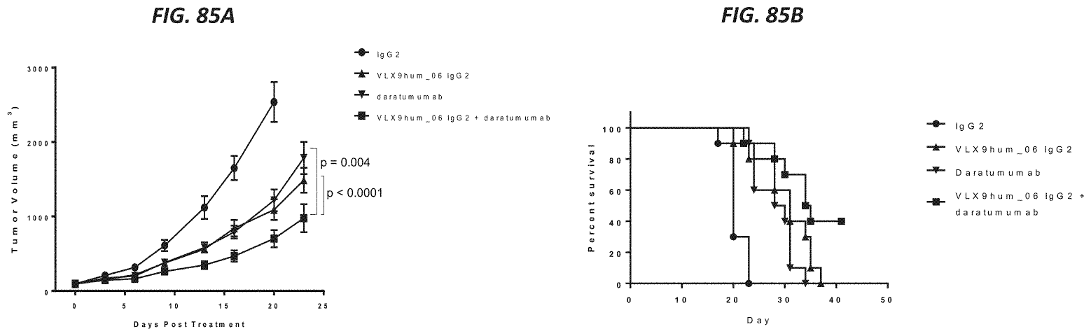

MM.1S Primary Assessment of Efficacy Based on Tumor Volume. As shown in FIG. 85A, the primary efficacy assessment based on tumor growth inhibition (TGI) resulted in statistically significant anti-tumor activity in all groups when compared to the hIgG2 vehicle control group. Both the 25 mg/kg VLX9hum_06 IgG2 (59.3% TGI) and daratumumab (54% TGI), demonstrated single agent anti-tumor efficacy in vivo. Combination treatment resulted in statistically significant decreases in tumor volumes when compared to single agent treatment with VLX9hum_06 IgG2 or daratumumab (p=0.02 and p=0.001, respectively by 2-way ANOVA) with 75.2% TGI as shown in FIG. 85A.

As shown in FIG. 85B, the secondary assessment of efficacy was assessed by increased survival in treatment groups compared to the hIgG2 vehicle control. All treatment groups resulted in a statistically significant increase in survival when compared to the hIgG2 vehicle control group (p<0.05, Log rank test). Combination treatment with anti-CD47 mAb VLX9hum_06 IgG2+daratumumab resulted in a statistically significant increase in survival (p<0.05, Log rank survival) when compared to their respective single agent anti-CD47 mAb group and to daratumumab alone group. Anti-CD47 VLX9hum_06 IgG2+daratumumab combination groups resulted in the longest survival as shown in FIG. 85B.

As shown in FIG. 85B, treatment with the anti-CD47 mAb VLX9hum_06 IgG2 mg/kg produced a median survival of 31 days. Treatment with daratumumab produced a median survival of 29 Days.

Treatment with anti-CD47 mAb VLX9hum_06 IgG2+daratumumab extended median survival to 35 days with 40% of the animals surviving to greater than 43 days following the imitation of treatment as shown in FIG. 85B.

### Example 33

### Anti-Tumor Activity of Anti-CD47 Antibodies in a Xenograft Mouse Model (NCI-H929) of Multiple Myeloma

The anti-tumor properties of a humanized anti-CD47 antibody (VLX9hum_06 IgG2) in a xenograft NCI-H929 multiple myeloma NSG mouse model were evaluated. NCI-H929 human multiple myeloma cells (ATCC #CRL-9068, Manassas, Va.) were maintained in RPMI-1640 (Lonza; Walkersville, Md.) supplemented with 10% Fetal Bovine Serum (FBS; Omega Scientific; Tarzana, Calif.) and 1% Penicillin/Streptomycin (Corning, Manassas, Va.) within a 5% CO2 atmosphere. Cultures were expanded in tissue culture flasks.

Female NSG (NOD-Cg-Prkdc^{scid}I12rg^{tm1Wjl}/SzJ) were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NSG mice were inoculated subcutaneously in the right flank with 0.1 mL of a 30% RPMI/70% Matrigel™ (BD Biosciences; Bedford, Mass.) mixture containing a suspension of 1×10⁷ NCI-H929 tumor cells. Nineteen days following inoculation, mice were randomized. The test articles human IgG2 (hIgG2) and anti-CD47 mAb (VLX9hum_06 IgG2) were administered by intravenous (IP) injection. hIgG2 (25 mg/kg) or an anti-CD47 mAb VLX9hum_06 IgG2 were administered weekly for 4 weeks.

Mean tumor growth inhibition (TGI) was calculated at Day 26 utilizing the following formula. Of note, mice exhibiting tumor shrinkage were excluded from the TGI calculations.

$$TGI = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

Individual tumor shrinkage (TS) was calculated at Day 20 using the formula below for tumors that showed regression relative to Day 0. The mean TS of each group was calculated and reported.

$$TS = [ 1 - \frac{( Tumor Volume ( Final ) )}{( Tumor Volume ( Day 0 ) )} ] \times 100 \%$$

All statistical analyses in the xenograft study were performed with Prism GraphPad® software. Differences in Day 26 tumor volumes were confirmed using a two-way ANOVA, unpaired, parametric with the Tukey's Multiple Comparison test with Welch's correction.

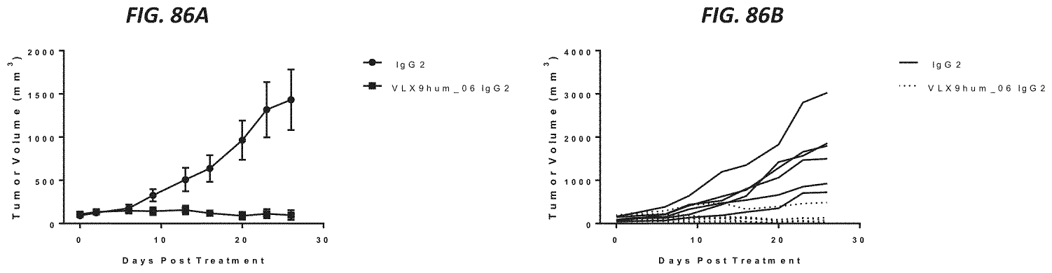

NCI-H929 Primary Assessment of Efficacy Based on Tumor Volume. As shown in FIG. 86A, primary efficacy assessment based on tumor growth inhibition (TGI) resulted in statistically significant anti-tumor activity in all groups when compared to the hIgG2 vehicle control group with 25 mg/kg VLX9hum_06 IgG2 (100% TGI, 9% TS) demonstrating single agent anti-tumor efficacy in vivo as shown in FIG. 86B. Efficacy in each individual mouse is shown in a spider plot as shown in FIG. 86B.

### Example 34

### Anti-CD47 mAbs Increase Phagocytosis

To assess the effect of anti-CD47 mAbs on phagocytosis of tumor cells by macrophages in vitro the following method is employed using flow cytometry.

Human derived macrophages were derived from leukapheresis of healthy human peripheral blood and incubated in AIM-V media (Life Technologies) supplemented with 50 ng/ml M-CSF (Biolegend) for seven days. For the in vitro phagocytosis assay, macrophages were re-plated at a concentration of 3×10⁴ cells per well in 100 μl of AIM-V media supplemented with 50 ng/ml M-CSF in a 96-well plate and allowed to adhere for 24 hours. Once the effector macrophages adhered to the culture dish, the targeted human cancer cells were labeled with 1 μM 5(6)-Carboxyfluorescein diacetate N-succinimidyl ester (CFSE; Sigma Aldrich) and added to the macrophage cultures at a concentration of 8×10⁴ cells in 100 μl of AIM-V media without supplements. VLX9hum_06 IgG2 mAb was added at various concentrations immediately upon mixture of target and effector cells and allowed to incubate at 37° C. for 3 hours. After 3 hours, all non-phagocytosed cells were removed, and the remaining cells washed three times with PBS. Cells were then incubated in Accutase (Stemcell Technologies) to detach macrophages, collected into microcentrifuge tubes, and incubated in 100 ng of allophycocyanin (APC) labeled CD14 antibodies (BD biosciences) for 30 minutes, washed once, and analyzed by flow cytometry (Attune, Life Technologies) for the percentage of CD14⁺ cells that were also CFSE⁺ indicating complete phagocytosis.

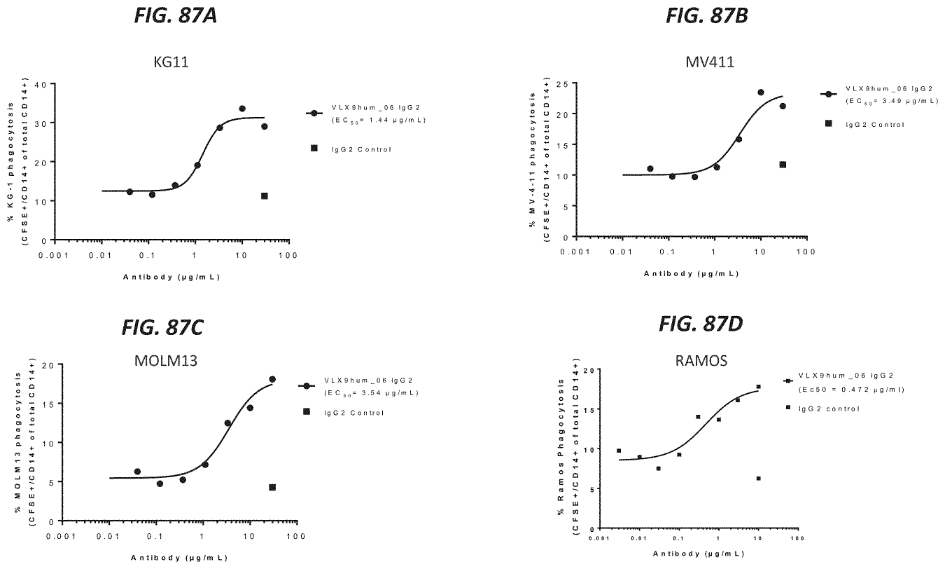

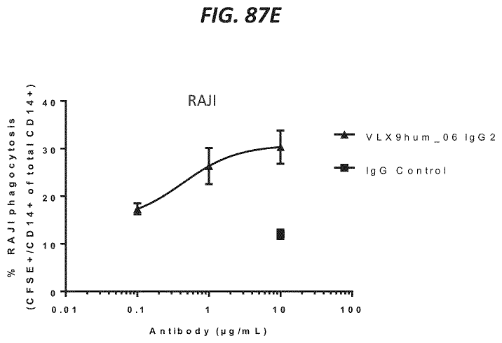

As shown in FIGS. 87A-87E, VLX9hum_06 IgG2 mAbs increased phagocytosis of KG1, MV411, MOLM13, Ramos, and Raji tumor cells by human macrophages in a concentration-dependent fashion compared to an IgG2 control antibody (Biolegend).

### Example 35

### Anti-CD47 mAbs Increase Phagocytosis when Combined with Anti-CD20 Antibodies

To assess the effect of anti-CD47 mAbs and anti-CD47 mAbs in combination with anti-CD20 on phagocytosis of tumor cells by macrophages in vitro the following method is employed using flow cytometry.

Human derived macrophages were derived from leukapheresis of healthy human peripheral blood and incubated in AIM-V media (Life Technologies) supplemented with 50 ng/ml M-CSF (Biolegend) for seven days. For the in vitro phagocytosis assay, macrophages were re-plated at a concentration of 3×10⁴ cells per well in 100 μl of AIM-V media supplemented with 50 ng/ml M-CSF in a 96-well plate and allowed to adhere for 24 hours. Once the effector macrophages adhered to the culture dish, the targeted human cancer Raji cells were labeled with 1 μM 5(6)-Carboxyfluorescein diacetate N-succinimidyl ester (CFSE; Sigma Aldrich) and added to the macrophage cultures at a concentration of 8×10⁴ cells in 100 μl of AIM-V media without supplements. Monotherapy of VLX9hum_06 IgG2 mAbs, monotherapy of anti-CD20 mAb (Rituxan, Roche), and a combination therapy of VLX9hum_06 IgG2 mAbs and anti-CD20 were added at various concentrations immediately upon mixture of target and effector cells and allowed to incubate at 37° C. for 3 hours. After 3 hours, all non-phagocytosed cells were removed, and the remaining cells washed three times with PBS. Cells were then incubated in Accutase (Stemcell Technologies) to detach macrophages, collected into microcentrifuge tubes, and incubated in 100 ng of allophycocyanin (APC) labeled CD14 antibodies (BD biosciences) for 30 minutes, washed once, and analyzed by flow cytometry (Attune, Life Technologies) for the percentage of CD14+ cells that were also CFSE⁺ indicating complete phagocytosis.

As shown in FIG. 88, VLX9hum_06 IgG2 mAbs increased phagocytosis of Raji cells by human macrophages when combined with anti-CD20 mAbs compared to either agent alone. Comparison of combination treatment to single-agent treatment of VLX9hum_06 IgG2 or bortezomib resulted in statistically significant increases in phagocytosis (**** p<0.0001 and *** p=0.0002, 1-way ANOVA)

### Example 36

### Anti-CD47 mAbs Increase Phagocytosis of Multiple Myeloma Cells

To assess the effect of anti-CD47 mAbs on phagocytosis of multiple myeloma tumor cells by macrophages in vitro the following method is employed using flow cytometry.

Human-derived macrophages were obtained by leukapheresis of human peripheral blood and incubated in tissue culture grade flasks in AIM-V (ThermoFisher, 12055091) with 10% fetal bovine serum (BioWest; Catalog #S01520), and 50 ng/ml macrophage colony-stimulating factor (M-CSF), for seven days after adherence. For in vitro phagocytosis assays, 3×10⁴ macrophages (effector cells) per 100 μL AIM-V media were plated per well in a 96-well tissue culture treated plate. Target cells were labeled with 1 μM 5(6)-carboxyfluorescein N-hydroxysuccinimidyl ester (CFSE) according to the manufacturer protocol (ThermoFisher, C1157). CFSE-labeled target cells at 8×10⁴ cells/100 μL AIM-V media without serum including an 8-fold serial dilution series of test antibody (0.04-30 μg/mL) or 10 μg/mL of the negative control were added to the macrophage cultures and incubated at 37° C. for 3 hours. Macrophages were washed twice with 1×PBS and detached from the tissue culture plate using Accutase® (Sigma, St. Louis, Mo.; SCR005). Cells were stained with Alexa Fluor 647 conjugated anti-human CD14 antibodies (BD biosciences) and analyzed by flow cytometry using an Attune NxT flow cytometer (Life Technologies) for the percentage of CD14-positive macrophages that are positive for CFSE.

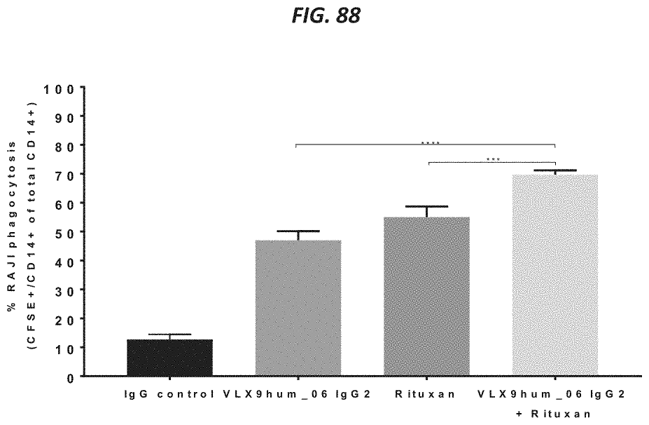

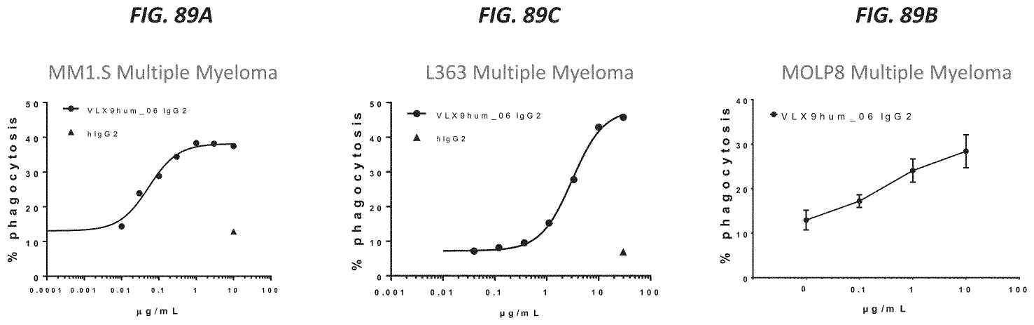

As shown in FIG. 89A-FIG. 89C, a soluble anti-CD47 mAb increased phagocytosis of MM1.S, L363, and MOLP8 cells by human macrophages in a concentration-dependent fashion compared to a human IgG2 control antibody.

### Example 37

### Anti-CD47 mAbs Mediated Cell Autonomous Killing of Multiple Myeloma Cells in Combination with Bortezomib

To assess the effect of anti-CD47 mAbs on cell autonomous death of multiple myeloma tumor cells in combination with bortezomib in vitro the following method is employed using flow cytometry.

Cell autonomous killing after treatment was assessed by cell surface phosphatidylserine exposure. To determine phosphatidylserine exposure after treatment with anti-CD47 mAb alone with or in combination with bortezomib (Takeda), 5×10⁵ human tumor cells were treated with increasing concentrations of anti-CD47 mAb alone, bortezomib alone, or anti-CD47 mAb and bortezomib in combination in complete media both containing 10% (v/v) heat inactivated fetal bovine serum (BioWest; Catalog #S01520), 100 units/mL penicillin, 100 μg/mL streptomycin (Sigma; #P4222). Cells were incubated for 24 hours at 37 degrees Celsius and 5% CO₂. As cell death occurs, the inner leaflet of the cell membrane is inverted, exposing phosphatidylserine (PS). Cells were stained with fluorescently labeled annexin V (BD biosciences). The percentage of annexin V+ of the total cell population was determined by flow cytometry (Attune NxT flow cytometer, Life Technologies).

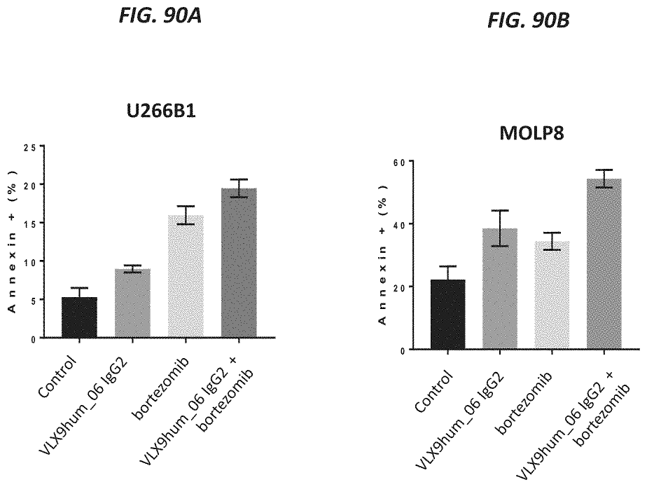

As shown in FIG. 90A-FIG. 90B, cell autonomous death by anti-CD47 mAbs in combination with bortezomib was determined by treating U266B1 cells with 10 μg/mL anti-CD47 mAb alone, 30 nM bortezomib alone, 10 μg/mL anti-CD47 mAb and bortezomib, or MOLP8 cells with 10 μg/mL anti-CD47 mAb alone, 42 nM bortezomib alone, 10 μg/mL anti-CD47 mAb and bortezomib for 24 hours at 37° C. Cells were stained with annexin V to measure externalization of phosphatidylserine (annexin V+) and measured by flow cytometry. Comparison of combination treatment to single-agent treatment resulted in statistically significant increases in the percent annexin V positive cells (p<0.0001, 1-way ANOVA).

### Example 38

### Anti-Tumor Activity of Anti-CD47 Antibodies Combined with Immunomodulating Drugs in a Xenograft Mouse Model of Multiple Myeloma

This disclosure demonstrates the anti-tumor properties of a humanized anti-CD47 antibody (VLX9hum_06 IgG2) as a single agent and in combination with immunomodulator drugs, in reducing tumor burden in a xenograft multiple myeloma NOD-SCID mouse model. MM.1S human multiple myeloma cells (ATCC #CRL-2974, Manassas, Va.) were maintained in RPMI-1640 (Lonza; Walkersville, Md.) supplemented with 10% Fetal Bovine Serum (FBS; Omega Scientific; Tarzana, Calif.) and 1% Penicillin/Streptomycin (Corning, Manassas, Va.) within a 5% CO₂ atmosphere. Cultures were expanded in tissue culture flasks. Female NOD-SCID (NOD.CB17-Prkdc^{scid}/J) mice were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NOD-SCID mice were inoculated subcutaneously in the right flank with 0.1 mL of a 50% RPMI-1640/50% Matrigel™ (BD Biosciences; Bedford, Mass.) mixture containing a suspension of 5×10⁶ MM.1S tumor cells. When tumors reached volumes of approximately 50-100 mm³, mice were randomized. The test articles, human IgG2 (hIgG2), anti-CD47 mAb (VLX9hum_06 IgG2) were administered by intraperitoneal (IP) injection and lenalidomide or pomalidomide (LC Labs, Woburn, Mass.) were administered by oral gavage (PO). hIgG2 (25 mg/kg) or an anti-CD47 mAb VLX9hum_06 IgG2 (25 mg/kg) were administered weekly for 5 weeks, while lenalidomide (25 mg/kg) or pomalidomide (10 mg/kg) were administered on 4 successive days, with 3 days off, weekly for 5 weeks.

Mean tumor growth inhibition (TGI) was calculated at Day 24 (the final day all mice were on study) utilizing the following formula. Of note, mice exhibiting tumor shrinkage were excluded from the TGI calculations.

$$TGI = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

All statistical analyses in the xenograft study were performed with Prism GraphPad® software. Differences in Day 24 tumor volumes were confirmed using a two-way ANOVA, unpaired, parametric with the Tukey's Multiple Comparison test with Welch's correction.

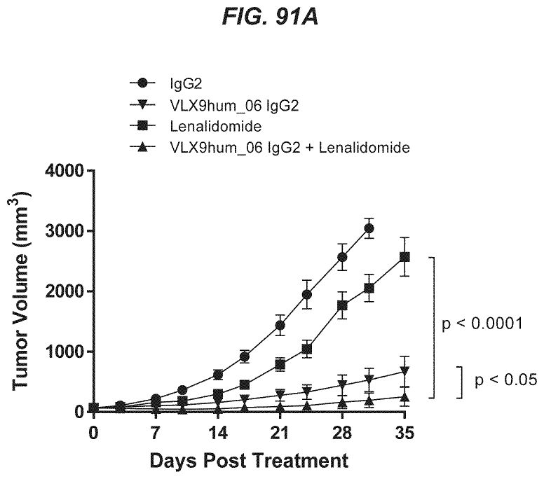

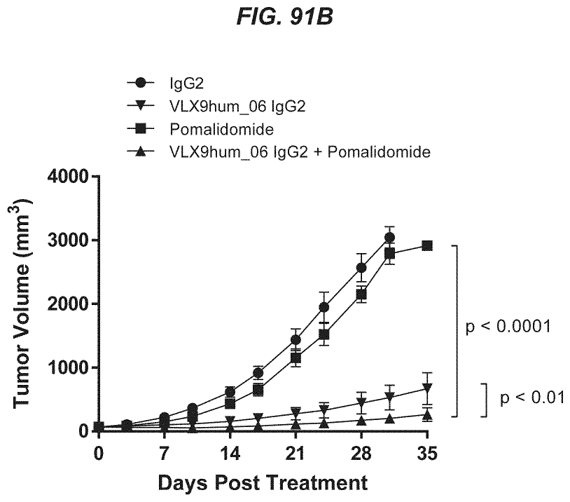

As shown in FIG. 91A, the primary efficacy assessment using tumor volume based on tumor growth inhibition (TGI) at day 24 resulted in statistically significant anti-tumor activity in all groups when compared to the hIgG2 vehicle control group with both 25 mg/kg VLX9hum_06 IgG2 (86% TGI) or 25 mg/kg lenalidomide (48% TGI), demonstrating single agent anti-tumor efficacy in vivo. Combination treatment resulted in statistically significant decreases in tumor volumes when compared to single agent treatment with VLX9hum_06 IgG2 (p<0.05, 2-way ANOVA) or lenalidomide (p<0.0001, 2-way ANOVA). Increased anti-tumor efficacy was observed in the combination group, resulting in complete tumor regression in 5/9 of mice compared to complete tumor regression in 2/9 mice in the VLX9hum_06 IgG2 alone group and no tumor regressions observed in the lenalidomide single agent group. As shown in FIG. 91B, primary efficacy assessment based on TGI resulted in statistically significant anti-tumor activity of 25 mg/kg VLX9hum_06 IgG2 (86% TGI) when compared to the hIgG2 vehicle control group, whereas 10 mg/kg pomalidomide treatment alone (23% TGI) did not show significant TGI. Combination treatment resulted in statistically significant decreases in tumor volumes when compared to single agent treatment with VLX9hum_06 IgG2 (p<0.01, 2-way ANOVA) or pomalidomide (p<0.0001, 2-way ANOVA). Increased anti-tumor efficacy was observed in the combination group, which resulted in complete tumor regression of 3/9 of mice compared to 2/9 mice in the VLX9hum_06 IgG2 alone group and no tumor regressions observed in the pomalidomide group.

### Example 39

### Anti-Tumor Activity of Anti-CD47 Antibodies Combined with Immunomodulating Drugs and Dexamethasone in a Xenograft Mouse Model of Multiple Myeloma

This disclosure demonstrates the anti-tumor properties of a humanized anti-CD47 antibody (VLX9hum_06 IgG2) in combination with lenalidomide or pomalidomide and dexamethasone, in reducing tumor burden in a xenograft multiple myeloma NOD-SCID mouse model. These data illustrate that the addition of dexamethasone to regimens consisting of the anti-CD47 antibody and immunomodulating drugs does not compromise the combinatorial anti-tumor activity of these two agents.

MM.11S human multiple myeloma cells (ATCC #CRL-2974, Manassas, Va.) were maintained in RPMI-1640 (Lonza; Walkersville, Md.) supplemented with 10% Fetal Bovine Serum (FBS; Omega Scientific; Tarzana, Calif.) and 1% Penicillin/Streptomycin (Corning, Manassas, Va.) within a 5% CO2 atmosphere. Cultures were expanded in tissue culture flasks.

Female NOD-SCID (NOD.CB17-Prkdc^{scid}/J) mice were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NOD-SCID mice were inoculated subcutaneously in the right flank with 0.1 mL of a 50% RPMI-1640/50% Matrigel™ (BD Biosciences; Bedford, Mass.) mixture containing a suspension of 5×10⁶ MM.1S tumor cells. When tumors reached volumes of 50-100 mm³, mice were randomized. The test articles human IgG2 (hIgG2), anti-CD47 mAb (VLX9hum_06 IgG2), and dexamethasone (Dex) were administered by intraperitoneal (IP) injection and lenalidomide (Len) or pomalidomide (Pom) was administered by oral gavage (PO). hIgG2 (25 mg/kg) or an anti-CD47 mAb VLX9hum_06 IgG2 (25 mg/kg) were dosed weekly for 5 weeks, while lenalidomide (25 mg/kg), pomalidomide (10 mg/kg), and dexamethasone (0.3 mg/kg) were dosed on 4 successive days, with 3 days off, weekly for 5 weeks.

Mean tumor growth inhibition (TGI) was calculated at Day 24 (the final day all mice were on study) utilizing the following formula. Of note, mice exhibiting tumor shrinkage were excluded from the TGI calculations.

$$TGI = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

All statistical analyses in the xenograft study were performed with Prism GraphPad® software. Differences in Day 24 tumor volumes were confirmed using a two-way ANOVA, unpaired, parametric with the Tukey's Multiple Comparison test with Welch's correction.

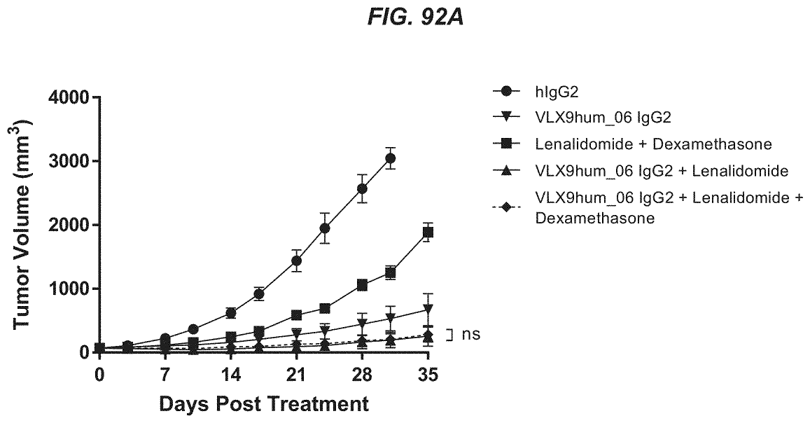

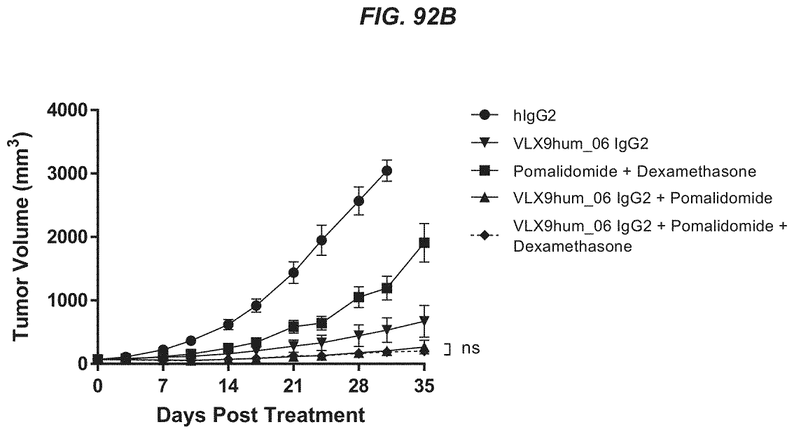

As shown in FIG. 92A, the primary efficacy assessment based on tumor growth inhibition (TGI) at day 24 resulted in statistically significant anti-tumor activity in all groups when compared to the hIgG2 vehicle control group with 25 mg/kg VLX9hum_06 IgG2 (86% TGI, p<0.0001, 2-way ANOVA), 25 mg/kg lenalidomide plus 0.3 mg/kg dexamethasone (67% TGI, p<0.0001, 2-way ANOVA), VLX9hum_06 IgG2 plus lenalidomide (98% TGI, p<0.0001, 2-way ANOVA) and VLX9hum_06 IgG2 plus lenalidomide plus dexamethasone (96% TGI, p<0.0001, 2-way ANOVA) all demonstrating anti-tumor efficacy in vivo. Complete tumor regressions were observed in 4/9 mice in the VLX9hum_06 IgG2 plus lenalidomide plus dexamethasone treatment group, compared to 4/9 in the VLX9hum_06 IgG2 plus lenalidomide group, 2/9 mice in the VLX9hum_06 IgG2 alone treatment group and 0/9 in the lenalidomide plus dexamethasone group. There was no statistical difference between the TGI percentages between the VLX9hum_06 IgG2 plus lenalidomide treatment group and the VLX9hum_06 IgG2 plus lenalidomide plus dexamethasone treatment groups, indicating dexamethasone can be added to VLX9hum_06 IgG2 in combination with lenalidomide with no detrimental impact on anti-tumor activity in vivo. As shown in FIG. 92B, primary efficacy assessment based on tumor growth inhibition (TGI) resulted in statistically significant anti-tumor activity in all groups when compared to the hIgG2 vehicle control group with 25 mg/kg VLX9hum_06 IgG2 (86% TGI, p<0.0001, 2-way ANOVA), 10 mg/kg pomalidomide plus 0.3 mg/kg dexamethasone (69% TGI, p<0.0001, 2-way ANOVA), VLX9hum_06 IgG2 plus pomalidomide (97% TGI, p<0.0001, 2-way ANOVA) and VLX9hum_06 IgG2 plus lenalidomide plus dexamethasone (96% TGI, p<0.0001, 2-way ANOVA) all demonstrating anti-tumor efficacy in vivo. Complete tumor regressions were observed in 4/9 mice in the VLX9hum_06 IgG2 plus pomalidomide plus dexamethasone treatment group, compared to 3/9 mice in the VLX9hum_06 IgG2 plus pomalidomide group, 2/9 mice in the VLX9hum_06 IgG2 alone treatment group and 1/9 mice in the lenalidomide plus dexamethasone group. There was no statistical difference between the TGI percentages between the VLX9hum_06 IgG2 plus pomalidomide treatment group and the VLX9hum_06 IgG2 plus pomalidomide plus dexamethasone treatment groups, indicating dexamethasone can be added to VLX9hum_06 IgG2 in combination with pomalidomide with no detrimental impact on anti-tumor activity in vivo.

### Example 40

### Treatment with Anti-CD47 Antibodies Induce Accumulation of CD68⁺ and CD11c⁺ Cells in a Xenograft Mouse Model of Multiple Myeloma

This disclosure demonstrates the properties of a humanized anti-CD47 antibody (VLX9hum_06 IgG2) to induce accumulation of macrophage (CD68⁺ staining) and dendritic cells (CD11c⁺ staining) in the tumor periphery in a xenograft multiple myeloma NOD-SCID mouse model.

RPMI-8226 or NCI-H929 human multiple myeloma cells (ATCC #CCL-155 and CRL-9068 respectively, Manassas, Va.) were maintained in RPMI-1640 (Lonza; Walkersville, Md.) supplemented with 10% Fetal Bovine Serum (FBS; Omega Scientific; Tarzana, Calif.) and 1% Penicillin/Streptomycin (Corning, Manassas, Va.) within a 5% CO2 atmosphere. Cultures were expanded in tissue culture flasks.

Female NOD-SCID (NOD.CB17-Prkdc^{scid}/J) mice were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NOD-SCID mice were inoculated subcutaneously in the right flank with 0.1 mL of a 70% RPMI-1640/30% Matrigel™ (BD Biosciences; Bedford, Mass.) mixture containing a suspension of 1×10⁷ tumor cells. When tumors reached volumes of approximately 100 mm³, mice were randomized. The control human IgG2 (hIgG2, 25 mg/kg) or an anti-CD47 mAb (VLX9hum_06 IgG2, 25 mg/kg) were administered by intraperitoneal (IP) injection. At 96 hours after administration, tumors from (n=3/group) mice were harvested, fixed in 10% neutral buffered formalin for 24 hours and then stored in 70% ethanol until immunohistochemical staining was performed.

Prior to staining with primary antibodies, heat-induced epitope retrieval was performed at pH 6.2 in a Biocare Decloaking Chamber at 110° C. for 15 min and then cooled at 90° C. for 10 min. The primary antibodies, either rabbit anti-mouse CD11c (clone D1V9Y, Cell Signaling 97585; Danvers, Mass.) or rabbit anti-mouse CD68 (Abcam ab125212; Cambridge, Mass.). were diluted 1:1000 and 1:350, respectively, and incubated for 45 minutes at room temperature. Localization of the primary antibodies was detected with HRP-polymer using a Biocare MACH4 HRP-Polymer Detection System.

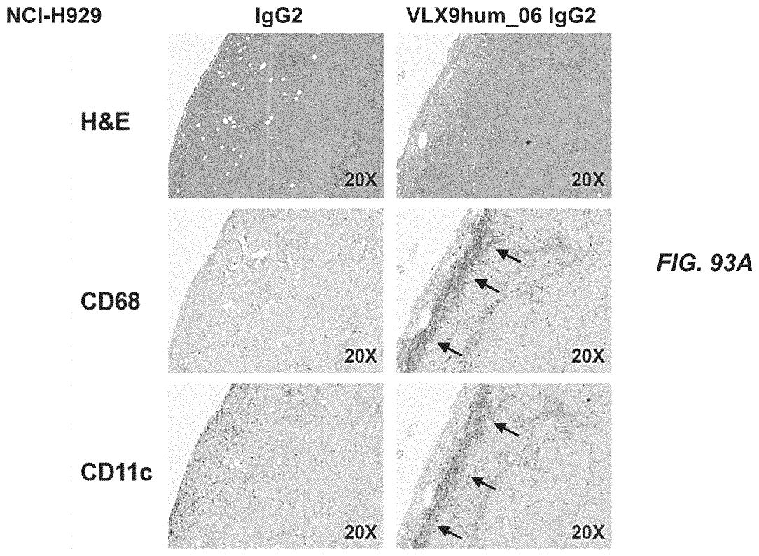

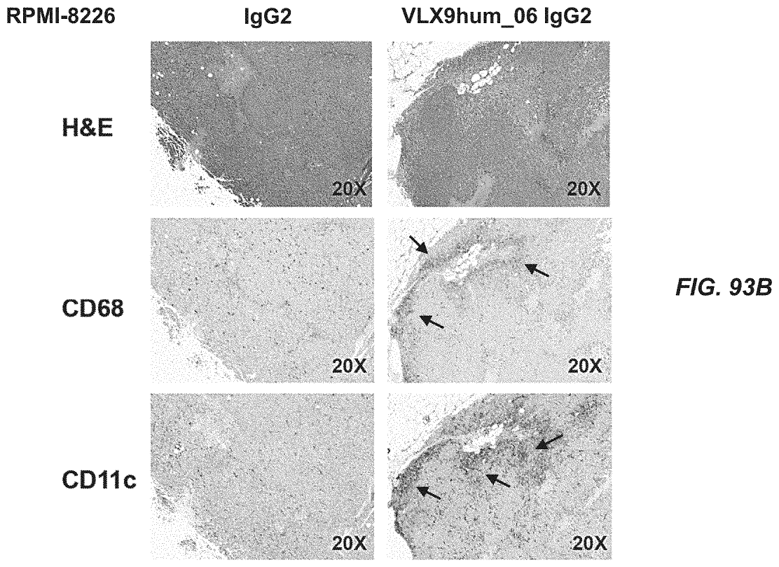

As shown in FIG. 93A, staining for CD68 and CD11c showed an accumulation of positive cells (arrows) on the periphery of RPMI-8226 tumors at 96 hours following treatment with anti-CD47 mAb (VLX9hum_06 IgG2), compared to minimal peripheral accumulation of cells in hIgG2-treated tumors. As shown in FIG. 93B, staining for CD68 and CD11c showed an accumulation of positive cells (arrows) on the periphery of NCI-H929 tumors at 96 hours following treatment with anti-CD47 mAb (VLX9hum_06 IgG2), compared to minimal peripheral accumulation of cells in hIgG2-treated tumors. Representative images are shown for each stain.

### Example 41

### Anti-Tumor Activity of Anti-CD47 Antibodies at Multiple Doses in a Human Multiple Myeloma Xenograft Model

The anti-tumor properties of various doses of a humanized anti-CD47 antibody (VLX9hum_06 IgG2) in reducing tumor burden were evaluated in a xenograft multiple myeloma NOD-SCID mouse model.

NCI-H929 human multiple myeloma cells (ATCC #CRL-9068, Manassas, Va.) were maintained in RPMI-1640 (Lonza; Walkersville, Md.) supplemented with 10% Fetal Bovine Serum (FBS; Omega Scientific; Tarzana, Calif.) and 1% Penicillin/Streptomycin (Corning, Manassas, Va.) within a 5% CO2 atmosphere. Cultures were expanded in tissue culture flasks.

Female NOD-SCID (NOD.CB17-Prkdc^{scid}/J) were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NOD-SCID mice (n=6/group) were inoculated subcutaneously in the right flank with 0.1 mL of a 50% RPMI-1640/50% Matrigel™ (BD Biosciences; Bedford, Mass.) mixture containing a suspension of 10×10⁶ NCI-H929 tumor cells. When tumors reached volumes of approximately 75-125 mm³, mice were randomized. The test articles human IgG2 (hIgG2) or anti-CD47 mAb (VLX9hum_06 IgG2) were administered by intraperitoneal (IP) injection. hIgG2 (25 mg/kg) or an anti-CD47 mAb VLX9hum_06 IgG2 (1, 3, 10, or 25 mg/kg) were dosed weekly for 13 weeks

Mean tumor growth inhibition (TGI) was calculated at Day 16 (the final day all mice were on study) utilizing the following formula. Of note, mice exhibiting tumor shrinkage were excluded from the TGI calculations.

$$TGI = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

All statistical analyses in the xenograft study were performed with Prism GraphPad® software. Differences in Day 20 tumor volumes were confirmed using a two-way ANOVA, unpaired, parametric with the Tukey's Multiple Comparison test with Welch's correction.

Increase in survival fractions were confirmed by the log rank test with a comparison of each group to the vehicle control group.

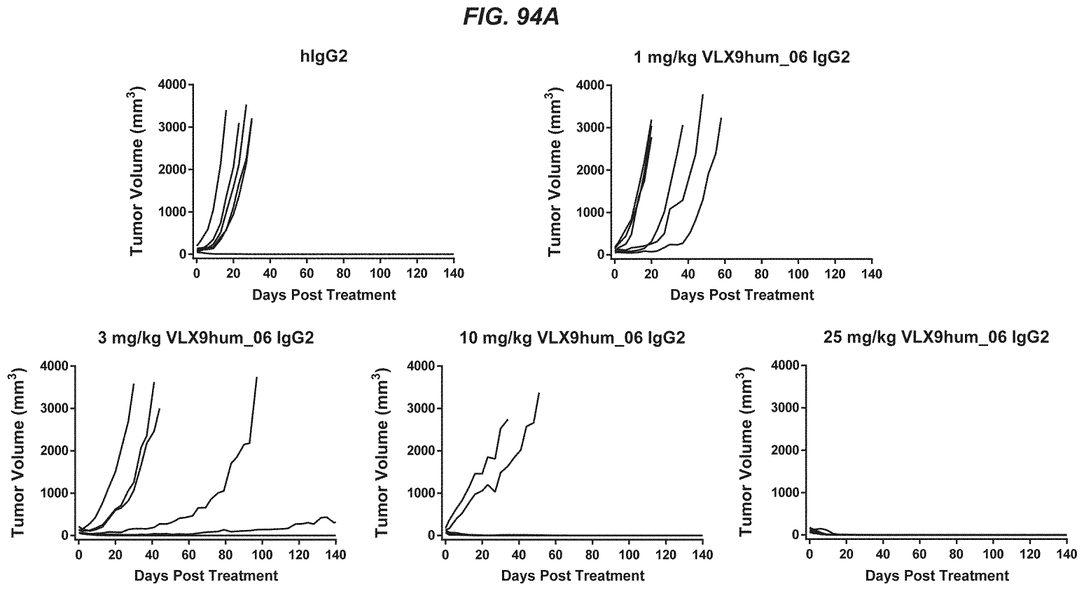

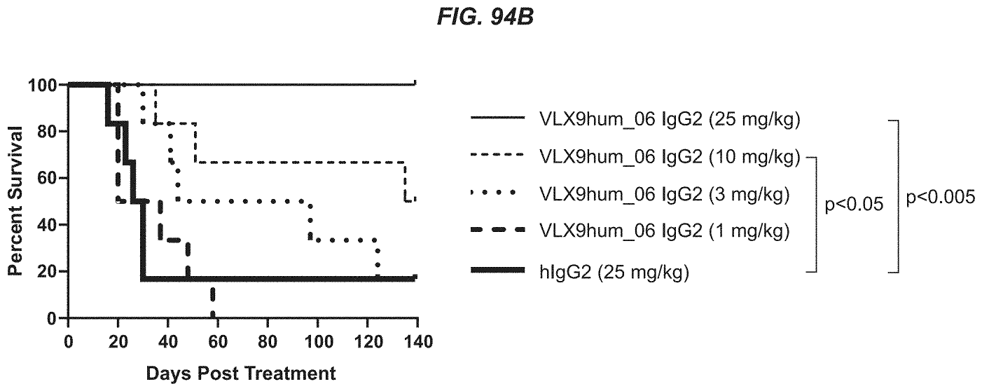

The primary assessment of efficacy was based on both tumor volume and the number of complete tumor regressions (CR). The primary efficacy assessment based on tumor growth inhibition (TGI) at day 16 post-treatment resulted in statistically significant anti-tumor activity at 3 out of 4 doses when compared to the hIgG2 vehicle control group, with 3 mg/kg VLX9hum_06 IgG2 (74% TGI, p<0.0066, 2-way ANOVA), 10 mg/kg VLX9hum_06 IgG2 (66% TGI, p<0.0183, 2-way ANOVA) and 25 mg/kg VLX9hum_06 IgG2 (99% TGI, p<0.0001, 2-way ANOVA) demonstrating single agent anti-tumor efficacy in vivo (FIG. 94A). By day 140, VLX9hum_06 IgG2 at 3 mg/kg showed 1/6 CR, VLX9hum_06 IgG2 at 10 mg/kg showed 4/6 CR, and VLX9hum_06 IgG2 at 25 mg/kg showed 6/6 CR, demonstrating that CRs can be achieved at doses of VLX9hum_06 IgG2 below 25 mg/kg.

### Secondary Assessment of Efficacy Based on Survival

The secondary assessment of efficacy was assessed by increased survival up to 140 days in treatment groups compared to the hIgG2 vehicle control (FIG. 94B). Doses of VLX9hum_06 IgG2 at 10 mg/kg and 25 mg/kg resulted in a statistically significant increase in survival when compared to the hIgG2 vehicle control group (p<0.0235 and p<0.005 respectively, Log-rank test).

### Example 42

### Anti-Tumor Activity of Anti-CD47 Antibodies in a Human Multiple Myeloma Xenograft Model with Advanced Tumor Burden

The anti-tumor properties of a humanized anti-CD47 antibody (VLX9hum_06 IgG2) in reducing tumor burden were evaluated in a xenograft multiple myeloma NOD-SCID mouse model of advanced disease. NCI-H929 human multiple myeloma cells (ATCC #CRL-9068, Manassas, Va.) were maintained in RPMI-1640 (Lonza; Walkersville, Md.) supplemented with 10% Fetal Bovine Serum (FBS; Omega Scientific; Tarzana, Calif.) and 1% Penicillin/Streptomycin (Corning, Manassas, Va.) within a 5% CO2 atmosphere. Cultures were expanded in tissue culture flasks.

Female NOD-SCID (NOD.CB17-Prkdc^{scid}/J) were obtained from Jackson Laboratory (Bar Harbor, Me.) at 5-6 weeks of age. Mice were acclimated prior to handling and housed in microisolator cages (Lab Products, Seaford, Del.) under specific pathogen-free conditions. Mice were fed Teklad Global Diet® 2920x irradiated laboratory animal diet (Envigo, Formerly Harlan; Indianapolis, Ind.) and provided autoclaved water ad libitum. All procedures were carried out under Institutional Animal Care and Use guidelines.

Female NOD-SCID mice (n=6/group) were inoculated subcutaneously in the right flank with 0.1 mL of a 50% RPMI-1640/50% Matrigel™ (BD Biosciences; Bedford, Mass.) mixture containing a suspension of 10×10⁶ NCI-H929 tumor cells. When tumors reached larger volumes of approximately 200-1600 mm³ (increased from typical tumor volumes of 50-100 mm³) the mice were randomized, and treatment initiated. The test articles human IgG2 (hIgG2) or anti-CD47 mAb (VLX9hum_06 IgG2) were administered by intraperitoneal (IP) injection. hIgG2 (25 mg/kg) or an anti-CD47 mAb VLX9hum_06 IgG2 (25 mg/kg) were administered once weekly for 7 weeks.

Mean tumor growth inhibition (TGI) was calculated at Day 17 and 21 (the final day all mice were on study) utilizing the following formula.

$$TGI = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

All statistical analyses in the xenograft study were performed with Prism GraphPad® software.

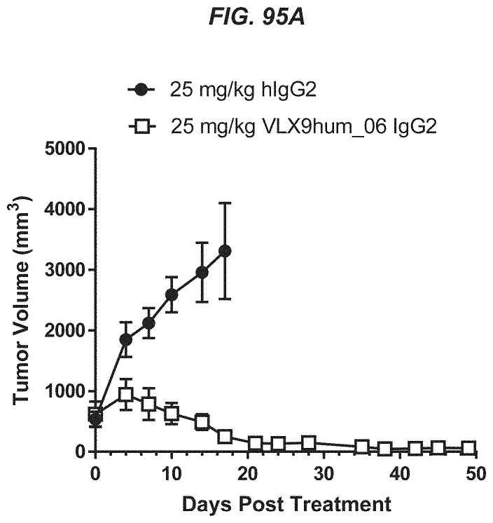

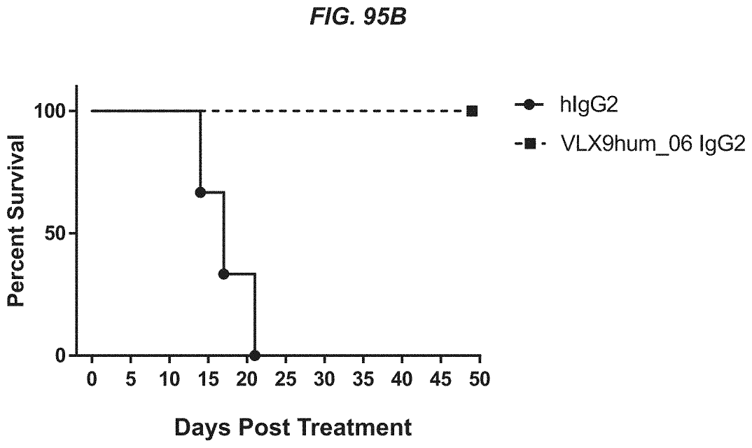

The primary assessment of efficacy was based on both tumor volume and the number of complete tumor regressions (CR). Tumor growth inhibition (TGI) at day 17 post-treatment resulted in statistically significant anti-tumor activity in the 25 mg/kg VLX9hum_06 IgG2 (92% TGI, p<0.0001, 2-way ANOVA) compared to the 25 mg/kg hIgG2 group (FIG. 95A). By the end of study at day 49, the VLX9hum_06 IgG2 treatment group at 25 mg/kg showed 4/6 CR, with the remaining tumor-bearing mice in the VLX9hum_06 IgG2 treatment group showing very small tumor volumes of 8 mm³ and 352 mm³. In contrast, in the 25 mg/kg hIgG2 treatment group there was significant tumor growth with 0/6 CR and all mice dead by day 18. This demonstrates that VLXhum_06 IgG2 is capable of achieving significant tumor growth inhibition and CRs in MM models with advanced disease burden.

### Secondary Assessment of Efficacy Based on Survival

The secondary assessment of efficacy was assessed by increased survival in the treatment group compared to the hIgG2 vehicle control (FIG. 95B). Treatment with 25 mg/kg of VLX9hum_06 IgG2, once weekly, resulted in a statistically significant increase in survival when compared to the hIgG2 vehicle control group (p<0.0007, Log-rank test) with 100% of the anti-CD47 antibody treated animals alive at the end of the 49 day study.

### Example 43

### Anti-CD47 mAbs Increase Phagocytosis when Combined with 5-Azacitidine or Venetoclax

To assess the effect of the anti-CD47 mAbs in combination with 5-azacitidine or venetoclax on phagocytosis of tumor cells by human monocyte-derived macrophages the following in vitro method was employed using flow cytometry.

Human monocyte derived macrophages (MDMs) were differentiated from CD14 monocytes. CD14 monocytes were purchased from Astarte Biologics. After thawing, they were seeded onto 96-well flat bottom plates at 5×10⁴ cells/well and differentiated into MDMs in vitro for seven days in AIM-V medium (ThermoFisher, 12055091) supplemented with 10% FBS (BioWest, S01520) and 50 ng/ml M-CSF (Biolegend, 574802).

The day before the in vitro phagocytosis assays were run human acute myeloid leukemia (AML) cancer cell lines HL-60, MV4-11, or KG-1 were labeled with 1 μM 5(6)-Carboxyfluorescein diacetate N-succinimidyl ester (CFSE) according to the manufacturer's protocol (ThermoFisher, C1157) and were treated with 5-azacitidine (Selleckchem, S1782) or venetoclax (Selleckchem, S8048) at concentrations specific to each cell line. Prior to setting up in vitro phagocytosis on the day of the assays, human MDMs were cultured in AIM-V medium without supplements for 2 h. The treated AML cells were washed and added to the macrophage cultures in 96-well plates at a concentration of 8×10⁴ cells/well in AIM-V medium without supplements. VLX9hum_06 IgG2 (3 or 10 μg/mL) or 10 μg/mL of the IgG2 isotype control (Bioxcell) were added immediately upon mixture of either treated or untreated target and effector cells and allowed to incubate at 37° C. for 4 h. After 4 hours, all non-phagocytosed cells were removed, and the remaining cells washed three times with PBS. Cells were then incubated in Accutase (Innovative Cell Technologies, AT-104) to detach macrophages, collected into 96-well V-bottom plates, and incubated in 100 ng of CD14 monoclonal antibody (TuK4), allophycocyanin (APC) labeled (ThermoFisher, MHCD1405), for 30 minutes, washed once, and analyzed by flow cytometry (Attune, Life Technologies) for the percentage of CD14 cells that were also CFSE⁺ indicating complete phagocytosis.

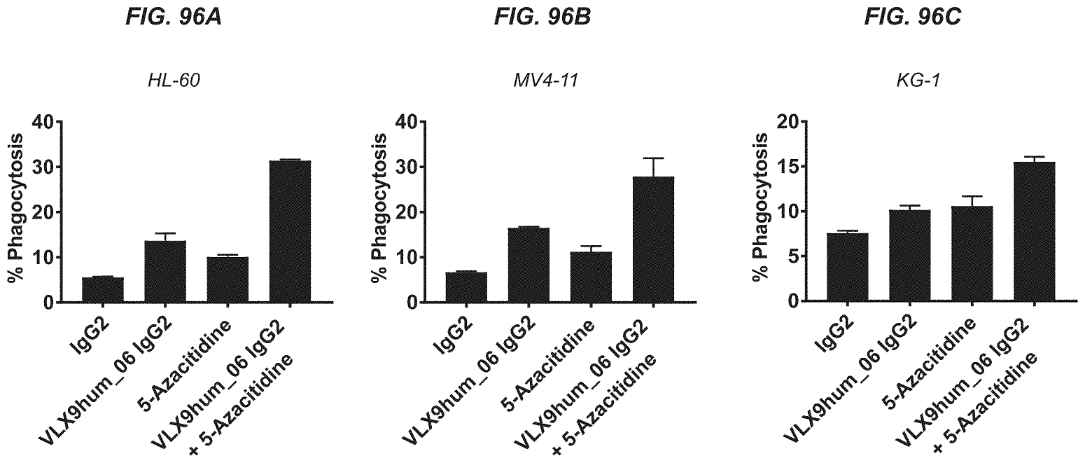

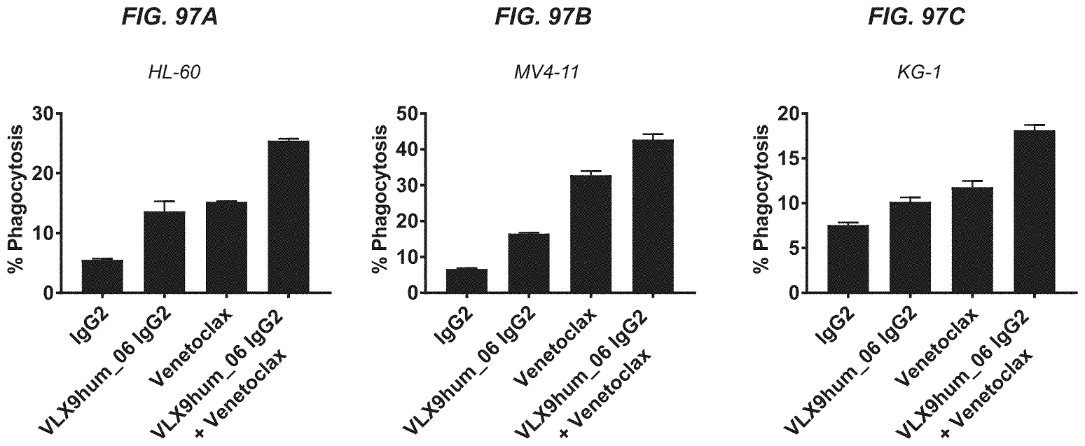

As shown in FIG. 96A-FIG. 96C, phagocytosis by VLX9hum_06 IgG2 in combination with 5-azacitidine was determined by pre-treating HL-60 cells with 3 μM 5-azacitidine for 24h at 37° C. followed by co-culture with human MDMs and treatment with 3 μg/mL VLX9hum_06 IgG2 for 4h at 37° C. Alternatively, MV4-11 cells were treated with 0.63 μM 5-azacitidine and 10 μg/mL VLX9hum_06 IgG2, and KG-1 cells were treated with 0.63 μM 5-azacitidine and 3 μg/mL VLX9hum_06 IgG2. All three cell lines were also treated with 10 μg/mL of the IgG2 isotype control. Phagocytosis of the acute myeloid leukemia cells in the combinations was increased to a greater degree than either agent alone. Similarly, as shown in FIG. 97A-FIG. 97C, phagocytosis by VLX9hum_06 IgG2 in combination with venetoclax was determined by pre-treating HL-60 cells with 3 nM venetoclax for 24h at 37° C. followed by co-culture with human MDMs and treatment with 3 μg/mL VLX9hum_06 IgG2 for 4h at 37° C. Alternatively, MV4-11 cells were treated with 10 nM venetoclax and 10 μg/mL VLX9hum_06 IgG2, and KG-1 cells were treated with 0.5 μM venetoclax and 3 μg/mL VLX9hum_06 IgG2. All three cell lines were also treated with 10 μg/mL of the IgG2 isotype control. Phagocytosis of the acute myeloid leukemia cells in the combinations was increased to a greater degree than either agent alone.

### Example 44

### Anti-CD47 mAbs Enhances Cell Killing in Combination with Azacitidine and Venetoclax

To assess the effect of the anti-CD47 mAbs in combination with azacitidine or venetoclax on inducing killing human acute myeloid leukemia tumor cells the following in vitro method was employed using flow cytometry.

Human acute myeloid leukemia (AML) cell lines HL-60, MV4-11, or KG-1 at 4×10⁴ per well were treated with VLX9hum_06 IgG2 alone or in combination with either 5-azacitidine (Selleckchem, S1782) or venetoclax (Selleckchem, S8048) in complete media containing 10% (v/v) heat inactivated fetal bovine serum (BioWest, Catalog #S01520), 100 units/mL penicillin, and 100 μg/mL streptomycin (Sigma, #P4222). The cells were incubated at 37° C. and 5% CO₂ for 18-24 h. Cell autonomous killing of AML tumor cells following treatment was assessed by analysis of cell surface phosphatidylserine exposure and a DNA intercalating dye to assess viability. The treated AML cells were transferred into 96-well V-bottom plates and washed once with Annexin V binding buffer (BioLegend, 422201). The cells were then stained with PE-labelled Annexin V (BD Biosciences, 556421) for 20 mins followed by a wash in Annexin V binding buffer. Next, the cells were resuspended in SYTOX Blue Dead Cell Stain (ThermoFisher, S34857) in Annexin V binding buffer to assess viability and analyzed by flow cytometry (Attune, Life Technologies) for the percentage of either Annexin V+/Sytox−, Annexin V+/Sytox+, or Total Annexin V+ cells.

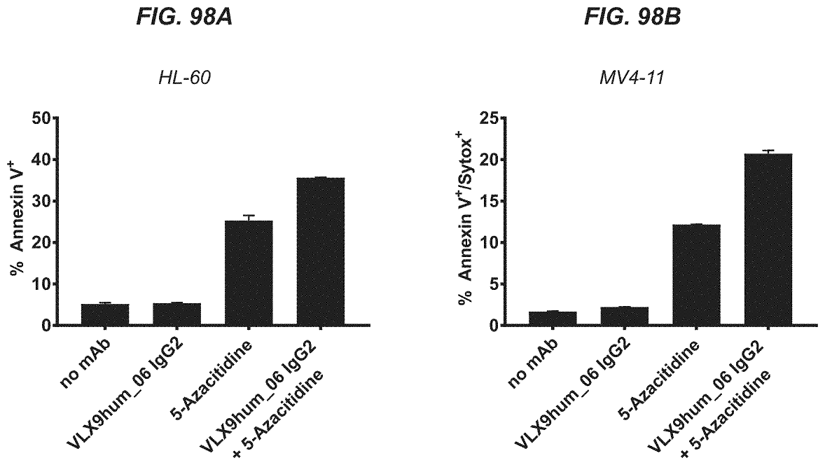

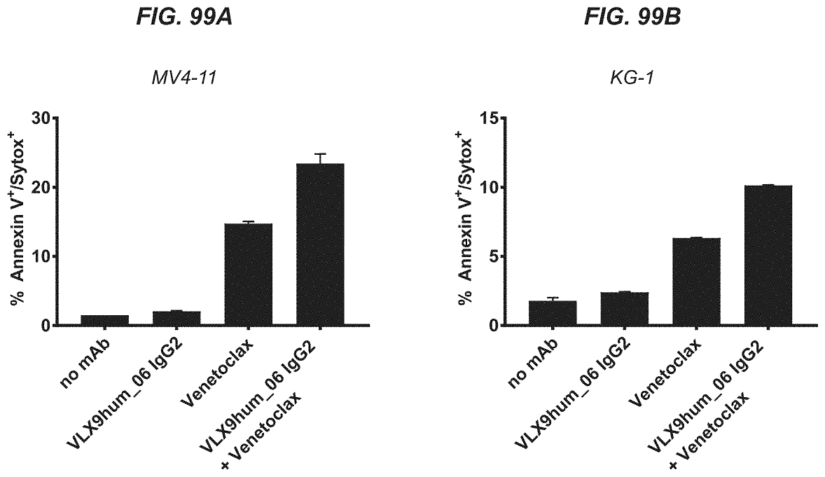

As shown in FIG. 98A-FIG. 98B, cell autonomous death by anti-CD47 mAbs in combination with 5-azacitidine was determined by treating HL-60 or MV4-11 cells with 100 μg/mL VLX9hum_06 IgG2 alone, 5 μM 5-azacitidine alone, or VLX9hum_06 IgG2 and 5-azacitidine for 24 hours at 37° C. Cells were stained with Annexin V to measure externalization of phosphatidylserine (Annexin V+), as well as SYTOX Blue to assess viability and measured by flow cytometry. Comparison of combination treatment to single-agent treatment resulted in increases in the percent (%) Annexin V+ cells in HL-60 or in the percent (%) Annexin V+/SYTOX+ cells in MV4-11. Similarly, as shown in FIG. 99A-FIG. 99B, cell autonomous death by anti-CD47 mAbs in combination with venetoclax was determined by treating MV4-11 cell with 100 μg/mL VLX9hum_06 IgG2 alone, 0.3 μM venetoclax alone, or VLX9hum_06 IgG2 and venetoclax or KG-1 cells with 100 μg/mL VLX9hum_06 IgG2 alone, 2.5lM venetoclax alone, or VLX9hum_06 IgG2 and venetoclax for 24 hours at 37° C. Cells were stained with Annexin V to measure externalization of phosphatidylserine (Annexin V+), as well as SYTOX Blue to assess viability and measured by flow cytometry. Comparison of combination treatment to single-agent treatment resulted in increases in the percent (%) Annexin V+/SYTOX+ cells in MV4-11 and KG-1.

### Example 45

### Anti-CD47 mAbs Enhances DAMP Induction Alone and in Combination with 5-Azacitidine

To assess the effect of the anti-CD47 antibody in combination with 5-azacitidine on increasing surface exposure of DAMPs on tumor cells the following in vitro method was employed using flow cytometry.

The human acute myeloid leukemia (AML) cell line, HL-60, was treated with VLX9hum_06 IgG2 (10, 30, or 100 μg/mL) alone or in combination with 5-azacitidine (5 μM) (Selleckchem, S1782) and incubated at 37° C. for 18-24 h. The treated AML cells were transferred into 96-well V-bottom plates and washed once with PBS/2% FBS. After blocking Fc receptors with Human TruStain FcX (BioLegend, 422302), the cells were then stained with mouse anti-human calreticulin monoclonal antibody (FMC 75), DyLight 488-labeled (Enzo Life Sciences, ADI-SPA-601-488-F) and mouse anti-human PDIA3/ERp57 monoclonal antibody (Map.ERp57 (GRP58)), Alexa Fluor 647-labeled (Novus, NBP2-59689AF647) for 20 mins followed by a wash in PBS/2% FBS staining buffer. Next, the cells were resuspended in SYTOX Blue Dead Cell Stain (ThermoFisher, S34857) in PBS/2% FBS buffer and analyzed by flow cytometry (Attune, Life Technologies) for the percentage of either calreticulin (CalR)+/Sytox− or PDIA3+/Sytox− cells.

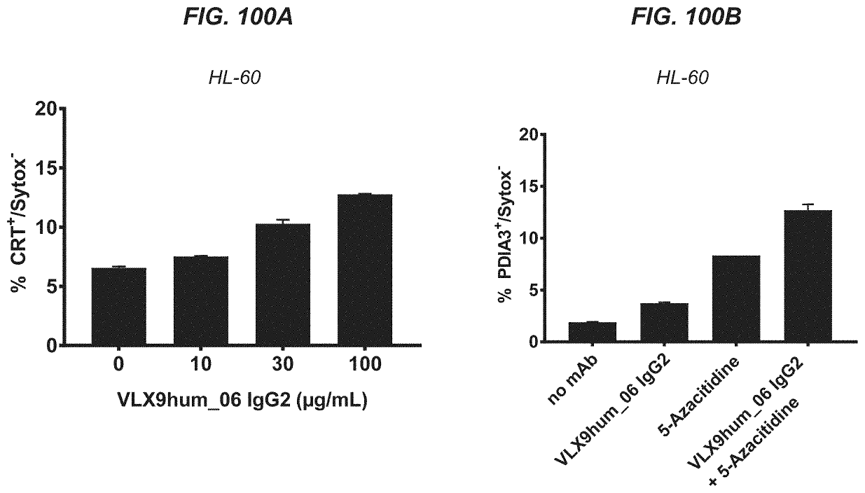

As shown in FIG. 100A, VLX9hum_06 IgG2 induced an increase in cell surface calreticulin exposure alone in a concentration dependent manner on HL-60 cells. As shown in FIG. 100B, VLX9hum_06 IgG2 induced an increase in cell surface PDIA3 exposure alone and in combination with 5-azacitidine on HL-60 cells.

What is claimed:

1. A method of treating a cancer comprising administering an effective amount of a monoclonal antibody or antigen-binding fragment thereof to a subject that specifically binds CD47 and comprises:
a variable heavy chain CDR1 amino acid sequence (HCDR1) amino acid sequence set forth in SEQ ID NO:3;
a variable heavy chain CDR2 amino acid sequence (HCDR2) amino acid sequence set forth in SEQ ID NO:6;
a variable heavy chain CDR3 amino acid sequence (HCDR3) amino acid sequence set forth in SEQ ID NO:10;
a variable light chain CDR1 amino acid sequence (LCDR1) amino acid sequence set forth in SEQ ID NO:14;
a variable light chain CDR2 amino acid sequence (LCDR2) amino acid sequence set forth in SEQ ID NO:17;
a variable light chain CDR3 amino acid sequence (LCDR3) amino acid sequence set forth in SEQ ID NO:18, and
a second anti-cancer agent which results in increased immunogenic cell death (ICD) of tumor cells and/or cell death of tumor cells in the subject as compared to the administration of a monoclonal antibody or antigen-binding fragment thereof that specifically binds CD47 alone.

2. The method of claim 1, wherein the monoclonal antibody or antigen-binding fragment thereof, further comprises a heavy chain variable domain (V_{H}) and a light chain variable domain (V_{L}), selected from:
(i) a heavy chain variable domain comprising the amino acid sequence of SEQ ID NO:36 and a light chain variable domain comprising the amino acid sequence SEQ ID NO:51;
(ii) a heavy chain variable domain comprising the amino acid sequence of SEQ ID NO:36 and a light chain variable domain comprising the amino acid sequence SEQ ID NO: 52;
(iii) a heavy chain variable domain comprising the amino acid sequence of SEQ ID NO: 38 and a light chain variable domain comprising the amino acid sequence SEQ ID NO:51;
(iv) a heavy chain variable domain comprising the amino acid sequence SEQ ID NO:38 and a light chain variable domain comprising the amino acid sequence SEQ ID NO:52;
(v) a heavy chain variable domain comprising the amino acid sequence of SEQ ID NO:39 and a light chain variable domain comprising the amino acid sequence SEQ ID NO:51; and
(vi) a heavy chain variable domain comprising the amino acid sequence of SEQ ID NO:39 and a light chain variable domain comprising the amino acid sequence SEQ ID NO:52.

3. The method of claim 2, wherein the monoclonal antibody or antigen-binding fragment thereof further comprises an IgG isotype selected from IgG1, IgG1-N297Q, IgG2, IgG4, IgG4 S228P, and IgG4 PE.

4. The method of claim 3, wherein the monoclonal antibody or antigen fragment thereof further comprises one heavy chain and one light chain selected from:
(i) a heavy chain comprising the amino acid sequence of SEQ ID NO: 81 and a light chain comprising the amino acid sequence SEQ ID NO:71;
(ii) a heavy chain comprising the amino acid sequence of SEQ ID NO: 81 and a light chain comprising the amino acid sequence SEQ ID NO:74;
(iii) a heavy chain comprising the amino acid sequence of SEQ ID NO: 82 and a light chain comprising the amino acid sequence SEQ ID NO:71;
(iv) a heavy chain comprising the amino acid sequence of SEQ ID NO: 82 and a light chain comprising the amino acid sequence SEQ ID NO:74;
(v) a heavy chain comprising the amino acid sequence of SEQ ID NO: 83 and a light chain comprising the amino acid sequence SEQ ID NO:71; and
(vi) a heavy chain comprising the amino acid sequence of SEQ ID NO: 83 and a light chain comprising the amino acid sequence SEQ ID NO:74

5. The method of claim 4, wherein the monoclonal antibody or antigen binding fragment thereof comprises a heavy chain comprising the amino acid sequence of SEQ ID NO:82 and a light chain comprising the amino acid sequence SEQ ID NO:71.

6. The method of claim 1, wherein the immunogenic cell death (ICD) characteristics comprise:
a. an increase in adenosine triphosphate (ATP) release; and
b. an increase in cell surface calreticulin expression on human tumor cells.

7. The method of claim 1, wherein the second anti-cancer agent is a proteasome inhibitor.

8. The method of claim 7, wherein the proteasome inhibitor is chosen from bortezomib, carfilzomib, and ixazomib.

9. The method of claim 1, wherein the second anti-cancer agent is selinexor.

10. The method of claim 1, wherein the second anti-cancer agent is an immunomodulatory agent.

11. The method of claim 10, wherein the immunomodulatory agent is lenalidomide.

12. The method of claim 10, wherein the immunomodulatory agent is pomalidomide.

13. The method of claim 11, wherein lenalidomide is further administered in combination with dexamethasone.

14. The method of claim 12, wherein pomalidomide is further administered in combination with dexamethasone.

15. The method of claim 1, wherein the second anti-cancer agent is a Bruton's tyrosine kinase (BTK) inhibitor.

16. The method of claim 15, wherein the Bruton's tyrosine kinase (BTK) inhibitor is chosen from ibrutinib (PCI-32765), acalabrutinib, and zanubrutinib.

17. The method of claim 1, wherein the second anti-cancer agent is a BCMA-targeting agent.

18. The method of claim 17, wherein the BCMA-targeting agent is chosen from JNJ-4528, teclistamab (JNJ-7957) and belantamab mafodotin (GSK2857916).

19. The method of claim 1, wherein the second anti-cancer agent is a CAR-T cell.

20. The method of claim 19, wherein the CAR-T cell is chosen from an anti-CD19 CAR-T cell or an anti-BCMA CAR-T cell.

21. The method of claim 1, wherein the second anti-cancer agent is an inhibitor of the B-cell lymphoma-2 protein (BCL-2).

22. The method of claim 21, wherein the B-cell lymphoma-2 protein (BCL-2) inhibitor is venetoclax.

23. The method of claim 1, wherein the second anti-cancer agent is a chemotherapeutic agent.

24. The method of claim 23, wherein the chemotherapeutic agent can be chosen from anthracyclines, platinums, taxols, cyclophosphamides, topoisomerase inhibitors, anti-metabolites, anti-tumor antibiotics, mitotic inhibitors, alkylating agents, and demethylating agents.

25. The method of claim 24, wherein the chemotherapeutic agent is an anthracycline.

26. The method of claim 25, wherein the anthracycline is chosen from doxorubicin, epirubicin, daunorubicin, and idarubicin.

27. The method of claim 24, wherein the platinum is chosen from oxaliplatin, cisplatin, and carboplatin.

28. The method of claim 24, wherein the taxol is chosen from paclitaxel and docetaxel.

29. The method of claim 24, wherein the topoisomerase inhibitor is chosen from irinotecan, topotecan, etoposide, and mitoxantrone.

30. The method of claim 24, wherein the anti-metabolite is chosen from 5-FU, capecitabine, cytarabine, gemcitabine, and permetrexed.

31. The method of claim 24, wherein the anti-tumor antibiotic is chosen from daunorubicin, doxorubicin, epirubicin, idarubicin.

32. The method of claim 24, wherein the mitotic inhibitor is chosen from vinorelibine, vinblastine, and vincristine.

33. The method of claim 24, wherein the alkylating agent is temzolomide.

34. The method of claim 24, wherein the demethylating agent is 5-azacitidine.

35. A method of treating a cancer comprising administering an effective amount of a monoclonal antibody or antigen-binding fragment thereof to a subject that specifically binds CD47 and increases phagocytosis of human tumor cells and comprises:
a variable heavy chain CDR1 amino acid sequence (HCDR1) amino acid sequence set forth in SEQ ID NO:3;
a variable heavy chain CDR2 amino acid sequence (HCDR2) amino acid sequence set forth in SEQ ID NO:6;
a variable heavy chain CDR3 amino acid sequence (HCDR3) amino acid sequence set forth in SEQ ID NO:10;
a variable light chain CDR1 amino acid sequence (LCDR1) amino acid sequence set forth in SEQ ID NO:14;
a variable light chain CDR2 amino acid sequence (LCDR2) amino acid sequence set forth in SEQ ID NO:17;
a variable light chain CDR3 amino acid sequence (LCDR3) amino acid sequence set forth in SEQ ID NO:18,
in combination with a second antibody directed against a cellular target chosen from CD70 (Cluster of Differentiation 70), CD200 (OX-2 membrane glycoprotein, Cluster of Differentiation 200), CD154 (Cluster of Differentiation 154, CD40L, CD40 ligand, Cluster of Differentiation 40 ligand), CD223 (Lymphocyte-activation gene 3, LAG3, Cluster of Differentiation 223), KIR (Killer-cell immunoglobulin-like receptors), GITR (TNFRSF18, glucocorticoid-induced TNFR-related protein, activation-inducible TNFR family receptor, AITR, Tumor necrosis factor receptor superfamily member 18), CD20 (Cluster of Differentiation), CD28 (Cluster of Differentiation 28), CD40 (Cluster of Differentiation 40, Bp50, CDW40, TNFRSF5, Tumor necrosis factor receptor superfamily member 5, p50), CD86 (B7-2, Cluster of Differentiation 86), CD160 (Cluster of Differentiation 160, BY55, NK1, NK28), CD258 (LIGHT, Cluster of Differentiation 258, Tumor necrosis factor ligand superfamily member 14, TNFSF14, herpesvirus entry mediator ligand (HVEM-L), CD270 (HVEM, Tumor necrosis factor receptor superfamily member 14, herpesvirus entry mediator, Cluster of Differentiation 270, LIGHTR, HVEA), CD275 (ICOSL, ICOS ligand, Inducible T-cell co-stimulator ligand, Cluster of Differentiation 275), CD276 (B7-H3, B7 homolog 3, Cluster of Differentiation 276), OX40L (OX40 Ligand), B7-H4 (B7 homolog 4, VTCN1, V-set domain-containing T-cell activation inhibitor 1), GITRL (Glucocorticoid-induced tumor necrosis factor receptor-ligand, glucocorticoid-induced TNFR-ligand), 4-1BBL (4-1BB ligand), CD3 (Cluster of Differentiation 3, T3D), CD25 (IL2Rα, Cluster of Differentiation 25, Interleukin-2 Receptor α chain, IL-2 Receptor α chain), CD48 (Cluster of Differentiation 48, B-lymphocyte activation marker, BLAST-1, signaling lymphocytic activation molecule 2, SLAMF2), CD66a (Ceacam-1, Carcinoembryonic antigen-related cell adhesion molecule 1, biliary glycoprotein, BGP, BGP1, BGPI, Cluster of Differentiation 66a), CD80 (B7-1, Cluster of Differentiation 80), CD94 (Cluster of Differentiation 94), NKG2A (Natural killer group 2A, killer cell lectin-like receptor subfamily D member 1, KLRD1), CD96 (Cluster of Differentiation 96, TActILE, T-cell activation increased late expression), CD112 (PVRL2, nectin, Poliovirus receptor-related 2, herpesvirus entry mediator B, HVEB, nectin-2, Cluster of Differentiation 112), CD115 (CSF1R, Colony stimulating factor 1 receptor, macrophage colony-stimulating factor receptor, M-CSFR, Cluster of Differentiation 115), CD205 (DEC-205, LY75, Lymphocyte antigen 75, Cluster of Differentiation 205), CD226 (DNAM1, Cluster of Differentiation 226, DNAX Accessory Molecule-1, PTA1, platelet and T-cell activation antigen 1), CD244 (Cluster of Differentiation 244, Natural killer cell receptor 2B4), CD262 (DR5, TrailR2, TRAIL-R2, Tumor necrosis factor receptor superfamily member 10b, TNFRSF10B, Cluster of Differentiation 262, KILLER, TRICK2, TRICKB, ZTNFR9, TRICK2A, TRICK2B), CD284 (Toll-like Receptor-4, TLR4, Cluster of Differentiation 284), CD288 (Toll-like Receptor-8, TLR8, Cluster of Differentiation 288), Leukemia Inhibitor Factor (LIF), TNFSF15 (Tumor necrosis factor superfamily member 15, Vascular endothelial growth inhibitor, VEGI, TL1A), TDO2 (Tryptophan 2,3-dioxygenase, TPH2, TRPO), IGF-1R (Type 1 Insulin-like Growth Factor), GD2 (Disialoganglioside 2), TMIGD2 (Transmembrane and immunoglobulin domain-containing protein 2), RGMB (RGM domain family, member B), VISTA (V-domain immunoglobulin-containing suppressor of T-cell activation, B7-H5, B7 homolog 5), BTNL2 (Butyrophilin-like protein 2), Btn (Butyrophilin family), TIGIT (T-cell Immunoreceptor with Ig and ITIM domains, Vstm3, WUCAM), Siglecs (Sialic acid binding Ig-like lectins), i.e., SIGLEC-15, Neurophilin, VEGFR (Vascular endothelial growth factor receptor), ILT family (LIRs, immunoglobulin-like transcript family, leukocyte immunoglobulin-like receptors), NKG families (Natural killer group families, C-type lectin transmembrane receptors), MICA (MHC class I polypeptide-related sequence A), TGFβ (Transforming growth factor β), STING pathway (Stimulator of interferon gene pathway), Arginase (Arginine amidinase, canavanase, L-arginase, arginine transamidinase), EGFRvIII (Epidermal growth factor receptor variant III), and HHLA2 (B7-H7, B7y, HERV-H LTR-associating protein 2, B7 homolog 7), inhibitors of PD-1 (Programmed cell death protein 1, PD-1, CD279, Cluster of Differentiation 279), PD-L1 (B7-H1, B7 homolog 1, Programmed death-ligand 1, CD274, cluster of Differentiation 274), PD-L2 (B7-DC, Programmed cell death 1 ligand 2, PDCD1LG2, CD273, Cluster of Differentiation 273), CTLA-4 (Cytotoxic T-lymphocyte-associated protein 4, CD152, Cluster of Differentiation 152), BTLA (B- and T-lymphocyte attenuator, CD272, Cluster of Differentiation 272), Indoleamine 2,3-dioxygenase (IDO, IDO1), TIM3 (HAVCR2, Hepatitis A virus cellular receptor 2, T-cell immunoglobulin mucin-3, KIM-3, Kidney injury molecule 3, TIMD-3, T-cell immunoglobulin mucin-domain 3), A2A adenosine receptor (ADO receptor), CD39 (ectonucleoside triphosphate diphosphohydrolase-1, Cluster of Differentiation 39, ENTPD1), and CD73 (Ecto-5′-nucleotidase, 5′-nucleotidase, 5′-NT, Cluster of Differentiation 73), CD27 (Cluster of Differentiation 27), ICOS (CD278, Cluster of Differentiation 278, Inducible T-cell Co-stimulator), CD137 (4-1BB, Cluster of Differentiation 137, tumor necrosis factor receptor superfamily member 9, TNFRSF9), OX40 (CD134, Cluster of Differentiation 134), TNFSF25 (Tumor necrosis factor receptor superfamily member 25), IL-10 (Interleukin-10, human cytokine synthesis inhibitory factor, CSIF), BCMA, CS1 (SLAMF7), CD79A, CD79B, CD138, and Galectins.

36. A method of treating a cancer comprising administering an effective amount of a monoclonal antibody or antigen-binding fragment thereof to a subject that specifically binds CD47 and increases phagocytosis of human tumor cells and comprises:
a variable heavy chain CDR1 amino acid sequence (HCDR1) amino acid sequence set forth in SEQ ID NO:3;
a variable heavy chain CDR2 amino acid sequence (HCDR2) amino acid sequence set forth in SEQ ID NO:6;
a variable heavy chain CDR3 amino acid sequence (HCDR3) amino acid sequence set forth in SEQ ID NO:10;
a variable light chain CDR1 amino acid sequence (LCDR1) amino acid sequence set forth in SEQ ID NO:14;
a variable light chain CDR2 amino acid sequence (LCDR2) amino acid sequence set forth in SEQ ID NO:17;
a variable light chain CDR3 amino acid sequence (LCDR3) amino acid sequence set forth in SEQ ID NO:18,
in combination with an opsonizing and/or targeting monoclonal antibody that targets an antigen on a tumor cell.

37. The method of claim 36, wherein the opsonizing and/or targeting monoclonal antibody is chosen from rituximab (anti-CD20), trastuzumab (anti-HER2), alemtuzumab (anti-CD52), cetuximab (anti-EGFR), panitumumab (anti-EGFR), ofatumumab (anti-CD20), denosumab (anti-RANKL), pertuzumab (anti-HER2), panitumumab (EGFR), pertuzumab (HER2), elotuzumab (CS1/SLAMF7), atezolizumab (anti-PD-L1), avelumab (anti-PD-L1), durvalumab (anti-PD-L1), necitumumab (anti-EGFR), daratumumab (anti-CD38), obinutuzumab (anti-CD20), blinatumomab (anti-CD19/CD3), and dinutuximab (anti-GD2).

38. The method of claim 36, wherein the opsonizing monoclonal antibody targets an antigen on a tumor cell chosen from CD20 and CD38.

39. The method of claims 1-38, wherein the cancer is a leukemia, a lymphoma, multiple myeloma, ovarian cancer, breast cancer, endometrial cancer, colon cancer (colorectal cancer), rectal cancer, bladder cancer, urothelial cancer, lung cancer (non-small cell lung cancer, adenocarcinoma of the lung, squamous cell carcinoma of the lung), bronchial cancer, bone cancer, prostate cancer, pancreatic cancer, gastric cancer, hepatocellular carcinoma, gall bladder cancer, bile duct cancer, esophageal cancer, renal cell carcinoma, thyroid cancer, squamous cell carcinoma of the head and neck (head and neck cancer), testicular cancer, cancer of the endocrine gland, cancer of the adrenal gland, cancer of the pituitary gland, cancer of the skin, cancer of soft tissues, cancer of blood vessels, cancer of brain, cancer of nerves, cancer of eyes, cancer of meninges, cancer of oropharynx, cancer of hypopharynx, cancer of cervix, and cancer of uterus, glioblastoma, medulloblastoma, astrocytoma, glioma, meningioma, gastrinoma, neuroblastoma, melanoma, myelodysplastic syndrome, and a sarcoma.

40. The method of claim 39, wherein said leukemia is systemic mastocytosis, acute lymphocytic (lymphoblastic) leukemia (ALL), T cell—ALL, acute myeloid leukemia (AML), myelogenous leukemia, chronic lymphocytic leukemia (CLL), chronic myeloid leukemia (CML), myeloproliferative disorder/neoplasm, myelodysplastic syndrome, monocytic cell leukemia, and plasma cell leukemia; wherein said lymphoma is histiocytic lymphoma and T cell lymphoma, a B cell lymphoma, including Hodgkin's lymphoma and non-Hodgkin's lymphoma, such as low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, and Waldenstrom's Macroglobulinemia; and wherein said sarcoma is selected from the group consisting of osteosarcoma, Ewing's sarcoma, leiomyosarcoma, synovial sarcoma, alveolar soft part sarcoma, angiosarcoma, liposarcoma, fibrosarcoma, rhabdomyosarcoma, and chondrosarcoma.

41. The method of claims 1-40, wherein the cancer is multiple myeloma

42. The method of claims 1-40 wherein the cancer is ovarian cancer.

43. The method of claims 1-40, wherein the cancer is gastric cancer.

44. The method of claims 1-40, wherein the cancer is acute myeloid leukemia (AML).
